SEQUENCE LISTING (118 sequences)

SEQ ID NO: 1 - DNA, 1212 bp, Mus musculus
aaatcagtttctagacagaatctggacccctctctcttccattctgtctctttctacctctctctcattctttcaccatggaatttggaaagcatgaaccaggaagctcactaaagaggaacaagaacttagaggagggagtgacgtttgagtacagtgatcatatgaccttcagctctgagagcaaacaagagagggtccagaggatactggattatccgtcagaggtcagtgggaggaattcacaacaaaaggaattcaatacaaaggaacctcaaggaatgcagaaaggtgatctcttcaaagcagaatatgtttttattgtggattctgatggggaagatgaagctacatgcagacaaggtgaacaaggccccccagggggaccaggcaacatagctactcggcccaagtctctggctatttcttctagtctggcttctgacgtggtgcgtcccaaagtacgaggggctgatctcaagacctcatcacatcctgaaattcctcatgggatagcccctcagcaaaagcatgggctggcactagatgaaccagccaggactgaaagcaactccaaggccagcgtgttagacctaccagtggagcattcttctgattctccttcacggcccccacagacaatgttgggttctgaaacaatcaaaactcctacaactcatccaagagcagctggtcgagaaaccaaatacgcaaatctttcttcatcatcctcaacagcgtctgagagccaactgactaagcctggagtaattcgtccagtacctgtaaaatccaaactactcctgagaaaggatgaagaagtttatgagcccaaccctttcagtaaataccttgaagacaacagtggcctgttttctgagcagtaaggaagctggagtggaagtggacaccggtctgctgaagagttttggaatgatgccatggccaactacttgctaaacttacctgatgctttgttagaaggagtgctctgctcagtccagcagaagcacctgaatggtttgccacagccacatagcattaccacactctgggaaacccagagcaggatcatagcccttctgtttcttgcgttgccgttcaagcctataatgccttctattaagtcaacagcaatactaatgttcccctatatttagcagtcaaataaagaagaatgatagctgaatacagaaaaaaaaaaaaaaaaaa

SEQ ID NO: 2 - DNA, 3187 bp, Mus musculus
tcactgcggcagacactggaaaataaaattgttaagtacatcctagctgagagggagagacggaaggctccgtgttcaatcaaaggtttgcaataataggagtcatttaagaaagaaagaaagaaagaaaaaaaaaaagacagatgggattaggaaatgttgctgcggtgagactgtcatgagaggcacaggcagcctgccttttgtggacctgcacaatgatcacagagccagactggcttaggagaccctgggactagggctccagagagaggccacgggctcccggacaccctgcagggcagggggctgagaccatgcatcagatctacagctgcagtgatgagaatattgaagttttcaccacggtgattccttccaaggtgtccagttcatccaggagaagagtcaagagctctcaccacctcttggccaagaatgtggtgatcgagtccgacctgtacccgccaccaaggcccctggagctactgcctcaacgctgtgagcgcagggacacaggtgaccgcagatggttgcagactggccggctgcagactgccaggccacccggggcgcatcccaccaaaacgccctccagacctgtggggatttctgaacccaaaacatcaaatctgtgtgggaatcgagcatatgggaagtcgttgattcctccagtggctaggatctcggtgaaagctccagcaggggcggaggtggcagccaagggctcagaacatggagctgttctgggaagaggatccagacacctcaagaagatagcagaagagtacccagcccttccccagggagcagaagcctccctgccattaacaggcagtacttcctgtggcgtccctggcatcctacgaaaaatgtggaccaggcacaagaagaagtctgaatatgtgggagccaccaacagcgcctttgaagccgactaaactcgacatttcatgggcaccttgcattggtcaaggttcggaggaagatagaagagttgaggactgggactgagccaccctcccctctgctggttgctggtccaaacacatcatcattccttatactctgacatggggcatggaaagtaacatcctcagaaggcaagaaagctgttcctcagaactgctaaagccattggtcttaaagtcgtattggtcaattacaaaggttatatacctacttttaggcaaagctataccaaaagcaaactttcctggcctgtttaaaagcctccaaggaaaacagaaggcagttgatctgtcttctttgtgagttttcccaaaacgtatggtttctggtgtaaatgtaaaagtttgattctgaggtactcagaacacaacagttcttacttttcccatcccatgtctgttttcccttgatgaaatacaaaatgcttcatctttgctttgttctaatatctacttaacagcaaccattgccaatctgctttgctaatcatgggcatgactgcatgagctctctctcttctttaggtgcattcttgtctatagaaaagcacttaaaatcccaatgttaattttaatgtctaatattttgtgatgtggtgcaattgacaagctttgtatagtgactttaatccagagagcattctcccatcattgtctcttctcaccattacaaaccctctgataagaaagcactgtggtccccaacctacagattgggacactagagcatctggatggcagtatgtgacttaacagcagcttgtgggactgtcaccaggtctgagcatctctaaaataactgatttaagaaagtctttaaatggagagaagaatctgacaatgttggaacaaagaagtgattcgaatgaaatacatcattgtgtattagataattaagacgggtgcagagaacagggaccccaacacgtaaagaggttcagacaggaggatcacatgtttgggacatgcctggacagcctagcaatatcctgtctcaaaaacaaaacaaatcatgcatggacacacacacaaacagagagggagagagattatgcatagatgaaaccagaagaaatcgccatttttgtaggttgaaacagctgaaaatcaatttcctgtggctcaaactaatatttactatcatttaaaaatgatcatgtaagaaataatggtaatcaaagctatcatttgttaaatgcctacctgccttgtgctgagcacagtgttgaacaccacgcatgaattaagctattctttctaacaactaacttgggtggattttctcactgtatatgttgatactaattcaaaaaagttaagtgattagattgaagtcgcctaactctaggtgtctaactttaatgcccatattgtatcttctccttcacacaaaataaggaaaagggaaggatggaaatcagcaaaacgtcttcctctgcaatggcctgggaagaggctacctggagcacagcgtggagatgaggtatcagagtgcagagactggttagtggtatctgcctgtgaagtggtcggtaacatagaccttatatatttctcatctgtggcttaaattctgccccccggaagtcttgttccctaattaagaggtttaaattaatccgaccttcttaatgaaagcagaaactccgtggtaaactctaccctaaggttatgttgagactccgcaggtgttggcacaagcacgagtttgaactgtttgggtatcaggcttgcttctgctgttctgtggattctgctttcctgttcctgatgctctggataaactgaaacatggcggtaagtcaaacccagacttccaggctcttgccttgctgattgctgccctcccacctctgctttagaccctgagcatctgaccctcatgtccaaacatagtctggacacttgggcatcaagtgctttgtcccagtgaaccatctaatgtcatatacaatattaagttggaatccagaacaaagttagggataaaacatgtcacagagtctccaacgattgaatttatttaacttaaaattgatgtcttaaatgtgtgtgtgtggctcctggagatttattttatatgtagactgggactcatttatttatctttaatttaaatatttaatggtgaaatgtttgccttctgtagaacatttcattcaaataaaaataaaggatgccttgttagtgacattaataaaccacttgaagattgt

SEQ ID NO: 3 - DNA, 3713 bp, Mus musculus
gagtcacgcgatttccgggaacccgtcaggaaggacataaacaaaacaaaccgaggcagcatggagacggcccgcggccgcgtaagcgcggccggatccagtgcctgaaccgccttcagcctcagaaccgcaaatttatttttttttaaaaagtggtgacccaagcagttgaactgaaggtattctgggaaaatctgctgtttattgtgaaaatcatctttgatcttggaattaaaagtaaagctggaaaggaatttacaaacaagaaaaagaagaagtttggaattggactcacaggatctgggcttggaaatgcctcagcccagcgtaagcggaatggacccgccttttggggatgcctttcgaagccacaccttttcagaacagactctgatgagcacagatctcttagccaacagttctgatccagatttcatgtatgagctggatagagagatgaattatcaacagaatcctagagacaacttcctttctttggaagactgcaaagacattgaaaatctggagactttcacagatgtcctggacaatgaggatgctttaacttcaaactgggaacagtgggatacatactgtgaagacttaactaagtacacgaagctcaccagctgtgacatttgggggacaaaagaggtggattacctgggtcttgatgacttttctagcccttaccaagatgaagaggtcatcagtaaaactccaacactggcccagctcaatagtgaggactctcagtctgtttccgattccctttattatcctgactcactcttcagtgtcaaacaaaatcccttgcccccctcctcttttcctagtaaaaagatcacaaatagagcagctgcccctgtgtgttcttcaaagacacttcaggctgaggtcccatcatcagactgtgtccaaaaagcaagcaaacctacttcaagcacacagatcatggtgaagaccaacatgtatcataatgaaaaggtgaattttcatgttgaatgtaaagactatgtaaaaaaagcaaaagtcaagatcaaccctgtgcaacagggccggcccttgctgagccaggtccacatagatgcagcaaaggagaacacctgctactgtggagctgtggcaaagagacaggagagaaggggggtggagccgcatcagggtcggggcactcctgctttgcctttcaaagaaacccaggagctattacttagtcctctgacgcaggatagtcctgggttggttgccacagcagagagtggcagcctttctgccagcacttctgtttcagattcatcccagaaaaaagaagagcacaattattctctttttgtctctgacaacatgagagaacagccaaccaaatacagtcctgaagatgatgaggatgatgaagatgagtttgatgatgaggaccatgatgaagggtttggcagcgagcatgagctttctgaaaatgaagaggaggaagaagaggaagaggattatgaggatgacagagatgatgatatcagcgacacgttctctgaaccaggttatgaaaatgactctgtagaggacttgaaggagatgacgtccatatcttctcggaagagagggaaaagaaggtacttctgggagtatagtgagcagcttacaccatcacagcaagagaggattctgaggccttctgagtggaatcgagataccttgccaagtaatatgtaccagaaaaatggcttacatcatgggaaatacgcagtgaagaaatcacggagaactgatgtggaagaccttactccaaaccctaaaaaactacttcagattggtaatgagctgcgcaagctgaataaggtgatcagtgacctgactccagttagtgagcttcccttaacagcaaggccaaggtcaaggaaagaaaaaaataagctggcatccagagcttgtaggctaaagaagaaagcccagtatgaagctaataaagtgaagttgtggggcctcaacactgaatatgacaatttattgtttgtaatcaactccatcaagcaagacattgtaaaccgagttcagaatccaagagaagagagagaacccagcatggggcagaagcttgaaatcctcattaaagatacactgggtctcccagtcgctgggcaaacctcagaatttgttaaccaagtgttagggaagactgctgaaggcaaccccactggaggccttgtaggactaaggataccagcatcaaaagtgtaatcagcctcattggaccactggtcagaaatgtctgtttttgtcatgttatccattgtaaattttcattctgttttgcatgtcaattagcattatgtaaacatttataattaggttacattgttttaaaaacaatagcataagtgaagcatgatccaaaatacttgattattgcattttcagagcataaaccagtgaccctgctgctggcatgagaaagaagctcacacattaagtaaatatgaggtacagattgtaaacatttgttgaagcagagtgttttgggtgagtgaatatattagtataatgctgagtgttaaggtgggtttatgctctgaaccacacaaaaataccgaggaagcattttttttcaaagtccatttagattgtttttagaatgactgctttttgttctaattttttacagccattaatctcacatgtacatggcgcacccagcactcacgtgtgtaccatgtttagatgtttttcagaactcaatatgatatataaaaatacatatatatatatatatatatacatacatatatatatatagaattgtctgtgcaagtaagaaaaagcatactctttgtgccttgtattttggggaaactctaaaactggtaatattttgtatgatgaaaatcctaatgaggaaaaccaagatatatagatgagaaaattatggggtttaaatgtctttttgttccaactctttttcagatttttttgaatgtatataggactatgtcaaaatgtagatatatgccacagagtctgtgtattgtataaaaaaaaaaacaaaaaacaaaaacaaacaaaaaaagatggctctagagaactcctatttcggtacttgaccggaagaaaatacttgcacattattgcgattgttttattttttctaccaaagacaaatgcaactggtatggcagactgccagtctaagtaaagttttgcacagcttacatgatactgtatgaatgtatgaaacagagaaaaaattaaaaggtcagggttagggatcttactcaactgtgaactttatttctgtttgggtccaattatctacagaaggagcatccatacatccaaatattattttgctgtcctctagtttgcttccatagtagataagttggtggccacttaggtgtcttttatttctgcagttattgtaggaaattttaatatatttcatattagtaagctattgataaaatagtttttgactttgaaaattaaagtttatttagcttattgtagtatacttccaccaaacaaccaaaatacagattatttttatcgtattatgtatatatatatgtaaagagataaaaaagctaaaaatatctaatactttagttgccacttttccaattgatgttattgtgcatgtaatattttcaaagatcaacacaagcttaaaacaaatttataaatttttatatttttgtacaggtattttcttcaaactt

SEQ ID NO: 4 - DNA, 5632 bp, Mus musculus
gctgctccgcagtgcaacagtttgcacctgccttttgggagaggcaagggagctgtgctgcctgccgcgggtcctccggcttggtcctctgccacagcctctgggccctggggccagggcccggggaaggctggagacaagcactgtgcctggggaaaggaaacaggatcaaagaagaccagagacactctccctggggccactagctctggcccgcttgcttggcggcagttgctccctcagtctttggctggagagctcactccctccagcgctaaagagcagttggggtggtgtgggttcctgtccacgtcttggctggtgggaaccgtgtgtccaacagaggaagcctggtgggcctggcccctctcagcccagcgccgatgagtgccagggcgccgaaagagctgaggctggccctgccgccttgtctcctgaaccggacctttgcttcccacaacgccagtggaggcagcagcgcaggtctccgcagctcaggcgcaggtggtggcacttgcatcacgcaggtgggacagcagctcttccagtctttttcatctacgctggtgctgattgtcctggtcactctcatcttctgtctcctcgtgctgtccctctccactttccacatccacaagcgtaggatgaagaagcggaagatgcagagggctcaggaagaatatgagcgagatcattgcagcggcagccacggtggtggggggctgcctcgggcaggtgttcaagctccaacccatggaaaagaaacccgactggagaggcagccccgggactccgctttctgcaccccctccaatgctacttcttcttcctcctcctcctcctcatcccctggtctcctgtgccagggtccctgtgcgcctccgcctccactgccagcccccactccacaaggagcacccgcagcttcctcctgcttggacacacctggcgagggccttttgcaaacggtggtactgtcctgattgcgcagcccctctcctgaccctttcctcgtctccagcatcttgaccgtcctcgcttttgttcctccttccttccttttccgttctcctttggcccctgttttctctgccctctttccttacctggccaccctttcactgtctctcctccgccgaggcactgtgcggtatttgtaaatattgggcgaggaaagtctcagaggaagaaataacgctgataatactttactgatatttatagtaattattatactactaataacacagtccaagcgcatgacaaatcacataatttctcattgttaatgaaggctgcatcttccctccccaccccgtgcccccctcaataaggagaagaaaccgcacaagctaccaaatatttaagacattgacaccgaagcaaagggatgggagggggccggtgatgttgtatatagctgtcaagtgaaggtttagtcgcctttctgcccctccctcacgggctttcacttttcctttagcttgtctcccctcccctttcctcaccttcagctgggctcaggcaatagtatattataaaggcaacatctacattaagcaccagagaccacatagaatgtcccagaaaaacgtttaggacaggtaacccctctctagtcagtcatcatttcactttttgctttccccctggcagaattagagttttacttttagaaatatcactcgctggtcgagcacagaaaaagaaaacaaaaaacaaaaacatactatctgatttgctgctcattagggcccatttgtacacctgactggtagtcgttttggtttggtttgtttgtttggttttttttttttccctttggggaatttttttttttttttttttttttttttgagcctcagagaaaccgggagctgtccagccaggtctggtgctgaatatgtctcaccttcatggttacttcctctggttgtgcagaccaaagaaggagctgttttggaaagtgattgtcttggttttgattggtttctttcttcttttttctacaattggattgttttttcttatcactatacattgcataagttacctttatgtaaaaaaaaaaagtattaggcaatgtgcagttctgaaaatgcagtatctaaccaactagtatgtttctgttttatttttagaacaagtgcacctttgttatatacttattatattggtaccaaatacagaagaaaactatagttctgtgatatgtcctccaaactgtatatttttgttcttctgactttccagctgttgatataatggttgccactggctgaggaagtcagtggtgtaggcctggcttctgctgtttccggaagtgttcttttgtattttacgctgtagtagactattataaaacgatgacacccatgtttccccctttttcttttgtgaataacagaaacaaccacaacagaaaacaaataatggatgtgctggaatgccatctattaaaaacatggttaatatttaaacagtgcctgtggttctctgcatgcagttgccacctggaggcagtgctgtgtgtgcttgcttgtactgtatgtgtttgggggaaagactggtggagatgttgggcaatttggatgacaggacatgacaatttccaagttaaatctgtaaacgcttacaggataaaactgtttacagcttgtttagttatgactccatgcctgcatctgatatacagcaaaggggatctttcttcttcccaagtctggcctaattaacctccctgaacacataggaaatgttaagggaaaaggaaagcatgagagaagataaatctcttgtcctctctttaaatgtcagataagtccctctatgttagactctgctgtttagtgaagggcagtgggacccctacatatatccatctcccaagccactagctcccctctatgctctgtcttatttcaagttgtatgtggttattatcccgagaaatgattgcctctaatgttttggttacatataaagttttccaagctcagtctgtatctttataaaataatttaataaggttgatttagtcacccatagatatcatccaagtcctttctgaagcacagaagaccatcgtttaagcatgccatgttgtatcattaggaagatcaggtataatctttggatacaatatattaaacaatgaaccagattctctccagtgccttagtcacttcctagtaacaggtcagagtgcattcagtccctcgggccaccaaggatgctgttagtgtatcagagctctacactgtacaacagaatggctaaggcactgtgaaggagaatatccattaatctctttaacttgccctcatccaactgtagctcttaataccgtcttacagaatgggttttagatgtgaaatctgaataggatcaggaacccagaaggaaggttcatcatttcagtgagctctacataagtgcatagatattacttttttccattatttggtgcactctttttacagtaaataatttcccattttattaaagcaataagatattctgttttgagcagcgctagatgctcattccacttccttggtgctgaagcaactcatatgttcttgccttatgaatcacagtgcattcaaggcatgcaataataatccccttccaagaagcagcctgcacaccctagggggcaatgtcctacatacttttccccaaaagaaatagagcaaacaagaaataaactaattatgtgtattttaaaaaaaacatcttgatacctctaaccataagcacacatctgtaatgtgctatcattgtgcacctttaagtgtatatgccttttccatcaattgactagggattaatattttaatagtgtcctgtgtaaagatgatgcagcttatcaatcacatttcatactgagtttaatatgctgtgaacctggtggcaccacacaagatttctgttagttagagtgataattacatgaaattctagtaggccagatcccacaccaaattattgtaataaagatacgacaatgcaaatttctatagagtctgcttagattgctacttagagagcgcaactgacccatatgacattcgagttttcatttttatgagacaaaaggcattatgaaaatagctaaatttactctaaggatcttgtttactgatgtcccgtcaaactaattgctccaaccttctatagctacagctcagctcctgcacttgctattcagtattaataaagtagcatgcttgattcttactattttaaaaatgagaaaaatagagagagaatgccgatatgtcaactatatgggactctgactcttgaagatgaagatggatataatctttagaatttatatacacccatagatatgtatttatatatgcatacattttgtacaaatttacaatggactttttgtattctcttttctgtcattataagtatgagactgaaaccaaatagttgttccatcctctgatccagagaggatctgaggagtcaggtgtatgcatgtacttggttctctgaggtcaaatcagaatggctttctctttatttagtggaaggggagagtcttccttggcttggggaattggtaattagataagcttcctttcctatattagtgactcaaatgtagcaatgataacgaagtcatgaccatctctatgtggtccagctatttgatggatcaaaaatctatctacacgagcattggagccatctagaccacaatcatggattgcaatctgatttttcctcttcactttcaacttacatgttgtatgagatacaacatatacctgcatagctaaaggtaaaatgaaaatatacctactatttgtttagtttgaagagtagctttttggaaaatatggacaatttagctttaaaaattactgggcatttgactttttaaccctccctatctgtaaccattgaattgattagatactgactaaaatttctttttccaatgcataggcatagattttaagtgcttttaatggataattgctaagaaatgactaactagtattacatgagagttatgggctaaattggaaccagaaactttaatatggcttctaaaattcactcccatggaagtctaggctttggactcgaatctgaaaagccacttattcatgtttggaaattgtggtgtggtccatagatgttcatgtaagaacggattgccccatctacacaggaaggtatttcatcactgggctgaatataaatctgattctggtttagtttttccttatttgataacattttggaagcagacgtgatggcttcacatcaatacttatcaatgtcaccagcccaatggtgcaattggtgcagtgtcagaatgcttagtggagaataagaacttacttagctttaattgagagacagtgcattattcgggttgctttacccatttgaggaaggatatttcactgagacttgtggtctcacaggattgctgctacagagaggataacgacagtgatggcatcaacagaagaagtgatttttgaactcgagtatatccaatagatagaataaaacaggtattagaaattcatgaaaaatttgttttgcttttatatcaataaaagagttttcttgttaaaaaaaaaaaaaaaaa

SEQ ID NO: 5 - DNA, 4153 bp, Mus musculus
agacagagggagtcaaagcctcccggtccagccgtcccattttactgcttaactcagcctggagtgtcagaacccatctttgcctgccttctctgtgctttgtacagtggggccagtcgctcccaacggccagcccgctgagtggaacaggggtctagggtggactagcagggctctgcccgttggggtgactttcgaacattatctttagtgtattttaacagtacagagcttgtggtgggactcagagagggagaagctttgattgctttttaaaattattttattttattttattttttggctttttttttttttttcttctggtgatctggatttgtttcctcgggcccctccccttgttccgttctttctcactcccgcctttggcgaagtgacacaggcgacacctgctcgcttgtgtctgctgctggaactcgcacctccaaggtggtgaaggtgccggcgcgctcgtgacttggggggacagcagaggggttccctcccttggagcacacgatgcggagagtggcggcgggtgggatgcgaggagctctaccggctgcacttggaggcgcgatcgaggggctgcagctggccgggagttgctgctaagtggacgcgactcgacggcgcccaggtgtccgagagggcacccgcgggacccgagtgcgagctgcgggaacgcaggcgtctcccggggaggacgccggccgagcgcacagcccgcgagcctgctcagaaccgctccacaccgggagcctgcagacctgggagagccggggaactcgaggagtgtgctcggcggcggaggctgctctgctgaaggtgaatcaccgcgtccaattgcctttccctagagaacccgggggagggcgggagagggggaacgtgtgagcgcgcgcgtttgtgtgcaagggagagccgacgcggggcgagaggaaaagtcctcgctcgccgctcaaagcaaacaaaccggagatggaataagagcggcggtggctggagcccgcctggatcctcgcagtcgcgggagccgagcgcaccaccgcgcgcagcgcatcgccggggacagcgggcgaccgcgggcgccgccgggggctgcggagactttggctctccccctcggtcgacgacccttccgattactttgacactgtgggataaagagcagagcccggggaccgacctcgcgcgctgcaccttccttttgcttcggggaagggggaccccagcttgtaggtgaagacgctgccgagcccccctttagccttcggaggaggcagcactcacactcgctcgccctctgcgaacacacaccggcggacttgaggtccctatgcccctggatggtgccagcggaccggcatctcggaagcgatgcaggagcggtgaggctggcgtcggcccgcggaagctaccggaccatcgctaggtgctgccgcccccgggacgcccggctgcagggtctctcactggacgtggaaataagctggtgaccagcaagccctagcgtctctgtccagtgactgttacaggagcacaggaccatgctgcccatcagttttatgtggacactgggactgaactggtacctgtgcttgtgcagcgagggctcttacccactgcgctgtctcttcagcttgcaatgagtatctattcagtgaatgatcaccaagatgaataagagatacttgcagaaagcaacacaaggaaagcttctgataattatttttatagtgaccttgtgggggaaagccgtttccagcgccaaccatcacaaagctcaccatgttagaactgggacttgcgaggttgtggcgctgcacagatgctgtaataagaacaagatagaagaacggtcccaaacggtcaagtgctcctgcttccctgggcaggtggcaggcactacccgagctgctccgtcttgtgtggatgcatccatagtggaacaaaagtggtggtgtcatatgcagccatgcctggagggagaggaatgtaaagtccttccagatcgcaaaggatggagctgttcctctggaaacaaagtaaaaacaactagggtaacccattaaccactcctaaatcaaataatactgatggctgggttcatggagacacaagaatagaaactggactcctgccgtgaccttgaagatttttatactgcttagaagagacatttaaaatccatttccaaggaattctatggcttttcatctacttcttagtgaaactaagactttacagaagtctacagtgaacgttgggtcctgaagacttcatccgctgtgaactaacgcttggctcacaacacttctgagggaagggggcgcagttctccacggagctgggatagagttggttttctggggtgacaccagagactgtcacctttaaggttccttgcttcagctgtgactgttcttgtgtctgaagctgcttcccaagctgactgtcctgtcattgatgtgttcctggctcttttgctccttgtctagtaggactatgcacagctttgatgacgttccctttgtaaactcttccagcatggcgtagacaggggcaattttattggtattctaaccttgaaactctgaaagcctacatgttgtaatgtcttactcttgcctctgtgaaaggaatagaagtatttacccatcgataatgataatcattggcaaatcacaatgatctgagtatatccctcatataacaatgtgtaggcgacctgacacgtttccccaaggctaacacgtgactgcagctctctgacggttgaacagatagcagttaggattggattccaattcccattacacagtgctctgtgcctctagtgcacccttccttccagggttggttggttttttttgtttttgtttttgttttttttaagtgtactttccttttactttatttcacagaactcatcagtatacagtcaagttagcagagagcttttgatttaaaataataaaataaaataaaacccgccagggatgcatgtgcttctctgatttctcatcacttacatttttctttctgtctcattttaaggtcgtctcttgctcgatcccaatgactgcttgaatgcttatgtatttgttcagtctgtggctagaaaaaaacaaaacctgttgattttccttgaccacaaaggtctaaatactcacttacggttgttccattcaaacaattcttggccagtattttgtccatattttctcaccctaaaacttgtgatttttagttcttcgtggtttttcttacaaatatttaaaggccttaaacgttgatttacctttttctaagttattacagaatgtataattttgtacggcgtttattttgtttcacttgtgatgtgggggaagggaacagtgggtactgagttgccaccctataataataataccatgtaaacttatagtttgaaggcatataaaagcaagggttttccatgtcttaattattttagcttgatcaaaagatgtttcacagatcatcctattagggggtccatcattttagtaaaagagtgaagatgtgtgggacttcttgtatttctaggactcactgacaaagccagttaaccttaggttggtttctaggatggaggttcatatattctagaggagaatttgtacctgttagtctgtaacaatactcaaagggtcgcacagtaactaggacctttggtgggaagaacagctaaaagtgcaaaaatctgttaaaaaattaaattggaaaacgacacttttaatattgattgaaagtcaactgcctcatgaaactgtgggacaaagtgattcctgactcacttttaattgcataaaaccaactgggtggttctacgtgggttttgttatggatcagttctacacagagtcaaaatgtaaggagtaacagttatgttggactttctctgtcaaacaaattatcactactttctttggttcaaaatgaaaaactatcattttggatattatgagtttatcctaggttggtttgaaattatggatcatgcttctcattgcgaaatctatgtcatttaaaacaacactggaaattctgtattatataaaagtgtaatacatgcgtatcaatagaaaaaaataaatgaaatttcaaatataaaaaaaaaaaaaaa

SEQ ID NO: 6 - DNA, 4382 bp, Mus musculus
ctctgcaaggactggcgctgctagggacctgctagggacctcggagtcatggaccccattcagctgctcttctacgtgaatggccagaaggtggtagaaaaaaatgtcgatcctgaaatgatgcttttaccatacctgaggaagaatctccgactcacaggaactaagtatggctgtggaggcgggggctgtggggcctgcacagtgatgatctcgcggtacaaccccagcaccaaggcgatcaggcatcatcctgtcaatgcctgtctgacccccatctgctctctacatggtacagcagtcaccacggtagaaggcttaggcaacaccaggaccaggcttcatcctattcaggagagaattgccaagtgtcacggcacccagtgtggattctgtactcctgggatggtgatgtccatgtacgcactgctcaggaaccatccagagcccactctagatcagttaactgatgcccttggtgggaatctgtgccgctgcactggatataggcccataattgatgcctgcaagactttctgtaaagcctctggctgctgtcaaagtaaagaaaatggggtgtgctgtttggatcaagaaataaatggattggcagaatcccaggaagaagataagacaagtccagaactgttctcagaagaggaatttctgccactggacccgacccaagagctgatatttcctcccgagctaatgagaatagctgagaaacagccaccaaagaccagagtgttttatggtgagagggtgacatggatttcccccgtgactctgaaggaacttgtggaagctaaattcaagtatccccaggcccctattgtcatggggtacacttctgtgggacctgaagtaaagtttaaaggtgtcttccaccccatcataatttctcctgacagaattgaagagctgggtgtcataagccaggccagggatgggctgaccctgggtgctggcctcagcctggatcaggtgaaggacattctggctgatatagtccagaagcttccagaagagaagacacagacataccgtgctctcctgaagcacctgagaactctggctggctcccagatcaggaacatggcttctctagggggccacattgtgagcagacatctggactcagatctgaatccccttctggctgtgggtaactgtaccctcaacttactgtccaaagatggagaacggcggatccctttaagtgaagagtttctccgaaagtgtcctgaagcagatcttaagcctcaggaagtcttggtctcagtgaacatcccctggtccaggaagtgggagtttgtgtcagccttccgtcaagcgcaaagacaacagaatgcactagcaatagtcaactccggaatgagagtcctttttagagaaggaggtggcgtcattgaagagttatccattttgtatggaggtgtcggttcaactatcatcagtgccaagaactcctgtcagagactcattgggaggccctggaatgaagggatgctggacacagcctgtaggctggttttggatgaagtcacccttgcagcctcagctcctggtgggaaggtggagttcaagaggaccctcatcatcagcttccttttcaagttctacctggaggtgtcacagggtttgaagagggaggacccaggtcactctcctagcctggcaggcaaccatgagagtgctttagatgatcttcattcaaaacatccctggagaacattaacccaccagaatgtagatccagcacagctgcctcaggaccccattggacgtcccatcatgcacctttctgggattaaacatgccacgggcgaggccatctactgtgacgacatgcctgcagtagaccgggagcttttcctcacttttgtaacaagttcaagagcacacgctaagattgtgtccattgatctgtcggaagctctcagcctgcctggtgtggtggacatcattactgcagatcatcttcaggaagcaaacaccttcggcacagagacatttctggccacagatgaggtacactgcgtgggccatcttgtctgtgctgtgattgcagattctgagacacgggcaaagcaagcggcgaagcaagtgaaggtggtctaccaagacttggcgcctctgatcctaacgattgaggaagctatacaacacaagtccttcttcaagtcagaacggaagctggagtgtgggaatgttgacgaagcatttaaaatcgttgatcaaattcttgaaggtgaaatacacataggcggccaggaacatttttatatggaaacccaaagcatgcttgttgttcccaaaggagaggatggagagattgacatctatgtgtctacacagtttcccaaatatatacaggatatagtcgctgcaaccttgaagctctcagccaacaaggtcatgtgtcatgtaaggcgtgttggcggggcatttggagggaaggtgggcaagaccagcatcttggcagccatcactgcatttgctgctagcaaacacggtcgcgcagtccgctgcattctggaacgaggagaagacatgttaataactggaggccgccatccttaccttggaaagtataaagctggattcatgaatgacggcagaatcttggccctggacgtggagcactactgcaatggagggtgctccctggatgagtcactatgggtgatagaaatggggcttctgaagctggacaacgcttacaagtttcccaacctacgctgccggggctgggcctgcagaaccaaccttccatccaacactgctctgcgtgggtttggctttcctcaggcagggctggtcaccgaagcctgtatcacagaagtggcaatcaaatgtggcctgtcccctgagcaggttcgaaccataaatatgtacaagcacgttgatactacccattacaagcaagagttcagcgccaaggccctctctgagtgctggagagagtgcatggccaagtgttcctactttgagaggaaagcagccataggaaaattcaacgcagagaattcctggaagaagagaggaatggctgtgattcccctgaagtttcctgtgggtattggatcagtagccatgggacaggcagctgccttggttcatatttatttggatggctctgcactggtctctcatggtggaattgagatggggcagggtgtgcacactaaaatgattcaggtggtcagccgggaactaaggatgccgatgtccagtgtccacctgcgtgggacaagcacagaaaccgtccccaacacaaatgcctctggaggctctgtggtggcagatctcaatggactggcagtaaaggatgcctgtcagacccttctaaaacgccttgaacccatcatcagcaagaatcctcagggaacttggaaggattgggcccagactgcttttgaccaaagcatcagtctctcggctgttggatatttcaggggttatgagtcgaatatagactgggagaaaggggaaggtcatcccttcgaatactttgtgtttggagctgcctgctcagaggttgaaatagactgcctgactggggaccataagaatatcagaacaaacatcgtgatggatgttggccacagcataaacccagcccttgacataggtcaggttgaaggtgcatttattcaaggaatgggactttacacaatagaggagctgagttactctcctcagggcactctatacagtcgtggtccaaaccaatacaagattcctgccatctgtgacatccccacggaaatgcacatttcttttttgcccccatctgaacactcaaacaccctgtattcatctaagggcctgggagagtctggggtgtttctgggatgttcggtattttttgccatccatgatgcagtgaaggcagcgcggcaggagagaggcatctctggaccatggaaactcaacagtcctctgactccagagaaaatcagaatggcctgtgaagataagttcaccaaaatgatcccaagagatgagcctggatcctatgttccctggaacatacctgtgtgagtcaaacatgaacctctggaggaattggctgagcaactacagaccgtacctcctgcctgctctgctctaagatgctaaatgcgaaagccagagtttcacagcccagaatcatctacagcactgctttacatgaagccgactcggaagattctcttgaggatactccagatacacctgagcaattataaatcatatattaaattgcacaaatatttaaatcgtttgctctaaggtggtttcaatcattattctgtcccttggatccgtcaagctaactggactatatgacacctgagcaattataaatcatatattaaattgcacaaatattt

SEQ ID NO: 7 - DNA, 5133 bp, Mus musculus
gggggcggcgagttccagccggctgacggggtggcggccgtgagcgttaagcgtccgggacgcgggatggagccccacggatttcagtttttctgactgttaaatgagaggatgattgctcacaaacagaaaaaggcaaagaaaaagcgtgtttgggcatcaggccaaccttctgctgctattacaacttctgaaatggggctcaagtccgtaagttccagctccagttttgatccggagtacatcaaggagctggtgaatgatgtcaggaagttctcccatatgttgctatatttgaaagaagctattctttcagactgttttaaagaagtcattcatatccgtctggatgagcttctccgtgttttaaagtcgatattgagcaagcatcagaacctcagctccgtagatcttcagagcgctgcagaggtgctcactgcaaaagtgaaagctgtgaactttacagaagttaatgaagaaaacaaaaacgatatattccgagaagtcttttcctccattgaaacattggcatttacctttggaaacatcctcacaaacttccttatgggagacgtaggcagtgactcgatactacgtctacctatttctcgagaaagtaagtcttttgaaaacatttctgtggactcagtggacttaccccatgaaaaaggaaatttttctcctatagaactagacaacttgctgttaaagaacactgactctatagagctggctttgtcctatgctaaaacatggtcaaaatataccaagaatatagtgtcgtgggttgaaaaaaagctcaacttggaattggagtccactagaaatattgtaaaattggcagaggcaactagatctagcattggtatacaagagtttatgccactgcagtctctatttaccaacgctcttctcagtgacatccacagcagccaccttctacaacagacaattgcagccctccaagccaataaatttgtgcagcctctacttgggaggaagaatgagatggagaagcaaaggaaagaaataaaagacctttggaagcagcaacagaataaattgctcgaaacagagacagctctcaaaaaagcaaaattgttgtgcatgcagcggcaagatgaatacgaaaaggcaaaatcgtccatgtttcgtgcagaagaagagcagctaagttcaagtgttggtttggcaaaaaatctcaacaaacaactggaaaaaaggcggaggttggaagaagaggctcttcaaaaagtagaagaagcaaacgaacactacaaagtctgtgtaacaaatgttgaagaaagacggaatgatctagaaaatacaaagagggaaattttaacacagcttcggacacttgttttccagtgtgaccttacacttaaagctgtaacagttaacctctttcatatgcagcagctacaggctgcatcccttgccaacagtttacagtccctctgtgacagtgccaaactctatgatccaggtcaggagtacagtgaattcgtgaaggctacaagctcaagtgaattagaagaaaaggttgatgggaatgtaaataaacaaatgaccaacagtccgcagacatctggctatgaacctgctgactccttagaggatgttgcccgccttcctgacagctgtcataagcttgaagaggacaggtgctccaacagtgcagacatgacaggtccttctttcgtaagatcatggaagttcggaatgtttagtgactcagagagcactggaggaagcagtgagtctagatctctggattcagagtctataagtccaggagactttcatcggaaacttccacggactccatccagtggaaccatgtcttctgctgatgatctcgatgagagagagccaccgtccccttcagaagctggacccaattccctcggagcatttaagaaaactttgatgtcaaaggcagctctcactcacaagtttcgcaagttgagatccccgacaaagtgcagggattgtgacggcatcgtaatgttcccaggcgtcgagtgtgaagagtgtctccttgtttgtcatcggaagtgtctggagaatttagtcattatttgtggtcatcaaaaacttcagggaaaaatgcacatatttggagcagaattcatacaagttgcaaaaaaggaaccagatggcatcccttttgtactgaaaatatgtgcctcagaaattgaaaatagagccttgtgtctccagggaatttatcgtgtttgtggaaacaaaataaaaactgaaaaactgtgccaagctttggaaaatgggatgcacttagtagacatttcagaattcagttcacatgacatctgtgatgtcttgaaattatacctgcgacagcttccagaaccatttattttattcagattgtacaaggaatttatagaccttgcaaaagagatacaacatgtaaatgaagaacaagaggcaaaaaaagatagccctgaagacaagaaacacccacatgtgagcatagaagtcaaccgcatccttctgaagagcaaagacctgctgagacagctgccagcgtcacatttcaacagcctccattacctcatagcacatctgaggcgagtggtggatcatgcagaagagaacaagatgaattctaagaacttgggggtgatatttggaccaactctcattaggccaaggcctacaacggctcctgtcaccatctcgtcccttgctgaatattccaatcaggcacgattagtagagttccttattacttactcacagaagatcttcgatgggtccctccagcctcaagctgttgttatatctaacacaggtgctgtggcacctcaggttgatcaaggctatcttccaaaacctctgttatcaccagatgagagagacacagatcattctatgaaaccactctttttttcttcaaaggaagatatccgtagttcagattgtgagagcaaaagttttgaattaactacatcttttgaagaatcagaacgcagacaaaatgcattggggaaatgtgacgctcctctcctcgacaacaaagtacatttgctttttgaccaagagcatgagtcagcgtcccaaaagatggaagatgtctgtaaaagccccaagctgctgctgctgaaatccaatagggcagcaaacagtgtgcagagacatactccaaggaccaagatgagacctgtaagcttgcctgtagaccggctgcttcttcttgccagttctcctactgagagaagcagcagggatgtaggaaacgtagactcagacaagtttggcaagaaccctgcctttgaaggactccatagaaaggacaactcaaatactactcgctccaaagttaatggctttgaccagcaaaatgtacagaaatcctgggacacacaatatgtacggaacaattttactgccaagactacgatgattgttcccagtgcctaccctgagaagggattgacagtaaacactgggaataacagggaccatcccggcagtaaagcacatgcagagccagccagggctgcaggagatgtgtcagagcgcaggtcctctgactcctgccccgccactgctgtcagagcacccagaacactgcagccccaacactggacaacattttacaaaccacctaatcccaccttcagtgtcagggggactgaggagaaaacagcattaccctcaatagctgtacctcctgtcctggtgcatgctccccagatccatgtgacaaaatcagacccagactcagaggccacattggcctgtcctgtgcagacaagtggtcaacctaaagagagctctgaggagcctgccctgcctgaggggactccaacttgccagagaccacgactaaaacgaatgcagcaatttgaagaccttgaagatgaaatcccacagtttgtgtaggattgtcaaaatttagatttttctgttttattttgttctgtggtgtcattttgtgagagaatgtttggacagggcccttttgtataggattgccaaagctgtttgtcagtgtggtgtttgttgctcatgtgggatgggagagtgtcctgacaaggctccgtttagcctcactggaatgatctttgaagctgtaaagaaaaatgggtgtttttgtgttttttagagttgattttttcctgaagaatgatccatttaaatgcatcactgatacatgatacaatttttagcagtaggtgcaattggggaaaatcagctttagtgtggagagtgagcccaagtgcatatttataaagtatttctgaacacaagtggtgttcatgtgctgtggttcctcacagtttcataggacatctttgaccatttgtgcttctgtaattgtaagtcagcttctatttttcaattggaaattcaccttttaatatttacataatggccaaagggtttaccagtctgtatttaattataattgccaatttttacatatggcagttaaattgtaccccccaaaatgcacttaaacctgaactgtgtagttcagttacctcttatttgactttagatggatttaatacatttggtaggggctggggggtttgttttgtttttgttttatcccttagcctcgtgtgtataaactctcatttccaacagttctgaacacatttatacagtggtccaggaaagatgctgtttttcagaagtttttaaatttgatacatttcctctttttactacctgggtttgtttaaactgttcttttataagaaatgttttgacttacagatcattttatattctttttctacctgactttcaaccattgaaaatgtgtagttctttcaaatggagtgaagatttatttaagttaatcctaagggtacacgtcgtgtttagaaatgtgagaaggcgtagatgagcagatcagttttgtttaaagagcaaactaacaacctagttttcagaacttgtgcactcctgttcctctctgcatcattgtttgtctgaatgggatgtaaaagggacagcacacacagtagccttccgtacgtgtgaattatgttatgcttttgtatgaccttgttatatttgataaatatatgtatatatccacttcttaaaaaaaaaaaaaaaaaaaaaaaaaaaaaaaaaaaaaaaaaaaaa

SEQ ID NO: 8 - DNA, 5510 bp, Mus musculus
acttccgtcctcaagactttctccctagaggccgggtatcagagaccagctgctggctctctgagctacttcccaggtttggcgcctggaaaagttcacgttctgcattctcctgcttctggctccgccccggctccagaccctgcgttcttctggccttacccgggacgggccacccctttccccgctgcctctggcgcgagggtgctgggacatctctgagccagctctgggcccaaccaaggttggcagcaaatatcaagtgtcgctcttctagaggaacacggatactcgcttcagagactgtcttctgagcgcagacctttctgagtagtgctgagcgcagcgggggagttctttgacaccgttgtcgctcagtgcttggaaggcccgggacgcagcacatatggtgtcccactgagtcaacagcgggactgcgcgggaacgtgaacttggagacactttggagcctcgtcaatcagaaagggggacttagcaacccagctgatcccccaaagcacagccgcggtccccaagttaccaagaaggtgcactgggggcgcagccggacagctgagctggggtgctccagaggacttttcactgcgccgggagcaccaaggatccgctcagggcggactctcaggcagcctcctccctagccctcgggattgtcctcaggccacgaggaggagcttgctggtgatttcgaggctgtccggagccagagagccgaagcgcagtgtctcccgccttcagctgggaagggggagtggcgctggcgggttggagctgagatctcagctagtcactgacctccttcctcctcttccttagcctctttgagacttggactcctgaggaagattctagagacggtaaagggacctggacctcttgtttcccaaaaggctggggatggagctcctgtttctgcctccgcagggacacttggagtgcgctggtggcgcgtgaacggggcactgctttctaccttcctcggcgagccccgcctggcagttttcccctactctactttggccacttgttttctcaggtcacagtctcccgctatctaggagggaagacaagaaggtggccttcagacccagccctgccgagatgtccgcgcagagcctgctgcacagtgtcttctcctgctcctcgcccgcgtcgggcggcacggcctcggccaagggattctccaaaaggaagctgcgccagacgcgcagcctggacccagctctgatcggtggctgcgggagcgagatgggcgccgagggcggcctgcggggctccacagtaagccgcctccattctccacagctcttagcagagggtctcggttcccgcttagcttcttctccccggagtcagcaccttcgggctacccggttccagactccgagacctctgtgctcgtctttttccacaccaagtaccccgcaagaaaagtcgccttctggcagcttccactttgactacgaggtcccactgagtcgcagtggtctcaagaagagcatggcctgggacttgccttctgtcctggccgggtccgggtccgctagtagccgcagtcccgcaagcatcctcagttcctccgggggaggccccaatggcatcttctcttctcctaggagatggctccagcagaggaagttccagcctccacctaacagtcgcagtcacccttacgtcgtgtggaggtccgagggtgacttcacctggaacagcatgtctggtcgtagcgtgcgcctgaggtcagtccccatccagagcctctcagagctggagcgggcacgactgcaggaagtggctttttatcagttgcagcaggactgtgacctgggctgtcagatcaccatccccaaagatggacaaaagagaaagaaatctttgagaaagaaactggattcactagggaaggaaaagaacaaagacaaagaattcatcccacaggcatttggaatgcccttatcccaagtcattgctaatgaccgggcatataaactgaagcaagacttgcagagggaggagcagaaggatgcatcatcggattttgtgtcttccctcctcccatttgggaataaaaaacaaaacaaagaactctcaagcagtaactcatctctcagctcaacctcagaaacaccaaatgagtctacatcaccgaatactccagaaccagctcctcgggccaggagaaggggcgccatgtccgtggattccatcactgatctggatgacaaccagtctcgactcctagaagctttacaactctccttgcctgctgaggctcagagtaaaaaagaaaaggccagagataagaagctgagtctgaatcctatttacaggcaggtccccaggctggtggacagctgctgtcaacatctggaaaaacatggcctccagacagtggggatattccgagttggaagctcaaagaagagagtaagacaattgcgtgaagaatttgaccgtggggttgatgtctgtctggaagaggagcatagtgttcacgatgtggcagccttgttaaaggagttccttagagacatgcctgacccccttctcacaagggagctatacactgcatttatcaacactctcctgttggagcctgaggaacaactgggcaccttgcaactcctcatttaccttctacctccctgcaactgcgacaccctccaccgcctcctacagttcctctccattgtggccaggcatgctgatgataatgtcagcaaagatggacaagaggttactgggaacaaaatgacatctctgaacttagccactatatttggacccaacctgctccacaagcagaagtcatcagacaaagaatattctgttcagagctcagccagagctgaggagagcacagccatcatagctgtggtacagaagatgattgaaaattatgaagccttgttcatggttcccccagatctccagaatgaagtgctgatcagccttctagagacagatccagatgttgtggactacttgctcagaagaaaggcttcccaatcctcgagccctgacatacttcagacggaagtttccttttccatgggagggaggcattcatctacagattccaacaaagcctccagtggagacatctccccttatgacaacaactccccagtattgtctgagcgctccctgctggctatgcaagaggacagggcccgggggggctcggagaagctttataaagtgccagagcagtatacactggtgggccacttgtcatcgccaaagtcaaagtcaagagaaagttctcctggaccaaggcttggaaaagaaatgtcagaggagcctttcaatatctggggaacttggcattcaacattaaaaagtggatccaaagacccaggaatgacaggctcttatggcgacatttttgaaagcagctccctccgaccgaggccttgttctctttctcaagggaacctttccctgaactggcctcggtgtcaagggagcccgacagggctggacagtggcactcaggtaattcggaggactcagacggcggccaccgtggagcagtgcagtgtccaccttccggtgtcacgtgtctgcagcactccccacatccaggacggcagcagggggaccaggcggcctgcagccagctctgatccatttttgtccctaaacagcacagaagatctagctgagggcaaggaggatgttgcctggctgcaaagccaggcccgacctgtgtaccagagacctcaggagagtggaaaagatgacaggcgcccccctcctccttacccggggtcagggaagcctgccacaacctctgcccagctgccactagagcctcccctgtggaggctccagaggcatgaagaaggttcagaaacagctgtggaaggaggccagcaggcctcaggggagcatcagaccaggccaaaaaaactgagcagcgcctactccctctcagccagcgagcaggacaaacagaacttaggggaagccagctggctcgactggcagcgagagcggtggcagatctgggagcttctatcaactgataaccccgacgccctcccggaaaccctagtataagcccgccagcagctggagcccacccttccaaaacacatcttccggtccagacccggaaaccttgcctatggacaattggacacttacttgtttttcttttttgtttttccacactttgaaaaagcaacacaaaagaaagtccacttattgattcacttctacccctgccatttatggtaagattctattgcatagccagccttaggaaaaaaacaaataaaccaacaaacatgacaattcccaagctcaaaacaacccacattggctctatgtaagaaactcttgcttcgttatagcttaattgtatttgtgtcttcaattttgactattgtatattctgtaacaaattatgtatatcaatatgatatattcacagagaagacagaacaattaaaaatcactgcacttatattacacactgagatatattaagcaaccagattctatatgctctggaatatgcacaagcgggtatctgtgctttttgccatcaccttttaactgggggcagcccctcccttcaatgcctaaggaaatactaaccaaacaagagagaaaatgagaagccatatttttatatagtattgagacacaaaagttgtagtcactgaatgctttttcatagcaagtatgttttaaggaaatattaaatttgatacattgtgaaatatatttttagaatctgtttagaaaggactcagaaaatcaaatcagagacaggtgggacccaagagtacttaagagagtttctattccactctaggtcaaatttaattttatataggccactaataatatatatttataatggattacttttatgtatttttcaaagctaccaactgaaatccaattttaaaaagctttaaaatccaaatacacattcaaattatagatcatttcccccatctgcccagttatcaatattagctcaattacaagcaattccttgtaaagtaaatcctatggggggggagcaaaaaagctacatctttgcgcttacattgtaccaaaggctgaggaaatgtgtcttgagtatcttcagtaatattgtgtgtattgtaacgtatgtgttactacagtaaacagtacttcaacaatttcaagtgttacaactgcaaaaccacttttgaccagcaggtggcagtttgcttcagtattttccattgttttgttttgtttttcaaatcagaagggtcagtgtattatatactaagtgggatatatatgacgtgttactcttaatcttcatgttggcagtgaaatttttcagtggtgtttattaaaattctaccttgtgccatgatgagtaaaatgttaagtaaagatttgttgtcagctcttagttttcatgttggcaatgaaatttttcagtggtgtttcttaaaattctatcttgtgccacaatgaataaaatgttaagc

SEQ ID NO: 9 - DNA, 1570 bp, Mus musculus
cgggacagggaagcttccagagaggcccatgaccaggctggggctggcaaccaaaagcccctggaccctgtaaacctgccaggcaccacagaggcagagaggatgagcaagaggagagtggcagcggacttgccctcgggaaccaactccagcatgcccgtgcagaggcacagggtgtcatccctcaggggaacacactctccatcctccctggatagccccccagcatccaggaccagtgctgtgggtagcctcgtccgtgcccctggggtctatgtaggagtcgcacccagtggtggcataggtggtctcggtgcccgagtgacccgccgggccctgggcatcagcagtgtctttctacagggcctgcggagttcaggccttgccaacgtgcctgctccgggcccagaaagggatcacactactgttgaggacctggggggctgcctagtggaatatatgaccaaggtgcatgctctggagcaagtcagccaggaactggaaacacaactgcgggctcacctggagagcaaggccaagagctctggaggctgggatgccctccgcgcctcctgggccagcagctaccagcaggtgggagaggctgtcctagaaaacgcccggctcctgctgcagatggagacgatccaggccggtgcggatgactttaaagagagatatgaaaacgagcagccattcaggaaggcagcggaagaggaagtaagttccctgtacaaagtcatcgatgaagctaatttgacaaagacggatctggagcatcaaatagaaagcctgaaagaagaactgggctttctgtcaagaagctatgaagaggatgtgaaggttctgtacaaacagctggcagggtctgagctggagcaagcagatgtccccatgggcaccggtctggatgatgtccttgagacgatccgagttcagtgggagagagatgtggaaaagaaccgagcagaagcaggagccttgctccaagctaagcaacagacagaggtggtccacgtatcccagacccaagaagaaaagctggctgctgccctcagtgtagagttacacgacacttcacgccaagtccagagtctccaggctgagacggaatctttacgggctctgaaacgaggcctggaaaacagcttgcacgacgcccagcactggcatgacatggaactgcagaacctgggtgccgtggtgggcaggctggaggcagagctggcagagatccgctcagagacagaacagcagcagcaggagcgggcacacctgctggcgtgcaagagccagctacagaaggatgtggcatcctaccacgccctgctggacagagaggagaacaactaatgggaaaaccaaaaaacgacttcctcttttcacaaagaaaactctgccttcctcggcagcccaccggtgacgtctgaagaacctcagtggctgctggactccctagctgactcagacggagctccctgggggtggagagaattctgctcccatttctgtagtctgtagcttgaacaaccgaggcctctctgaataaatactttgcgtgtggctccca

SEQ ID NO: 10 - DNA, 2389 bp, Mus musculus
ctctctccacgaactgcccaggagcgagcagctgctcccggttggccctgacggacagacaaaccgacagcctgacaacctagtccaccaactaagcagcctgcacctggctgcttgtccctccccaggaacattgaccatgtgtcccctgtggctactcaccttgctgctggccctgagccaggccttgccctttgagcagaagggtttctgggacttcaccttggatgatgggctgctcatgatgaatgatgaggaggcttcaggttcagacaccacttcaggtgtccccgacctggactctgtcacacctaccttcagtgccatgtgtcctttcggttgccactgccacctgcgggttgttcagtgctctgacttgggtctgaagactgtgcccaaggagatctcacctgacaccacactgctagacctgcagaacaatgacatttctgagcttcgcaaggatgacttcaaaggcctccagcacctctacgccctggtcttggtaaacaataagatctccaagatccatgagaaggcctttagccctctgcggaagctgcaaaaactctacatctccaagaaccacctggtggagattcctcccaacctgcccagctccctggtagaactacgaatccatgacaaccgtatccgcaaagtgcccaagggcgtgttcagcgggctccggaacatgaactgcattgagatgggcgggaatcccctggagaacagtggctttgaaccaggagcctttgatggcctgaagctcaattacctgcgcatctcagaggccaagctcactggcatccccaaagatctccctgagaccctgaacgaacttcacctggaccacaacaaaatccaggctattgagttggaggacctacttcgatactccaagctgtacaggttgggcttaggtcacaatcagattcggatgattgagaatgggagcctgagttttctgcctaccctgagggaacttcacttggacaacaacaagctgtcccgggtgcctgctggcctcccagatctcaagctcctccaggttgtctatctgcactccaacaacatcaccaaggtgggcatcaatgacttctgtcctatgggcttcggagtcaagagggcctactataatggcatcagcctcttcaacaaccctgtgccctactgggaagtgcagcctgccaccttccgctgcgttactgaccgcctggccatccaatttggaaattataagaagtagaggcagtggttgccaccatggtggccttggtgagagtctctgaggaacatagccagatgaagaagcaacacctttgttccccaatattaactcactgccccaccacagcttccccctgactcctaagcatgcatatatgcacatggcctggccctctcacccattcccctcaacctttgaaatttaacattcatcaaccatgtccactcagagactccctataaatctttcttcttgctcatcctgaaactcagatgtttttggcaagaggggctaggaaagatggatagagcacactgccaccgccattgttccatccaggcatgtgttcctcctcttccttgctcatgtctgacttccagctctcctgggctctgcttgctgcccttatcctctggtgttctctcttcaacaagttcactacctgtcaatcccagctacaacctggctgtactaactcctggatctttccctctctccaaccctgttatgcttcctgacacttttcttccttctggagttattgacctgtccccttccatctctggacctaggtcatatttctccatctttgtctctttctgtatctccttgcctatatctctgtctgtctctatttctgtctctctgtctctgtgtatctctctatctttgtatctgtctctctctgacaacacacacacacacacacacacacacacacacacacacacacacacacacggatcatctgccccaggctgctttctgcttcacaggtctctagccagtccctccacaaacaaatatggggcaactatcttcctgattgccctacccagaacttgaccccccaaccctggaggaagctggaaggtggaggcccagaatcctgtccattttgtccaggaaagggttcatactctgctatcaagacgaggatcaaggagcttcctagcccctggagaggctcagcaggccatcagagccgccagaaccagtttgcattggcccctgctctctccccaagatggctaggtcccctccctcacccctgggtccctgatgtggtaggaggtgatggtcagttgcacccagcaagagggagtgctgcttatgaggtcagttgtctctcaattaaagaaacactgtgcaatacga

SEQ ID NO: 11 - DNA, 1221 bp, Mus musculus
agctatcgaggggcaagctgagacgagtttgagaagaaaaggcccgtggagaggtctgcaaacagcatgtacacacccatccctcagagtggctctccattcccggcctcagtccaagacccaggcctacacatatggcgtgtggagaagctgaagccggtgcccatagcacgagagagccatggcatctttttctctggggactcctacctagtgcttcacaatggcccagaggaggcttcccatctgcacctgtggataggccagcagtcctcccgggatgagcagggggcctgtgcagtgctggctgtgcatctcaacaccctgctgggggagcggccagtgcagcaccgtgaggttcaaggcaatgagtctgacctcttcatgagctacttcccacgaggcctcaagtaccgggaaggtggtgtagagtcggcatttcacaagacaacctcgggcgccaccccagcagccatcaggaagctctaccaggttaaggggaagaagaacatccgtgcgaccgagagggctctgagttgggacagcttcaacactggggactgcttcatcctggacctgggtcagaacatctttgcctggtgtggtggaaagtccaacatccttgagcgcaacaaggcgagggacctggccctggccatcagggacagcgagcggcagggcaaggcccaggtggaaatcatcactgatggagaggagccagccgagatgattcaggttctgggccccaagcctgctctgaaggagggtaaccccgaggaagacattacagctgaccagaccaacgcccaggctgcagccctgtataaggtctctgatgccactggacagatgaatctgaccaaggtggctgactccagcccttttgcctctgaactgctaattccagatgactgctttgttctggacaacgggctgtgtggcaaaatctacatctggaaggggagaaaagctaatgagaaagagcggcaggcagccctccaagtggctgatggcttcatctctcgaatgaggtattccccaaacactcaggtggagatactgccccagggccgagagagtcccatcttcaagcaattcttcaagaactggaagtgagggtgggtgtcccccatctctgctctcctgcctcccacccctgcctgctgggtcagcactgaggtgccctctggatgctcaataaaggacacattccattccctg

SEQ ID NO: 12 - DNA, 1433 bp, Mus musculus
actctgtcaagctgtcttcacggtgcgaaagaactgaggctttttctcatggctgaaaacaaacaccctgacaaaccacttaaggtgttggaacagctgggcaaagaagtccttacggagtacctagaaaaattagtacaaagcaatgtactgaaattaaaggaggaagataaacaaaaatttaacaatgctgaacgcagtgacaagcgttgggtttttgtagatgccatgaaaaagaaacacagcaaagtaggtgaaatgcttctccagacattcttcagtgtggacccaggcagccaccatggtgaagctaatctggaaatggaggaaccagaagaatcattgaacactctcaagctttgttcccctgaagagttcacaaggctttgcagagaaaagacacaagaaatttacccaataaaggaggccaatggccgtacacgaaaggctcttatcatatgcaatacagagttcaaacatctctcactgaggtatggggctaactttgacatcattggtatgaaaggccttcttgaagacttaggctacgatgtggtggtgaaagaggagcttacagcagagggcatggagtcagagatgaaagactttgctgcactctcagaacaccagacatcagacagcacattcctggtgctaatgtctcatggcacactgcatggcatttgtggaacaatgcacagtgaaaaaactccagatgtgctacagtatgataccatctatcagatattcaacaattgccactgtccaggtctacgagacaaacccaaagtcatcattgtgcaggcctgcagaggtgggaactctggagaaatgtggatcagagagtcttcaaaaccccagttgtgcagaggtgtagatctacctaggaatatggaagctgatgctgtcaagctgagccacgtggagaaggacttcattgccttctactctacaaccccacatcacttgtcctaccgagacaaaacaggaggctcttacttcatcactagactcatttcctgcttccggaaacatgcttgctcttgtcatctctttgatatattcctgaaggtgcaacaatcatttgaaaaggcaagtattcattcccagatgcccaccattgatcgggcaaccttgacgagatatttctacctctttcctggcaactgagaacaaagcaacaagcaactgaatctcatttcttcagcttgaagaagtgatcttggccaaggatcacattctattcctgaaattccagaactagtgaaattaaggaaagaatacttatgaattcaagaccagcctaagcaacacagtgggattctgttccatagacaagcaaacaagcaaaaataaaacaaaaaaaaaatttaccaaaagagaaatttgttttatttatttgtgtacataaataaaaagaaagcaaataatta

SEQ ID NO: 13 - DNA, 2487 bp, Mus musculus
acaagatcttccttcctcagttctcttaaatcacagcccagggaaacctcctcagagcctgcagccagccacgcgccagcatgtctgggggcaaatacgtagactccgagggacatctctacactgttcccatccgggaacagggcaacatctacaagcccaacaacaaggccatggcagacgaggtgactgagaagcaagtgtatgacgcgcacaccaaggagattgacctggtcaaccgcgaccccaagcatctcaacgacgacgtggtcaagattgactttgaagatgtgattgcagaaccagaagggacacacagtttcgacggcatctggaaggccagcttcaccaccttcactgtgacaaaatattggttttaccgcttgttgtctacgatcttcggcatcccaatggcactcatctggggcatttactttgccattctctccttcctgcacatctgggcggttgtaccgtgcatcaagagcttcctgattgagattcagtgcatcagccgcgtctactccatctacgtccataccttctgcgatccactctttgaagctattggcaagatattcagcaacatccgcatcagcacgcagaaagagatatgagggacatttcaaggatgaaaggtttttttccccccttactatttccttggtgccaattccaagttgctctcgcagcagcaaatttatgaatggtttgtcttgatcaagaacaaagaattcattcccaccattctcatatatactacttgtctcttctaagctactgcatctatgtttgacagtctggaatgtttaaacccattcctgctctctcttttatatgtgaatcattgtttcattggctaaaatataaacatattgttgaaagatgatttgagaaaaataggaaggactgggaggcagggaagagtaccaacaacctcaactgcctactcaaaggtgatgatgtcatacaaagggaagagattcaggttacggccatttgtttaggggcatgaaggaacgtttttaatatatgccagttatctaaggaattggttgctgtcctcactcttaacaatccagttagatttagggatttagggatcaccatcaatttggagactataatcttcatgataccaacaatgttttacttatcctggcattttaacctgttattttgtatgcctgaatatttgctatactgagaataagacctacgtgccttctaatttttcatgttttttttttttccaaataggatctaactcatctacttgcatgatgccggcagctttcctaaaaacaaaacatacaaattgcacttgctagttctctgtacttgtttctgactctgaaatacagaacctgttgatgttgatatctgtgctcagctatgtagcatctttctctctgttaagcctggtcaacattaacccaatgaaatgatttgaagcagacaaatgggggtgagacctctctggactggcagaagtagaagccagctttccctgccactcagcaactgaatgaggccagcgtgtctattcagtttcactcattttcaagaataatcacaggttcctgactctaagccagcccctcaccaggatcaaggtttagtgactgactgggatgatttaggagctcaacattgtacttccttttcagctgatgagtgaacctccagggaggggtgtcaaaaaggaggctgctaaaccgagactgccaagcctgttgtaaacatgaccccttttatgcaaagcccttgcaatagtctgcaatgctgtgaagctcgacctttccccctgcaaggaacctttgacctaatccaaccatcattttgttcagaaaggtgggggaagggtggtaacaaaagcttgaggtaatgttcttgctgtaataaattcaagtttttctgaacccaaactgaggaatttcacctgtgtacctgagtctccagaaagctgcctgcctgggacacccaaaagccttttacttcccagctcacattacagctctgcccttggggatatttttaaaattccagataggctttcattttcactttcatacatgtattggaaccctgcttgacttgttttctccttcagtcttgccgacactttaccaacctgctacctactttgattgtttgcatttaaaacagacactggcatggccacagtttgaattttaaactgtgcacataactgaaagtgtactagactgtatacctttttacatgtagagatattctttatctttatataaggagaatcacttgggaaatgattctacaattcagtctgtaaactgtgtgttccaagacatgtctgttctccctagatactcagttttatacaagtcaattgctgatccaaaaggttactgaaattttatatgcttactgatatattttacacttttttatgctgcatgtcctataaagatttcaaatctgcacaataaaattgtttaacagtta

SEQ ID NO: 14 - DNA, 3979 bp, Mus musculus
ccctccctggctctctcctcagctctgggctctgactgcagcaagcagagacaacctctcactctgcctttcccagcgcccaccctgaccctggcccacatttgacggtgactcgcaggccagccagaaacatgaggctggcccacgctctgctgcccctgctgctacaagcctgctgggtggccacacaggacatccagggctccaaagcgattgccttccaagactgccctgtggatctattcttcgtgctcgacacctcggagagtgtggccttgaggctgaaaccttatggggccttggtggacaaggtgaagtccttcactaagcgcttcattgacaacctgagagacaggtactaccggtgtgaccgcaacctggtttggaatgcgggtgcgctgcactacagtgacgaggtggagatcatccgagggctcacgcgcatgcccagtggccgcgatgagctcaaggccagcgtggatgcggtcaagtacttcgggaaaggcacctacaccgactgcgccattaagaaggggctggaggagctgctcatagggggctcccacctgaaggagaacaagtacttgatcgtggtgaccgacgggcatcctctagagggctacaaggaaccatgcgggggtctggaagatgcagtaaatgaggccaaacacctgggcatcaaggtcttttctgtggccatcacacctgaccacctggagccacgtctaagtatcattgccacagaccacacataccggcgcaatttcacggcagctgactgggggcatagccgcgatgcagaagaggtcatcagccagaccattgacaccattgtggacatgattaaaaataacgtggaacaagtgtgttgttcttttgagtgccaggctgccagaggacctccagggccccgaggcgaccctgggtatgagggggagcgaggaaagccaggtcttccgggagagaagggagaagctggagaccctggacgacctggggatcttggaccagtcgggtaccagggtatgaagggagaaaaggggagccgtggagagaagggttccagaggaccgaaaggttacaagggcgagaaaggcaagcgcggaatcgacggggtcgacggcatgaagggagagacggggtacccaggactaccgggctgcaagggctccccaggatttgatggcattcaaggacccccgggtcccaagggtgatgctggtgcctttgggatgaagggagaaaagggtgaagctggagcagacggtgaggctgggagaccagggaactcagggtcacctggagatgagggtgatcctggagagcctggtccccccggagaaaaaggagaggccggtgatgaaggaaatgctggcccagacggtgcccctggagagaggggtggccctggtgaaagaggacctcgggggacccctggtgtgagaggaccaaggggagacccgggtgaagctggaccacagggtgaccaaggaagagaggggcccgtcggcatccctggagactcgggtgaggctggccccattggacctaaaggataccgaggtgatgagggtcctccaggtcctgagggcctcagaggagccccaggacctgttggtcctcctggagaccccggactgatgggtgagagaggtgaggatggaccaccaggaaacggcacggaaggtttccccggcttccctgggtatccaggcaacagaggccctcctgggctaaatggcacaaaaggctaccctggcctcaagggggatgagggtgaagtgggagacccaggagaggataacaacgacatttcaccccgtggggtcaaaggggcaaagggataccgaggcccagaaggaccccagggacctccaggacatgtgggaccacctgggccagatgagtgtgagatcctggatatcatcatgaaaatgtgctcctgctgtgagtgcacatgtggacccattgacatcctcttcgtgctggacagctcggagagcattggcctacagaactttgagattgccaaggacttcatcatcaaggtcattgaccggttgagcaaggatgagctggtcaaatttgagccagggcagtctcacgcgggcgtggtacagtacagccacaaccagatgcaagagcacgtggacatgcggagccccaacgtccgcaacgcccaggacttcaaagaagctgtcaagaagctacaatggatggctggtggcacattcaccggagaagcgctgcagtacacccgggaccggctactcccacccacacagaacaaccgaattgccctggtcattacggatggacgttctgacactcaacgggacacgacacctctcagtgtgctctgtggtgcagacattcaggtagtttctgtgggaatcaaggatgtgtttggctttgtggcgggctccgaccagctcaatgtcatttcctgccaaggcttatcgcaaggtcggccaggtatctccctggtgaaggagaactatgcagagcttctcgatgacggctttctgaagaacataacagcccagatctgtatagataagaagtgtccggattatacctgtccaatcacattctcctccccggctgacatcaccatcctgctagacagctcagccagtgtcggcagccacaacttcgaaaccaccaaggtcttcgccaagcgcctagctgagcgattcctgtcagcaggcagggcggatccttcccaggatgtgcgggtggccgtggtacagtatagtggccaggggcagcaacagccaggtcgggcggctcttcagttcttacagaattacacagtgctggccagctctgtggacagcatggatttcatcaacgacgccacagacgtcaacgatgctctgagctacgtgactcgtttctaccgggaagcctcgtcaggtgccaccaagaagagagtgctgttgttttcagacggcaactctcagggggccacagcagaggccattgagaaggctgtgcaggaggcccagcgtgcaggcattgagatctttgtggtggtggtgggaccccaggtgaacgagccccacatccgtgtgcttgtcactggcaagactgcagagtacgacgtggcctttggcgagcgccacctattccgtgtaccaaactaccaggccctgctacgtggcgtactctaccagacagtctccaggaaggtggcactgggctagagggccacacacgtggctggacacacatggcatggagacacatttcaacaggccttcccgcccttcccactgacaaaacaggaataggaaatgtgacccaactggtcaactcaactgtcttaaagggaacgctgagatgcacactctttgctttgtgtaatgtcccctgtggctcacctgagctcctatctagatcccgcccttggtttgtacatcatggtggccatcttgctgacccctcccccatctgggtccagccatctcgtcttcctcctcactgcccctaacctatccgtggtgtcttcacaccatcactgcagtttccgtctgtgttctgtcttccatgctcaacatgaagcagaccttctcatgagttcagcttgctggattatggcttttaggaaattgaacacaggaggagttccaaacacaaacttggaggagacccctcctcttcatcaggtgcttgtcagtgacctacatgcatcttggtctggtccttagtggctagtccttccactctgaaagcaaaggtgctatctatctgtaagggctctctctacacacccagaggcttagcttggacagttcacactcaagtgtcctgtcagaatcaatccagagctttctccctcaaaatagtgacttgtctccccctggtccccaaaggctcccctttagttagtttcttcatggctcccccacattccccgtaatctgatccaagccagctatctctgctaataaaggtttccatttttcaaaaaaag

SEQ ID NO: 15 - DNA, 3729 bp, Mus musculus
agagttaaagtgggaggcccctggcttggtcccctcccgttcagtcccgggccgcgcctgggtcccctccctcctacccactcggcgcccgcacctcgggccgtcaggacccgggctgtcctcgggaagtacccaggcatcttctccaagccaggacatcagggcacatgactactatcaagatgctccagggtcctctttctgtgctcctgattgggggactcttgggggtcctccatgcccagcagcaggaagccatctcaccccaggagcaggaagctgtctcaccagacatctccaccactgaaaggaacaacaattgtccagagaaggccgactgcccagtcaacgtgtatttcgtgttggacacctcagagagcgtggccatgcagtccccgacagacagcctgctctatcatatgcagcagttcgtaccgcagtttatcagccagctgcagaacgagttctacctggaccaggtggccctgagctggcgctacggtggtctacacttctcggaccaagtggaggtgttcagcccaccgggcagtgaccgggcctccttcactaagagcctacaaggcatccgctccttccgcaggggcaccttcactgactgtgcattggctaacatgacgcagcagatccggcagcacgtaggcaagggggtggtcaacttcgccgtggtcatcactgacggccacgtcacgggcagtccgtgtgggggcatcaagatgcaggctgagcgtgcccgtgaagagggcatccggctcttcgctgtggcccctaacaggaacctaaacgaacaaggcctgagggacatcgctaactctccacatgagctctaccgtaacaactacgccaccatgcgacccgactctaccgagattgaccaggacaccatcaaccgcatcatcaaggtcatgaaacatgaagcctatggagagtgctacaaggtgagctgcctggagattcctggaccccacggacccaagggttaccgaggacagaagggtgccaagggcaacatgggtgaaccaggagagcctggacagaaaggacgacagggagaccccggcatcgaaggccccattggattcccgggaccgaagggtgtgcctggcttcaagggagagaagggtgaatttggatcggatggtcggaagggagcgcctggcctagctggcaagaatggaacagatggacagaagggcaaactgggccgcattgggcctcctggttgcaagggagaccccggaagtcggggccccgatggataccctggagaagctggaagcccaggcgagcgaggagaccagggtgccaagggggactctggccgcccaggacgcaggggaccaccaggagatcctggagacaaaggaagcaagggatatcaaggcaacaacggagcccctggaagcccgggagtgaaaggaggcaagggagggcctggcccccgtggaccaaaaggagagcctggacgcagaggagaccccgggaccaagggcggccccggcagcgatggtccaaagggagagaagggagaccctggtcctgaggggcctcgaggcctggctggagaagttggcagtaaaggagccaagggagacagaggtttgcctggacccagaggcccccagggggctcttggagagccaggaaagcagggatctcgaggagaccctggtgacgccggacctcgaggggattcaggacagccgggccccaagggcgatcctggaaggcctggattcagctacccgggacctcgagggacacccggtgaaaaaggcgagcccggtccaccaggccctgagggaggccgaggagactttggtctgaaaggaacacccggacggaagggagataaaggggagccagctgatcctggtccccctggtgaacctggccctcgggggccaagaggaatcccaggacctgagggagaacccggccctccaggagaccctggtctcacggaatgtgatgtcatgacctatgtgagggagacctgtggatgctgcgactgtgagaagcgctgtggtgccctggatgtggtcttcgtcatcgacagttctgagagtattggctacaccaacttcaccttggagaagaactttgtcatcaatgtggtcaacaggctaggtgccattgccaaggaccccaagtcagaaacaggcacacgtgtgggtgtggtgcagtacagccacgagggcacctttgaggccatccggctggacgacgagcgagtcaactccctgtctagtttcaaggaggctgtcaaaaaccttgaatggatcgccggtggcacttggacgccctctgccctcaagtttgcctataatcagctcatcaaagaaagccggcgccagaagacccgggtgttcgcagtggtcatcacggatgggcgccatgacccccgagatgatgacctcaatcttcgggcactgtgtgaccgagatgtcactgtgacagccattggcatcggtgacatgttccacgagactcatgagagtgagaacctctactccattgcctgtgacaagccacagcaagtgcgcaacatgacgctgttctctgacctggtggccgagaagttcatcgatgacatggaagacgtcctttgtccagacccccagatcgtgtgtccagaacttccctgccaaacagagctctatgtggcccagtgcacacaacggcccgtggacattgtcttcctgctggatggctcggagcggctgggcgagcagaacttccacaaggtgcggcgcttcgtggaggacgtgtcccggcgcctgactctggcccggagggatgatgacccactcaacgcccgcatggctctgttgcaatatggcagccagaatcagcaacaggtggccttcccactgacctacaacgtgaccaccatccacgaggccctggagagggccacctacctcaattccttttctcacgtgggcacgggcatcgtacacgccatcaacaacgtggtgcggggggcacggggtggggcgcggcgccacgcagagctctccttcgtcttcctcacggacggtgtcaccggcaatgacagcctggaggagtcagtgcactctatgcgtaagcagaacgtggtgcccactgtggtcgctgtgggcggcgacgtggacatggatgtgcttactaagatcagcctgggtgacagggcggccatcttccgggagaaagactttgacagtctggcccagcccagcttctttgacaggttcatccgctggatctgttagcaccgccatgctcggccacctctccatcccatctgtggtgctaataggaccctagccctgccggtcccagctagacggtacacttgggtctttctagaaagtgaaagcccttctcccaaaatcaggacagaaggactctgaacccaaagccccttacctactttcagctctcttggcttcccctaccccaagtctccatcctacctataccttgccctcaagcattggaggaccccagagtcttcccactgcctgtttcccacagcctctgcccccttacttccctttcccccttcatgcatccactagtcccttctgaaagctgtctgctggcctgcaccagtcctgcccaaggctctgtctttctctgcctgttatttcctatctcaggagatcagacctgagagccccatatcacatgcccaatggcccaataaaggttttgagcctccctgtt

SEQ ID NO: 16 - DNA, 974 bp, Mus musculus
agctgacccagcagtaggcaccaggcaccatgtcaccaaagacactacctctgttgctgctgctggtggtggtggtgatagcctggcctctggcagtacagtccgcgccccccacctgctactctcggatgctgaccctgagccgtgagatcatggcagacttccagagcctgcaggcttcagagcctgaggattcctgtgtgaggtacttgccccggctttacctggacatccataactactgtgtgctggccaagctgagagacttcgtggcttctcctcagtgctggaagatggccgaagtggacactctgaaggacagagtgcggaagctgtataccatcatgaactccttctgcaggcgggacttggtattcctctcagatgactgcagtgccttagaagacccaattcccgaggccacgggtcctccagactggcagagctaagcaggtggaccagaagaacaacccagaggtctgaagctgggccagttgtccagagttacaccccccacacacacacccaggtctacttttagtgccactgttagacctgccacatgtctctagcttctgaaacaccagtgagggtcctacctctgagcatgctttgtgcacaggttggaagctcagctcagctcctaggtgtctcattggaatgtaagaggcacaaagaggaaagtgcacactggcttcgctttggagagcaagcaccttaggaacagcaaaatctcatgcctttgtgactgttttaatgaactaatgggaccactcttctttctggtctctgcttacacctacaggggcttcaactttatgcttccttcttcctgtgcaagctttcctgcctctctctcattttaaagtgtttttactgcttttgcgatacatttacaaggcttttatgtagtgtaaacgagccaccctttcgctgaagggtgatgaaaaccaaataaacctctgtcgtgagaa

SEQ ID NO: 17 - DNA, 5690 bp, Mus musculus
agcgatgatgccccatttaccctttctcttcagatgcaggaaattttcactctgttccccagctgattggagctttttctaggtgcttccctgggagttacctccctagagatcagcaggcagggctgtcacgcttgggtagcagccagctcccagtgaattccttctgtggcctacttgtccttatgaagtccgagttttaattttgcacaggtaggaggtctcttttgctatggatagggcggataacggtgctaccattagaaaacaggcttctgttttctaggaaggcaagaggaaccccaggtaggggaccttgtgagaccaggtgacttggctcctcagccttgcttctacagaaaccaggagtgcttccccccactcttccctatttttgacgtcaagctcaaccagccagcagaggagcctcacggcttgggcggtggagagagagcccagggagagtggcagggaggggaagccatctcagcaacagcttggagagggagctgctatcccttgcccgcaaaacacggactaaagccaggctgaagaagacctgcgggctcgggctcggggatccgcggggttactgcaaagaggggcggggaaaaggcgggggcgctgcatgcagcgcgctggttccagcggtgcccgcggggaatgtgacatcagcggcgccgggcgcttgcggctggagcaggcagctcgcctcggtggccgcacggtgcacacctcgcccgggggaggacttggagcccggcaggcggccgggatgtcggcgaaggagaggccaaagggcaaagtgatcaaggacagcgtcaccctcctgccctgtttttatttcgttgagttgcctatattggcatcatcagtggttagcctctacttcttggaactcacagatgtcttcaaacctgtgcactctggattcagttgctatgataggagtcttagcatgccgtacattgagccaacccaggaggccataccattccttatgttgcttagcttggcttttgctggacctgcaattacgatcatggtgggtgaagggattctatactgctgcctctccaaaagaagaaacggagctggattggagcctaacatcaacgccggaggctgcaacttcaactcctttctcaggagagccgtcagattcgttggtgtccatgtgtttggactgtgctccacagctctcattacagatatcatacagctctccacaggatatcaggcaccatactttctgactgtgtgcaagccaaactatacctctctgaatgtatcctgcaaagaaaactcctacatcgtggaagatatttgttcaggatctgaccttacagtcatcaacagtggcagaaagtcattcccatcccaacatgcgaccctcgctgcctttgccgctgtgtatgtgtccatgtacttcaattccacattaaccgattcctctaagctcctgaaacctctcttggtcttcacatttatcatctgtgggatcatctgcggactaacacggataactcaatataagaaccatccagtcgatgtctattgtggctttttaataggaggaggaatcgcactatatttgggcctgtatgctgtagggaattttctgcctagtgaagacagtatgcttcagcacagagatgccctcaggtcactgacagacctcaatcaagaccccagcagggttttatcagctaaaaatggtagcagtggtgatggaattgctcacacagagggtatcctcaaccgaaaccacagggatgcaagctccttgacaaatctcaagagggccaacgctgacgtagaaatcatcactcctaggagccccatggggaaggaaagcatggtgaccttcagcaacacgctgcccagggccaacaccccctccgtggaagacccagtgagaagaaatgcgagcatccatgcctctatggattctgcccggtccaaacagctccttacccagtggaagagcaagaatgagagtcgtaagatgtccctacaggttatggacactgaaccagaaggccagtcaccacccaggtccatagaaatgaggtccagctcagagccctcgagggtgggggtgaacggagatcaccatgtcccgggcaatcagtacctcaagatacagcctggcacagtccccgggtgcaacaatagtatgccgggagggccacgcgtgtccatccagtcccgccctggctcttcccaattggtgcacatccccgaggagacccaggaaaacataagcacctcgcccaagagcagttctgcgcgagccaagtggctgaaagcagctgagaagaccgtggcctgtaaccggagcaacaaccagccacgcatcatgcaggtcatcgccatgtccaagcagcagggcgtgctgcagagcagccccaagaatgccgaaggtagcactgtcacctgcacaggctccatccgctacaaaaccctgactgaccatgagcccagcggcatcgtgcgagtggaggctcatcccgagaacaacaggcccatcattcagatcccgtcgtccactgagggtgaaggcagcggctcctggaagtggaaagctccggagaaaagtagtctgcgccaaacctatgagctcaacgacctcaacagggactcagaaagctgtgagtccctcaaagacagctttggttctggagatcgcaaaagaagcaacatcgacagcaatgagcaccaccaccacggcatcaccaccatccgagtgaccccggtggagggcagcgagataggctcagagacgctgtccgtgtcctcctcacgcgactccaccctgcgcaggaagggcaacatcatcttgatcccggaaagaagcaacagccctgaaaacacaagaaacatcttctacaaaggaacctcccccacgcgggcttataaggattgagagatggcggcccttcttgtcatcattttgatgacacccccacctccccatcccccaccctcaccccaagaccactcgtttattgtaccttgtgctcttttgggttttttgttttgttttgtttgggggccttttttttttccctagaagatatggagagccttcttgtccaactagattgttcaccatcagcctggaactctcactgaaccaccacagaaatcgtggcgattttacaccaagggaaaggaaaagcacaaagcaagacccgaactaaactcatcatcagaacagttcttaagacacaggctttgcagaaggtagtattaagataaagtggtttcctccgatgtatagtatttaactttctgaatgtgccaacttaatggagtttttttttttcattataattagctgtgggaacccaaaacacataggttttcccaacagcagaggccatgcggtattatatattattcatttttgcagactctgcaccagaagagcagactgggtggtgctgattatcacagtgcatctaccatttaaactctcaaactctatgtagctgtgaaatagtggtgtgcaactcctcgtcagagaaatgctacttcattcagaagacgccagtgactttgtgttagaatagaccattcttggcttccctgtagtggctctctcacagttgaaaagaaaagaaaagaaaagaaaaagaaaaagaaagagagagagagaaagagagaaagaaagaaagaaagaaagaaagaaagaaagaaagaaagaaagaaagaaagaaagaaagaaagaaagaaagaattggatgaattggacagggctttgagcatttctttgaaagatgctttttttcaacatctgaaagcttgtaggaatgttttcagtgaaacagaataactagttctctgcatcgtttttcttctttttatttaagtattggtaatgctgctttctggttttttgttttttgttttagtgagtgcatttgcatatttaaaatacattgttttagagaatattttgaaattattatgattacattttccattttatggctttaccttagtttattaagttttctgaggttacacatattcttctattttaagaaagcaaaagtgacaacttgcattctttgtgcaaaatacactgctgtgaggtcctacactagaaatctgagccaaaggttgaaactgtgcgtgccaatgccagatacgctggtcaaggtcaagatgtctccaatccgatggcataggttatcacatcagtaagtaatcccaaaatttcattttgttccagagcatttcattttcatgttatcttgataatcaccatattggagccacagtgggggtgagtttgactccctttcctgacacacttttaactgcacaccaacagtaagaatctaggcaaatgctaattgataaatagatgtgtatcacagtataagtttagaaagcatatcttcaaaatgtcagaccaggtaaagctttcgtgcttagagtataaccaacagttttggatgtctgtcttgaatctagaaccttaagcctaaatcaaaggaaaccttactgttgatagcaagaagataacaacatatttttgaagtggttttccaagctagctgtttaaagtgtggagaaggatttggttcttgaaatttggtattaaccttttttcatgccatgtcttaagaattataatgtacactcaacgattgccaagagagggggagggggaggaaaacagccaacagcagagctggttggtctgaactcagtgcagttttcaatgagaacaacagctgtccagcaaggaatcatatcatccattctcagcttctacattcaaagggcagagctttttagaaaactcaacctcctaaggcattaggaactgagctgaaaccagcagaattgaaaactctggcaataaaatatagactcaatcgtaacccttctggcaagttccttctcagagaaggaagtgggagtaaaatgtggccttccccacttctttacatcacccctgtcacaatgtccccgctggcctggccagtttcgagagggaagggtggactggttttagtactctgaagaaaacccaagctgcagtatttgaggtgcagtataatatttcctaatctttcctatttcttaacaaaaaaagattttaaagtacttctctactcattgaatttttgttctttacatactattgatatattctttttctactcaaaagtgccaaaggctacagtttttaatgacttaacaaattgtaccacattgttaaggaaatataatgatagacactagaattcagacctctgcatgtatatttgataacacatcttttgtaaaaaataaataattacaaaaaatttgtttacattccacaggtaccttaatttaaaataaatcagactaacaggtggtatctcttcttagtgttctatttatcttatttgctaatgagaacaattcttcttctgttaggctgtgctttattgataaaaccaagtattgaataaagagagttaattatctttttaaagtaaatgaaattataaatatataatatatataaagtattgtgtttaataaaatgttatgcaatgttttccaaactgataaagtttgtaaagtgctataaatgtattttgttaagtacagatcaaagctatcgtgtgagtatattgtgctaacatcatagaaataaagattagatttcttcatcaaaa

SEQ ID NO: 18 - DNA, 2999 bp, Mus musculus
ttgctcttctctggcactcgaattagcatgaaaatgaaggctaacagctacctcagttccaaattcctaattctggtgccacaatttggtaagagagagaggctctccgcacttcccagagcactgctgtcattcatgacaaagaccttcaagctgcctggctgtctctggctgcagcaatcctctactgcacaggccagatagccaaaggcaactctccgcagctgacaccctcattaaacaggttctctccaggaaagcccgttgatctcagcctcctaaggctcaagcacagctggttcgtcaccaccttctcactggcacttcattcccatttctggatgcacggactgcttcacatcttaccaccttctgccttcatttggctcacaaacaccttcctaaactcatcctgagtcttccggaaagggacctgcccaggtctgacctctcacctcataacctgagagcctacagccaccctgtctttaccagccacctttgaacaccccaactgagcaagtttcaggactcaaggatccactcgaccaacgtctcggcaccccgcactactgctggccattcccaaagcaccatgacaggcttttgggtcctctgtttcgtccttttcccctcctccttatcctatccggaaagctggatgccccttgtaaacctcactcaccacatcctacgtgataccaactcttccctgttttccaactgttgggtctgcttgtctacccaaacccagcggtccttagcagtcccagcccctctgtccatttggacagatacacccatgaagcttcatcttacctactcagtcaggcccttctctggctccttttccattagcgacattgaaagacgcctccgtctcttccgcccactgactgcctcctattctttccacaatcctgacagaagggcgattgcttttcttcaactcgtcagctcaacaggcatatttcggatcatcacccggataacctctgtgatatatccccataaggaccgtttcttcgaatctgcccaacgccctctctggggaccactctttactgagaccgtgctcaggtcgcaggccccactctgcatatctcgctttttcaaggtctcagcatatgccacttttgtaggcaacctctctgcctctctctgcaactacaccatgcatatttcaccttctaccagtcatgaaaacctagatctttccaccacccatacgttcaaacaggcaatgaaaagaccggatgccaaatggaaaaacccgctccgtttttccgggcccccctccctcatcttctcgaagccggcttactatccctgcccaacagacatcaaacactgccatacctctccggccactccctggatgcactgtcctcaggctcccttcggcacctgctataacctcactttatttgaaccagacaactcaacccaccctgttaccatgtcagtgaaccctacccacttcaaggtcaaactccaggggcacagagacccctatccgctctcccattaccagcccctcacgggagctgccctgtctggacaatattcagtctgggagaacgagatcactgtccaagaaaactgggacatcacctccaacattttctcacatcttctcagcttctcgtacgccttctgcctcaactcttcaggcgttttcttcctctgcggaacatcgacttacatctgcctcccagccaattggtccggtgtctgtaccctggtcttccaatacccggatattgaacttctccccaataaccaaacggtgcctgttcccctttttgcttcagttctttcctcagactcagttcttcgcccaaagaggtcccctcacctctttcccttccttgcaggcctgggtatctcttctgcccttggtacggggatagctggcttggccacctcgactctctatttccaacagctttctaaggttctttccgaaaccttggaagaaatagctgcctctatcactaccctccagaaccaaatagactcgctcgcaggtgttgttctacaaaaccgccgagctctggacctcatcactgctgagaaagggggcacctgtctcttcctccaggaagagtgctgcttctacgtaaaccagtctggaatagtccgggacgcggcaaggaaactccaagaacgagcatctgaactcggccagcattctgactcttggggacagtggcctgaccttggacgttggttgccctggctgactccctttctgggacctcttctcttcctcttcttcctactgacatttgggtcttgtcttctgaactgcctaacccgttttgtgtcccagagacttggctcctttgttcaagacactgccaaaaggcatgtggacagcatcctccaaaatttccaatataaaaaactgccccaagactccccagatgaggacaccattcctacataacagggaaaagttgagagagcaccaagtataccctcccttctacccagttaatagaatccaagtcgggagacttagttatggcctcatgtttgtatcagggttaattccagaaactatagtaatttggcatccagaaaatttcttctttttctagatctgagctccttctgctccttctagatcttctccttatcagtagcccccatctcagacatggcctctggagttgtttcacctgacattctttaatccatcactcaccttgctgcttagctactcttagcaatggccaaagaaggaatgttttcagtgccctgcctgtaggtggagagtgggctctcttatacagagatctctgctcccaggcctgtgctgccagggagtggcttgttcctccgttgtctttcttgctttctgacctcatacgaagaacattgaggtgtcagggagcttccagaagctggaagggggccagcaaggattctctatgacagctttgagagagagagagagagagggagagagagagagagattattttttttaa

SEQ ID NO: 19 - DNA, 1947 bp, Mus musculus
aactgtcaccaaggagagagagagagcaagagagcgaatagagaggaggcgactccagctgcctttttcaacatggattcccgtgaattccggaggagaggcaaggagatggtggattatatagctgactatctggatggcattgagggtcgtccagtgtaccctgatgtggagcctggctatcttcggcccctgatccctgccactgccccccaggagccagaaacatacgaggacataatcaaagacatcgagaagataatcatgccaggggtgacacactggcacagtccctatttcttcgcttacttccccacggctagctcatacccagctatgcttgcagacatgctgtgtggtgctattggctgcattggtttctcctgggctgcaagcccagcgtgcacagagctggagaccgtgatgatggactggctggggaagatgctggagctgccagaggcctttttggctggaagagctggggaagggggaggagtgatccagggaagtgccagtgaagccaccttggtggccctactggctgctcggactaaagttatccgccagctgcaggcagcctccccagagttcacacaagctgctatcatggaaaagctggttgcttacacatctgatcaggcgcattcctctgtagaaagagctgggttaattggtggaataaagctaaaagcagtcccttcggatggcaacttttccatgagagcttctgcccttcgggaagccctggagcgggacaaggcagctggcctgattccattctttgtggtcgctacactggggaccacatcctgctgttcttttgacaatctcctggaagtgggtcccatctgcaaccaggagggtgtgtggctgcacattgacgctgcttacgcgggcagtgcctttatctgtcctgaattccggtatcttctgaatggtgtggagtttgcagattcctttaactttaatccccacaagtggcttttggtgaactttgactgctctgccatgtgggtgaagaggaggactgacttaaccggagcctttaatatggaccctgtttatctaaagcacagtcaccaggactcaggattcatcactgactacaggcactggcagatcccactggggcgacgatttcgctctttgaaaatgtggtttgtttttagaatgtacggagtcaaggggctgcaggcttacatccgaaagcacgtggagctgtctcatgagtttgagtcactggtacgccaggaccctcgctttgaaatttgcacagaagtcattcttgggttggtctgcttccggctaaagggctccaatgagttgaacgaaactctcttacaaagaataaacagcgccaaaaaaatccacttggttccatgtcgtctccgagacaagtttgtgctacgctttgctgtgtgcgctcgcactgtggagtctgcccacgtgcagctggcctgggaacacatcagtgatctagcaagcagtgtgctgagggcagagaaagaatgaaagcagagctgcttcagagatcaaaagttgaaaagaagtttatctgaaaactggaaagagaaaaataactaccactccgtcttcgtgaaatcatgattacatgtggcgtcatgtgtgtctccaacattaaccagaaacctctgactgactttttggtgacttatcaatgaagaaatattttctgtattgtccagggaaaagtattttctgtgtggaaagctattgtcagtggctctagcttctgttctttgtgtggccgtgacttctgttgataataagatgtctttgtgctcataaggtcattggtggcaggataggcttatagaaatagtttccagggcagtctttggtcttaccttcagagtatatctatggctgttaacttatcctctgtgtggctaaatactaaataaacaacctatgtgcaatact

SEQ ID NO: 20 - DNA, 2337 bp, Mus musculus
tttttttttttttttttttcgtccctggccttgcctaaactcttctgtcggtctgtaaacattacctgtgaatttcccagccgaaacggctgttggggcaagaaacttcttgttaaaacttcccaccccttggactctccacagcccctctcaccgtcccaatcttctgagacgctttttacctctccgccagagcagagtttatcttttttttctttttcttttttttttctttttcctcccatttttcctcgccctgtcctttacatctgaaaggagatcagttcaagagtgaccaggtgggacgcctccttttccttatttagtttattattgtttgggggagttttctttctatctttttttaattcctgtccggggagttttgtccaccgcctcctaccacctccccctgtaccccgctcctccgcgcggaggatggtgtggaaatggctgggcgcgctggtagtgttccctctgcaaatgatctatttggtaaccaaagcagccgtgggaatggtgttgccccccaagcttcgggacttgtcgcgggagtcagtcctcatcaccggcggtgggagaggcatcggacgccacctcgctcgggagttcgcagagcgtggcgccagaaagattgttctctgggggcggactgaaaaatgcctcaaggagacgacagaggagattcggcagatgggcacagagtgccactacttcatctgtgacgtgggcaaccgggaagaggtgtaccagatggccaaagctgtccgagagaaggtgggtgacatcaccatcctggtgaacaatgccgctgtggtccatggaaaaagcttgatggacagtgacgatgatgccctcctcaagtcccagcatgtcaacaccctgggccaattctggaccaccaaggcctttttgccacgtatgctggaactccagaacggccatattgtgtgcctcaattccgtgcttgcactgtcagccatccctggcgccatcgactactgcacgtcaaaagcatcagccttcgccttcatggagagcctgaccttggggctgttggactgtcctggtgtcagcgccaccaccgttctgccctttcacaccagcaccgagatgttccagggcatgagagtcaggtttcccaacctcttcccgccactgaagccagagacagtagcccggaggacggtagatgctgtgcaacaaaaccaggcccttctcttgctcccgtggaccatgaatatcctcattatcttgaaaagcatactcccacaagctgcactggaggagattcacaggttctcggggacttacacctgtatgaacacctttaaggggaggacatagaggcaggaggaagacacacctgaggagctatggagcctgagggggagccacagcagccgggcacacaatcctgtgcctgtgcattagcacatctgctgggtgaacaggactgttcttgtccccagggaagattttgcagctccccaggtcaactccaggacctttgtgcaagactgatgggtttaactctgacccccatggggaggcaagaagccggcagccacccaacaactttgtacatttctcattctgtagcgtttgtcatgaaattgcttctccagtctaacccgcctgatgtgcatctactatttccaggagagtctgctcccagacactctgcctttccctccaaaaccctctcactcccagctcgtgcaaactggttacacagcagaaacgcaaaataaagaggtggctttcgcagcttccttcgttcacgtgtttgggagggaggcagctgggaaggaacctgccccaaccacaaagacccatcttttgagagagaaggggtctgccttggggtctacaaagagcaggagaggaactgcagcccagtccaagaagagagacaggagggagggaggtatggccaggcccctccagatacttgtcttccctggtaggatccatggaagagttgaccgcatctgtccttctttggtcccactgggccaccaatgtaaaagtaaagtcagactccactgagcacctgcttctgaccctacatgggggaaccaagatgatactagcaaaagatgccattcatctgtggaagaaagaatcatgagtcaccagaaacaatggcaacgcatcacttagggtcagcgctgtggagtgttacagcacatctggttgtgggagacggaaaacccagagaatggaaggagctggcatggtctcagtcaagcaagggtagaggtgcccatggttctctgccgtgattctcatactgagcacattgaataaatgtcactgtagtctgtgtggagc

SEQ ID NO: 21 - DNA, 716 bp, Mus musculus
gaaaagagtcaagccgcagaggaagatgaagggcctctaccaggctgctggcaggaccctggttactctggggggcctcagcatcttctcaggagccattgccttcttccctgtcttttcctgcaagctttggtacacaggatggagcgtttggattgcctgtcccatctggaacggggctttggctgtcacagctggatcacttgtgctgctggctcacagagagtggacccagagacacttgtgggaagccgtgttcaccttcgtaattctgagcattctgggatgtccacttcatttcacagtagccttgcaatctgccctccttggtccatattgcttctactctttctcaggggttgcggggaccaattaccttggttacgtggttacctttccttttccgtacacgaagttcccgtcggtctgtgtggacccgctccactatgaagagtatcacctgacccttcaggtcctggacctgtgcctgagcctcatcctattctgtgtgtccctggcagtgttcatcaagctttctgcaagactgatgcagaccggatacataaatggtccagagaatccacaataaatttgaacctgtgttccccttaaattggccttatttgtggtcattattttgaaatatttacaaagcaattttgttctttaaacttcctaccttctgggttcttatgaaataaaatggaaatgattgt

SEQ ID NO: 22 - DNA, 3065 bp, Mus musculus
gcaagggcacagctgtgccagctcaccctggcagactcctggcagcatggcagcaaagctctggaccttcctgctgggctttgggctcagctgggtgtggccggcttctgcccaccggaagctcctggtgttgctcctggatggttttcgctcagactacatcagtgaggatgctctagcatccttgcctggcttcagagagattgtgaacagaggcgtcaaagtggattacttgactccagactttcccagcctctcctatcccaattactacaccctcatgactggccgccactgtgaggtccaccagatgatcggcaactacatgtgggatcccagaaccaacaagtcatttgacatcggggtcaaccgagacagcctgatgcccctgtggtggaacgggtcagaaccgctgtggatcactctgatgaaagccaggaggaaagtctacatgtattactggcccggctgtgaagttgagattcttggtgtcagaccaacttactgcctagaatataaaactgtcccaacagatatcaactttgcgaatgcagttagcgatgctctcgactcattaaagagtggccgagcggatctagcagccatataccatgagcgcatcgatgtagaaggtcaccactacggcccttcatcacctcagagaaaagatgccctcagagctgtggacactgtcctgaagtatatgatccagtggattcaggaccgaggcctgcagcaggacctaaacgtcatcctcttctcagaccatgggatgactgacatcttctggatggataaagtgattgagctgagcaactacatcagcctggacgacctgcagcaagtgaaagaccgagggcccgttgtgagcctgtggccagttcctggaaaacactctgagatatatcacaaactccgcacagtggaacacatgaccgtgtatgagaaagaatcaatacccaacaggttctattacaagaaaggaaaatttgtctctcctttgaccctggtggctgatgaaggatggttcatagcagagagtcgagagatgcttccattttggatgaacagcacgggcaagcgtgaaggctggcagcgaggatggcacggatatgacaacgaactcatggacatgagaggcatcttcctggccatcggacctgatttcaagtccaacttcagagctgctccaatcagatccgtggatgtctacaacatcatgtgccacgtcgcaggcatcaccccactgcccaacaacgggtcctggtccagggtggtatgcatgctgaagggccagaccagctctgctccacccaccccactgaacagctgtgcactggtcttgattctcctcttatactttgtatagctggccctatggctcattccaaagcactgttgcagtaaagcctgcttccaacatgggacagttttcattttctttatggaataatagctttattaacacaatcaaggccgttaaagttgtgaatatattattcttgggtgattctacccacaaaagtcccttctggggaaaaaaactgcaaaattcgtatactttgttttacctaaaaagtttgaaatttgcatcttctcattcacttttctacatagttctctgctttgtttatacatccttactgaagatgaacagtgagagccatgctttgcccctgcaacaggcaaacattaaacgggtatcctgtagtcatcttggaccctctcatactgccttgccctgtcaagcaggctctctgtgatctgtccacatgtaagagctcacacttggaggtctgcagccataatgttatattttctattctattccagatagagacagtctatctacaggaaagcaatatgtgttgtctggttcttccccaatgcatggtggtttttttttgttttttgttttttgtttttttaacaacacaatctcagaaagcacatagaccagtagaaatcacaaccaattgaagtatcaataacaattagacaactcatagaactggcagaggccatgacacctctcaaaatttgagaacagaatagcttctccccttcattgaatgccaagtttcaaggttctatgtaactaaaaacacaatctctcataatacaaagccttgtgagccacaggccctgaacattgacatccacatctgcctgtcggtgaccactaacttccctcagacactgctactcttttagtttttgagtcaaggcacatgtctgatatgtcagtgaaggcttggaaaggaagccttagtatcatagtatgaacatcataaatttaactctttgctaggaatagtgatatatcaaatgcaagttccatacttcagtagttcagaaggaccttgtaaaacaaggactgattgtacttttaaacaattagaagagagcctacatgtacacacacatgcacacacatttacacataaacacatatacacatgcacacacacatgtatgcacatgcacagacataaacatatacatgaatataaacacacacacacgtgtgtgaacacatgccctccacatattatggtgatacctagaatttttatcttttttatgatgaagaagcatcataaagcatcaaattcaaaagaatttgagttttgaatttgtccattttctcagctactgttatgccatgtgacactgtctcaccaggctggccagaagcagcaacaaaccggagctcccaaccagccacatgatcctggctgtgggcctcaatatcctagagcacacggtgccgctaaactatgagggtctgtagggtggtgtatgaaagtcctgttgactaaataatagtttcagttcatgataggcttatgggcatgtgacctcatcctatgttgaggagattatgaatcctgttgcctttgaaatgagttggcactaaatgggtcattaaaagtgacactgtgtcaggaaaagacaaaacatcagctctcaggaggaggaagcctttatttggtgcttgccctgaaaaaaaaacaaaaaacaaaacaaaacaaaacaaaaaaccccaaaaccgaaaacaaaac

SEQ ID NO: 23 - DNA, 2039 bp, Mus musculus
gcgggactcccgggctgtgtgcctcaggtcggaactcggggctagtgcctgtagagagaccgaagcactcggttcccccaggggggcctcagcctgggtgtgtgggggcgcaggccccggggatgctgggctcagtgaagatggaggctcatgacctggccgagtggagctactacccggaggcgggcgaggtgtattctccagtgaatcctgtgcccaccatggcccctctcaactcctacatgaccttgaacccactcagctctccctaccctcccggagggcttcaggcctccccactgcctacaggacccctggcacccccagcccccactgcgcccttggggcccaccttcccaagcttgggcactggtggcagcaccggaggcagtgcttccgggtatgtagccccagggcccgggcttgtacatggaaaagagatggcaaaggggtaccggcggccactggcccacgccaaaccaccatattcctacatctctctcataaccatggctattcagcaggctccaggcaagatgctgaccctgagtgaaatctaccaatggatcatggacctcttcccgtactaccgggagaaccagcaacgttggcagaactccatccggcattcgctgtccttcaatgactgcttcgtcaaggtggcacgctccccagacaagccaggcaaaggctcctactgggccttgcatcccagctctgggaacatgtttgagaacggctgctatctccgccggcagaagcgcttcaagctggaggagaaggcaaagaaaggaaacagcgccacatcggccagcaggaatggtactgcggggtcagccacctctgccaccactacagctgccactgcagtcacctccccggctcagccccagcctacgccatctgagcccgaggcccagagtggggatgatgtggggggtctggactgcgcctcacctccttcgtccacaccttatttcagcggcctggagctcccgggggaactaaagttggatgcgccctataacttcaaccaccctttctctatcaacaacctgatgtcagaacagacatcgacaccttccaaactggatgtggggtttgggggctacggggctgagagtggggagcctggagtctactaccagagcctctattcccgctctctgcttaatgcatcctagcagcgcaattgggaacgccatgatgggcgtgggctgcaacgttcttgggctctgatctttctggttacactttgcttgtcccattaattaacatcttatttggtctattactgtgatatgacccattggctactgtggtaactgccatggactctttggtaggcctagggttggggtattaggaaggcagatgcgtttggaagtgctgcgaaggtggtcatgttggacatattgtgaaggcagttagactggtgtactatgaaagctgccatattaagtgaagccattgggtgattgatccactgggtgcctgatggtcgtgatgttggatgacacatgtctggtcctttggatgatgtgttggacatcttgattgaccttttgagtatgtgacagaacacatcttctttggctcattttatcctgggatcgcctcttttttttcctcttctttttctttttctttttctttttttcttttccttttttcttttttttttcttttttggcagacttcttggttcagcagatgccaaattggccaccatatcacatggtgtcttttttgacattctggatgcatggaaggtcactgtattggcaaggtgacatctcagcatgctgctatgcaccaagatagatggttaccacaggcctgccatcaccatctccttggtggaggttgggtgaggggaagaggtgagcagaccctatgagttttctctgaagcccatccccaccctgtctgtgagaaagggctagtgtgggtgtcgggagttcctactgaggtcaagttcttgtctggggcttgggaatactgcctgtgtttggccattaaaaaggcaccatctccat

SEQ ID NO: 24 - DNA, 2739 bp, Mus musculus
gaaacttttcccaatccctaaaagggactttgcttctttttccgggctcggccgcgcagcctctccggaccctagctcgctgacgctgcgggctgcagttctcctggcggggccccgagagccgctgtctccttttctagcactcggaagggctggtgtcgctccacggtcgcgcgtggcgtctgtgccgccagctcagggctgccacccgccaagccgagagtgcgcggccagcggggccgcctgccgtgcacccttcaggatgccgatccgcccggtcggctgaacccgagcgccggcgtcttccgcgcgtggaccgcgaggctgccccgagtcggggctgcctgcatcgctccgtcccttcctgctctcctgctccgggcctcgctcgccgcgggccgcagtcggtgcgcgcaggcggcgaccgggcgtctgggacgcagcatgcaggcgcgttactcggtatcggaccccaacgccctgggagtggtaccctatttgagtgagcaaaactactaccgggcggccggcagctacggcggcatggccagccccatgggcgtctactccggccacccggagcagtacggcgccggcatgggccgctcctacgcgccctaccaccaccagcccgcggcgcccaaggacctggtgaagccgccctacagctatatagcgctcatcaccatggcgatccagaacgcgccagagaagaagatcactctgaacggcatctaccagttcatcatggaccgtttccccttctaccgcgagaacaagcagggctggcagaacagcatccgccacaacctgtcactcaatgagtgcttcgtgaaagtgccgcgcgacgacaagaagccgggcaagggcagctactggacgctcgacccggactcctacaacatgttcgagaatggcagcttcctgcggcggcggcggcgcttcaagaagaaggatgtgcccaaggacaaggaggagcgggcccacctcaaggagccgccctcgaccacggccaagggcgctccgacagggaccccggtagctgacgggcccaaggaggccgagaagaaagtcgtggttaagagcgaggcggcgtcccccgcgctgccggtcatcaccaaggtggagacgctgagccccgagggagcgctgcaggccagtccgcgcagcgcatcctccacgcccgcaggttccccagacggctcgctgccggagcaccacgccgcggcgcctaacgggctgcccggcttcagcgtggagaccatcatgacgctgcgcacgtcgcctccgggcggcgatctgagcccagcggccgcgcgcgccggcctggtggtgccaccgctggcactgccatacgccgcagcgccacccgccgcttacacgcagccgtgcgcgcagggcctggaggctgcgggctccgcgggctaccagtgcagtatgcgggctatgagtctgtacaccggggccgagcggcccgcgcacgtgtgcgttccgcccgcgctggacgaggctctgtcggaccacccgagcggccccggctccccgctcggcgccctcaacctcgcagcgggtcaggagggcgcgttgggggcctcgggtcaccaccaccagcatcacggccacctccacccgcaggcgccaccgcccgccccgcagccccctcccgcgccgcagcccgccacccaggccacctcctggtatctgaaccacggcggggacctgagccacctccccggccacacgtttgcaacccaacagcaaactttccccaacgtccgggagatgttcaactcgcaccggctaggactggacaactcgtccctcggggagtcccaggtgagcaatgcgagctgtcagctgccctatcgagctacgccgtccctctaccgccacgcagccccctactcttacgactgcaccaaatactgaggctgtccagtccgctccagccccaggaccgcaccggcttcgcctcctccatgggaaccttcttcgacggagccgcagaaagcgacggaaagcgcccctctctcagaaccaggagcagagagctccgtgcaactcgcaggtaacttatccgcagctcagtttgagatctcagcgagtccctctaagggggatgcagcccagcaaaacgaaatacagattttttttttaattccttcccctacccagatgctgcgcctgctccccttggggcttcatagattagcttatggaccaaaccccatagggacccctaatgacttctgtggagattctccacgggcgcaagaggtctctccggataaggtgccttctgtaaacgagtgcggatttgtaaccaggctattttgttcttgcccagagcctttaatataatatttaaagttgtgtccactggataaggtttcgtcttgcccaactgttactgccaaattgaattcaagaaacgtgtgtgggtcttttctccccacgtcaccatgataaaataggtccctccccaaactgtaggtcttttacaaaacaagaaaataatttatttttttgttgttgttggataacgaaattaagtatcggatacttttaatttaggaagtgcatggctttgtacagtagatgccatctggggtattccaaaaacacaccaaaagactttaaaatttcaatctcacctgtgtttgtcttatgtgatctcagtgttgtatttaccttaaaataaacccgtgttgtttttctgcccaaaaaaaaaaaaaaaaa

SEQ ID NO: 25 - DNA, 3105 bp, Mus musculus
ttttaaaagctctgtgctccaagttaaaaaacgcttttacgaggtatcagcacttttctttcattgggggaaaggcgtgagggaagtacccaacagcagcagactttgaaactttaaacagacaggtctgagagcccgaactctccttttcctttgacttcagcctccaaggagttccaccactttggcgtgccggcttcactttcattaagtgaaagagaggtgcccagacatgggtgactggagcgccttggggaagctgctggacaaggtccaagcctactccacggccggagggaaggtgtggctgtcggtgctcttcattttcagaatcctgctcctggggacagcggttgagtcagcttggggtgatgaacagtctgcctttcgctgtaacactcaacaacccggttgtgaaaatgtctgctatgacaagtccttccccatctctcacgtgcgcttctgggtccttcagatcatattcgtgtctgtgcccacactcctgtacttggctcacgtgttctatgtgatgagaaaggaagagaagctgaacaagaaagaagaggagctcaaagtggcgcagaccgacggggtcaacgtggagatgcacctgaagcagattgaaatcaagaagttcaagtatgggattgaagaacacggcaaggtgaagatgagaggtggcctgctgagaacctacatcatcagcatcctcttcaagtctgtcttcgaggtggccttcctgctgatccagtggtacatctatgggttcagcctgagtgcggtctacacctgcaagagagatccctgcccccaccaggtggactgcttcctctcacgtcccacggagaaaaccatcttcatcatcttcatgctggtggtgtccttggtgtctctcgctctgaatatcattgagctcttctatgtcttcttcaagggcgttaaggatcgcgtgaagggaagaagcgatccttaccacgccaccaccggcccactgagcccatccaaagactgcggatctccaaaatatgcttacttcaatggctgctcctcaccaacggccccactctcacctatgtctcctcctgggtacaagctggtcactggtgacagaaacaattcctcctgccgcaattacaacaagcaagccagcgagcaaaactgggcgaattacagcgcagagcaaaatcgaatggggcaggccggaagcaccatctccaactcccacgcccagccgtttgatttccctgacgacagccaaaatgccaaaaaagttgctgctggacacgaactccagcccttagctatcgtggatcagcgaccttccagcagagccagcagccgcgccagcagcagacctcggcctgatgacctggagatttaaacaggcttgaacatcaagctgccaatcgattgtggaggagaaaaaaaagggtgcttgcagaacgtgcacctggggtgttcatttcgttcccgtggaggtggtactcaacaacctcagtaatgaggcgtagaaaacaaagacattacaatatctaggttccttggggggtgttttgggatagctaggcggcaaaagtagggaaaggggaggtatgtaacggtatttaatgtagaagattcaaagagcttaaattctagtaagagtctcattggatgaaacatagatagggctttctctctctgccccccaactgaaccttaagaatggttctgtatacatgagtgagtgggtgatatatattttttttaatttttgttttactgagattctgccatagagctttgagcaggaatccaagtcctcaacatggcatttcctttatgaaaagacaggttgtcctacatccccgctaaaaaacattccagtgtttaaaaacttggcagtttgcaggcgagcttccctggcctgaccctctaggtgtggatggaccttatgctactatacacgattttcattcttggtaggtatcaattcgaagttcagacaaggttcaaagaaaaagattgcccatgtatttgcatctcagtgggttctttttcaaatctgtcccacctttgtgtcttccatatattatcctcagctggtcctcaccctcaccaaatgatttctatcgacatttttaaaacagtgagaaagtcttttttttttttttttttgagttagcatcagggaggcaagccatgctcaatatttaacaatcgcttctgtctatgtgtgggtgtgcaagtgtgtaagcgtgtgttttgtcattattggtacaagcagaggcagtataaactcacagatttgaatcgaattcacacagtgttcaaatttgaaccttcctcatggatctttgtggtgtgggccaacgtggtgtttacattatagaattcctgccgtgcaaaagtgtaaagcacacactttttccctaaaatattttttccacgtatcctattatggatactggttttgttaattatgatttttttttcttttttagaatgtagcagtaatagccattactgaaatgaatgatttcctttttctgaaatataatcattgatgcttgaatgatagaattttagtactgtaaacaggctttagtcattaatgtgagagacttagaagagggttgcttagagtggactatcaagtgagcctaaaggaactttgtagtaactggtaatctggtaatttttgtcctacttaactacacattaactcagaacttgtattctgagtttaacagtcttttagattgacgagcaacttggatgtttgcactaagattttctttgagatactagagggggtgaaggagttttcagcagtgcacatgtaactaatttatttgaactgtaagctaaagacacctaccagtttcttcaagtgacttaaaaaaactcatcacagatgattgaaatgtcgagttatcatgtttcctcttgcgcgccagctacacaaggagtttttggacaatgagaaactaatttgtttgacattccatgttaaactactgtcatgttcagcttcattgcatgtaatgtagacctagcccatccaatcaatgtgctcgggaaagtgttctttattcaataaaattttaatttagtataataaag

SEQ ID NO: 26 - DNA, 5205 bp, Mus musculus
aggagctgtggactctcctgctctccttaaatagtcaagccttcctcctacagctacgaggagtccacctggagcaccatctagagcaccctctggagcaccacccacggccagctcacagcttacctgcttccccccagtctcctgtcctttccttcctgggtcttctgcaagagccacaagatgaacggtctggaggcagccctaccgagtctgactgacaactcctccctggcttactctgagcaatgcggacaagagacccccctggagaacatgctcttcgcctgcttctaccttctggacttcatcctcgcttttgtgggcaatgctctggccctgtggcttttcatatgggaccacaagtcaggcactccggccaatgtcttcctcatgcacctggctgtggccgacctgtcctgcgtgttggtcctgcctacccggttggtttatcacttctctgggaatcactggccatttggggagatcccatgccgactcactggcttcctcttctatctgaacatgtatgccagcatctactttctcacctgcatcagcgctgaccggttcctggccattgtgcaccctgtcaagtccctcaagcttcgaagacctctctatgctcacctggcctgcgccttcctgtggatcgtggtggctgtggctatggccccactgctagtcagcccacagacagtgcagaccaaccacacagttgtctgcctgcaactgtaccgggagaaggcctcccatcacgccctggcatccctggctgtggcttttaccttcccattcatcaccacggtcacctgctacctgctgatcattcgcagcctgcgccagggtccccggatagagaagcacctcaagaataaagccgtccgcatgattgctatggttctggccatcttcctgatttgttttgtgccctaccacatccaccgttcagtctatgtgcttcactaccgtggtggtgggacttcgtgcgctgctcagcgtgccctggccctggggaaccggatcacctcctgcctcaccagcctcaacggggccctggatcctgtcatgtacttctttgtggctgagaagttccgccacgccttgtgcaacttgctctgcagcaaacggctcacaggtccacctcccagcttcgaagggaaaaccaacgagagctccctgagtgcccgatccgagctgtgagcctctgggaggtcctacaccaggccagctgtagactggtgcaggaagaccagctatcaactggggcacatgctaccagagccagctaaagaagtctatcttccttcactattcctgagcaaacaaacggaaacatcgggagttctcaccctgcttcaaggcctcaactgcaaggccatccagtctcagcgaatccatcaagaggcaggactaaccacagggatgccctgcccacccctccacaggactgggttggcctggcttctgtacagctcccagacactcagtgacttcactcgtgctaaatagggaagagagccacagggacatttctggaacaatgggaatctttcttctctaataaatttctagcttctttcatactacagatgcccacagaaacaaagccctacagaataacccagaaagcaagctgcccaaggtccgggagaagaggcagcacaaatgtcaatggaactagatggatatttaatatttcctttgaagtgtatggtatatcgaatattgcttaagatgcctttgcctcataatctctgcctgagtttaggggacacagactctagtgatagctacatgtgagtataattcagactggttgcttgtgaacccagcgaatatagttctgggctcagctcctcctgttgacagagggggcaaagacccagacaggaaggttagtctcgtgagaagctggagggtctctggaagtcagcacgccatgtccccacaccagcctcaagcctggcactgttcagagcttgtcattagaatgtggcctagtcaggcatatggcatggacggacagacattagagcccaggcagagagcctgatgcgagaacttgtctccaactgcgcccagaagggacatcctgagccccactccctcccaagaggcttctgccaccctggctgcctggctctgagctgttgaatgtgccagcatgtggtgaccgctctaatgtacttgatagcaatactcttaaacataactgagtcttaagatgaaggaaattatcatgctggtccaacatgacacatgatgttctccccctgaaatcctcgtcctttccacccaagaatgaccaagtagggcaacatcttcctggttgtggttctggtggccttgtgacatgtctgtccaggcatgtgcgttaagggagctctcaaacggatccctccagctgcatcctgcctttcaccagagaaaccctaagatggccctgatgctcagcactgcttctatctggtgctgatctgtccacccccatggccacaacaacctgcggagtacaagactgtgccagccaggaccagcacgggaccatgtgtctcttcgacagaagagcaaagggacaagagtaccgcagtctgtgacaggagggacagagggggcaaagacccagacaggaaggacagtctcctgggaaactggagggtctctggaaattagcactgcctttcctgacacaggggttcagaagcagacttgggtcagaggaggagaaatttgcagctcgatttgggacctgttacattagaatcttaaacagagatgccacactggtaggagacttgagcaaaggagagatctggaggggatacctggattgtccagatgtatcattgagaggcccaagacttcatcctcaaaggctgacatggatatggggcccaaagaacatctctgtgccaaggaagaagctgagggaagctagcacaacacagaaatctcactgaaagaggagggctctgctgtgagggcactcggaggtcaaggggaggacctggtgatacccaggcttgacctgagtcgttgagaagctgtgctggggagggcttcggggcacaaaggcgttgctgggtgctccccccaaagctttcccacccaggagggagataagctcacggctgctggggaggtatagagcccacagtgagggaggcaggggtaagaagaaagagtaaggccttctggaaggggaactggcagagtaccagtgaggcttacagtcctgaatttggagtaaattgggcaacctggtccagccatgtgaatgctgggaggaaggcagacagatggggtcagcgtggtgcagcagtggtgggtgggatgtcatgagtaattgggattcgccaacttaaaactcctcatctattcattgccacttaaagcccttcaagccttctgtcccccaaagaccaccctatgcccagagggagctgtctgtccttactctcctgcctccacccttatccatcccctgggggctgtgttctccatctagcaggggtaggacaaagcccacaaccagatcagttgagatgccacagagcctactgggcaggaccacagctcttagagtagggacaaggggtcctcctctctgcccatgactgcagctggctcacctgcctgcgttcttcacaaagcccaggtcagccagctccacatcacacaattcacagcggaagcagccaggatgccagttggcgttcatggccttgatcacgcggccaatgacaaattcacctgcaagaggagagacctcagctgtcagggcgcacaccccacccttcagctcccacggtctttgtaaccagtgttcttccgggtccacccttgtccaagtacccctcttttaggggcctgaagacaatgctcaggaagccaggaaaagtccccaggctaaggaacttcagtactggctgttaccccacttgtccccgtcctgcttgctaagtgctgtcaccagctcttaccacagaatccgcagcatggagcaaatagcatttggaagtcatgttcacagtacttccggccctcaaactgcaaatgggggccacagaagaaaagatacatgagtcattcaacttctctgtaagtcttggccccccaataaatagcaggggtctgaggtcctgagaaggggggcttccaaagctgtagtgggggtctagcccatgtgacttagcagagtgcaaagtggggggttcacagagatactgagggtccgagacagcttttggggattctgctgtctgctgagggagaggggacctgtggatcctgacaacaggccacgcagaggtgggagccggggctgggaaaggatgggtcaaatcttgagttatctgactggctttgtcctctgtgtctgaccagctcagtccctccagagactacaaaagacccaaagagggaagacggaagacagaattcctcagggaggggaattgggagtgacagcagcgcttcagtcagccaccatctgaagcttgcttttccctcccttaaagatttgctttcttaagcaaagtctgggcggtagctcagtttgtagagtgcttgcctaccattcacagaagtcctggttgggttcacagaactgtgtgaatagggtacagcagtgcacatctgtaacctcagcaccggaggatggaggcaggatgcgccggaagttcgtcatctttgataacttccttcccctaaagccacatgcagctctccactgtgagccagaggggagggggcaacatctcccaaacacctcctgtgctaggcttggttgtgctctaaaagccctcgcagagctctgcccaaccctctgatgcctcccagccagcatctctcaggagcctggaacgtgacaggagatcctcatctaactccatttctgcatcaatcaatcaatgatgcaaacagctcacaggagagcccagtccccttgagctctggtcccccccctccactgcagcccagtggaatggcagccagtgtttaaccagtccttgcctctgggtctgccagatgctggggtgtaaatcttactgctagactgatgtcaccacacagacaaataatctctgatgcataaacgtgaaagtctaacaaataatgggaagttggatgtcttatatacttgttatcattttaataaagtattatatgtaaaaaaaaaaaaaaaaaaaaaaaaaaaaa

SEQ ID NO: 27 - DNA, 1383 bp, Mus musculus
tggccgtgcatgcgactcttcgttccctaccgttttttttttttttttttttttaggttggaaatcccagctgttaagggcctagtccaaggcactagggtgccacctacgcgccgatgccccgagtgttcacagggcttccagctaactatgctgcacctaccttggcgctgtccttgctactaccattattgctggtggtgtggacccagctacccgttagcgcgaggccgtccacaggccccgattacctgcggcgaggctggctgcggctgctagccgagggcgagggttgtgctccctgccggccagaagagtgcgctgcgccgcggggatgcctagcaggccgggtgcgggatgcgtgcggctgctgctgggaatgcgcaaacctggagggccagctctgcgatctggaccccagcgctaacttctacgggcgctgcggcgagcagctcgagtgcaggctggacgcgggcggtgacctgagtcgaggagaggtgccggagccgctgtgtgtctgccgctcgcagcgcccgctctgtggttcggacggccgaacctacgcgcagatctgtcgcctgcaggaggccgcccgcgctcggctggacgctaacctcactgtggtgcatccggggccctgcgaatcggagccccagatcctgtcgcagcctcacaatatttggaatgtgaccggacaggatgtgatctttggctgtgaggtgtttgcctaccccatggcctcgattgagtggaggaaagatggcttggacatccagctgccgggggatgaccctcatatctctgtgcagtttaggggtggacctcagaagtttgaggtgactggctggctacagattcaagctttgcgccctagtgatgagggcacctaccgttgccttgcccggaatgctctgggccaagcggaagcttctgcaaccctcacagtgctcacaccagagcagctgaacgccacgggattctcccagctgcaatcacggagtttgtttcctgaggaggaggaggaggcagaaagtgaagagttgggcgattactactaggtccagatctctgctttgcaggtgtgggcatgtggacagagccctgcatccttgctgtctagaaagcccggagaagactggaaaaggcgagcagggtccttacatggattgtttaatgctcagtgtagcctcagctcatctttcctcaaaactcatctttcagaagcgtccgcggtagagatgagcgcagcggaactgatttagttcagtacagggagaggggtggggtagctccgtgtcttatacactctaggggacaaaacccacctagcatacgactagacacagtgggcaccaataaaaaaatatataaacaaaccgaaaa

SEQ ID NO: 28 - DNA, 4779 bp, Mus musculus
aagactgggatggatactggagaaggaatgcaggcttaacaagtgatcgctgctgtctaggattttgagtctttttcggagaaccttgacttccgttcccagcccatgtctgctgtgccgaactccagaggaaccagaaatctccggggtctaccttggggcgtccccaatctccacctctgggctccagtaacgaggactctgcaataccccctagccccctggccaagacaaccgaacttgttccgtggatatttgggatcctccacctgccaaacctgagcgattttttttgtactgcgcccccacccccaatgattctgcccctcctccagctgttgcagcgtggaaaaggggaaacaaatcaccgggggggatttttttcgtctatttttatttttcgcacttgctgggaatggtgaagtgcttcttgtgaatgattctagccaaaggatgctcttcatttcctgctttctatggagacctcagtgttgagtttgcctctgctggaactgcgtctaccaccttctctaccttccaaggtctttgcctctatctacaacctggcattgtctgtgggtccatgaaggcttggatgaccttagagggaaggtctgggagtccaccctcatagactaagcagcaatggctgggcatattttaagccgcattttaacatgggtcaagccatcagtagaaggcaagtgctaagactaaagacttatttgaattttatttaaattagatggactgggccttggccaatttccatgcaagaaaaagtatatttcattttctaggcacaacttctgagtgtcagatacttgctgtctttgagtcttgtggcgtcatcaccggacagcatcccagacagacttccagatttgaacatctaccccccaacacgtaggtgtatgggagaccacatcatttcatgacttatgtttgaggaacactaggctgttgtctagacgaggcaagctctggaaagcaacgccgagtctctgagaagagggagcataggctgtgctgatttaaaaacagaaaatgcaaagttggactgaaaatatcccacgtcttctaagcaatctgcttaaggcttccaaacttaccttaatttggtaagaaaataagctgccctatttttctttcttcttctcttacaactggaagcagccatttccccaaaccaccaccatggaagtggcgatggtgagtgccgagagctcagggtgcaacagccatatgccttatggttatgcagcccaggccagggcccgagagcgggagagactcgctcactccagggcagctgcagctgctgctgtcgcagctgccacggctgctgtcgaaggcactgggggttctggtggaggcccccaccaccatcatcagacacgtggggcctactcctcccatgatcctcaaggtagccgtggaagtagaaggaggaggcgtcagcgaactgagaagaagaaactccaccacaggcagagcagttttcctcattgctcagacctgatgcccagtggctctgaagagaagatcctgagggagctaagcgaggaagaggaagacgaggaggaggaagaggaggaggaggaggagggaaggttttactatagtgaagaagaccatggggatgggtgttcgtacacagacctgctgccacaggatgatgggggtggtggcggctacagttcagtccgctatagtgactgctgtgaacgtgtggtgataaatgtgtctggtctacgcttcgaaacccaaatgaaaactttggcccagtttccagaaactctgttgggagaccctgagaagaggactcagtacttcgaccctttgcgcaatgagtatttttttgatcggaaccgacccagctttgatgccattttgtattattaccagtcaggaggccgcctgaagagaccagtcaatgtcccctttgatatcttcaccgaggaggtgaagttctatcagttgggagaggaagccctgctcaagttccgggaggatgagggctttgtgagagaagaggaggacagggctctgccagaaaatgaatttaaaaaacagatttggcttctctttgaatatccagagagttctagccctgccaggggtatagccattgtgtctgtcctggtcatcttaatctctattgtcatattttgcctggaaaccttgccggagttcagggatgatagggaccttattatggccctcagtgcaggcgggcacagcagattgctgaatgacacctcggcaccccacctggagaactcagggcacacaatattcaatgaccctttcttcatcgtggagacagtgtgtattgtgtggttttcctttgagtttgtggttcgatgctttgcttgtcccagccaagcactcttcttcaaaaacatcatgaacatcattgatatcgtctccattttgccttacttcatcactctgggcactgacctggcccaacagcaggggggtggcaatggccagcagcagcaggccatgtcctttgccatccttaggatcattcgtctggtccgagtattccggatcttcaagctctccagacactccaaaggcctgcagatcctgggccacaccctaagagccagcatgcgggaactgggccttcttatctttttcctcttcatcggggttatcctcttttccagcgctgtgtattttgcagaggcggatgaacccactacccatttccaaagcattccagatgcgttttggtgggctgtggtaaccatgacaactgtgggctatggggacatgaagcccatcacagtcgggggaaagattgtggggtccctgtgtgccattgcgggtgtcttaaccattgctttgcctgtgccggtgattgtgtctaactttaactatttctaccacagagagactgaaaatgaagaacagacccagctgacacaaaacgcagtcagttgtccatacctaccttctaatttgctcaagaaatttcggagctccacttcttcttccctgggggacaagtcagagtatctagagatggaagaaggggtcaaggaatcattatgtggaaaggaggagaagtgtcagggaaagggagatgagagcgagacagataaaaacaactgttctaatgcaaaggctgtggagactgatgtgtgaattgttctctccacctgccactgtccccccatctccaaatatattcatacatagagaatgcagttatgaaaatgagatatgcaaacgattgcactgcatacagtgatatgctgtttaatggtaatacatggcataattgtgactaaacgtgtattgcatatcaaataaatgatacatcttggagaagagggaggcattaaaaacagcagatctatctttatattttttaatagaatgcaagaattttgcacataatgggaaaaatgttaatagtaaaggtggttctgaggagagtgagtgtgtgtgtgtgagagagcgagagagtgtgtgtacctgggtatgtaagtaaattgtcaacactgttgggaattgtgccgtgatggaaaaagttggcattctgaagtatttactatgtaagaactaatgaatttgagcagtcttttaccagtgttttaataacatctcctatgtctttggattctgtagttgttttttagaaattataagaattactgtgtagaaaaaagagaaagtaaattatttaatagaatataggtcacaatttaatcttggatttaattaaagtttatttttaactggaaattaacttttgaaaaggctgcaggggcctttagaaattgattatattttattattaattttggggagatataatagcaaatgcctaacattctggaggaaatgtaacaagttttgttcacaggtcttaaaactggatttttttttcttttgcactactttctatgccgaagcccgagagagacttcatactatgaatgtttactaatgcaccaatcagttcaatgacaatcattggaagaatgggttcttcgtctcatttattgttctcttcctttcgtgagactaatggccacacaaataacagcacatgattcctgctttaaaatccgaacaactcatctacaaagggactatgaagtaacgttcagcagccgaatcttttgaaattggtttgttacgatgatgcttcagaaaccatactattttcaatactcttctgccttttaagtccagaataatttaaccaaagttattgcatgcacagagagaattcttggagaaataaggcacccagcagctacagcaattatggcttaagatctttttattaaatatgcaacaaaatgctagatttaagtcccttttgtgtatgtgtgtgtgtgtctgtgtgtgtctgtgtgtgcagctgcaaaaacaaaacagaacacggaatgtggggattcgcctcaaatggtaaagcactctgctaaatcagagttagggaagaatggttttagagcatttggtttcccaatggtcattgtaaatcattatcatctttatcacaggggcttcctggggattatacttccatttacctcacaatagctattcttgagtttggagtaggaacaaggaatgcaccacgaacaactgtagcaccataaaatacagctcagaacactcagtataaacacataggaggcattactaaacaattcattaaaacaaaacaaaatcttctttataaatggttgccctgagcattgggaagaaatcccatgtccactatttcagtccatagcaaacgtgagc

SEQ ID NO: 29 - DNA, 2308 bp, Mus musculus
gccgcgcgcgcgccgcccacgcgcgatcgtcgctatcgagggctgccggctggcctggcctcgcgacacggagaccctgccaaccatggcccagctcggcgaacagactctgcctgggcccgagaccacggtgcagatccgtgtcgccatccaggaggctgaggatctggaggatctggaggaggaggacgaggggacctcggcgcgggcagcgggggacccagcccggtacctcagtcccggctggggcagcgccagcgaggaggagccgagccgcgggcacagtagtgccacgacaagtgggggcgagaacgatcgcgaggacctggagcctgagtggaggcccccggacgaggagctcatcaggaagctggtggatcagattgagttctacttttcggacgagaacctggagaaggacgccttcctgctgaagcacgtgcggaggaacaagctgggctacgtgagcgtcaagctgctcacctccttcaagaaggtgaaacacctcacccgggactggaggaccacagcacacgccttgaagtattcagtcaccctggagttgaacgaggaccaccggaaggttaggaggaccacccctgtgccactgttccccaatgagaacctccccagcaagatgctgctggtctatgacctacacctgtcccctaagctctgggccctggccacaccccagaagaacggaagggtgcaggagaaggtgatggagcatctgctcaagctctttgggacttttggcgtcatctcatcggtgcggatcctaaaacctgggagagagctgccccctgacatccggaggatcagcagccgctacagccaggtggggacccaagagtgcgccattgtggagttcgaggaggtggacgcggccattaaagcccatgaattcatggtcactgaatctcagagcaaagagaacatgaaggctgttctgattgggatgaagccgcccaaaaagaaacccctcaaagataagaaccatgacgatgaggccacagcaggtacccacctaagcagatccctgaacaagagagtggaggaacttcagtacatgggggatgagtcttccgccaacagctcctctgaccctgagagcaatcccacctctcccatggccggccggcggcacgcggccagcaacaagctcagcccttcgggccaccagaatatttttctgagccccaatgcctccccgtgctcaagcccatggagcagccccttggcacagcgcaagggtgtctccagaaaatccccgctggctgaagaaggtagactgaacttcagcaccagccctgagatcttccgaaagtgcatggattattcttccgacagcagcatcactccctcgggcagcccctgggttcgcagacgacgccaggctgagatggggactcaggagaaaagtccaggggcgagtcccctgctgtctcggaggatgcagaccgcagatgggttacctgtgggggtgctgaggctgcccagaggccccgacaacaccaggggcttccacggtggacatgagagaggcagagcctgtgtataatgccttctattttttaataccagctccatcggaaaccgtctttgttttcgagatcctcactaatagctagcatgacagagaatggagttcagtccccttagaaagcttttgtatccatgtagacctcttaatttatatatttgtaaggtatacaaactgtctggtgggccatgggtttaggatcgtcttctggctggggctgttgctctcagcaaggccactgttctgtcaatgcttggcatgtgttagtgtggtggctctgaagggctgtgggacagaggatctctggaaagatctagtagtgtcggaccgtttttttcttacaatgactgagctgtctttggcaggccgcgcaagggctcctcttaagacctcaaaggagatgtgctttatggtaaatcctacagtcaatagcatggtgtctcataggactgagtgtgtctgttccctgtcaagtgaataaataataaaacacccactggaagtcctttatctgaggtcacagagatgccttttcaagacagaacccaacggggacatgttccctctctctagctcacggtgtccctgcagggcagtcccggactctcctggaggcttctggcagcctgggaggtctgtcatctctaggagtctcgctgaacaggcagcttagacagttcctgctgtctccacagcgttccagactaacagtgtttgacatcacctgagaacttgcaagaaataaaagtttctaggccccag

SEQ ID NO: 30 - DNA, 590 bp, Mus musculus
gagcacaaacctctaggagcaactgggagagctgtggccaggagctgtcaccatgtcggagaaatttgaggtcaaagacctgaacatgaaaccagggatgtccctgaagattaaaggcaagatccacaatgatgtggaccgcttcctcattaacctggtccaggggaaagaaaccctcaacctgcattttaaccctcgcttcgatgaatccaccattgtctgtaacaccagtgaaggtggccgctggggacaagagcaacgagaaaatcacatgtgcttcagtccagggtcagaggtcaagatcaccatcaccttccaagataaagacttcaaggtgacgttgcctgacggacaccagctgaccttccccaacaggctgggccacaaccaactgcactacttgagcatgggtgggctccagatctcctccttcaaactggagtgagcggcacctcagaagaccttagcccccagaaccagcttcagccagccctccagcaagcccccaccagaaccacagcctttagggtctgtgcttttccccaaggtcaaggtcaaataaaagaatccacatttattatttccacagc

SEQ ID NO: 31 - DNA, 614 bp, Mus musculus
atgtaggagcctctcccctctgctgctgctgctgcgccagacacagaggcagactcacaggacacccgagacaccatgaagagcctgctccctctggccatcctggctgcgctggccgtggcaaccctgtgctacgaatctcacgaaagcatggagtcctatgaaatcagtcccttcatcaacaggagaaatgccaacacctttatgtcccctcagcagaggtggcgagctaaagcccaaaagagagtccaggaacgcaacaagcctgcctacgagatcaacagagaggcctgcgatgactacaagctgtgtgagcgctacgccatggtctacggctacaacgctgcctacaaccgctacttcaggcagcgccgaggagccaaatattagcgcgaagaaacagtcatttggttgtggagtttcgttttatatctcctgcagtagcattactgaagtatacagacacgcatgtgttgcttgctccttacatgatctcctagctggctggcccactccttccttctgcgggttgaaagtaatgaaagaacagtattaagaagtgtgtttatatataataaaattctggtttgatacgttcaaaaaaaaaaaaaaaa

SEQ ID NO: 32 - DNA, 583 bp, Mus musculus
gcggagacaggatcgagaacacaggtttccttgatattcagcctggaaggagggcaggaggagcccagagacctcgttcttcacttggtcattctcagtccatgatggtgtggtccccagtgctccttggcatcgtcgtcttgtctgttttttcagggcctagcagggctgatcgagctatgcccaagctggctgactggaagctgtgtgcggacgaggaatgcagccatcctatctccatggctgtggccctccaggactacgtggcccctgattgccgcttcttgactatatataggggccaagtggtgtatgtcttctccaagttgaagggccgtgggcgccttttctggggaggcagtgttcagggaggttactatggagacctggcagcccgcctgggctatttccccagtagcattgtccgggaggacctgactctgaaacctggcaaaattgatatgaagaccgatcaatgggatttctactgccagtgagctcagcctaccgctatccctgcagttacccttccggctctatgcaaatacagcagccaatggcaaactaaaaaaaaaaaaaaaaa

SEQ ID NO: 33 - DNA, 822 bp, Mus musculus
aaactgcagacactcggagggtggcgagtggccccagggcagcaagatggagtcagaccgagaaaccatccaccttcaacacaggcactccatgcgcggagggaaccagcgcatagacctgaacttttatgccaccaagaagagtgtggcagagagcatgctagacgtggcgctctttatgtccaatgccatgcggctgaaatcagtgctgcagcaagggccattcgcggagtactacaccaccctagtcaccctcatcattgtctctctgctcctgcaagtggtcattagcctccttcttgtgttcatcgccatcctgaacctgaatgaggtagagaaccagaggcatttaaataagctcaacaatgctgccaccatcttggtcttcataaccgtggtcatcaacatttttatcactgctttcggagcacaccatgcagcctccatggctgccaggacctccagcaatcctatttgatgactacctaggtcccaggagctgggtctagagccacttcagcctttgtccctgacttgtcaggataactagcatttcccacagcctccaggagagcttcaagggctacgaagaaacccctgcctcttgtccacagcaccagaattaaagtgggcctctttcctgggtgacgtaattgcactttggtctggaggcccgagctgcttccagcagcagtaactcggtctgttaaggcagctcctgcacagcctgcacactctgcactgccttcttttcctgtgctccaggcctcaatgttccctttctgcaaaatggaatctatctataaagatatctgaaaattc

SEQ ID NO: 34 - DNA, 6002 bp, Mus musculus
aaacattggatttaaacctgctcagaattcagcacagaggaagcagcctcggtagcagcagcagtagcagcagcaccagcaggagctagccgggccgccgcgcaccacagcctcgagatgtaccatcccgcctactggatcgtcttctcggccaccactgccctgctcttcatcccaggagtgccggtgcgcagcggagatgccacctttcccaaagctatggacaacgtgacggtccggcagggggagagcgccaccctcaggtgtaccatagatgatcgggtcacccgggtagcctggctaaaccgcagcacaatcctctatgctgggaatgacaagtggtccatagaccctcgagtgatcattttggtcaacacgcctacccaatacagtatcatgatccagaatgtggatgtatacgatgaaggtccatacacctgctctgtgcagacagacaatcaccccaaaacctcccgggtccatctcatagtgcaagttcctccccagataatgaacatctcgtcagacattaccgtgaatgaaggaagcagtgtgaccctgttatgtcttgcaattggcagaccagaaccaacggtgacatggaggcacctgtcagtcaagggccagggatttgtgagtgaagatgaatacctggaaatctcagacatcaaacgtgaccagtctggggagtacgagtgcagcgccttgaatgacgtcgctgcacctgatgttcggaaagtaaaaatcactgtaaactaccctccctatatatctaaagccaagaacactggtgtttcagtgggtcagaagggcatcttgagctgcgaagcctctgctgtcccgatggctgaattccagtggttcaaagaagataccaggttagccactggcctggatggggtgagaattgagaacaaaggccgcatatccactttgactttcttcaatgtctcagagaaggattatgggaactatacctgtgtggccacaaacaagcttgggaacaccaatgccagcatcaccctgtatgggcctggagcagtgattgatggtgtaaactcggcctccagagcactggcttgtctctggctctcagggaccttctttgcccacttcttcatcaagttttgataagaaaccataggtcctctgagcatcgcctgcttctccatatcacagactttaatctacactgcggagggcaaaccagtttgggcttctttttgtttatttttcgttcttcttgactattttggtttttggtttgatttcttggattttcaattttatttgatttttctttttttttcttctttttcttcttcttcttttttttttttttttttttgaatgagtggggttgggatgggcagggttctaccaagggtagggtaatcattcattggtatgcccccaaacggaatctattcctgctaccttggtcttccttttctctacttctcttcttaccaccattaacacacacacacacacacacacacacacacacaccctaaaaataaaaatgggctaaaaaatgtcccatgataagtaccctgatggtacaccttggctcacaatgcagtacacaataagagttgcatctacatgtcctattttctttgtcctttaagctttcaataagacagttttaaaagtgcatatccttatccccatgctaatagcacctatcccattaggcttcacatcttgtctttctaagaagctgcctaactgcatccttaaatgtgtacacacatacaaatatatgtaaaaaatttccatcttcactggccattctcctctatatgctttttgccactagctgtaagacttacagaattgagactatatatgtacccaacgctacaaatttaggagtcaagtaaacaaatgagggaagtctatttaggatagtacttcccttaaaacgctgttgcaactcataaaaaactgatcaatagctggctaattatattaagctttcaaagcaatcatactattatccatttactcaattgatttgtggctccatctagttctacataacctgtctttttctcttataatctatttgatctatttaactaatcattctttttctcttcccactacacactatcaattcatcccatattaatctctaatcatattgtgtctatgccactatctccatatctctactaccatcaatagacatttccaccttcaaaattgcctagcaacttcttatgtgaaagccagtggtcttgcaggctaactacccagaagaacaatttcctatgccatggatccttgagaatgcagtaacccatccacccaaattagaccttgtgaacagatggaccaaagtagcaatctaaagatcagtcactcatgattttcagagaggctgttccctaagccaccttctcaggaggcaggtcagccctgggaaagccttgattatgctgcatttctcctttaacagctggaaaattaaggtaccaaccccgtgcttctctcagcctttcaagaaaagtacatgtcaggaacttggggaaacttcttcgtggctggctttcattagcagaaagaacctgacctccctccaccacacccccccccccccagaaaagcatatatctttcccttcaatgtaaagacagtggtccatcagccaaagcgtgataaccagagctcagcatctccctactgctccagttttgatttgaattgtttgaaaattatccagccaagggctgatggaggccaattacgtggcgtgtgtgttgacaactctggtatttgtttcagaaagctcttctgagctgagggcacttgagctactgacttaatttccaagcacttgattaacacaacatggcaaacagaggggaagtgtaagtaatactgttttcctgggctgtttctctcgaagctttggggataaatgtccccaaattcctatgttcaaagcaggcctcctgtacaaagaagatctgattccacctctcagcattgactcttgggaaaagaagagcccagccaacaagagagcctacagttcaaacatcattaaaggtcacaaggggctctggaggaactggcttactgagaagttgagaaggagcaccagcagtgattttcttaaagacatgtcctgcctccctcagggacctttttcaggtccacctctaaaaactaaatgactgtgggctccattagagttcatttcccttagagcttaactgatcacttgttacagtataactgcccactctgtggcactatggccacagaagtacaaaacccacagtagggacatatatacctgtgtatacatattcacatatatacaaatgcaggcttctttggcatacaaaaagctcagcactgtcataagagatagagctggggtgagcaaaattaatactcttcttgccagctgtaaacagacatctgcatttcctagtgagctgccaagaatgagactctgggagcataaatgatgtgccaaaaacagtctatttattccaacttccagaagcaatcaggagcaggcacaaagtcagaattacaggtcggatatatgaatttcagatctgtcaccacgctcagcacctgggattgggttggtgtcaagcattgtatgagacataaacatggcatttgcgttttctactaatactacttttgttgctttggagaaaaggaaatgtggatgccatggaaactgaaaagtgttgggtgatgtgctgcttgaaatggacaagcccaaattggtcttcactaccttgtgcaagcagatcatcaaaaccccagttgagtatggactcagggtcatcctgcatctctgatgtgcctactttcactggatactagaaatttctgtggtggtaacctactccagccaaacttccgaaggaaacacatgctgcagtcaatcctcctctgtctgggagaaaaatcaaaagccaagtagaaatgactaaaggcaactgagtaccagatataaaagtgacttcctcacagcaaatgtgccccagctcttgttatttgagtgactttggtaaggatgactccatgtgatgggagctgacaggacagcagctgcttcctaaaaacaacatgtatacttacacatgcctttccaaccatatacatgaagggtggacaatcaggcccactagacagatttgtttagcttgccaagggtgggtgtgaggaaggtataaggttaggaaaaaggaatcacattagtctaagtagctggtaaaatgttgtgggttttctaagtgaaacccaaatacaggttatcaagagattaggtaaaagaaagtcagcaaaggtagttattctaaaacttctccccttcccaaaccaatgctaatttgttcttccaccaaagtatgccaatgaaagtgctagtgttctgcctctggcaaagcctgttttttgaatagtttaatgtcaagtgcctgatacagtcatctgcaagtttaatcaagagtgtttggattttctttttttgttctcattggttaggttggagacatagtagattagttgtcaaaacatatacagctctgacacagagagctaggtatgtggctcttctgctgtgggcgaagctgtgttcaacagatggaaatggacatctgtatgtcaccaagatggctcatgcctgtccttacactgctttgggctgttgttcacaggttgggaagttagttttcaaaatatggtcataggtttggtttggaattctaggaccttcataactgaggctgcattttaatgatctctttctatatccatctggtcacattgtcctcagcaagaaggaatagcaaacctgcctacaataggaaaaatatcaaaagagcagagccccaccttccccaagtggacactggatcccagagagtttatcacaggcactggataaagaaaagttggaaatttaatacaaatgatttgattgatacttccagggacaaagaacacatgctcttcgtagcctatatggctaataccagcctcataagacagtgggggaggaatagcttatgaatatttgagggaatatctgcccccaatttgatcctgagtttttaccaggatcaaaaaaaaaaattgtttgacaatacccagaatacccttttcccctccagagctactcttgttggaattagaggaagcaacataattcttatttttaattttttggggcaaaatgtatttttccccaacagcaagaatatttgggttttctttggcataaaccttatttctagaaatcctcatgtccaattgctttcccccttattatccaaagcttcagtctctctttctttcaagacaaatatgtttaagtagctgcagtcacacctcacaggtgttcaaagagaagggcaactctatacaggataccatatgattagagcttctaatgaccacacagaaggcaaacaaataaaaatgccaagctcatttccctcatttcttactcaagagaagagaagtaaatgaaaggaaggagaactagatttgcaattacaaatgtggcaaaaagatacagcagaaccggtgcaatttagccttcaggtgcagaatatggaggccaaaagaatgtggagtggacttaattagatgcaattgtcttcatagtgaaagtagtcagctaaacccaatctccagcattttggaagagacttcctgcccctcctccccggagtggtgccctctttaggcacatggttgttccacaccactaggtggagaaggaaagattgagagctactcacaatccttgtggagctccattctaggttatttggcagagcataagaatctcaataataacagtggtaagtaatagctgcccttgtgttagtgaagaggaacatttttaatcattcagaaattttcgtgacatgtaaagtgcaattgtgaggaatgtgtgggtgtacgaaaatgtatctgtcaagttcagagtcctctagattaaaaaaaaaaaa

SEQ ID NO: 35 - DNA, 3635 bp, Mus musculus
attcccattcacacccacctcacagaccttcacagactctgcagctcattcattcaccccaatggccagcaaagccagtttgtaaccgagtattctcaacatcagatatcatgtcttggaggaagttacctaaactctgaagaattatcatgtctgcaaatttcaaaatgaaccataaaagagaccagcaaaaatccaccaatgtggtctaccaggcccatcatgtgagcaggaacaagagaggacaagtggttggaaccaggggaggattccgaggatgtaccgtgtggctaacaggtctctctggtgctgggaaaacaaccataagctttgctttggaagagtaccttgtatctcacgccatcccatgttactctctggatggggacaatgtccgtcatggccttaataagaacctgggattctctgcgggggaccgagaagagaatatccgccggatcgcggaggtggccaggctctttgccgacgccggcctggtttgcatcaccagctttatctctccttttgcaaaggatcgtgagaatgcccgaaaaatccacgaatcagcaggactcccgttctttgagatctttgtagatgcgcctttaaatatctgtgaaagccgagacgtaaaaggactctacaaacgagcccgagcaggagagattaaagggtttacaggcatcgattctgactatgagaaacctgaaactccagagtgtgtgctgaagaccaacctgtcttcagtaagcgactgtgtgcaacaggtggtggaacttttgcaggagcagaacattgtaccccacaccaccatcaaaggcatccacgaactctttgtgccagaaaacaaagtcgatcaaatccgagctgaggcagagactctcccatcactaccaattaccaagctggatctgcagtgggtgcagattctgagtgaaggctgggccactcccctcaaaggctttatgcgggagaaggagtacttgcaaactctacacttcgacactctactggacggcgtggttccccgtgatggagtcatcaacatgagtattcccattgtattgcccgtttctgcggatgacaaggcacggctcgaagggtgcagcaaatttgccttgatgtacgaaggtcggagggtcgctctattacaggaccctgaattctatgagcataggaaagaggagcgctgttctcgtgtgtggggaacagccactgcaaagcacccccatatcaaaatggtgatggaaagtggggactggcttgttggtggagacctacaggtgctagagagaataaggtgggacgatgggctggaccaataccgccttacgcctctggagctcaaacagaagtgtaaagacatgaatgctgatgccgtgtttgcattccagttgcgcaatcctgtccacaatggtcatgccctcctgatgcaggacacccgccgcaggctcctggagaggggttacaagcacccagtcctcctgctccaccctcttgggggctggaccaaggacgatgacgtacctctggaatggaggatgaaacagcatgcagctgtactggaggaaagggtcctggatcccaagtcaactattgttgccatctttccatctcctatgttatatgctggtcccacagaggtccagtggcattgcagatgccggatgattgcaggagccaatttctacattgtgggtagggatcccgcaggaatgccccatcctgagacaaagaaagacctatatgaacccacccacgggggcaaggtcttgagtatggcccctggccttacctctgtggaaataattccgttccgagtggctgcctacaataaaattaaaaaggccatggacttttatgatccagcaaggcacgaggagtttgacttcatctcaggaactcgcatgaggaagctcgcccgggaaggagaagatcccccagatggcttcatggccccgaaagcgtggaaagtgttgacagattactacaggtctctggagaagaccaactaggtgctcctggctctggcttcttcctcaagtgctctctgacgattttttttttctatttttgtgatttagctgctctgtatccaattgcttctcggtgactttttaaagctagtatttttgcaatgaagtaaaaagctgtaaccataatttaaaaccaagttcattatgtctatgaagctcacactggagactgagtcttaggtgaaagcaatatcgttgtacgttccagaatgaaagcatttgcactgtacacttcatctcagccactatgtctcagatttcttaaaaatgtaactgtgtggctagctgactagcccagaacagaatcatactttccagcttacttgtgccagcctggaatctcaatgtcacttaccaaaagaaaacacacacacacacacacacacacacatacatacacacacacacacacacatatacatacacacacactgtcgctttgaaggaaatgtgttatatcaggactcttacttcataactacacataccatcactgcccaatgtacgagctgaagatgcaactggaagaaaaattcatgtgaagatgtaaattaagtcttaaagaacaatgtaatttatgtctgccatgaaaatgtcatcatccaatagagagagacgctctaatggccttctatgaatagttactctagtttggctggcacttttcaaaagcatggtatgcttggatcaaaacaaccccccccccagactaaaccaccagggtatattgtagactactttccccacactttgctgtggccatagttattatagaagcacctaagtgatcgctgtaatcaaaaccaacccacattattaatgagaagctaaaacttgatttctgtagcaaagataaaatccaaacatcatcctagtgaagatatatacacataagctttgagagctgctgagcactgtaatatacaaagttgacattagtgaatactgccgacactgaaagactgaccatcctcctaagctagggccctgcctaccctagacaacacaggaagaggttccgatttcctgaagctacttagtgaagctctccttgaggatgctaagaagcaaccaccttggagaccgaaggtttagggtatcccaaactcttgatgttataagtttccagaatgaacctgttgtgagatgtcctttgatggtttatttgctaagagggaattttggtattttatgcatggttcattgttgccaagaaatacataagagaaaaggcatctttacatacaggtttctttggaagagaccacagactgaaaagagaaactgatttttttcatactgccacgttttttaaaaatgctaggaaaagctctactgagagtaagttatatactgtgaccacagcaaagtgagtcttcgactgtatggcgcctgtgctcacttacatcagagctatttattcactgctgcctttaaaactatgtcttcactgaaaacgtgacagggttgtagtgccttcttagcatatgaagttgtgattttaagacagtagagtgcctttctgctgtagcacacagcataactgtttcagtgtcccgctggtacctgttcttattcaataaatctctcaaaagacca

SEQ ID NO: 36 - DNA, 503 bp, Mus musculus
aaacctctctattcagcacttcctctctcttggtctggtctcaacggttaccatggcaagacccttggaggaggccctggatgtaattgtgtccaccttccacaaatactcaggcaaagagggtgacaagttcaagctgaacaagacagagctcaaggagctactgaccagggagctgcctagcttcctggggaaaaggacagatgaagctgcattccagaaggtgatgagcaacttggacagcaacagggacaatgaagttgacttccaggagtactgtgtcttcctgtcctgcattgccatgatgtgcaatgaattctttgagggctgcccagataaggaaccccggaagaagtgaagactcctcagatgaagtgttggggtgtagtttgccagtgggggatcttccctgttggctgtgagatagtgccttactctggcttcttcgcacatgtgcacagtgctgagcaaattcaataaaaggttttgaaactaaaaaaaac

SEQ ID NO: 37 - DNA, 719 bp, Mus musculus
ccagagtttcgagtcacgtgccagaagggaaaactaaacacggaattagagaaaacttgatgcctctggcttgcactggtctcctttgggcccgttagggcccgctaaactccctcattccgctcctaatcctggacagtccaggcaacaggggcgtggaaagttgagggggctgggatgttcgtttgccttgcctcaggcgctgggtggggtcggggcgtgccagcactccctgggcggacctcacggatgctggccactataaggccggccagactgcgacacattccatcccctcgaccactcctttggcgcttcgctgtcgaccgtgcgcttcttctagcccagtgatcagtcatggcatgccctctggatcaggccattggccttctcgtggccatcttccacaagtactctggcaaggaaggtgacaagcacaccctgagcaagaaggagctgaaggagttgatccagaaggagctcaccattggctccaagctgcaggatgctgaaattgcaaggctgatggatgatctggaccgtaacaaggatcaggaagtaaacttccaggagtatgtcgccttcctgggggccttggctttgatctacaatgaagctctgaaataaaatgggaccgttgagatgacttccgggggcctctctcggtcaaatccagtggtgggtagttatacaataaatatttcgtttttgttatgcct

SEQ ID NO: 38 - DNA, 2161 bp, Mus musculus
gtggagtaaagctacgcccaggcccgcgtccgctggcggcgcaggaacttcagcacccgcggggcggacagcgcctaccgcacctgctcacctgctctgggcgccagaagagcctgcatcctccttccagcccggagcaactgcgccgggaggcgcccagaccctctcccttcccgcacccaggctcctgtcccttccagcttcttaactccccttctcattcataacaaaagctacagctcaggggcccagcgccaagctctttccagcaaagcacagaagagcaagaaagaatggggttcctttggaccggctcttggatactggtgttggtgctcaacagcggcccaattcaagctttccccaaacccgaaggcagccaagacaaatccctgcataatagagaattaagtgcagaaagacctttgaatgaacagatcgctgaggcagaggcagacaagattaaaaaggcattcccttcagaaagcaagccgagtgaaagcaattattcttctgtcgataacttgaatctgctgagggcaataacagaaaaggaaaccgttgagaaagagagacaatccataagaagccccccgtttgataaccaactgaacgtggaagacgctgattcaaccaaaaatcggaaactgatcgatgagtacgattccaccaagagtggactggaccacaagtttcaagatgacccagacggccttcatcaactggatggaactcctttaactgctgaagacatcgtccataagattgccaccaggatttatgaggagaacgacagaggagtgtttgacaaaattgtttctaaactgctgaatcttggcctgatcactgaaagccaggcacatactctggaagatgaagtagcagaagctttacaaaaactgatttcaaaagaggccaacaattatgaggagaccctggataaacccacaagcaggaccgagaatcaggatgggaaaataccagagaaagtgactccggtggcagcagtccaagatggcttcactaaccgtgaaaacgatgagacggtgtctaacaccttgaccttgtccaatggcttggaaaggagaactaacccccacagggaagacgactttgaggaactccagtatttccccaacttctatgcactactgacaagcatcgactcagaaaaagaagcaaaagagaaagaaaccctgatcaccatcatgaagacattgattgacttcgtgaaaatgatggtgaaatacggtacgatatctccagaggaaggcgtgtcctaccttgaaaacttggatgaaacaattgctctgcagaccaagaacaagctagaaaaaaatactactgatagcaaaagtaagctattcccagctccaccagagaagagtcaggaagaaacagacagtaccaaggaagaagccgccaagatggaaaaggaatacggaagcctaaaagactctacaaaagatgataactccaacctaggaggaaagacagatgaagccacagggaagacagaagcctacttggaagccattagaaaaaacatcgaatggctgaagaaacataacaagaagggcaacaaagaagattacgacctttcaaagatgagggactttatcaaccaacaagctgacgcttatgtggagaagggcatcctcgacaaggaagaagccaacgccatcaaacgcatctacagcagcctgtgaaaatggcgggcagcttgagccttcctgttgttccagcaaaaacaatatagcttacaaactaattcggcggttaaagggttaccagcccagaagtattaggatgtgctgaatttatagtagttaatcccttagaaatgagtaaaatagagctctcttgccataaataccttatgaaaagcaaagctgtagagaagccgaggtttttctatatagaatccttatttcctcttgaatttacattttgtaatcagagatgtgctgctctggaaaagactctaatgggttgaacataagtctgaacctactccccactgtcctcagccccctgaagctctgagaggccctgtctcggcatgctagacacctgagcacctcactggatgtttgtcataggatgtcgtttccactagtcgatctctgttgggcacggaaataaacccacgtctcttcat

SEQ ID NO: 39 - DNA, 1227 bp, Mus musculus
ccgaggtttcggagggaggtgacgcaggagctatccagcacgtcagctccctgttttggcctagattgcaagaagctcgcccgcggccacacggttaaaaatggcctcaaggctggtctctgctatgctatctggccttttgttttggttgatgtttgagtggaatccagcattcgcttatagtccacggacccctgaccgggtctcagagacagacatccagaggctgcttcatggggttatggagcagctgggcattgccaggccacgcgtggagtacccggctcaccaggccatgaatcttgttgggccacagagcatcgaagggggagctcacgagggtcttcagcatctgggtccttttggcaacatccccaacatagtggcagagttgactggtgacaacattcctaaggacttcagtgaggatcaaggctacccagaccctccaaatccctgtcctcttgggaaaactgctgatgatggatgtctagaaaacgcccccgacactgcagagttcagccgagaattccagttagaccagcacctttttgatccagaacatgactacccaggtttgggcaagtggaacaagaaactcctttatgagaaaatgaagggaggacagaggcggaagcggaggagtgtcaatccctatctacaaggaaagaggttggacaatgttgtggcaaagaaatctgtcccccacttctcagaagaggagaaggaagcagaataaagagaagacagtatgtagaaacccatccaatgcttatgtgcatgttcatagagcccgtgagtgacagcatgcattttacatatttatggatgaaaagcagctgtccttgcctccataccaatgcctgtgctttctgctacattagaataaaagctccttctctttgggggatttttttgatgtggatctgcaagaaacattacaattaaaatgtatatgtcaagtataataaaaacacggatatgaaatactcagatttcttgcagttttgggttatgctgtttgggccaagtctgtaaaacgctagcatttgattttgattatgtagtgtatctagcccttgggccttgttacacaccaataaagaagtttgtactcaagcagagggggtgacacatctcactctgctgcttcttaataaatgtatgtgcgagcattggcttgggaagtttttatataacagtataaaatcaatttctttgcttaat

SEQ ID NO: 40 - DNA, 2938 bp, Mus musculus
ctccacccagagcttgaaaccagtgacagtcacacttcccctcttctgcagcagacagcactagctcctctaatcctcttgcttccccctcccccaaccatttcttggggaataacaaatatagctttggggataatatagctttaagacgacttttggcaaatgtaaatgtcctaacatctgggcagtgttaccagaatcccggaggccctgacagaccaggagccactggttctaggaatgttaaagtacaagggctttttcccacccccgactgactgatgagaggagcagagagcaaagaaaaagaagagagatgaacgagaccacaaaaatcataaaataaaaagcagatgcatgttcctgctcttttcaaagcttccagtagaacgatagctccctccgatgtcatatgcctgcttcctctcacttttgggagttcatagctggttctgctctgaaagtcattcccctttatcctggtacttctggccacatagccctagtcttgtcttctgaagcttccctgtcgacgatgatgaccagtccactgactcagagaggtgctctctcactgctgctcctcctaatgcccgcagtgacaccaacatggtacgcaggctcaggttactctccagatgaaagctacaatgaagtatatgcagaagaggtccccgctgcccgtgcccgtgccctggactacagagtcccccgatggtgttacacattgaatatccaggatggagaagccacatgctactcaccaaggggaggaaattatcacagtagcctaggcacacgttgtgagctctcctgtgaccggggctttcgattgattggacggaagtcagtgcaatgtttgccaagccggcgttggtctggaactgcctactgcaggcagataaggtgccacacactgccattcatcactagtggcacctatacgtgcaccaatggaatgctgcttgactcccgttgtgactatagctgttctagtggctaccacctagaaggagatcgcagccgcatctgcatggaagatgggcgatggagtggaggcgagcctgtatgtgtagacatagatccacccaagatccgttgtcctcactctcgtgagaaaatggcagagccagagaaactaactgctcgagtatactgggacccacccttagtgaaagattctgctgatggtaccatcaccagggtgacacttcggggtccagagcctggttctcacttccctgaaggagaacatgtgattcgttatactgcctatgaccgagcctacaaccgggccagctgcaaattcattgtaaaagtacaagtgagacgctgtcccattctgaagccaccacagcacggctacctcacctgcagctcagcgggggacaactatggtgccatctgtgaataccactgtgatggtggttatgaacgccaggggaccccatcccgagtctgccagtcaagtcgacagtggtcaggaacaccacctgtctgtactcctatgaagattaatgtcaatgttaactcggctgctggcctcctggatcagttctatgagaaacagcgactcctcatagtctctgctcctgatccctccaatcggtactacaaaatgcaaatctctatgctgcagcaatccacctgtggactcgacctgcggcatgtgaccattattgagctggtgggacagccacctcaggaggtggggcgcatccgggagcaacaactatcagcaggcatcattgaggagctcaggcaattccagcgcctcactcgttcctacttcaacatggtgttaattgacaagcagggtattgaccgggaacgctatatggaacctgtcacccctgaggaaatcttcacattcatcgatgactacctgctgagcaatgaggagctggcacggagagtggagcagagggacctatgcgagtgaccttgagtcagggtgtggctgaagcacctgtcctagggagcttaaactaaggaggaaatgtcgtgtcctcccccccccacacacacacacacacactgttgggagcagccctcgggtgggggtgaggtttgccaaatctaggacagttccaggcagcattttagggcagaatattcatttccctaagcaagtctccacttcgggtagcactggaggagcctatgaaactgaggacatgccctgggtggtgagttgtagaccgaaggccttcttcacctgcctggagcctctctctctagactcttcccaaagcctttcagataagtaaataccaaattcattcctttacggtgttgtaaatggttcctctaccctacaataacagcagggggcagcattgcaacagacaaacaagacactttgaccaagtataaatagatttccctcacagttagttatgtgaggaacaggagagaggagactaggatctacaaaacattcttgaagctgctcgtcatcctctaggatgctggccttaaaaacaatgttgcttgagccatttcttcatcaagagttagaaaaacattttctccagggggagtttgggaagcaacgtctagccatagctggctgttccctcctagatgctccaactagcttgtaggcaagaacttctaataactgaggtgtttagtaccctggtgacagctcttccttagaggattctgcagtctgtgaacaacattacctctaagaattagggagatggctccgttggtgaagtgcttgctatcaagcactgagacccgagtgattcccagaacccatgtaaaatagccagatgtggtggtgtgcacttggaatcccagcagagaggtagacataaacagatctgtggggatcactgcttagcaagccaagtctaatgatgagtaccaggtcatgtaagaaacactatctcaaaaaccatggtaaatggcataaaaaaaaaaaaac

SEQ ID NO: 41 - DNA, 2811 bp, Mus musculus
tgcccgtcccgggagccggcgaggccgggttggggctgcccggcgcgggagtaccgcagggagtaccggctggagtaacgcgggggcggcggcccagcgccccgaagtttgccgcgcccgctcgggctgccgtggtttgttttcttgaaaaggctccaggcttcggcttggaaaacccaaccgccaaaattgagcccagcagctggagcggcagcgagagccctgccgaaaacatggaaaggatgagcgactcggcagataagccgattgacaacgacgcggagggcgtctggagtcctgatattgagcagagtttccaggaggccctggctatctatccgccgtgtgggaggagaaaaatcatcttatcagacgaaggcaaaatgtatggtagaaatgaattgatagccagatacatcaaactcaggacgggaaagacaaggaccaggaagcaggtgtctagtcacattcaggttcttgccagaaggaaatctcgtgattttcattccaagctgaaggtaacaagcatggatcagactgccaaggacaaggccctgcagcacatggctgccatgtcatcagcccagatcgtctcggctactgccatccacaacaagctggggctgcctgggattccacgccccaccttcccggggggtccggggttctggcctgggatgatacagacaggacagccaggatcctcacaagacgtcaagccctttgtgcagcaggcctaccccatccagccagcagtcacagcccccattccagggtttgagcctacgtcagccccagccccctcagttcctgcctggcagggccgatccattggcacaaccaagcttcgcctggtggaattctccgctttccttgaacagcagagagacccagactcgtacaacaaacacctcttcgtgcacatcgggcatgccaaccattcttacagtgacccgttgctcgaatctgtggacattcgtcagatatatgacaaatttcctgaaaagaaaggtggcttgaaggagctgtttggaaagggccctcaaaacgccttcttcctcgtcaaattctgggcggacttaaactgcaatatccaagacgacgccggggccttttatggtgtgagcagtcagtatgagagttctgagaacatgacagttacctgttccaccaaagtgtgctcctttgggaaacaagtagtagaaaaagtagagacggagtatgcgaggttcgagaatggtcgattcgtgtaccgaataaaccgctcgccaatgtgtgaatatatgatcaacttcatccacaagctcaaacacctaccagagaaatatatgatgaacagtgttttggaaaacttcaccatattattggtggtaacaaacagggatacacaagaaactctgctctgcatggcctgtgtatttgaagtctcgaatagcgaacacggagcacagcaccatatctacaggcttgtgaaggactgaacctggttatttatatagagagatatctgtatatacacacacatatgtgcacacacacactctccattatcgaacgactgactgtaaacctcaccgcacagggtgctaccctggccccccaggtcccaccctgacctttctaatcctgttcgagtgaacttattttttttttccatgtgttcatgctatcattgtagctgtgaagttgtgatgcagttttttgtgtattcctcagatctgcaacccacaattccttggggaaggtgaatcttaccagcctaagctaagcctctctgcagccctgtttcctgttgtggtagaaaatactgagacagaggattggccacggggcattgactgcctttatacaaatgtatttagttctttttgtgtttttccaacataaaattcttgttttaagatacaagtaaaattaatctttaaatgtaaatgtaaattagtacaggaaaactaagattctttagacttatctttgtaactaattaggatggaagttatggaagaatgtaattcactaaattattttttaaatgaaatctttctttctttttaaaaccaaatgttaaactatagccttaagacatgcttggttgaactatcctaatgagacaaatttataccttcccccagcccccaaggttaaaactaataccctaattcattaaactcttgaaacaggtgtcacaaaggaggaagacatcacccctcacccttaacatatatagtatatttaaaaatgtaaaattgtattgtactaatgtgataattcattatttaatgaaaaagaaaagatggctctttttgcaataagtaggtaatactgagacaagtctaagcttacaacgttatagtcttgtgtgtgcagttacattttatatggtcaaccaaattttttacagagacgagttcacgtttgaaccactgaaatttaatagcaaatttaaaaattggcatgaatactgcactgttgagttgcaaaggatgcagagtcctgtggctggaagaaacttgtcatcaaccgagcaagcagaaggctcatcaggcttggtgtctctgctccccagaatgtcactgtcacaagcttcctggccagcctgtggatagattgttgctggtaacccagcgtgcataggaagcatctgtggctctagctctgtgttcttgcgcatgtggacattcagctctctgagctcctgtttcttgtcaatttgctagttgctcattgcctctctgcattatagacatggatgtattttctctaagggcatctttccataagcctcagtggcactttacacatatattcatt

SEQ ID NO: 42 - DNA, 3142 bp, Mus musculus
gggcagtcggagctggctcgcagccttgccaggcgagcgtggggtgtccgcgcgctcctctgctcccatagcccgagggactgaaacttccgtgggcggctcccaccggagcgcactgccgggggcgcaccagcgacagggagaaaggagaccagacgcgggctgcagtaggagccggggagggcagcccgctggcccggcgcccaacagcagccgcgcggccgccgccagccgggcgggctccgggactgcaggggaggtgcgggcactcgcagcgtcccgagcggtggccggagccatgaggaccgagcacagagtggcggaggcagagcgcagccggccgctgctgcacttgccgccgggagcactgcgcgcactgcccgcctccttcgctgcttctcgcaggggaagtgaatagagggggaaagcagccaccagctccggactctgatgctgaaacgctccagccgcggcctggctcctggtcggcagcgaggcgtcccctcgaggatgcccaaagaaggtcggatcacaagccaagctttcagaaacgtttcttctgtgatgcccctgtgaaggccgaacctagcagggacgtggtcactcgcaaggagggatgacttagaccctggctctgcagcctgggcttcgcctcagaggggagtgttcctcacggaaagccccagggatcgccgaaccataacttcttgggggagatctgtgacctctagagacatcaccggtgcccagggcagtgccatgtggggcggacgctgctcaccttccacctcttccaggcaccgcgcgtcgctgctgcagctgctgctggccgcgctgctggcggcgggggcgcgggccagcggcgagtactgccacggctggctggacgcgcagggcgtctggcgcatcggcttccagtgccccgagcgcttcgacggcggcgacgccaccatctgctgcggcagctgcgcgctgcgctactgctgctccagcgccgaggcgcgcctagaccagggcggctgcgacaacgaccgccagcagggcgtaggggagcctggccggacagaccgagaaggcccagacagctcggcagtccccatctacgtgccgttcctcatcgttggctcagtgttcgttgccttcatcatcctcgggtctctcgttgccgcatgctgttgccgatgtctacgtccaaagcaggatccccagcagagcagagccccaggggccaaccgcctgatggagaccatccccatgattcccagtgccagcacttccaggggctcatcctctcgtcagtccagcaccgctgccagctccagttccagtgcgaactcgggggcccgggctcccccgacgagatcacagaccaattgctgcttgcccgagggaaccatgaacaatgtgtatgtcaacatgcccacaaatttctcagtactcaactgtcagcaggccacccagatcgtaccccaccaagggcagtacctgcatactccatatgtaggctatgccgtacagcatgactctgtgcccatgacgccagtgcctccgttcatggatggcctgcagcctggctacagaccagtgcagcccccctttgctcacactaacagtgagcagaagatgttccctgcagtgactgtatagcctgcacggcacagattagactcctttacgagactgaacaacatggggcctgattcttgcaccacaagtctgctcaagttggtggtgtacccgccgatgcgcttccggatgacgtcattcacctctaacctataaggggacatctccacagcagcgtgtctgtgtgtgtctccagatgcaaaattgaaagccacagcccctggagttgccacctgtgtcctcaagcgtgtgacaaaagcttgagccccttaagtgcccttgaggtgtggctgccagagtcaggggttgaaggatgtctttattcctttctgtttcagcggcgggcacaggagaatgcccattaccgcccctttacctgggcttttttaaaatcagtgttcaaggctgaaaggagatgtaaattatataattattatgaaaagaagcgatcttgaactccgaccctagtcatgagcctcaatcatcagaggcatgtcttaatttcattgtaaaagatatagtctgtctatttttatgcttttgggggaggggaggctattatgtgcttttgttgataaccatgtgtagagatcttaagtgatttttctacagtacagaaattgaaagaaaatgtccatttctaaggaagacattcaacattctgggggtggagtggcattgaagccattcctctccttttgatggtgtctttttatttcctggctctacagaagaagcaattggatgtggtttttgttttgtttgtttgtttgtttgttttccattgaaaacatgagctgtcatctgctgctctccaagttactttcaactttgcctcatgatacagcctggtggttcatttgttctgcccagttttgggatccccttgtctaaatagtcccaagggcaggaaattatcaatgggccgatgaagatcagtaaaaaaaacaactgtactgaggagatgctcagataggttgtccgctgtcactgggacatgaggtggcacttaaacctcaaacgtgaaccactttgcaaatcagaaggcttttttatgctcttgttatcagatggtttatttttccagcagcaggagctatgaaatccagagaaaagctccccagggagacatggcatcccttttctcctcagccttcaacaagctttgtgttcactgccaaggttttaatttatcaaattcaactgaatttgtacattggttacttatggaaaaagtttaaatacattgtaagtgaattttcaactgctagcaaagccaaaatttgttaaataactgtgaagtttggtgttttataccatcctacccacagggtcttcttcttgccttctgtcatctgtttctcatctcatgatgacagcgataatgttacattatcaatggcaattggtaccaacagataagaatcaaagattttaataaccaaagcaggagaaaatcattttaaatttttaataaatattttatggtgtgaaaaaaaaaaaaaaaaaaaataat

SEQ ID NO: 43 - DNA, 3578 bp, Mus musculus
ggggtgtgagccctacgctaggcagctgcgacaggcggcgggcgtgcggctctcagccagcctgaatcccattggccctcgggacgcgctccgagctgctcctctcccccagggcgcagaggctgcccactggtccgcgagctcggtttggagaaattctttgcctggctagcaaggagagcaagactgggctctggcgactccctagagcacgcttcatggtggagactcagggaggagaaacccagggatgacaggcagaaggcaacacgggcactgacaggactccaagaaggctaccggccaccagcagccagatgaacaggctgggtcaaatgctctagctaggtgcatgcgcctggtggagcgcgcaggtcggactgtgctctccgcggtccgccctgcgctggtgcccggccggtaccaggatgggcactggagccccagggccgcgcccgctacagcccggcgtccctcctgcgtcctgagctcatggacggcgactccaggctggcagcggcgcgcccccgggctgtgaatgcgactcgctcatcgtccgcgctccccgcccgcccgcccgccgggacgtggtaggggatgcctagctcctctgcgatggcagttggcgcgctctccagttccctcttggtcacctgctgcctgatggtggctctgtgcagtccgagcatcccgctggagaagctggcccaagctcctgagcaacccggccaggagaagcgcgagcacgcgtctcgggacagcccggggcgggtgagcgagctcgggcgcgcctcgagggacgagggcagcagtgcccgggactggaagagcaagggcagccgcgcgctctcgggccgggaggcgtggagcaagcagaagcaggcctgggctgcccagggcgggagcgccaaggccgcggactggcaggtccggcctcgcggggataccccacagggggagcccccggccgccgcccaggaagctatcagcctggaactcgttcccacaccggagctgcctgaggaatacgcctacccggactaccgcgggaagggctgtgtggacgagagtggcttcgtgtatgcgatcggggagaagttcgccccaggtccctcagcctgcccgtgtctgtgtacggaggaggggcccctttgcgcacagccagagtgcccacggcttcacccgcgctgcatccacgtcgacaacagccagtgctgtccgcagtgcaaggagaagaagaactactgcgagttccggggcaagacctaccagactttggaggagtttgtggtgtctccatgtgaacggtgccgctgtgaagccaatggtgaagtgctgtgcacagtgtcggcgtgtccccagacggaatgtgtggaccccgtatatgagcctgatcagtgctgcccgatctgcaaaaacggtccaaactgctttgcagaaaccgctgtaatcccagctggtagagaggtgaagactgacgagtgcaccatatgtcactgcacttatgaagagggcacgtggagaattgaacggcaagccatgtgcacgagacatgagtgcagacaaatgtagacttcacaatacagaaactctcatgtttttctagaatattttactaatgtgacattctagatgactctgagaaatatcaatcaaaaacagaagactttggataaggagtcaaggaaaatggttggtacttttgtttttcttggtaacaaatacagcaagagacagaaatggatgtatttcaaaacatcaataagaactttgggcatactccctctctaaataaatgtgctattttcacagtaagtacactaaagttcactattatatattaaatgtgtttctataatccctctattagagtcttatatattaaaaaaatgctgttgtcaaccgtcctcactgtgtaccggtatatctttgttagccttcactgcatcacgaaagggtgctagtggttccaggaaactcacctgcacatggaatgctctactccacaaaaaactccagaaaataatgagtgctttatatcaaaatcctgttcaaatgctatgtatctaatgcttaaaacacaatctaatttacacaaacagattattttagtaacccatttcttttaatagtttattcttagtcatttgtaatatggaggtataaagattactaacaggtactttctcagttctcattgcagcccatgaataaacacttatgtgcatttcattttcatccatggtacctattcttctaaacatgacatcaattcattttctgagcaaaaggaaggctgggatatctgtgttagtgtccgtggagctgggatgttataaaaaggagggaaagggagtctttggattctctggaaaatagttaagaaaagtcaaatttctgaacaagtttatgcttgaaatataaaatatgccacagacatctcaggaatcagcacagtgttcctatactgctagttacgatagctatctgtcctcagtagtagacatagtgagatgataaatgattgtgcatcaagagcagtgtgagcaagctattctttgtccagttatgcctaagcattggaaaataaaggctactaagactctcctgcagaaatatatcacccaaattatcttatttgaatttaaatgataacactgtcattaggtggtgctcactgaaggaacagttccctttctgcgagtgacatcaacagttggtacatgagttagagaggcagtcagggtcttaatatgtgtaaacaaaactaaagtaactatttgcaaactttaagaacattttcctccatttattttttttctaaattttaaagttaagtacctatcacacaacaaatcttgtgaagacaaagggaaattagggctggtttgttttcttcatgagtatctgtttctgtagcataacaggactataaaatcacaatgtatgtgattatatataaatcaaattagaggctagaaaatgaagatttctaaaaagcacaatttatacccccaaaccctgccagtatacaagaatttcagtaattattgaatgacagccaaggaaaatcaatgtattttcatcattaaaatgttaatcattatataccttaacaaatacaaaaattaccaatacatttgcatatgcttatattttaccttgattctgaagaatattttgggtagcacagcataggcagagtagcatgtacatcaatttcatgaaaatactcaggggtattattggctagttactctcaggtacctgttttcagctttccaacacttaaatgttagtatgaaagcctgttaggcaccatctgtccttccattactcaacagtagatttcctttaaaacaaaggtcatctaaggttaccatcttgccaacaaatgacttatcctcctaacatacatgggctattgagccagttgtaattacatacaatctgcctcttttctccacataagacatggtgaattacaattgcagtgtatgaggcccaattttcttattttccttggcaatagaattcctacagaatggaaaaatgggctctcagattattgcagtggg

SEQ ID NO: 44 - DNA, 4273 bp, Mus musculus
agtccctggaagcagacgtttcggccacagacccagagaggaggagctgacaatcaggaggcgtgagccgcctggagtctgcagaattcgtggtgtgaatgaactgggggcatcttgggcacagggattgccccccctccttccccgcctcgggccacagttgagtagtggggcattttttttcaccttcttgtgaagaattttttttattatttgttgtaaagtcttttgcacaatcacgcccacatttggggttggaaagccctaattaccgccgtcgctgatggacgttagagagggagcgcctcgccgcggaacagtcgcctgcgcgccctcgtcggacccgcggctcctgcactgtgtccccgctcggccctgcgcttgctgctcgcccgcgcgcgccggcgccctctcggttcctgggcacatttccacgctataccaactcctctgcccgagtccgggcgccagtgctcgcttccgctccgggtcgctgcgcccacccgacgcgcccaggaggactccgcagccctgctttggattgtcccccaaggcttaaccccgacgcttcgcttgaattcctcggccgccttcgctcgggtggcgacttcctctccgtgccccctccccctcgccatgaagaagcccattggaatattaagcccgggagtggctttggggaccgctggaggtgccatgtcttccaagttcttcctaatggctttggccacgtttttctccttcgcccaggttgttatagaagctaattcttggtggtctctaggtatgaataaccctgttcagatgtcagaagtatatatcataggtgcacagcctctctgcagccaactggcaggactttctcaaggacagaagaaactctgccacttgtatcaggaccacatgcagtacattggagaaggtgcgaagacaggcatcaaggaatgccagtaccagttccggcatcggagatggaactgcagcacagtggacaatacttctgtctttggcagggtgatgcaaataggcagccgagagacggccttcacgtacgcggtgagcgcagctggggtggtgaacgccatgagccgagcatgccgggagggcgagctgtctacctgtggctgcagccgcgctgcgcgccccaaggacctgcctcgggactggttgtggggcggctgcggagacaacatcgactatggctaccgcttcgccaaggagttcgtggacgctagagaaagggaacgaatccacgctaagggttcctatgagagcgcacgcatcctcatgaacttacacaacaatgaagcaggccgtaggacagtatacaacctggcagatgtagcctgtaagtgtcatggagtgtctggctcctgtagcctcaagacgtgctggctgcagctggcggacttccggaaggtgggcgatgccctcaaggagaagtatgatagcgcggcggccatgaggctcaacagccggggcaagctggtgcaggtcaacagccgcttcaactccccgaccacgcaggacctggtctacatcgaccccagtccggactactgtgtgcgcaacgagagcactggctcgctgggcacgcagggacgcctgtgcaacaagacctcagaggggatggacggctgcgagctcatgtgctgtgggcgtggctatgaccagtttaagacagtgcagaccgaacgctgtcattgcaagtttcactggtgctgctatgtcaaatgcaagaagtgcacggagattgtggatcagttcgtgtgcaaatagtggtgtgcctgcccttcacccagtcccactcccaggacccacttatttatagaaagtacagtgcttctggttctttttatttctcccccaagaattgcagctggaaccatgtgttttgttttgttttattttgttttttcttttctgttaccatctaagaactctgtggtttattattaatattataattaatatttggcaatagtgggggaaactaagaaaaatatttattttgaggatctttgcaaagttagtacaaaatttctttcttctgatgctacaggataaaggggaaaaactatgtattcgaacttagctgtgcagttgggggttcacatctagaaggtgtaggagccattttcttctcaaacagagagtcctttgagatgggtggtatccaggtgaaggaggaggtacagacccatgaataacagttcctgtgaccaaaatgaattgcaggtgctctggtacaaaagatcttaaatatagatatattaaatatacatatatgccaaaaatacagaatatgagacactccctaacccagaggttaccagcctggttttgtgggttttttgttttgttttgttttttctttttttgggttttgtttgtttgtttgtttgtttgtatttttggtgtgtgtgtgtgtgtatttctagaatgatcttttagaaggtacaagcaagaatctcatatcttcagaagcaggcatatcatgtatgttactgtgtcccacctacagatactccattcatgaatgggcctttttctaacagttcatgaatattggggagccggtgggctgggggagggaggtccccagaaattagaaaacttgaagtttcctacattgaggccataatcttgtgttagcccagctgattcttaataccagacttttagatccataaaggaatttttgactaaaaaaaaaaaatcttgttttgaaagccatcttattttcttaaaaatgaaaaattacccatgaatcccatttgcaacccctcacccccacaggcaacaagaaagtcccatgtagttgagcactgcgaacacctctgtgaggagatgatggcagccatcttcctgcatgatcccatgccctttctggactctctgctggccatgcttccgaatggcagccctggtggacactcactgctggtagggcagaaaatgtacacgaggagccatgttcagaaccagccacttaggggttgttctctgaggcttttctttggaggtacggtaacttgatgtgttttgatgatatctcttggcccagggagtccacagaggtgttgcagctgtttggttgttatcctcctgcgtttagactttccatttgtgcttttcctattaccctgcaggtgtaccctaaaactgttcctagtgtacttgaacagttgcatttataaggggggatgtggtttaatggtgcctgatatctcagtttttttgtatataacatatatataaatatacatatataaatatagatataattatatctcagtgcagtctgggatttagacctacagttttctctgggcttgctctctgcctggagtatcgtccttcattgcagtccaattgggatttctttttttccaaaaattttgagtcttaacattgacctgtgacaggatcctaccacgaataccaggaagcaagctaagactcggaggaagctctcagggctcatgtcctgaatgtatgttggttagaaagtagcctttctgcttcctgcccatggccagttctccaccctctctttggtgttctttgtggggagggcactgtggtttgtcgcagccctggacttcgagaggctcccagaacccaggatcaccagcctcctgtctgtttgcttcactcctttcccagggaggacttgggactgtcctgtctgacaggacggatctgagttcccgaagcaaaccagctcaccacatagatagctagtttaaacaatgttttaaaataagggcacctctgtttcaaaagtgacatctgctgtgttgttttcgaggcctgatactcttacaaggtttgaaaaaaaatgtgtgtatccattcatgggcttggtagccttctggtcacctcagtcctgtggctcttaacttattgcccaacaatattcatttcccctcagctacaatgaattgcaagcaaaagatgttgaaaaaaagcactaatttagtttaaaatgtcactttttggtttttattctacaaaaaccatgaagttctctctctctctctctctctctcttatttgttaaatcagattatgttctttttttgtttttgtttttagtgattcatgtttatgagcagagtggagtttaacaatcctagctttaaaaaaaacctatttaatgtaagatattctacgcatccttcagatattttgtatatcccctatggcctttattctgtacttttaatgtacatatttctgtcttgtgtgatttgtatatttcactggt

SEQ ID NO: 45 - DNA, 2637 bp, Mus musculus
ccgactggcggccggagcggagcccacaagttgccggcagcggagcgccgcctcgtcccgctctcagcccttgcgcccaccgggctgcggccggcgccaggacgctcagcctgcagacgctgaccccagatgtgacagcgggaccgcagatctctctccttccgggcgcaacccacagagtctcgccagcgtctcctctgccaaaaccccacggctggaagatgtggcagccggctactgagcgcctgcagcacttccagaccatgctgaagtctaagttgaatgtcctaacactgaaaaaggaacctataccagcagtcctcttccatgagccggaagccattgagctgtgcaccaccacacccctgatgaaggcaaggactcatagtggctgcaaggttacctatctgggaaaagtctctaccacaggcatgcagtttttgtctggttgcaccgagaagccagtcattgagctctggaagaaacacacactggctcgagaagatgttttcccggccaacgccctcctagagatccggccattccaagtgtggcttcatcaccttgaccacaagggtgaggcaacagtgcacatggataccttccaggtggctcgaattgcctactgcacagctgaccacaacgtgagccccaacatcttcgcctgggtgtacagagagatcaatgatgacctctcttaccagatggactgccatgcagtgcaatgtgagagcaagctggaggccaagaagctggcacacgccatgatggaggcattcaagaagactttccacagtatgaagagtgatgggcggatccataggagcagctcatctgaagaggcttcccaggaattggaatctgatgatggctgaaggaacttaagatgttccagcgaaggcagcatttgggcaaggagtttcagaagctagacatgtctgcaatgattcaaatttggtacgaaaggactgccaacctattggctgatcatgctttttaaattcagaagagtgattctaaatctaaagaatcatatcattaattatgtgacattgaaacctgctgctgccgtgatcttgaggagagtacagatggggaggaaggttccagactctcctctcactttttttttcctctcctattccaacacttcctgctgggagatctccacgcctattttcaccattctcaggcaaatactccatggctgtagtttgacggactgttccaatctgccttatgaaatccaacaagaatgttagtggcatctctgtggtccctaggcaggcaggaggtatggggaggtgctgttggcgtggaggctgcaggatggaagggacacgtccaggtgactgactggttgctcctcccccttggcatgtttgcagatcctctcctcagtccgtgaatcagcacagcttggattgagctttacaactaacagcgcgctagatggcagttaattcgcagttaaagataatgctttttatttacacgaatatatcaagtagtaccctcctattgtattcacttcttctattttcttagaattcttgcaactaatgattgttcccctccttctcccaccaccaagttctttgtccttccaacctcccggaaagggacaaaggctcaacatactgcaggactccaaaagggaagaaaataacaaattaagtaaataaacgatcaggaaaccaaccaggaatctcagacatcagaaaccctagtaaagaagtgaatgagtggccaaagtctatagaggcttccctttagcaagccctgagatatagtaacttccttggttatgagtcttgttgaatgactccttttggaaagtttaaactctgacatggcattgccctggaagtcttgccccatcatttaacaaggaactcaagtaggaaatgagtgacatcaagtcagtttgcctagaaatcccatgtcttcctggaaacctaaagggaaacatgatcttgtcctgaaaatgatattttagagtctgagtgacccctttgactatcagttctgaagagcaattctcactggtaacttgacatctctcaatttcaggatctttgcctctcattaaaagtcatggaaaatatacacatttaaggtgaatagacacattaaggtggcaaatgatctactttttcaagtaatattgctcctttaaaaggtgtgtttttatttctggtgatgtcaggggcaacggggtatcttagaagccttaaatgagagctaatataatccagacagcaatggtgttagtatttttggtctgtgtacccacgtgcccatgtatatgtttgtgtgtgtgtatgtgtgagtgtgtgtgtatgtgtgtgtgtatgtaagtcccctgtgtgggtccagtttcaaggcatgtacaataagcatggagtcgtattgatgaggacttacctcctgaagatatgcttgttgctttatgatatatgtaaactattctttagaaaaatgcattcactctttaataaaagtatgtttgtgttaataaagccagggcgtttcacactttatatgaacttcccttttttttaagaaatggaaatttgacatgtaaaataaatcaaacttggtaaaaaaaaaaaaaaaaaaaaaaaaaaaaaaaaa

SEQ ID NO: 46 - DNA, 1660 bp, Mus musculus
agcaaattgcgggcggggacccggagctcgctctaccgcttgtcgcggtcctctcgcgcaggaagcgcgcgatgaaggcggtgagcccggtgcgcccctcgggccgcaaggcgccgtcgggctgcggcggcggggagctggcgctacgctgcctggcggagcacggccacagcctgggtggctcggcagccgccgccgccgctgcggcggccgcgcgctgcaaggcggccgaggcggcggccgatgagccggcgctgtgcctgcagtgcgatatgaacgactgctacagtcgcctgcggaggctcgtgcctaccatcccgcccaacaagaaagtcagcaaagtggagatcctgcagcacgttatcgactacatcctggacctgcagctggcgctggagactcaccctgctttgctgagacagccgccaccgcccgcgccacctctccacccggccggggcttgtccggtcgcgccgccgcggaccccactcaccgcgctcaacactgacccggccggcgccgtgaacaagcagggtgacagcattctctgccgctgagctgcgatggatggccaggtgtgcggccgcctgagcaccagcgagccaggagccctaggaagggagggccagagcagaaattaagagaaacaagccaccggaggaaagggggggaaatcttcagcaaatctagaaacgtcgtctcgtcttgtcattccaagagagagagagagagagagagagaaggggaaaaataaaacttaaattcacttttactttttttgcacgttcacgagcattcaccgtacgtattctctttcgtttcttcttttatgaccgctgtgaattgtacgtttctgtggttatttttatcacccttttgaaggtgcagttaaacttcgaagcttaagtgttgtcgaccagactgctaagtagaagagcaatcgtgaatccaaccttagaggctacattgtgacaagggaactgtttttgtttttgaagctttactaatataccagagcactgtagatatgttgttttacatctattgtttaaaatagatgattataacagggcggggaactttttctctgcaagaatgttacatattgtatagataagtgagtgacatttcataccctgtatatatagagatgttctataagtgtgagaaagtatatgcgctttaatagactactgtaattataagatatttttaattaaatattttttgtaaatattatgtgtctgtttttttaaaatcgatgggaatatttcttttggaaaattatttttcagctcccttgcagagcttttgctatctggatgttttctgtttggccaggctgttgatttggtttttttgttttgttttgcccagtatccagtttctgaggctcagaggtaacagctctctggtactggtcttgcatgattgcatgaggtgttcaatcacaaaataaaccagttacgagtcctttcaaatgtgctttctttaacctagatggaaacctttgtatttgacgtgtacatggtaaaaacctaccttctcgggttttaagtacagggttttatagtgtaatatataaagaattaagtgtgtggggtttttattattattattctttttgaactgaggtcaaaaatagattctgagtgaattc

SEQ ID NO: 47 - DNA, 24 bp, Artificial Sequence
  Description of Artificial Sequence Synthetic primer
tttgagggctgcccagataaggaa

SEQ ID NO: 48 - DNA, 24 bp, Artificial Sequence
  Description of Artificial Sequence Synthetic primer
cacatgtgcgaagaagccagagta

SEQ ID NO: 49 - DNA, 24 bp, Artificial Sequence
  Description of Artificial Sequence Synthetic primer
actacagcgaactggacacacaca

SEQ ID NO: 50 - DNA, 24 bp, Artificial Sequence
  Description of Artificial Sequence Synthetic primer
agtaatagggctgtatgctcccga

SEQ ID NO: 51 - DNA, 24 bp, Artificial Sequence
  Description of Artificial Sequence Synthetic primer
atctagatcccgcccttggtttgt

SEQ ID NO: 52 - DNA, 24 bp, Artificial Sequence
  Description of Artificial Sequence Synthetic primer
cggaaactgcagtgatggtgtgaa

SEQ ID NO: 53 - DNA, 24 bp, Artificial Sequence
  Description of Artificial Sequence Synthetic primer
gctgaccaatcacaccttcagcaa

SEQ ID NO: 54 - DNA, 24 bp, Artificial Sequence
  Description of Artificial Sequence Synthetic primer
tcatttccatggagggtcagcact

SEQ ID NO: 55 - DNA, 24 bp, Artificial Sequence
  Description of Artificial Sequence Synthetic primer
aacaacatcaccaaggtgggcatc

SEQ ID NO: 56 - DNA, 24 bp, Artificial Sequence
  Description of Artificial Sequence Synthetic primer
agtagggcacagggttgttgaaga

SEQ ID NO: 57 - DNA, 24 bp, Artificial Sequence
  Description of Artificial Sequence Synthetic primer
aacgagtgcggatttgtaaccagg

SEQ ID NO: 58 - DNA, 24 bp, Artificial Sequence
  Description of Artificial Sequence Synthetic primer
ttggcagtaacagttgggcaagac

SEQ ID NO: 59 - DNA, 24 bp, Artificial Sequence
  Description of Artificial Sequence Synthetic primer
tggtcctcaccctcaccaaatgat

SEQ ID NO: 60 - DNA, 24 bp, Artificial Sequence
  Description of Artificial Sequence Synthetic primer
aatattgagcatggcttgcctccc

SEQ ID NO: 61 - DNA, 24 bp, Artificial Sequence
  Description of Artificial Sequence Synthetic primer
tgtaccgtgcatcaagagcttcct

SEQ ID NO: 62 - DNA, 24 bp, Artificial Sequence
  Description of Artificial Sequence Synthetic primer
gtgctgatgcggatgttgctgaat

SEQ ID NO: 63 - DNA, 24 bp, Artificial Sequence
  Description of Artificial Sequence Synthetic primer
agagagcctgatgcgagaacttgt

SEQ ID NO: 64 - DNA, 24 bp, Artificial Sequence
  Description of Artificial Sequence Synthetic primer
tcaccacatgctggcacattcaac

SEQ ID NO: 65 - DNA, 24 bp, Artificial Sequence
  Description of Artificial Sequence Synthetic primer
tgtggtgatcttggaacccaggaa

SEQ ID NO: 66 - DNA, 24 bp, Artificial Sequence
  Description of Artificial Sequence Synthetic primer
tttacatgatgctgtgggatgccg

SEQ ID NO: 67 - DNA, 24 bp, Artificial Sequence
  Description of Artificial Sequence Synthetic primer
cccttcatcaacaggagaaatgcc

SEQ ID NO: 68 - DNA, 24 bp, Artificial Sequence
  Description of Artificial Sequence Synthetic primer
cttgttgcgttcctggactctctt

SEQ ID NO: 69 - DNA, 30 bp, Artificial Sequence
  Description of Artificial Sequence Synthetic primer
gtttaaacaaacaaaccgaggcagcatgga

SEQ ID NO: 70 - DNA, 32 bp, Artificial Sequence
  Description of Artificial Sequence Synthetic primer
gtttaaacgcagtctgccataccagttgcatt

SEQ ID NO: 71 - DNA, 24 bp, Artificial Sequence
  Description of Artificial Sequence Synthetic primer
tgagcaagaaggagctgaaggagt

SEQ ID NO: 72 - DNA, 24 bp, Artificial Sequence
  Description of Artificial Sequence Synthetic primer
ttctgatccttgttacggtccaga

SEQ ID NO: 73 - DNA, 1825 bp, Homo sapiens
acaacttggaatgtcctgcaatatctaacatgtaagcacgtcgaatcattttctagacagaatctgaacctctgtgtctctcagtctttctctctttctcattctctttcaatatggaacttgaaaagcgtgaaaaaagaagcttattaaacaagaatttagaggagaaactgacggtctctgctggtggttctgaagccaaacctctgatcttcacatttgtccccactgtcagaagactaccaacccatactcagttggctgacacctctaaattccttgttaaaattccagaagaatcaagtgataagagtccagaaactgtaaataggtctaaatccaatgactacttgaccttgaatgctgggagccaacaagagagagaccaagcgaaattgacttgtccttcagaggtcagtggaacgattttacaagaaagggaattcgaagcaaacaaacttcaagggatgcagcaaagtgacctcttcaaagctgaatatgtccttattgtggactccgaaggggaagatgaggctgcaagcagaaaagttgaacaaggccccccaggggggattggcaccgcagctgtccggcccaagtctctagctatctcgtccagtctggtctctgatgtagtgcgtcccaaaacacaggggactgatctcaagacctcatcacatcctgaaatgcttcatgggatggcccctcagcaaaagcatgggcagcaatacaagaccaagtcaagctacaaggcttttgcagcaatccctacaaacacattgcttttggaacagaaggcactagatgaaccagccaagactgaaagtgtctccaaggacaacacattagaaccaccagtggagctctattttcctgcacagctcaggcagcaaactgaagagctctgtgctaccattgataaggtcttacaggattccttgtctatgcattcttctgattctccttcaaggtccccaaagacattgttgggttctgacacagtcaaaactcctacaactcttccaagagcagctggtcgagaaaccaaatatgcaaatctctcctcaccatcttctacagtatctgagagtcagctgactaagcctggagtaattcgcccagtacctgtaaaatccagaatattactgaaaaaagaggaggaagtctatgaacccaaccctttcagtaaatacttggaagataacagcgacctcttttctgaacaggatgtaacagtccctcccaagcctgtctcgctccatcctttatatcagactaaactctatcctcctgctaagtcactgctgcatccacagaccctctcacatgctgactgtcttgccccaggacccttcagtcatctgtccttctccttgagtgatgaacaggagaattctcacaccctcctcagtcacaacgcatgcaacaagctgagtcatccaatggtggctattcctgaacatgaagctcttgattccaaagagcaatgaagttggagcagaggctgaaaacacaggctgctgaagttttttggaatgctggtgctaaccacttgctagatttaacttttttttttttttccagaatgagtgctccctttatgagctgcagtgcagcagaaccaaaaaaaaagtttgctgcaattatatagcatcacagtgctctgctaacagccagcatagaagagatttacctacagctttttgcaccactgttctagcctttaatgccttctacttaatattaagctgaccgcaatactaacgtgcccctatatttggcagccaaataaagaagaatcgtgggtaaatagaaaaaaaaaaaa

SEQ ID NO: 74 - DNA, 3186 bp, Homo sapiens
gtcttttgtggacccgcacaatgatccctgagccagactggattaggatgcctcgcgactaggggtccagagacagaggcctccagttcccaggcacttcgggaagaggaggctgaaatgatgcatcagatttacagctgcagtgacgagaacatagaagttttcaccaccgtgattccttccaaggtatccagtccagccagaagaagagccaaaagctctcagcacctcttgaccaagaatgtggtgatcgagtcggacctgtacacgcaccagcccctggagctgctgccccaccgcggagaccgcagggaccctggcgaccgccgcaggtttgggcggctccagaccgcgcggccgcccacagcccacccggccaaagcctctgccagacccgtggggatttctgaacccaaaacatcaaatctgtgtgggaatcgagcatatggaaaatctctgataccgccagtgccccggatctcagtgaaaacttcagcctctgcctcattggaggcgacagccatgggcacagagaagggagctgttctgatgagaggatccagacatctcaagaagatgactgaagagtatccagcccttccccaaggagcagaagcctctctaccactgacaggcagtgcttcctgcggcgtccccggcatcctccggaaaatgtggacaaggcacaagaagaagtctgaatatgtgggagccaccaacagcgcctttgaggccgactaaaggtgaccctcttcaagtgccctgtgttggccaaggttccccggacaagaggaaaaaccttcaggattgaaactgagccacacgcacctctgctagtagctggtccaaacccattatcctccctcactcattgattaccctgggatagggcacaggaaagaaatgtccctcgaaggcaatataaaactgccccttcttagaattgctaaagccattggtctgaaagtgactttgggaggtcataaagtttgtatctctatctttaagcaaaaaattaaactttcccagctcattttaaagacctccaaggaaggaaaaaagcaattcctctgtcttccttgtgagttgctctaaagtgtgtgattttctagtgtaaatggactttgaggcacttgtaaacacaatggttcttactgtttccattactgcatttacttcaccttgacaaggtacaattttcaaggacaaagcactatatataaagttagtagttctaatatccacttgagagtatactcaagattgattttcatggccatttggcatgattgcatgaattctctattcttttatgtgcagtttttctatagaaaaacattaaaagttaaatgttgactgctaatgttttctgaagtggcatagtctagtggaatcaataatcccttgctttggtataataatttgaacttttgaaagtgctttctcatccgttatctcttatcttcacaaaacccctgtggaagaaagcacaattattcccaatctgcagagggggaaatggaggccttcagagattacaggaccagataccaggtagtggaaccagacagcaggtccttaacttctctccagtggactccctgctttgcctactctctagcaactgacttggggaagtttttaaatggtgagagtgtgtctctgggggcaaccattgaaaagaagactctgatatgttggaacaagtaagggatttgaatgaaatacactattatatcttatacgttagcttgaaccagaaaaagttgacatttttgtaggtcaaaaacaattgagcatcaatttcatatggttcaacctcatatatgatactatgcttttcttctatcagtgaaaatgtcatcatcaattccattgtcaacattagtagccttaaaacaataacataataataatgatgataatgatacttatagctatcatctattaaatgcctatgtttcaggcatgatactaggcaatttatatatatcatcctaatctttctaaaaactctattaggtggccctatccacttaatagatgccaattcaaagaggtttaaatgattagactaaggcacctaacttatgtgagtgtcaggcttcaatgcctgtgttagagctactccttcacacaaaatagttcagaacatagagaaggaccaaggttaataaatgattttcatcccaaacactaaacatgattgatgggtagaggctgcccgaagtactgtgtaaagatggaatctgagatagaagaatgctgtggtcaattagtaattcttgcccatggagggattagtgacacatgccttgtatatttgtcatctgtggcctaaactctgcccctgaaggtttgttttctaattcagaggtttaaattaatctagcccacttaataaaaccagagatcctatgggaaatttagcctaagacagtgctggaaattgccatatgttgatacaaagaagtgtttggccacattacaggtctcagactcaactgctatgtgtgactgccgctctgtgcctatgtcttgcttttttgctgagttccctatttccatatctccaggtgaatccatgagaagcgagagggtggctgagaggcctgggcctctgggattccaccttgctatctctgctcttcaaccattgttttagactctgaacaccagatcctcatatctgaaagtgatttggagacctgggcatcaagtgctcttttaagaaggggctatcccagaggactgttcaaaagtctcattcaatagagatgttggagtccagaacaaagttagggagcaaaccagtaacctatgctggtcgtaacagaggatcctacaattacgtttgtttttaagacaggattttgctgtgttgcccagactggtctcaaactcctgggttcaagagatccatcctcccacctcagtctcctgaaagctgggatgacaggcacatgccaccacacctagctccttacaaccatttattttaacttatttcatttataactggtatctttcatttgtatgtggcagctagagatttatataggatggaagtaatttatttttaatttaaatatttcatgttgaactgtttgccttgtatggaacattttacttggccaattcaaataaaaataaagtcagctttgtttgtgaaaaaaaaaaaaaaaa

SEQ ID NO: 75 - DNA, 5446 bp, Homo sapiens
cgggaacccgtcaggaaggacataaacaaaacaaacccgaggcagcatggagaggggccgtggcccctgcagcggaaccggacccagtccctgagccgcccctacacccacagacagcatcgcacagaattattttaaaaaaaagcagtgatccaagcaattgaattggaagcactctggggaaacctgctgtttattgtggaaatcatcttcgatcttggaattgaaagtaaagctggaaaggaatttacaaacaagaaaaaaaagaagtttggaatcggattcacaggatctgggcttggaaatgcctcagcctagtgtaagcggaatggatccgcctttcggggatgcctttcgaagccacaccttttcggaacaaactctgatgagcacagatctcttagcaaacagttcggatccagatttcatgtatgaactggatagagagatgaactaccaacagaatcctagagacaactttctttctttggaggactgcaaagacattgaaaatctggagtctttcacagatgtcctggataatgagggtgctttaacctcaaactgggaacagtgggatacatactgtgaagacctaacgaaatataccaaactaaccagctgtgacatctggggaacaaaagaagtggattacttgggtcttgatgacttttctagtccttaccaagatgaagaggttataagtaaaactccaactttagctcaacttaatagtgaggactcacagtctgtttctgattccctttattaccccgattcacttttcagtgtcaaacaaaatcccttaccctcttcattccctggtaaaaagatcacaagcagagcagctgctcctgtgtgttcttctaagactctgcaggctgaggtccctttgtcagactgtgtccaaaaagcaagtaaacccacttcaagcacacaaatcatggtgaagaccaacatgtatcataatgaaaaggtgaactttcatgttgaatgtaaagactatgtaaaaaaggcaaaggtaaagatcaacccagtgcaacagagccggcccttgttgagccagattcacacagatgcagcaaaggagaacacctgctactgtggtgcagtggcaaagagacaagagaaaaaagggatggagcctcttcaaggtcatgccactcccgctttgccttttaaagaaacccaggaactattactaagtcccctgccccaggaaggtcctgggtcacttgcagcaggagagagcagcagtctttctgccagtacatcagtctcagattcatcccagaaaaaagaagagcacaattattctctttttgtctccgacaacttgggtgaacagccaactaaatgcagtcctgaagaagatgaggaggacgaggaggatgttgatgatgaggaccatgatgaaggattcggcagtgagcatgaactgtctgaaaatgaggaggaggaagaagaggaagaggattatgaagatgacaaggatgatgatattagtgatactttctctgaaccaggctatgaaaatgattctgtagaagacctgaaggaggtgacttcaatatcttcacggaagagaggtaaaagaagatacttctgggagtatagtgaacaacttacaccatcacagcaagagaggatgctgagaccatctgagtggaaccgagatactttgccaagtaatatgtatcagaaaaatggcttacatcatggaaaatatgcagtaaagaagtcacggagaactgatgtagaagacctgactccaaatcctaaaaaactcctccagataggcaatgaacttcggaaactgaataaggtgattagtgacctgactccagtcagtgagcttcccttaacagcccgaccaaggtcaaggaaggaaaaaaataagctggcttccagagcttgtcggttaaagaagaaagcccagtatgaagctaataaagtgaaattatggggcctcaacacagaatatggtaatttattgtttgtaatcaactccatcaagcaagagattgtaaaccgggtacagaatccaagagatgagagaggacccaacatggggcagaagcttgaaatcctcattaaagatactctcggtctaccagttgctgggcaaacctcagaatttgttaaccaagtgttagagaagactgcagaagggaatcccactggaggccttgtaggattaaggataccaacatcaaaggtgtaatcagcctcattggaccactggtcagaaatgtctgcgttttgtcacgttatccattgtaaattttcattctgttttgcatgtcagttagcattatgtaaacatttacaattaggttacattgttttaagaactaagtagcataagtgaagcatgatccaaaatacttgattattgcattttcagagcataaaccatgattaaaactgctactggcatcagaattgaaaatcatatgtttaagtaaatgttaggtacagattacaaaaatctgttaaagcaaaacattttggaggagtgaaatagtaaaatgccaagtattgtggcagatttatgctctgaaccacacaaaaaaattgaggaagcatttttttaaacagtcggtttaaattgtttttagaattattgctttttgttctaattttccacaaccattaatctcacttgtatatggcacacccagcacttgtgcctgtgggccatattagatgttcattgtcagagctcaagatgatatatataaatatatatatatatatatatatatacacacacacacacaaatgtctgtgcaagtaagaaaaaaaaagcatattctttgtgccttgtattttggggaaactctaaaactggtaatattttgtatgatgaaaaccctaatgagaaaaaacaagatatatagatggaaaaattatggggtttaaatgtttttttgttccaactctttttcagattttttgaatgtatataggactatgttgaaatgtagatatatgccacagagtctgtgtattgtataaaaaacaaaacaaaaaacaacaaaaaaaagatggctctagaaaactcatatttcggtacttgaccggaagaagacaaatacttgcacattattgcgattgttttattttttgtaccaaagacaaatgcaactgatatggcaaactgccagtctaagtaaagttttgcacagcttacatgatactgtatgaatgtatgaaaaaaaaggagaaaaaaaagaaaaaaaaaggtcagggttagggatcttactgaactgtgaattttatttctgtttgggtccaattatctacagaaggagcatccatacatacaaatattattttgctgttcctctagttcgcttccatagtagataagttggtggccatttagatgtcttttatttctgcacttattgtaggaaattttaatatatttcattttagtaagctattgataaaatagtttttgactttgaaaattaaaatgtttatttagcttattgtagtatacttccaccagacaacaaaatagattatttttattgtattatgtatatatatatatgtaaagaaagaaaaaagctaaaaatatctaattctttagttgccacttttccgattgatgtattattgtgcatgtaatattttcaaagatcaacacaggctaaaacaaaaacaatttatagatttttatatttttgtacaggtattttcaaactagcttcttcaaacttaacatgtgacttattcttctatagtttctagaattgagaaacattaacacatttagtttttaggtgctcttttttgctcatataaaacagcttcattagtcagtgttttaactgtgttcaagctttacctcttgatgagaaatttcttatgtcaaggcagcattataaaccttcccccacagatttttccatcctgtctctcttactgttttattctcaaatcttgtgctttgaactctgaaaactggtggcttaaaaactaaaaaaagaaaaaaagcatatttagcaaggaaaaaaataccaaaaatttcaggcatagctgctggaaaaattatctatttctccattacccactgtaggatttcttttttaattatactttgactataaagtgtcaaagtataatttgttcttttcttttactttgttaccccatttgtaagctatagcatatgaagctatatatatagcttgtgaaggtttgatctagaacacccagtaacaaatgaacaatgttgcttacctgcttctttgacatcttaaaaaagaaatccaaggaggattgtaaggattgtcttaccaccttagctgaactgtgatgcacaagatttttctatgtgtttggtggaaatgtacctggtttgtacattcacgctaaacagatgataagctcaagtctgatggtttaatagaatgtaagttcatcgtttaaagcttttcctttttaggttggagaaggcaaaacacaggcttgcaagttggaagtatatgaagtcttgacagagtgtgtctggtaaattgaaaagtgtttcaaactatggcagttttgcaatcaggtgaaaatcacctcatgatattcagctgataaggtttataaaattgcccctttctagctgctctgttaggaattctggtttttgatacttttttcctgtctgcaaaccagaatttgattttttggtcttgcatttcaaaaaaaaaaagactttgaatctgtttagtagattccatatctttgagtttcagtgttttatatgtactacttaagttaaatagttaaaagcttttaaatagttgagctttttaatgttgacactttattttgtacctatttatatatgtatgtatatcttagaaaagcactttgttaaaaaaaaattgcattttatatgattcctgccatttgctgctaaatctgggctggtcagaatgctgcagcgatacttgatctatataaaaacctggcagtaaaatgtagagtgaaagttaaatcctcttgctgttttaactttatcataaagatgacataggcaagctgtgcagctttacattttaaccaggggactctgtggcatttaaaaccgtctagaaatggttgtactttaatgccagtaataatctgcttcctctattgtcattaaaatatatacgtttagtgtatcacacaaaccaatcttataagggtaatgtaaaaaccccaacaattgtacatgttctgtttttgaaaattgtggcatgtatttttgggtgaagatcattagagaagagttctctaaaggttttctgtgttcatacatggtatacagatagctcataatgaagtccagaatcttacttttaagtgaaggcattgtgaattcacctcaagtaaacccattgttccaaagcaattataaactttgactctagtactactatgatttaaaaaaaaa

SEQ ID NO: 76 - DNA, 2353 bp, Homo sapiens
ggcggcggcggcagcagcgcggctcggtctctggtccattcactccacgctttctgcagccgccactgcagccgcgcggcgggggctccctccttgcagccagccggcggtccagcctggtgcctctgcaaaggaaaggggagcgtggagacgtgttcgaggtggtatcggcgaggatctctcgggcgccgctcactccttggtcgccttgcttgccagcagttgctcccttagtccttggctcgctcgcacaccccctcccgctacagggagcagttttgggtggcgtgggctccgtcctcttcttggctggtaggaacggtgtgcccaagaggggaagcctagtgggcctggcccctcccagccccgcgccaatgagtgccagggcgccgaaggagctgaggctggcgttgccgccgtgtctcctcaaccggacctttgcttcccccaacgccagcggcagcggcaacacgggtgcccgcggcccaggcgcagtaggcagcggcacctgcatcacgcaggtgggacagcagctcttccagtccttctcctccacgctggtgctgattgtcctggttaccgtcatcttctgcctcatcgtgctgtccctctccactttccacatccacaagcgtaggatgaagaagcggaagatgcagagggctcaggaggaatatgagcgggatcactgcagcggcagccgcggtggcggggggctgccccgacctggcaggcaggccccaacccacgcaaaggaaacccggctggagaggcagccccgggactctcccttctgcgccccttccaacgcctcgtcgttgtcctcttcgtcccctggcctcccgtgccagggtccctgtgctcctccgcctccaccgccagcctccagtccccaaggagcacacgcagcttcctcctgtttggacacagctggcgagggccttttgcaaacggtggtactgtcctgatcgtctagcccctctcgttccccgtcctcgtttccagcatctttgccacccttgcttttttccttcttccttccttttccattttcctctggcccctctttcctcttcctggtttccttacctgccctccccttactcttgtttctcctccgccgaggcactgtgcggtatttgtaaatattgggcgaggaaagtctcggaagaagaaataacgctgataataatactttattaatatttatagtaattattataatactaataacacaatccaagcgcatgacaaatcacataatttctaactgtcaatggaggatgcatcttccctttccaccccgcgctcacctcaattaggaggagaaaccgcacaagctaccaaatatataaaaggcattgattcccggagcaaagggaggggagggggcccggtaatgtacatagctgtcaagttaagatttagtttccttccttcccctctgacccctaagctttcacttttcctttagcttccctttcctccccattcccaccctcagccgggctcaggcaatagtatattataaagaaaatgtctacattaagcaccaggactgcaagaggccatagagaatagtccccggaaagtgtttatgacaggtacccctcgccagtcggcccatttctcaccctttggttaaccattacattttccaggacagagcatttaatttactttttaaaatgaccctcgctggccgagcataagtacgttaaaatctttaaatgagttttttttaaaaagctaacgctttcattccctgccccgcccccacccgtacacctttgacttgtgacattttcaggatttacaaaggatctgggagctgtccagccaggtctggtgctgaagtcgcctcaccgttctgattacttcctctagttgtgaaggcagaggaggggctgttttggaaagtgactattctggcttttggttgggttctttcttctttttctacaatcgagttagcgtgtactattggttttcttattattaaacattgcataagttaccttttttgtaaaaaaaaaaaagtattaggtgatgtgcagtactgaaagtgcagtatctaaccaactagaacgtttgttttatttttagaacaagtgcacctttgttatatatttagtatattggtaccaaatacagaaaaaaactatagttctgtactatgtcctccaaactgtatattattgttcttaatttccagctgttgatataatggttaccactggatgagaaattcagtggtgcagacctggcttctgctgtttccagaagtgttcttttgtaccttattctgtagtagactgttataaaaagatgacacacaaaaaaaaaaaaaaaa

SEQ ID NO: 77 - DNA, 4069 bp, Homo sapiens
gcagttgctggggctaacagaactggctgttgggagagagaggcgaggcaacgccgcccggccctgccattccattttactgcttaactcaacctggagtgtcaggacctattctcctgcattctcgctgcactatacgggggagccaagtcactctgtactcactggacagccaggctgggtggaatagggttctagagtggagcaggggctctgccctttatggcgactttctaaaagattagcatcagtgtcttttgacagtatagcatctggggtgggacgcggagaggaggctttgcttgctaaacggcttctttctccgccggtgatctggatttgtttcctcggccccctccccccgctcctttctttctcccctcccgcctttagcaaagtgacacaggcgacacctgctcgcttgtgttcgatgttggaactcgcacctcctcggtggtgacggtgccagggcactgctaccagggggacatcggcgggtttcccttccttccagcccacgatgcctagaagggcagcgggtgggctgcgtggggctctgcccgcagcacttcgaggcgggattgaggggctgaagctggccaggagttgctgctaagtggattcgagtggaaggcgccaagtctccgcgagggcaccagcggggacctatctgcgagcagtgggaacaggggcacctggacggaggaacgcgggcgtctggaggggggacatcggctgagctcagagcctctcctccttcctgtcccgagcttcccagcaactccgccacgttggagacccggtacctgcgagagccagggaactcgagaagtgcgtggccgaggctgctttcctgaaggtgaatcattctgtccaattgcctttccccagagaacaagagggagggcgggagagagggaacggaggatgtgtgagtgtgtgtgtgtgtgagagggagagacgacgtgaggcgagaggaaaagtctttagtcggccttaaaagcaaacaaatcagagatggaataagagcggtaattgcagcaagaccgccttgattcttgcagtccagggagctgagcgcaccgcgcgcatccggcgaggacaggaggcgaccgcgggcgctgccaagggctgcgggactttggcttttcctcagtaaacaaatcttttgattactttgacactgtggaataaagaagcggggagaaggatcaggctcaccttcacccgcttcagggggattccagcttggatgtcagattcctgaaccgtctttgccatcggaggaggaaacacatccacacacgcgcgcgcacactcgcacgctcagggccacactcacacgccgccctccacgatcacacagcaccagacttcggttccctatgcccctggatggtgacggcggattggcatcttggaagcgatgcgagagcgataaggctggcgccggcccgcaaaagctgcaggagcatcgctaggtgttgccgccaccgggaagcggggctgcaggatgagtaagagatacttacagaaagcaacaaaaggaaaactgctaataataatatttattgtaaccttgtgggggaaagttgtatccagtgcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactagggtaacccattaacccaggagaaatcaagtgatcctcaaggctgatgacattgaacatgcgcatagaaacttaactcaactcctgaggtgatcttgaagatttttataccacttgaaagaggcgctcaatagtctatttccaagggatttcatggcctcttcttgaaatcaagactttttaaaagtcagacatgaacttgcatgtcatgaagatttcagcagatttgaactgtgttcaacttgtaaattgttaaaagaatttgaagtcactgtctgaggagctggtgaagagttgtttttttcagggtgatgttagagacagtcaccttttgagttattggctccagatgtgactacttttcttgtttctgcaagctgtatcccaagtgcactgtccttctgtcctggatgtgttcctgggtcctatgttcatttgctagtgggactacacatggctttaatgacatttcctttgagaacttttcctctggcatggtgtagactgagacaattttatttatatcctaatcttggagctcagaaagcctacatgttttaacatcttaaagttgcttttgttaaaggaatggaaatatatatccattggtaataatgttggcaagtaatagttatctgaataaatcaatcatataagaatgtatagacaagctgacatatttccctaaggctaacaacaccctgctgaagctctttgtcaaataggtagtagttagaactggattgccattttcattatataatactttgtacctctagagcactctccctttctgtttttttttaagtgagcttttctttaattttttatgtttacttattcccttcacagaaatcagcagtgagcagtcaagttaatgggtagccttcagtttcaaaaaaattgacagggatgcatgtgagtttctgatttcttagcttgaacattattcacttagatttcttccagtattttttaaaaaactgtcctatctcattttaaaagactttcttttgcttgatcccaatgactgtttgaatgcttatatatttgttcaatctgttgatagaaaaaattgttcattttcctcagtctcaaatttataaatatttgcttacagttttcctattcaaacaatttgttaggccaatattttgtgacatttttgtagcgattttaacgtttatggttttggttctacaggaaagtcataaatatttaaaggccttaaacatgtatgtactttttttttctaagttatagaatgtataattttgtactacatttattttgtttcatttgtgatatgaagggagagaagaaagaaaagtgcatagccattctgtaacaatattgtgtaaacctatagtttgaaggaatgcaaggagaaggatttctgtgttttactcattttaggctgttcagaagatgcttcaaaaattgtcctgttagaatttccatcatggaaggtggtatggaagaaggtatggaaatactttgtattctaaaaactcactgacgtggtcagttagacatacgttggtttccaggatagaggcccatatatcctggggagctttggtctattagtttgtgacaatattcaaaggccaaaacactactcagacactttcctgggaagagcaactaaaaatgtaaaattggttaaaaataaaatctgaaaagtatgtatctcacattgaactaaaatccactgtctcataagttcatggaatgaaatggctttctgcctccattttaatcatgcataaaatgaattagatggctttgagtggattttcacaatggctcaagactatatgaaattataaaaaaaaagttgccctggggtttctgcatcaattagaatatcattaatttctttgtaaccaagtgaaaaactatactttttggaaattatgaatttgtcctaggtttgtttgagatttgaaattatacatcatgcttctcattttttaaactatgttctttaaatcaacactggaaactctgtattatatacaagtgtaatacatgcatataatagaaaaaaaacatggaatttcaaatatactaactagattatccccagtagattaatgttgtgactattcagaaaaggtgaataaaattgggatataaaatggactctctttcataaaaaaaaaaaaaaaa

SEQ ID NO: 78 - DNA, 4949 bp, Homo sapiens
cgccccactcggcgggtcggtgccgccgggtcccaggtgcccgctacttcccagaacctccgcctcccgctccgggccctcgaaccagcgcggacaccacaatggaccgggcgtccgagctgctcttctacgtgaacggccgcaaggtgatagaaaaaaatgtcgatcctgaaacaatgctgttgccttatttgaggaagaagcttcgactcacaggaactaagtatggctgtggaggaggaggctgtggtgcttgtacagtgatgatatcacgatacaaccccatcaccaagaggataaggcatcacccagccaatgcctgtctgattcccatctgttctctgtatggtgctgccgtcaccacagtagaaggcataggaagcacccacaccagaattcatcctgttcaggagaggattgccaagtgtcatggcacccagtgtggcttctgcacacctgggatggtgatgtccatctacacgctgctcaggaaccacccagagcccactctggatcagttaactgatgcccttggtggtaacctgtgccgttgcactggatacaggcccataattgatgcatgcaagactttctgtaaaacttcgggctgctgtcaaagtaaagaaaatggggtttgctgtttggatcaaggaatcaatggattgccagaatttgaggaaggaagtaagacaagtccaaaactcttcgcagaagaggagtttctgccattggatccaacccaggaactgatatttcctcctgagctaatgataatggctgagaaacagtcgcaaaggaccagggtgtttggcagtgagagaatgatgtggttttcccccgtgaccctgaaggaactgctggaatttaaattcaagtatccccaggctcctgttatcatgggaaacacctctgtggggcctgaagtgaaatttaaaggcgtctttcacccagttataatttctcctgatagaattgaagaactgagtgttgtaaaccatgcatataatggactcacccttggtgctggtctcagcctagcccaggtgaaggacattttggctgatgtagtccagaagcttccagaggagaagacacagatgtaccatgctctcctgaagcatttgggaactctggctgggtcccagatcaggaacatggcttctttagggggacacatcattagcaggcatccagattcagatctgaatcccatcctggctgtgggtaactgtaccctcaacttgctatcaaaagaaggaaaacgacagattcctttaaatgagcaattcctcagcaagtgccctaatgcagatcttaagcctcaagaaatcttggtctcagtgaacatcccctactcaaggaagtgggaatttgtgtcagccttccgacaagcccagcgacaggagaatgcgctagcgatagtcaattcaggaatgagagtcttttttggagaaggggatggcattattagagagttatgcatctcatatggaggcgttggtccagccaccatctgtgccaagaattcctgccagaaactcattggaaggcactggaacgaacagatgctggatatagcctgcaggcttattctgaatgaagtctcccttttgggctcggcgccaggtgggaaagtggagttcaagaggactctcatcatcagcttcctcttcaagttctacctggaagtgtcacagattttgaaaaagatggatccagttcactatcctagccttgcagacaagtatgaaagtgctttagaagatcttcattccaaacatcactgcagtacattaaagtaccagaatataggcccaaagcagcatcctgaagacccaattggccaccccatcatgcatctgtctggtgtgaagcatgccacgggggaggccatctactgtgatgacatgcctctggtggaccaggaacttttcttgacttttgtgactagttcaagagctcatgctaagattgtgtctattgatctgtcagaagctctcagcatgcccggtgtggtggacatcatgacagcagaacatcttagtgacgtcaactccttctgcttttttactgaagctgagaaatttctggcgacagataaggtgttctgtgtgggtcagcttgtctgtgctgtgcttgccgattctgaggttcaggcaaagcgagctgctaagcgagtgaagattgtctatcaagacttggagccgctgatactaacaattgaggaaagtatacaacacaactcctccttcaagccagaaaggaaactggaatatggaaatgttgacgaagcatttaaagtggttgatcaaattcttgaaggtgaaatacatatgggaggtcaagaacatttttatatggaaacccaaagcatgcttgtcgttcccaagggagaggatcaagaaatggatgtctacgtgtccacacagtttcccaaatatatacaggacattgttgcctcaaccttgaagctcccagctaacaaggtcatgtgccatgtaaggcgtgttggtggagcgtttggagggaaggtgttaaaaaccggaatcattgcagccgtcactgcatttgccgcaaacaaacatggccgtgcagttcgctgtgttctggaacgaggagaagacatgttaataactggaggccgccatccttaccttggaaagtacaaagctggattcatgaacgatggcagaatcttggccctggacatggagcattacagcaatgcaggcgcctccttggatgaatcattattcgtgatagaaatgggacttctgaaaatggacaatgcttacaagtttcccaatctccgctgccggggttgggcatgcagaaccaaccttccatccaacacagcttttcgtgggtttggctttcctcaggcagcgctgatcaccgaatcttgtatcacggaagttgcagccaaatgtggactatcccctgagaaggtgcgaatcataaacatgtacaaggaaattgatcaaacaccctacaaacaagagatcaatgccaagaacctaatccagtgttggagagaatgtatggccatgtcttcctactccttgaggaaagttgctgtggaaaagttcaatgcagagaattattggaagaagaaaggactggccatggtccccctgaagtttcctgttggccttggctcacgtgctgctggtcaggctgctgccttggttcacatttatcttgatggctctgtgctggtcactcacggtggaattgaaatggggcagggggtccacactaaaatgattcaggtggtcagccgtgaattaagaatgccaatgtcgaatgtccacctgcgtggaacaagcacagaaactgtccctaatgcaaatatctctggaggttctgtggtggcagatctcaacggtttggcagtaaaggatgcctgtcaaactcttctaaaacgcctcgaacccatcatcagcaagaatcctaaaggaacttggaaagactgggcacagactgcttttgatgaaagcattaacctttcagctgttggatacttcagaggttatgagtcagacatgaactgggagaaaggcgaaggccagcccttcgaatactttgtttatggagctgcctgttccgaggttgaaatagactgcctgacgggggatcataagaacatcagaacagacattgtcatggatgttggctgcagtataaatccagccattgacataggccagattgaaggtgcatttattcaaggcatgggactttatacaatagaggaactgaattattctccccagggcattctgcacactcgtggtccagaccaatataaaatccctgccatctgtgacatgcccacggagttgcacattgctttgttgcctccttctcaaaactcaaatactctttattcatctaagggtctgggagagtcgggggtgttcctggggtgttccgtgtttttcgctatccatgacgcagtgagtgcagcacgacaggagagaggcctgcatggacccttgacccttaatagtccactgaccccggagaagattaggatggcctgtgaagacaagttcacaaaaatgattccgagagatgaacctggatcctacgttccttggaatgtacccatctgaatcaaatgcaaacttctggagaaaacagagtgcctcttcccagatggcaatctgtcctatctctgtgctggaagatgctagatctgaaagacagagtttccacagttcagaaatcatcccacagtgttgcttttctatggagctgatttaaagtattccatttagatttgatagatatgcttaagcaatctataaatcattttcaatgttataaacactaattggtttcctctagggtgatattcgtcattactctgtctcttcaatccatccagctaaatggaataggtgatgacttgcatgtgactcctacttggcttctatccaccaacagaaattataccatatagtgaaaggcaattttctaaataatttcattactaatatgaactgtgaagttgtcattttttcatttgtccttttctgctatcaccttcctcttgtcagaatgaatatagacactgtatctaagtgggaccaaagaaaaaatagcgaactttcaccaaagttttcatgaaaacccaaaagctttaaaagttactatcaagaaattgaaaggaaacccacagaataggataaaatatttgtaaatcatatatttgataaaagtcttgtaaccagatacataaagagctcttacaactcaataaaaggcaagtaatttaaaaataggcaaaagaattgctggatggtatggtagttctatttttagtttttaccctaactactctgacttgatcatttaacattctgtgtatgtaacaaaatatcacatgcataaatattatgtatcaataaaattttttaatgggcaaaaaaaaaaaaaaaaaa

SEQ ID NO: 79 - DNA, 9121 bp, Homo sapiens
gcgcaactgacggtcgcgttctgcgcgcgagctagttgcctcccgtacctgccgcggtcgccggccccgcccccgggagcgcgggccaatgggctgggctccagggggcggggctggcgggcgggcggtgcggccgtggcggtagctgcaggggcggtggcggctgcagtggtggtggtgcctgtggctgtggctgcggctgcggctgcggctgagatttggccgggcgtccgcaggccgtgggggatgggggcagcgagctccagccctcggcggtggcggcggccgtaggtgtggggcgggcgtccgcgtccggcacgcgagatggagcgccgtggatttcagtttttctgactgttacatgaaaggatgattgctcacaaacagaaaaagacaaagaaaaaacgtgcttgggcatcaggtcaactctctactgatattacaacttctgaaatggggctcaagtccttaagttccaactctatttttgatccggattacatcaaggagttggtgaatgatatcaggaagttctcccacatgttactatatttgaaagaagccatattttcagactgttttaaagaagttattcatatacgtctagaggaactgctccgtgttttaaagtctataatgaataaacatcagaacctcaattctgttgatcttcaaaatgctgcagaaatgctcactgcaaaagtgaaagctgtgaacttcacagaagttaatgaagaaaacaaaaacgatctcttccaggaagtgttttcttctattgaaactttggcatttacctttggaaatatccttacaaacttccttatgggagatgtaggcaatgattcattattgcgactgcctgtttctcgagaaactaagtcgtttgaaaatgtttctgtggaatcagtggactcatccagtgaaaaaggaaatttttcccctttagaactagacaacgtgctgttaaagaacactgactctatcgagctggctttgtcatatgctaaaacttggtcaaaatatactaagaacatagtttcatgggttgaaaaaaagcttaacttggaattggagtccactagaaatatggtcaagttggcagaggcaactagaactaacattggaattcaggagttcatgccactgcagtctctgtttactaatgctcttcttaatgatatagaaagcagtcaccttttacaacaaacaattgcagctctccaggctaacaaatttgtgcagcctctacttggaaggaaaaatgaaatggaaaaacaaaggaaagaaataaaagagctttggaaacaggagcaaaataaaatgcttgaagcagagaatgctctcaaaaaggcaaaattattatgcatgcaacgtcaagatgaatatgagaaagcaaagtcttccatgtttcgtgcagaagaggagcatctgtcttcaagtggcggattagcaaaaaatctcaacaagcaactagaaaaaaagcgaaggttggaagaggaggctctccaaaaagtagaagaagcaaatgaactttacaaagtttgtgtgacaaatgttgaagaaagaagaaatgatctagaaaataccaaaagagaaattttagcacaactccggacacttgttttccagtgtgatcttacccttaaagctgtaacagttaacctcttccacatgcagcatctgcaggctgcttcccttgcagacagtttacagtctctctgtgatagtgccaaactctatgacccaggccaagagtacagtgaatttgtcaaggccacaaattcaactgaagaagaaaaagttgatggaaatgtaaataaacatttaaatagttcccaaccttcaggatttggacctgccaactctttagaggatgttgtacgccttcctgacagttctaataaaattgaagaggacagatgctctaacagtgcagatataacaggtccttcctttataagatcatggacatttgggatgtttagtgattctgagagcactggagggagcagcgaatctagatctctggattcagaatctataagtccaggagactttcatcgaaaacttccacgaacaccatccagtggaactatgtcctctgcagatgatctagatgaaagagagccaccttccccttcagaaactggacccaattcccttggaacatttaagaaaacattgatgtcaaaggcagctctcacacacaagtttcgcaaattgagatcccccacgaaatgtagggattgtgaaggcattgtagtgttccaaggtgttgaatgtgaagagtgtctccttgtttgtcatcgaaagtgtttggaaaatttagtcattatttgtggtcatcagaaacttccaggaaaaatacacttatttggagcagaattcacacaagttgcaaaaaaggaaccagatggtatcccttttatactcaaaatatgtgcctcagagattgaaaatagagctttgtgtctacagggaatttatcgtgtgtgtggaaacaaaataaaaactgaaaaattgtgtcaagctttggaaaatggaatgcacttggtagatatttcagaatttagttcacatgatatctgtgacgtcttgaaattataccttcggcagctcccagaaccatttattttatttcgattgtacaaggaatttatagaccttgcaaaagagatccaacatgtaaatgaagaacaagagacaaaaaagaatagtcttgaagacaaaaaatggccaaatatgtgtatagaaataaaccgaattcttctaaaaagcaaagaccttctaagacaattgccagcatcaaattttaacagtcttcatttccttatagtacatctaaagcgggtagtagatcatgcagaagaaaacaagatgaactccaaaaacttgggggtgatatttggaccaagtctcattaggccaaggcccacaactgctcctatcaccatctcctcccttgcagagtattcaaatcaagcacgcttggtagagtttctcattacttactcacagaagatcttcgatgggtccctacaaccacaagatgttatgtgtagcataggtgttgttgatcaaggctgttttccaaagcctctgttatcaccagaagaaagagacattgaacgttccatgaagtcactatttttttcttcaaaggaagatatccatacttcagagagtgaaagcaaaatttttgaacgagctacatcatttgaggaatcagaacgcaagcaaaatgcgttaggaaaatgtgatgcatgtctcagtgacaaagcacagttgcttctagaccaagaggctgaatcagcatcccaaaagatagaagatggtaaaacccctaagccactttctctgaaatctgataggtcaacaaacaatgtggagaggcatactccaaggaccaagattagacctgtaagtttgcctgtagatagactacttcttgcaagtcctcctaatgagagaaatggcagaaatatgggaaatgtaaatttagacaagttttgcaagaatcctgcctttgaaggagttaatagaaaagacgctgctactactgtttgttccaaatttaatggctttgaccagcaaactctacagaaaattcaggacaaacagtatgaacaaaacagcctaactgccaagactacaatgatcatgcccagtgcactccaggaaaaaggagtgacaacaagcctccagattagtggggaccattctatcaatgccactcaacccagtaagccatatgcagagccagtcaggtcagtgagagaggcatctgagagacggtcttcagattcctaccctctcgctcctgtcagagcacccagaacactgcagcctcaacattggacaacattttataaaccacatgctcccatcatcagtatcagggggaatgaggagaagccagcttcaccctcagcagcagtgcctcctggcacagatcacgatccccacggtctcgtggtgaagtcaatgccagacccagacaaagcatcagcttgtcctgggcaagcaactggtcaacctaaagaagactctgaggagcttggcttgcctgatgtgaatccaatgtgtcagagaccaaggctaaaacgaatgcaacagtttgaagacctcgaaggtgaaattccacaatttgtgtagggatgtcaaatttcagggtttttttgttgttgttgtgttattttgtggtattgtgcttgttttgtgaaagaatgttttgacagggcctcttttgtataggactgccaaatcatgggttttgccttttgttgttgtatttatcctctgttggtaatactgaatggtagaatgttttgatagggtcacatttgtgcctcactggaattatctttaaattctgtatttttaaagttgtgaataagataggtggattcgtattttttaaagttcagttgactttccccaccaaatggtccatttgaatgcatccctaatatatgatatagtctcaactaataggtgcaatttgggaaaatcaggtttattttttggagtggaactgttataagtgcttatttataaaaggaatgtttctgaatgcaagtgcctaaaaagatctttgttggtatgcatatgttttgtcacacaattttatagtgcatctttcaccatttgtgcttttttaagatacgtatgtaagctcttatttttcaattggcaattcagttaatttttaaatgtttacataatggccagaaggcttgcaaatctgtatttaattgcattttaattaattgccagtttttacatgtgatagtcagttgtacaaagaaaatgcacttaaacctgtttctaaattatatattcagttatattatatttggctttagatggttttaatacatttgatagtttttcaccccttggctttattttatataaacttttgtttttcagcagttctgaactttttagtattttataaatggtccaaaaaatgcctgtttcagaagtttttgaattcagtgcatttcctcttgatttgtctgggttaaaaccattccttttgtatgaaatgttttgacttaggaatcattttatgtacttgttctacctggattgtcaacaactgaaagtacatatttcatccaaatcaagctaaaatttatttaagttgattctgagagtacaggtcagtaagcctcattatttggaatttgagagaaggtataggtgatcggatctgtttcatttataaaaggtccagtttttaggactagtacattcctgttattttctgggttttatcattttgcctaaaataggatataaaagggacaaaaaataagtagactgtttttatgtgtgaattatatttctactaaatgtttttgtatgactgtgttatacttgataatatatatatatatatatatcaacttgttaaattatttcatgttcccgtggcttcttttcagttgttgcctattacagtatgagagtttaaggttattaaccattggcttagaagtcaacacctaggatcatacatccgttgactacaatgtggaatgaattttatggaaacctgttttatagttctatgtgatgtaaaggtctagggagcaataaccagcccttttttttctgaaggcttgtttttagtcctcattgtacaaaatataaaataatcatattgctatgaatacctaaaaagaaaattagagcccatgtgtattgagccttctctgtgtgccaggaactttgccaaggtgctacatagattattttatttcaagttcacagcaacctataatgggtgggtaagcatgttattatttctgtgttacagataaggaaactaaggcttagttaaatgacttgcttcagtgttccacaggtgtaacaggtggagtcaaatctcaaacatagggctgtcagattttgatggttagtgctcttaatgactgtgattactaaataatatctggtagtttttaacaatagaaaatgcaactttaaaaaatttttattgtggtaaaatacacataacatttaccatttttaagtatattgtttgaaccatttttaagcatatagttcagtggcattaagtatattcacattgttttgcaaccatgagcacctattcatctccagaacattatcatcatcctatactaaaacttagaaaatgtacttttaaacttcttcagttctcttttaatatatggagcccacccaaaggttatatcatgtaataaactcatttctctgttttccctcacagcttaaaaatgagaatttcacttttgggtcctttctgcctttagggctccctggcactttgtccagcgtagagccttaagcctctagatgctttctagtttcctggggccctgaggcaagtatctcttgagaagaggttgctatttctagaggatccacagtgtgcactgtgttgtgctatctaatagtgtggtggaaatcatttatccagaagttgttttttgcaacatggaaagatacgtgaccaaaaggaaagggcaacagcaggagccctgttagtgctgccagaacaccaggaagccttgtgggaggcgtattgtccaagatgatgcgtattgtccaaacgactcagaagaagtcatttctgaagggttgatcataacttccctagccatgttttacctacagagaacttagttagaatttatgagtacagtatgttaaattacttttagtgtaccttaggcagtgtatttgttttgatacagagacaaagactatatgatccctgagacttgttgcctagtgaactcacaagcagatagaattgtacctcagttatttgggtgatttaaagatggtactatggctacactattcccagctatcgttgtgttacagtatttatacaactggaaacatctctctaaatgacaaccattgtatattgttaaataacttatagtcagtggaattctgtgggtttttctttcctacaggcaggaaattatggtgatgtgttgatatgattatatatgtagactattcagttaacatctaggagttagcaaatttgctacattcacaatcagtatgtaagtcagttgtattcctagatatcagcaactaacagaaatggaagtttttagaaagatctcttcaatatcattaaaaaatacaaaatactctacctaagaataagtctaacaaaagatgtacaagatcttgatggagaaaatgttagaccatgtcaggaagcatttaaggagatcttaaataaatgtagtactccataatgtacacgaattagaagactcagtcgtgtaaagatgttcatttacatttgtagattcaatgtaatcccaaccaaaatattttttgcattattacctctatctactgtgtgtttccttaagacaagggcattcctacgtaaccatagtacaaccatcaaacttaggagattgacattgacatcatgatatctaatccatagaccacattcaatctgctgattttgattgcctcagtaatgttcctgtgtttcagcatcccaatccaggaccacatagtgcatttagttgccctatctcttgtttcattcagtccagaacatcttttcagacttgtctttgaccttgacacttgaggaggattggccagtttgtagaaagttcctcagttttggtttatcattagataagacccataaggtttaggatagacatttttgtccaatactacagaaatgatgctctatactcaaatgtgtcatgccaggatgcacacaatgtcaatttgtcttatacctgatagtattaattttggtcacttggttcagatagtatataccaggtttctccattgtaaaattagaattaggaagtaatttgtgaggagattcttaaagattgtaaatgtattgtttctcatcgaattgtcacgggctagtctattgacaattcttgactgaaccaattactattttggttgtcaaatggtgagtccgtggcttctgccttggttggcatcctactactgtaaggaagagctcttccttctctattccttcattcacttaaatcagtatagattcatgaatgcctgctttattcagtgacagtgtattattgtcacttattttcatgctgaaattttccccgatttggtttttgggagtctcttcaagctggcttctgtgttccttagagatgcctcattgctttttcccctcttattttttttacgagctcatcttacactttttcctactccctaccccaagtctagaattagcccttttttccaaggagccctggtttctttaaatgggaaatggtatatggaaatgaagacctgagaaaggctcactgtcttattggtgaacagctcaaaggaaaacgtgtgtctacacacattcaagaatagtcacctacttctatatctatttatatacatacgtattgaaaactttgagttcacattgacaccttcaattcaatcctttgaggttaatctagtctccctttccatctttgtaacttccttctctcatagaaacctgactcccataatcctaaataatttgttcattcttcctccctccctcaacctcttgacatgccagctgctgtctcatcacacccagtcccaaatatgccaaataaatccgcagtcatagcagcgctggtcctgaccacaccaatccctaacatcctccccagcctggagcctggaccatggcagctgaatctttgaccccagtgatgtcagtcctgagcacaccattggaatcctaggctcctgtcatctggctggaagagagacaaattcacaaccttttgataatgagcaattattgaatactcactgaatttatcagagaaagtttaaaagtgtacgattatacatatttaccaggaatgtttaaacatagacatttatgaaatttatagaaaatattgaattgtggttctcactttttggtcttggctgtaacaatttttaaatattaatacttcctacaataactgtatcatgctatttttaagtgtcctcacactcaaccaatctactctgcatccataaaataagcatgcagttcagaaaatagattaaacgactaattggctaaaaa

SEQ ID NO: 80 - DNA, 5118 bp, Homo sapiens
ggctgggctgcgaatagcgtgttcctctccggcggaacacacacacccggccttggggctgtctcctgagctccctcctccacggagagcgctgagcgccgccgggaattccatcccaccgtgggcacgcagtctttggaggtcccgggcgcagcacgctcggtgtccccacactgcagcaagacagagaccccgcgggaaccttgagcttggaacaacccttgagcctctgcagtcggaagagtgggcgcagcagcccagcggaggccaggcgcgcaacctcgggcgccggggcaaggagagagtgcagggaggcgcagctcaggcgcccggctcaggagcgggaggaagttctcgcggcgccgggagcgcggtggacgcgccctgggcgcacgcccaggcagccttctccctggccctcgggactgtcctcgggccgcaaggaggagcttgctggagtcttagaggccatccagagccagcgagcaggagcgctgcgtctcccgcctcagctaggaagggggagtggcgctggcaggctggagctgggaacccagcgagcgcctgaccttcctcctcctcttcctgaccctcttcgcgtcttgggctccggaggaaggttctagcggctgcaggaggtccccagacccattttcctagaaggctggtgatggatctgctgctcctgccgccgccggggcacttggagcgcaccggcggcgcgtgagctgggctttgctctccactgccctgggcaaaccccgggccagccccgcctggcacctttgcctgagtccctttcggttcccgacccaaagccaccagcgtccagggagggaggaggaggtggtcctcaggtgcagccccgccgagatgtccgcgcagagcctgctccacagcgtcttctcctgttcctcgcccgcttcaagtagcgcggcctcggccaagggcttctccaagaggaagctgcgccagacccgcagcctggacccggccctgatcggcggctgcgggagcgacgaggcgggcgcggagggcagtgcgcggggagccacggcgggccgcctctactccccatcactcccagccgagagtctcggccctcgcttggcgtcctcttcccggggtccgccccccagggccaccaggctaccgcctcctggacctctttgctcgtccttctccacacccagcaccccgcaggagaagtcaccatccggcagctttcactttgactatgaggttcccctgggtcgcggcggcctcaagaagagcatggcctgggacctgccttctgtcctggccgggccagccagtagccgaagcgcttccagcatcctctgttcatccgggggaggccccaatggcatcttcgcttctcctaggaggtggctccagcagaggaagttccagtccccacccgacagtcgcgggcacccctacgtcgtgtggaaatccgagggtgatttcacctggaacagcatgtcaggccgcagtgtacggctgaggtcagtccccatccagagtctctcagagctggagagggcccggctgcaggaagtggctttttatcagttgcaacaggactgtgacctgagctgtcagatcaccattcccaaagatggacaaaagagaaagaaatctttaagaaagaaactggattcactaggaaaggagaaaaacaaagacaaagaattcatcccacaggcatttggaatgcccttatcccaagtcattgcgaatgacagggcctataaactcaagcaggacttgcagagggacgagcagaaagatgcatctgactttgtggcttccctcctcccatttggaaataaaagacaaaacaaagaactctcaagcagtaactcatctctcagctcaacctcagaaacaccgaatgagtcaacgtccccaaacaccccggaaccggctcctcgggctaggaggaggggtgccatgtcagtggattctatcaccgatcttgatgacaatcagtctcgactactagaagctttacaactttccttgcctgctgaggctcaaagtaaaaaggaaaaagccagagataagaaactcagtctgaatcctatttacagacaggtccctaggctggtggacagctgctgtcagcacctagaaaaacatggcctccagacagtggggatattccgagttggaagctcaaaaaagagagtgagacaattacgtgaggaatttgaccgtgggattgatgtctctctggaggaggagcacagtgttcatgatgtggcagccttgctgaaagagttcctgagggacatgccagacccccttctcaccagggagctgtacacagctttcatcaacactctcttgttggagccggaggaacagctgggcaccttgcagctcctcatataccttctacctccctgcaactgcgacaccctccaccgcctgctacagttcctctccatcgtggccaggcatgccgatgacaacatcagcaaagatgggcaagaggtcactgggaataaaatgacatctctaaacttagccaccatatttggacccaacctgctgcacaagcagaagtcatcagacaaagaattctcagttcagagttcagcccgggctgaggagagcacggccatcatcgctgttgtgcaaaagatgattgaaaattatgaagccctgttcatggttcccccagatctccagaacgaagtgctgatcagcctgttagagaccgatcctgatgtcgtggactatttactcagaagaaaggcttcccaatcatcaagccctgacatgctgcagtcggaagtttccttttccgtgggagggaggcattcatctacagactccaacaaggcctccagcggagacatctccccttatgacaacaactccccagtgctgtctgagcgctccctgctggctatgcaagaggacgcggccccggggggctcggagaagctttacagagtgccagggcagtttatgctggtgggccacttgtcgtcgtcaaagtcaagggaaagttctcctggaccaaggcttgggaaagatctgtcagaggagcctttcgatatctggggaacttggcattcaacattaaaaagcggatccaaagacccaggaatgacaggttcctctggagacatttttgaaagcagctccctaagagcggggccctgctccctttctcaagggaacctgtccccaaattggcctcggtggcaggggagccccgcagagctggacagcgacacgcagggggctcggaggactcaggccgcagcccccgcgacggagggcagggcccaccctgcggtgtcgcgcgcctgcagcacgccccacgtccaggtggcagggaaagccgagcggcccacggccaggtcggagcagtacttgaccctgagcggcgcccacgacctcagcgagagtgagctggatgtggccgggctgcagagccgggccacacctcagtgccaaagaccccatgggagtgggagggatgacaagcggcccccgcctccatacccgggcccagggaagcccgcggcagcggcagcctggatccaggggcccccggaaggcgtggagacacccacggaccagggaggccaagcagccgagcgagagcagcaggtcacgcagaaaaaactgagcagcgccaactccctgccagcgggcgagcaggacagtccgcgcctgggggacgctggctggctcgactggcagagagagcgctggcagatctgggagctcctgtcgaccgacaaccccgatgccctgcccgagacgctggtctgagcccgcacccagccgagccccccctgccccgagccccccgccctccagcccaggggggaccgtgggtggtggccactggcacacttagtgttcttctttcacacttctcaaaagtgacacaagagaaatccagttcacctacagaggtagagcactcacgcccccgccattgagaataaggttccattgcgtagccagccttaggaaaaacaaacagaacccaaaccagatggcaatgtccaatctaaaaacgtccctcttggctctataatataagatacaactcttgcttggtatagcctaaccgtatttatgtgtcttcggttttgactattgtgtattctgtaacagattatgtataatcatatatgatatattcacaaagagaaaacaaaaggaacttttaaaaaaaaaatcacttcacttatattaagcaatgagatatactaaacaatgagattctatagaatgttctagaatgtgcacaagcgggtttctgtgcttttgccatagctttataactggggataacccttccttcgataccaaacactaacaagaggaagcagaatatgagaagccatatttttacataggagtcagatacaaaaagaaaaatcactgaatgcttttagatattgaatacgttttcaggaaaatgctaaatctgatagattacgaaatatatttttagaacttgtttagaaaggattcagttaaccaaacaagaaaaaggcagtgcctcacaaagaaattaagaagttgtccgtcccacgttacatcaaattcagttttatataggccatatataatatatatttataatgtataatttttatgtatttttcaaaactacaaactggaatccaactataaagtgtttaagaatctacacagaatattcaaattatagaacatgttttttccctttgccccataatcagtatttgccaaattacatgcaattccttaaaaactaaatcacatttggtaaaaggcctacagctttgtacttacattgtgccaaaggctgaggaaatgttttctttcgtaattttatgtgtattgtaaaatgttctaccgtactttagtagtttgaagtttttcaagtgcataactatttttgaccagcagatggcgatacgcttcagtattttatgcaattttttttcacttctgaagggaaagtgtattataaaaaaagattttttttttttttataaaacatgctactcttaattttcatgttggtgatgaaattcccagtggtgtttcttaaggttctatcttgtgccatgatgaataaaaagttaagcaaag

SEQ ID NO: 81 - DNA, 1465 bp, Homo sapiens
atgagtgagaggcgggtggtagtggacttgcccaccagtgccagctccagcatgcccctccagaggcgcagggcgtccttcagggggccacggtcatcatcctccctggagagccccccagcctccaggaccaatgccatgagtggccttgtccgagcacccggggtctatgtaggaacagcacccagtgggtgcataggtggcttgggtgcccgtgtgacccgccgggccctcggcatcagcagtgtcttccttcagggcctgcggagctcaggcctggccaccgtgccggctccaggtttggagagggaccatggtgctgttgaggacctagggggctgcctggtggaatatatggccaaagtgcacgcccttgagcaagtcagtcaggagctggaaacacaactgcggatgcacctggagagcaaagccacacgctcgggaaactggggtgcsctacgggcttcctgggccagcagctgccagcaggtgggtgaggcagtcttggaaaatgcccggctcatgctgcagacagaaactatccaggccggagcagatgactttaaagagagatatgaaaatgagcagccatttcgaaaggcagcagaagaggaaattaactctctgtataaagtcattgatgaggctaatttgactaaaatggacctggagagtcaaatagaaagtctgaaagaagaacttggctctctatcaagaaactatgaagaggatgtgaagctgctgcacaaacagttggcagggtgtgagctggaacaaatggatgctcccattggcactggtctggacgacatccttgagacgatcagaattcagtgggagagagatgttgaaaagaaccgggtggaggcaggagccctgctccaagctaagcaacaggcggaggtggcccacatgtcccagacccaggaggagaagctggcagctgccctcagggtggagttacacaacacttcgtgccaagtccagagcctccaggctgagacagaatccttacgtgccctgaaacgaggcctggagaacaccttgcacgatgccaagcactggcatgacatggagctccagaacctgggcgctgtggtcggccggctggaggcggagctcagggaaatccgagcggaggcggagcagcagcaacaggagcgcgcgcatctgctggcccgcaagtgccagctgcagaaggacgtggcgtcctaccacgccctgctggacagggaggagagcggctgatggagaaacttcctctttttcatgaagaaaacacccttcctcaacagctgacccaagaagttgcttgaggagctttctcctgagctccagtccctgctggattccctggttaattcagcttgagctgaaaagcttcctggaagtggagagatccttctgctttaatctgagtagtctgtagcttgagcaatctccttgtcctcttccaataatgctt

SEQ ID NO: 82 - DNA, 2401 bp, Homo sapiens
agcctcccgcccgccgcctctgtctccctctctccacaaactgcccaggagtgagtagctgctttcggtccgccggacacaccggacagatagacgtgcggacggcccaccaccccagcccgccaactagtcagcctgcgcctggcgcctcccctctccaggtccatccgccatgtggcccctgtggcgcctcgtgtctctgctggccctgagccaggccctgccctttgagcagagaggcttctgggacttcaccctggacgatgggccattcatgatgaacgatgaggaagcttcgggcgctgacacctcgggcgtcctggacccggactctgtcacacccacctacagcgccatgtgtcctttcggctgccactgccacctgcgggtggttcagtgctccgacctgggtctgaagtctgtgcccaaagagatctcccctgacaccacgctgctggacctgcagaacaacgacatctccgagctccgcaaggatgacttcaagggtctccagcacctctacgccctcgtcctggtgaacaacaagatctccaagatccatgagaaggccttcagcccactgcggaagctgcagaagctctacatctccaagaaccacctggtggagatcccgcccaacctacccagctccctggtggagctccgcatccacgacaaccgcatccgcaaggtgcccaagggagtgttcagcgggctccggaacatgaactgcatcgagatgggcgggaacccactggagaacagtggctttgaacctggagccttcgatggcctgaagctcaactacctgcgcatctcagaggccaagctgactggcatccccaaagacctccctgagaccctgaatgaactccacctagaccacaacaaaatccaggccatcgaactggaggacctgcttcgctactccaagctgtacaggctgggcctaggccacaaccagatcaggatgatcgagaacgggagcctgagcttcctgcccaccctccgggagctccacttggacaacaacaagttggccagggtgccctcagggctcccagacctcaagctcctccaggtggtctatctgcactccaacaacatcaccaaagtgggtgtcaacgacttctgtcccatgggcttcggggtgaagcgggcctactacaacggcatcagcctcttcaacaaccccgtgccctactgggaggtgcagccggccactttccgctgcgtcactgaccgcctggccatccagtttggcaactacaaaaagtagaggcagctgcagccaccgcggggcctcagtgggggtctctggggaacacagccagacatcctgatggggaggcagagccaggaagctaagccagggcccagctgcgtccaacccagccccccacctcgggtccctgaccccagctcgatgccccatcaccgcctctccctggctcccaagggtgcaggtgggcgcaaggcccggcccccatcacatgttcccttggcctcagagctgcccctgctctcccaccacagccacccagaggcaccccatgaagcttttttctcgttcactcccaaacccaagtgtccaaggctccagtcctaggagaacagtccctgggtcagcagccaggaggcggtccataagaatggggacagtgggctctgccagggctgccgcacctgtccagacacacatgttctgttcctcctcctcatgcatttccagcctttcaaccctccccgactctgcggctcccctcagcccccttgcaagttcatggcctgtccctcccagacccctgctccactggcccttcgaccagtcctcccttctgttctctctttccccgtccttcctctctctctctctctctctctctctctctttctgtgtgtgtgtgtgtgtgtgtgtgtgtgtgtgtgtgtgtgtgtgtcttgtgcttcctcagacctttctcgcttctgagcttggtggcctgttccctccatctctccgaacctggcttcgcctgtccctttcactccacaccctctggccttctgccttgagctgggactgctttctgtctgtccggcctgcacccagcccctgcccacaaaaccccagggacagcggtctccccagcctgccctgctcaggccttgcccccaaacctgtactgtcccggaggaggttgggaggtggaggcccagcatcccgcgcagatgacaccatcaaccgccagagtcccagacaccggttttcctagaagcccctcacccccactggcccactggtggctaggtctccccttatccttctggtccagcgcaaggaggggctgcttctgaggtcggtggctgtctttccattaaagaaacaccgtgcaacgtgaaaaaaaaaaaaaaaaaaaaa

SEQ ID NO: 83 - DNA, 1460 bp, Homo sapiens
gacggcctggcatacccactgcccaccccagtgactgctcttctgcttcaggcctgctggcctcccagcactgcctgcccctccctgtcgggggacatcgcctccacaccggctggggaaggagcccaggggtggggctggtgggtggggctggtggttggggcagccagagaagtaagagggaagtgagaagccgggtggggcaggctggaaggaagacgaacctacgaagcagagatctgaagacagcatgtacacagccattccccagagtggctctccattcccaggctcagtgcaggatccaggcctgcatgtgtggcgggtggagaagctgaagccggtgcctgtggcgcaagagaaccagggcgtcttcttctcgggggactcctacctagtgctgcacaatggcccagaagaggtttcccatctgcacctgtggataggccagcagtcatcccgggatgagcagggggcctgtgccgtgctggctgtgcacctcaacacgctgctgggagagcggcctgtgcagcaccgcgaggtgcagggcaatgagtctgacctcttcatgagctacttcccacggggcctcaagtaccaggaaggtggtgtggagtcagcatttcacaagacctccacaggagccccagctgccatcaagaaactctaccaggtgaaggggaagaagaacatccgtgccaccgagcgggcactgaactgggacagcttcaacactggggactgcttcatcctggacctgggccagaacatcttcgcctggtgtggtggaaagtccaacatcctggaacgcaacaaggcgagggacctggccctggccatccgggacagtgagcgacagggcaaggcccaggtggagattgtcactgatggggaggagcctgctgagatgatccaggtcctgggccccaagcctgctctgaaggagggcaaccctgaggaagacctcacagctgacaaggcaaatgcccaggccgcagctctgtataaggtctctgatgccactggacagatgaacctgaccaaggtggctgactccagcccatttgcccttgaactgctgatatctgatgactgctttgtgctggacaacgggctctgtggcaagatctatatctggaaggggcgaaaagcgaatgagaaggagcggcaggcagccctgcaggtggccgagggcttcatctcgcgcatgcagtacgccccgaacactcaggtggagattctgcctcagggccatgagagtcccatcttcaagcaatttttcaaggactggaaatgagggtgggcgtcttcctgccccatgctcccctgccccccaccacctgcctgcttgcttctctggctgcctggtcagtgcagaggtgccccctgcagatgttcaataaaggagacaagtgctttcccagctcttttcctgcaccaccaaaaaaaaaaaaaaaaaa

SEQ ID NO: 84 - DNA, 1319 bp, Homo sapiens
atacatagtttactttcatttttgactctgaggctctttccaacgctgtaaaaaaggacagaggctgttccctatggcagaaggcaaccacagaaaaaagccacttaaggtgttggaatccctgggcaaagatttcctcactggtgttttggataacttggtggaacaaaatgtactgaactggaaggaagaggaaaaaaagaaatattacgatgctaaaactgaagacaaagttcgggtcatggcagactctatgcaagagaagcaacgtatggcaggacaaatgcttcttcaaaccttttttaacatagaccaaatatcccccaataaaaaagctcatccgaatatggaggctggaccacctgagtcaggagaatctacagatgccctcaagctttgtcctcatgaagaattcctgagactatgtaaagaaagagctgaagagatctatccaataaaggagagaaacaaccgcacacgcctggctctcatcatatgcaatacagagtttgaccatctgcctccgaggaatggagctgactttgacatcacagggatgaaggagctacttgagggtctggactatagtgtagatgtagaagagaatctgacagccagggatatggagtcagcgctgagggcatttgctaccagaccagagcacaagtcctctgacagcacattcttggtactcatgtctcatggcatcctggagggaatctgcggaactgtgcatgatgagaaaaaaccagatgtgctgctttatgacaccatcttccagatattcaacaaccgcaactgcctcagtctgaaggacaaacccaaggtcatcattgtccaggcctgcagaggtgcaaaccgtggggaactgtgggtcagagactctccagcatccttggaagtggcctcttcacagtcatctgagaacctagaggaagatgctgtttacaagacccacgtggagaaggacttcattgctttctgctcttcaacgccacacaacgtgtcctggagagacagcacaatgggctctatcttcatcacacaactcatcacatgcttccagaaatattcttggtgctgccacctagaggaagtatttcggaaggtacagcaatcatttgaaactccaagggccaaagctcaaatgcccaccatagaacgactgtccatgacaagatatttctacctctttcctggcaattgaaaatggaagccacaagcagcccagccctccttaatcaacttcaaggagcaccttcattagtacagcttgcatatttaacattttgtatttcaataaaagtgaagacaaacga

SEQ ID NO: 85 - DNA, 2704 bp, Homo sapiens
gggagaaacgttctcactcgctctctgctcgctgcgggcgctccccgccctctgctgccagaaccttggggatgtgcctagacccggcgcagcacacgtccgggccaaccgcgagcagaacaaacctttggcgggcggccaggaggctccctcccagccaccgcccccctccagcgcctttttttccccccatacaatacaagatcttccttcctcagttcccttaaagcacagcccagggaaacctcctcacagttttcatccagccacgggccagcatgtctgggggcaaatacgtagactcggagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaagattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcatcagccgtgtctattccatctacgtccacaccgtctgtgacccactctttgaagctgttgggaaaatattcagcaatgtccgcatcaacttgcagaaagaaatataaatgacatttcaaggatagaagtatacctgattttttttccttttaattttcctggtgccaatttcaagttccaagttgctaatacagcaacaatttatgaattgaattatcttggttgaaaataaaaagatcactttctcagttttcataagtattatgtctcttctgagctatttcatctatttttggcagtctgaatttttaaaacccatttaaatttttttccttacctttttatttgcatgtggatcaaccatcgctttattggctgagatatgaacatattgttgaaaggtaatttgagagaaatatgaagaactgaggaggaaaaaaaaaaaaaagaaaagaaccaacaacctcaactgcctactccaaaatgttggtcattttatgttaagggaagaattccagggtatggccatggagtgtacaagtatgtgggcagattttcagcaaactcttttcccactgtttaaggagttagtggattactgccattcacttcataatccagtaggatccagtgatccttacaagttagaaaacataatcttctgccttctcatgatccaactaatgccttactcttcttgaaattttaacctatgatattttctgtgcctgaatatttgttatgtagataacaagacctcagtgccttcctgtttttcacattttccttttcaaatagggtctaactcagcaactcgctttaggtcagcagcctccctgaagaccaaaattagaatatccatgacctagttttccatgcgtgtttctgactctgagctacagagtctggtgaagctcacttctgggcttcatctggcaacatctttatccgtagtgggtatggttgacactagcccaatgaaatgaattaaagtggaccaatagggctgagctctctgtgggctggcagtcctggaagccagctttccctgcctctcatcaactgaatgaggtcagcatgtctattcagcttcgtttattttcaagaataatcacgctttcctgaatccaaactaatccatcaccggggtggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccgtctgttatgctgtgacacatggcccctccccctgccaggagctttggacctaatccaagcatccctttgcccagaaagaagatgggggaggaggcagtaataaaaagattgaagtattttgctggaataagttcaaattcttctgaactcaaactgaggaatttcacctgtaaacctgagtcgtacagaaagctgcctggtatatccaaaagctttttattcctcctgctcatattgtgattctgcctttggggacttttcttaaaccttcagttatgatttttttttcatacacttattggaactctgcttgatttttgcctcttccagtcttcctgacactttaattaccaacctgttacctactttgactttttgcatttaaaacagacactggcatggatatagttttacttttaaactgtgtacataactgaaaatgtgctatactgcatactttttaaatgtaaagatatttttatctttatatgaagaaaatcacttaggaaatggctttgtgattcaatctgtaaactgtgtattccaagacatgtctgttctacatagatgcttagtccctcatgcaaatcaattactggtccaaaagattgctgaaattttatatgcttactgatatattttacaattttttatcatgcatgtcctgtaaaggttacaagcctgcacaataaaaatgtttaacggtt

SEQ ID NO: 86 - DNA, 4246 bp, Homo sapiens
gctctcactctggctgggagcagaaggcagcctcggtctctgggcggcggcggcggcccactctgccctggccgcgctgtgtggtgaccgcaggccccagacatgagggcggcccgtgctctgctgcccctgctgctgcaggcctgctggacagccgcgcaggatgagccggagaccccgagggccgtggccttccaggactgccccgtggacctgttctttgtgctggacacctctgagagcgtggccctgaggctgaagccctacggggccctcgtggacaaagtcaagtccttcaccaagcgcttcatcgacaacctgagggacaggtactaccgctgtgaccgaaacctggtgtggaacgcaggcgcgctgcactacagtgacgaggtggagatcatccaaggcctcacgcgcatgcctggcggccgcgacgcactcaaaagcagcgtggacgcggtcaagtactttgggaagggcacctacaccgactgcgctatcaagaaggggctggagcagctcctcgtggggggctcccacctgaaggagaataagtacctgattgtggtgaccgacgggcaccccctggagggctacaaggaaccctgtggggggctggaggatgctgtgaacgaggccaagcacctgggcgtcaaagtcttctcggtggccatcacacccgaccacctggagccgcgtctgagcatcatcgccacggaccacacgtaccggcgcaacttcacggcggctgactggggccagagccgcgacgcagaggaggccatcagccagaccatcgacaccatcgtggacatgatcaaaaataacgtggagcaagtgtgctgctccttcgaatgccagcctgcaagaggacctccggggctccggggcgaccccggctttgagggagaacgaggcaagccggggctcccaggagagaagggagaagccggagatcctggaagacccggggacctcggacctgttgggtaccagggaatgaagggagaaaaagggagccgtggggagaagggctccaggggacccaagggctacaagggagagaagggcaagcgtggcatcgacggggtggacggcgtgaagggggagatggggtacccaggcctgccaggctgcaagggctcgcccgggtttgacggcattcaaggaccccctggccccaagggagaccccggtgcctttggactgaaaggagaaaagggcgagcctggagctgacggggaggcggggagaccagggagctcgggaccatctggagacgagggccagccgggagagcctgggccccccggagagaaaggagaggcgggcgacgaggggaacccaggacctgacggtgcccccggggagcggggtggccctggagagagaggaccacgggggaccccaggcacgcggggaccaagaggagaccctggtgaagctggcccgcagggtgatcagggaagagaaggccccgttggtgtccctggagacccgggcgaggctggccctatcggacctaaaggctaccgaggcgatgagggtcccccagggtccgagggtgccagaggagccccaggacctgccggaccccctggagacccggggctgatgggtgaaaggggagaagacggccccgctggaaatggcaccgagggcttccccggcttccccgggtatccgggcaacaggggcgctcccgggataaacggcacgaagggctaccccggcctcaagggggacgagggagaagccggggaccccggagacgataacaacgacattgcaccccgaggagtcaaaggagcaaaggggtaccggggtcccgagggcccccagggacccccaggacaccaaggaccgcctgggccggacgaatgcgagattttggacatcatcatgaaaatgtgctcttgctgtgaatgcaagtgcggccccatcgacctcctgttcgtgctggacagctcagagagcattggcctgcagaacttcgagattgccaaggacttcgtcgtcaaggtcatcgaccggctgagccgggacgagctggtcaagttcgagccagggcagtcgtacgcgggtgtggtgcagtacagccacagccagatgcaggagcacgtgagcctgcgcagccccagcatccggaacgtgcaggagctcaaggaagccatcaagagcctgcagtggatggcgggcggcaccttcacgggggaggccctgcagtacacgcgggaccagctgctgccgcccagcccgaacaaccgcatcgccctggtcatcactgacgggcgctcagacactcagagggacaccacaccgctcaacgtgctctgcagccccggcatccaggtggtctccgtgggcatcaaagacgtgtttgacttcatcccaggctcagaccagctcaatgtcatttcttgccaaggcctggcaccatcccagggccggcccggcctctcgctggtcaaggagaactatgcagagctgctggaggatgccttcctgaagaatgtcaccgcccagatctgcatagacaagaagtgtccagattacacctgccccatcacgttctcctccccggctgacatcaccatcctgctggacggctccgccagcgtgggcagccacaactttgacaccaccaagcgcttcgccaagcgcctggccgagcgcttcctcacagcgggcaggacggaccccgcccacgacgtgcgggtggcggtggtgcagtacagcggcacgggccagcagcgcccagagcgggcgtcgctgcagttcctgcagaactacacggccctggccagtgccgtcgatgccatggactttatcaacgacgccaccgacgtcaacgatgccctgggctatgtgacccgcttctaccgcgaggcctcgtccggcgctgccaagaagaggctgctgctcttctcagatggcaactcgcagggcgccacgcccgctgccatcgagaaggccgtgcaggaagcccagcgggcaggcatcgagatcttcgtggtggtcgtgggccgccaggtgaatgagccccacatccgcgtcctggtcaccggcaagacggccgagtacgacgtggcctacggcgagagccacctgttccgtgtccccagctaccaggccctgctccgcggtgtcttccaccagacagtctccaggaaggtggcgctgggctagcccaccctgcacgccggcaccaaaccctgtcctcccacccctccccactcatcactaaacagagtaaaatgtgatgcgaattttcccgaccaacctgattcgctagattttttttaaggaaaagcttggaaagccaggacacaacgctgctgcctgctttgtgcagggtcctccggggctcagccctgagttggcatcacctgcgcagggccctctggggctcagccctgagctagtgtcacctgcacagggccctctgaggctcagccctgagctggcgtcacctgtgcagggccctctggggctcagccctgagctggcctcacctgggttccccaccccgggctctcctgccctgccctcctgcccgccctccctcctgcctgcgcagctccttccctaggcacctctgtgctgcatcccaccagcctgagcaagacgccctctcggggcctgtgccgcactagcctccctctcctctgtccccatagctggtttttcccaccaatcctcacctaacagttactttacaattaaactcaaagcaagctcttctcctcagcttggggcagccattggcctctgtctcgttttgggaaaccaaggtcaggaggccgttgcagacataaatctcggcgactcggccccgtctcctgagggtcctgctggtgaccggcctggaccttggccctacagccctggaggccgctgctgaccagcactgaccccgacctcagagagtactcgcaggggcgctggctgcactcaagaccctcgagattaacggtgctaaccccgtctgctcctccctcccgcagagactggggcctggactggacatgagagccccttggtgccacagagggctgtgtcttactagaaacaacgcaaacctctccttcctcagaatagtgatgtgttcgacgttttatcaaaggccccctttctatgttcatgttagttttgctccttctgtgtttttttctgaaccatatccatgttgctgacttttccaaataaaggttttcactcctctaaaaaaaaaaaaaaaaaaaaa

SEQ ID NO: 87 - DNA, 3455 bp, Homo sapiens
gcttactcggcgcccgcgcctcgggccgtcgggagcggagcctcctcgggaccaggacttcagggccacaggtgctgccaagatgctccagggcacctgctccgtgctcctgctctggggaatcctgggggccatccaggcccagcagcaggaggtcatctcgccggacactaccgagagaaacaacaactgcccagagaagaccgactgccccatccacgtgtacttcgtgctggacacctcggagagcgtcaccatgcagtcccccacggacatcctgctcttccacatgaagcagttcgtgccgcagttcatcagccagctgcagaacgagttctacctggaccaggtggcgctgagctggcgctacggcggcctgcacttctctgaccaggtggaggtgttcagcccaccgggcagcgaccgggcctccttcatcaagaacctgcagggcatcagctccttccgccgcggcaccttcaccgactgcgcgctggccaacatgacggagcagatccggcaggaccgcagcaagggcaccgtccacttcgccgtggtcatcaccgacggccacgtcaccggcagcccctgcgggggcatcaagctgcaggccgagcgggcccgcgaggagggcatccggctcttcgccgtggcccccaaccagaacctgaaggagcagggcctgcgggacatcgccagcacgccgcacgagctctaccgcaacgactacgccaccatgctgcccgactccaccgagatcgaccaggacaccatcaaccgcatcatcaaggtcatgaaacacgaagcctacggagagtgctacaaggtgagctgcctggaaatccctgggccctctggccccaagggctaccgtggacagaagggtgccaagggcaacatgggtgagccgggagagcctggccagaagggaagacagggagacccgggcatcgaaggccccattggattcccaggacccaagggcgttcctggcttcaaaggagagaagggtgaatttggagccgacggtcgcaagggggcccctggcctggctggcaagaacgggaccgatggacagaagggcaagctggggcgcatcggacctcctggctgcaagggagaccctggaaaccggggccccgacggttacccgggggaagcagggagtccaggggagcgaggagaccaaggcggcaagggggaccctggccgcccaggacgcagagggcccccgggagaaatcggggccaagggaagcaaggggtatcaaggcaacagtggagccccaggaagtcctggtgtgaaaggagccaagggcgggcctgggccccgcggacccaaaggcgagccggggcgcaggggagaccccggcaccaagggcagcccaggcagcgatggccccaagggggagaagggggaccctggccctgaggggccccgcggcctggctggagaggttggcaacaaaggagccaagggagaccgaggcttgcctggacccagaggcccccagggagctcttggggagcccggaaagcagggatctcggggagaccccggtgatgcaggaccccgtggagactcaggacagccaggccccaagggagaccccggcaggcctggattcagctacccaggaccccgaggagcacccggagaaaaaggcgagcccggcccacgcggccccgagggaggccgaggcgactttggcttgaaaggagaacctgggaggaaaggagagaaaggagagcctgcggatcctggtccccctggtgagccaggccctcgggggccaagaggagtcccaggacccgagggtgagcccggcccccctggagaccccggtctcacggagtgtgacgtcatgacctacgtgagggagacctgcgggtgctgcgactgtgagaagcgctgtggcgccctggacgtggtcttcgtcatcgacagctccgagagcattgggtacaccaacttcacactggagaagaacttcgtcatcaacgtggtcaacaggctgggtgccatcgctaaggaccccaagtccgagacagggacgcgtgtgggcgtggtgcagtacagccacgagggcacctttgaggccatccagctggacgacgaacgtatcgactccctgtcgagcttcaaggaggctgtcaagaacctcgagtggattgcgggcggcacctggacaccctcagccctcaagtttgcctacgaccgcctcatcaaggagagccggcgccagaagacacgtgtgtttgcggtggtcatcacggacgggcgccacgaccctcgggacgatgacctcaacttgcgggcgctgtgcgaccgcgacgtcacagtgacggccatcggcatcggggacatgttccacgagaagcacgagagtgaaaacctctactccatcgcctgcgacaagccacagcaggtgcgcaacatgacgctgttctccgacctggtcgctgagaagttcatcgatgacatggaggacgtcctctgcccggaccctcagatcgtgtgcccagaccttccctgccaaacagagctgtccgtggcacagtgcacgcagcggcccgtggacatcgtcttcctgctggacggctccgagcggctgggtgagcagaacttccacaaggcccggcgcttcgtggagcaggtggcgcggcggctgacgctggcccggagggacgacgaccctctcaacgcacgcgtggcgctgctgcagtttggtggccccggcgagcagcaggtggccttcccgctgagccacaacctcacggccatccacgaggcgctggagaccacacaatacctgaactccttctcgcacgtgggcgcaggcgtggtgcacgccatcaatgccatcgtgcgcagcccgcgtggcggggcccggaggcacgcagagctgtccttcgtgttcctcacggacggcgtcacgggcaacgacagtctgcacgagtcggcgcactccatgcgcaagcagaacgtggtacccaccgtgctggccttgggcagcgacgtggacatggacgtgctcaccacgctcagcctgggtgaccgcgccgccgtgttccacgagaaggactatgacagcctggcgcaacccggcttcttcgaccgcttcatccgctggatctgctagcgccgccgcccgggccccgcagtcgagggtcgtgagcccaccccgtccatggtgctaagcgggcccgggtcccacacggccagcaccgctgctcactcggacgacgccctgggcctgcacctctccagctcctcccacggggtccccgtagccccggcccccgcccagccccaggtctccccaggccctccgcaggctgcccggcctccctccccctgcagccatcccaaggctcctgacctacctggcccctgagctctggagcaagccctgacccaataaaggctttgaacccataaaaaaaaaaaaaaaa

SEQ ID NO: 88 - DNA, 1019 bp, Homo sapiens
cgaggctgcaccagcgcctggcaccatgaggacgcctgggcctctgcccgtgctgctgctgctcctggcgggagcccccgccgcgcggcccactcccccgacctgctactcccgcatgcgggccctgagccaggagatcacccgcgacttcaacctcctgcaggtctcggagccctcggagccatgtgtgagatacctgcccaggctgtacctggacatacacaattactgtgtgctggacaagctgcgggactttgtggcctcgcccccgtgttggaaagtggcccaggtagattccttgaaggacaaagcacggaagctgtacaccatcatgaactcgttctgcaggagagatttggtattcctgttggatgactgcaatgccttggaatacccaatcccagtgactacggtcctgccagatcgtcagcgctaagggaactgagaccagagaaagaacccaagagaactaaagttatgtcagctacccagacttaatgggccagagccatgaccctcacaggtcttgtgttagttgtatctgaaactgttatgtatctctctaccttctggaaaacagggctggtattcctacccaggaacctcctttgagcatagagttagcaaccatgcttctcattcccttgactcatgtcttgccaggatggttagatacacagcatgttgatttggtcactaaaaagaagaaaaggactaacaagcttcacttttatgaacaactattttgagaacatgcacaatagtatgtttttattactggtttaatggagtaatggtacttttattctttcttgatagaaacctgcttacatttaaccaagcttctattatgcctttttctaacacagactttcttcactgtctttcatttaaaaagaaattaatgctcttaagatatatattttacgtagtgctgacaggacccactctttcattgaaaggtgatgaaaatcaaataaagaatctcttcacatgagaaaaaaaaaaaaaaaaaaaaa

SEQ ID NO: 89 - DNA, 4988 bp, Homo sapiens
agaaaaacggggagcaggagccagactaggggaggaagaggactggcccgctcagggaatagctgggttgctgcaaaaaggggcggggagaaggcgggggcgctgcatgcagcgcgctggctccagcggtggccgcggggaatgtgacatcagcggcgccgggcgcttggggctggaggaggcagctcgcctcagctgcgctgtgcacacctcgcccgggggaggacgcagacccgggcaggcggcagggatgtcggcgaaggagaggccaaagggcaaagtgatcaaggacagcgtcaccctcctgccctgcttttatttcgtcgagttgcctatattggcatcatcggtggttagcctctatttcctcgaactcacagatgtcttcaaacctgtgcactctggatttagctgctatgaccggagtcttagcatgccgtacattgaaccaacccaggaggcaattccattcctcatgttgcttagcttggcttttgctggacctgcaattacgattatggtaggagaaggaattctctactgttgcctctccaaaagaagaaatggggtcggactagagcccaacattaatgctggaggctgcaacttcaattccttcctcagacgagctgtcagattcgttggtgttcatgtatttggattatgctctacagctctcattacagatatcatacagctgtccacaggatatcaagcaccttactttctgactgtgtgcaaaccaaactatacctctctgaatgtatcttgcaaagaaaattcctacattgtggaagatatttgctcaggatctgacctcacagttatcaacagtggcagaaagtccttcccttctcaacatgcaacccttgctgcctttgcagctgtgtatgtttcgatgtacttcaattccacattaacggattcctctaagcttctgaaacctctcttggtcttcacatttatcatctgtggaataatctgcgggctaacacggataactcagtataagaaccacccagttgatgtctattgtggctttttaataggaggaggaattgcactgtacttgggcttgtatgctgtggggaatttcctgcccagtgatgagagtatgtttcagcacagagacgccctcaggtctctgacagacctcaatcaagatcccaaccgacttttatctgctaaaaatggtagcagcagtgatggaattgctcatacagaaggcatcctcaaccgaaaccacagagatgctagctctctgacaaatctcaaaagagcaaatgctgatgtggaaatcattactccacggagccccatggggaaggagaacatggttaccttcagcaataccttgccgcgagccaataccccatctgtagaagaccctgtcagaagaaatgcgagcattcatgcctctatggattccgctcgatcaaagcagctcctcacccagtggaagaataagaatgaaagtcgaaagttgtccttgcaagttatagagcctgagcctgggcagtcaccacccagatccatagaaatgaggtcaagctcagagccatcgagggtaggggtgaatggagaccaccatggtcctggcaatcagtacctcaaaatccagcctggcgctgtccccggatgtaacaacagcatgcctggagggccaagagtgtccattcagtcccgtcctgggtcctcacagttggtgcacatccctgaggagactcaggaaaacataagcacctcccccaaaagcagctctgctcgggccaagtggttaaaagctgctgaaaagactgtggcctgtaacagaagcaacagccagccccgaatcatgcaagtcatagccatgtccaagcagcagggtgtcctccaaagcagccccaagaacactgaaggcagcacggtctcctgcactggctccatccgctataaaaccttgacagaccatgagcccagtgggatagtgagggttgaggctcacccagagaacaacaggcccatcatacagatcccgtccactgaaggtgaaggcagtggctcctggaagtggaaagcccctgaaaagggcagccttcgccaaacttacgagctcaacgatctcaacagggactcagaaagctgtgagtctctgaaagacagctttggttctggagatcgcaagagaagcaacattgatagcaatgagcatcaccaccacggaattaccaccatccgcgtcaccccagtagagggcagcgaaattggctcagagacgctgtccatttcttcttcccgcgactccaccctgcggagaaagggcaatatcattctaatccctgaaagaagcaacagccccgaaaacactagaaatatcttctacaaaggaacctcccccacacgggcttataaggattgagtgatgtccattccatcattagggctactcgcaaaagaccatatgttgattctacctgtgttctgttccagcgaattgggaagtctcaccaagctagattgtctaccatcagcccagaactctgtaacttttcagaactgctatactcaaacttgcagatctcacatcaaggagagggaaaagcacaatgcaagaacctaactaacgtgatgatatgaagagttttcttaagacctgtcgtcaaacttaaaaggttttgcagagggcagtatcaaaagaaagtggttttcttcaaatgtatactattttacttcctgaatgtgccaactttggggatttttctttatagtgagctgtgggaacccagaacacacacgttttccctacagcagaggccatgcagtattatatattcattttgcagaatctgcacctacagctcaatacgggtggtgctgattattatagtacatataccatgtaaactctcaaactctatttagctgtgaaatagtggtgtgcaattccttgttaaagaaatgctactttattaagaagatgctggctgctttgtgttagaataggacaccccgcagcttctctgtagtggctctgtcacagtcaaaaaatgaaaaggtttttgtgcgtttcttcaaaattctgctttcttcaacatcaaaaattgtgtagaaatattttcagtgaaagggaataactagtacttttctgcatagtttttcttctgcttactttttatttaagtataggtactgctaatgaatctgttttcttagtgagtaaatttgcataattttataaatattattttagagaatcttttgaaattgttgtgatcatattttgctttctatggcttctccttaacttattgattaattttttgaagttatagatatgttctcctattttaaaagcaaaaataacaattgacattccttgagcaaaatatactgctgtgaatttgcaaacaagaaatctgagccaaaacttgacattgtgggttacattgccagaaatgttggtcaagtttgcccttagatgtctacaactagctggcataggttgccatcttaacaagtaatctaaaagtcccattcggttctacattattaactttttttttctatatcctgatgaccagtaaattagagccacactggttaagtttgactcgtctctaaaacgtttttgttaattggacaccaagaggaagaatctgaaaaaaaaatgcatgttggtaagtaaaagtatctcacggtacaaattaagaatgactttcttcaaaatatctgaataggtgcagttttagtttaacatgcaaacaaccattgttgctacctatcctgaatcaagccttgagcctaaatcaaagcaaaccaataccattgataagaagaagataaaaacaaaatattttggagtgttttccaacttaaagtatgaagacatactcagttcttggaacttagtattaaaccttttttatgccatttcataagaattccgatatatacttgatgattgccaaggggatgaaaggaaacaacagagatggttgatctgatcttagctcactttccaataacagaaggagttgtttacagatgaatagtatcacatcattatcaatttccacatgaaaaaggtggagctttctagaaaaaccaacctctaaggcattaggaatttagctgaaaccagcagaattgaaaactctggcaataaaacatggactcaaccatatcccttctggcaatttccttctcagagaggggagtgggaataaaatgttgccttccccacttctcaccaccaccgccatcatgacgctcatactggcttttgcctgtttgtagaggaaaaggtgggctggttttagtactctgaaggacaaaaacaagcaaacaaaaacccctgctgcagcatttcaggtgcagtatgatatttcctaatctttcctatttcttaacaaaagattttaaagtacttctctagtcattgaagtttttttttctttacataaatattgatatattctttttctactcaaagtgccaaaggctacagtttttaatgacttaacaaattgtaccacattgttaaggacatataatgatagacactagaactcagacctctgcatgtatatttgataacatgtcttttgtaaaacaaaaattacaaaaaaatttgtttacattccactggtaccttaatttaaaataaatcagactaaaaggtggtatctcttcttagtgttctatttatcttatttgctaatgggagcacttcttcctttgttaggctgtgctttactgataaaaccaagtattgaataaagagagttaattatctttttaaagtaaataaaattatgaaaatatatatagtatatataaagtactgtgtttaaaaaaatgttatgcaatgttttccaaactgataaagtttgtaaagtgctataaatgtattttgttaagtacagataaaagctattgtgtgagtatattgtgctaaaatcatagaaataaagattagatttcttcatcaaaaaaaaaaaaaaaaaaaa

SEQ ID NO: 90 - DNA, 3206 bp, Homo sapiens
ggcacttggatctctcaaatggtgcagtgactcggataccttccctagtgccattacagtactggagactgccagctagatccatcacacccaagtgaagctgtggaaaagcccttaaactccagagccagaaccagcaacctcagctccggaatacacttgcaaggcactggaagatctaaaattcctctttaaacaaaaagataagtaatgccccaccaacatcctttcacctcaaagtaaggtgatcccaatactagaaattttactggcaattgctctgattgttatcactattttaaccctaacttgtacaccaccaggagttccattggcagctcgttttgtgaccagtttctcttaggtcaccatgggcctgctcctgctggttctcattctcacgccttcactagcagcctaccgccatcctgatttcccgttattggaaaaagctcagcaactgctccaaagtacaggatccccttactccaccaattgctggttatgtactagctcttccactgaaacaccagggacagcttatccagcctcgcccagagaatggacaagcatagaggcggaattacatatttcctatcgatgggaccctaatctgaaaggactgatgaggcctgcaaatagtcttctttcaacagtaaagcaagatttccctgatatccgccagaaacctcccattttcggacccatctttactaatatcaacctaatgggaatagcccctatttgtgttatggccaaaaggaaaaatggaacaaatgtaggcactcttccaagtacagtctgtaatgttactttcactgtagattctaaccaacagacttaccaaacatacacccacaaccaattccgccatcaaccaagattccccaaacctccaaatattacttttcctcagggaactttgctagataaatccagccggttttgccagggacgcccaagctcatgcagtactcgaaacttctggttccggcctgctgattataaccaatgtctgcaaatttccaacctcagctctacagcggaatgggttctattggaccaaactcgaaattctcttttttgggaaaataaaaccaagggagctaaccagagccaaacaccctgcgtccaagtcttagcaggcatgactatagccaccagctacctgggcatatcagcagtctcagaattttttggaacctccctcacccccttatttcatttccatatctctacatgccttaaaactcaaggagccttttatatttgtggccagtcgattcaccaatgcctccccagtaactggactggaacttgtaccataggctatgtaaccccagacatcttcatagcccctggcaatctctctcttccaataccaatctatgggaattccccgttgcccagggtgaggagggcaatccatttcattccccttctcgcgggactcggcattctagctggtacgggaaccggaattgctggaatcacaaaagcttccctcacctatagccagctctcaaaggaaatagccaacaacattgacaccatggctaaagccttaacgaccatgcaagaacaaatcgactctttagcagccgtagtccttcaaaatcgtcgaggactagacatgttaacggcagcacagggaggaatttgtttggccttagatgaaaaatgttgcttttgggtaaatcaatcaggaaaagtacaagacaacatcagacaactcctaaatcaagcctccagtttacgggaacgagccactcagggttggttaaattgggaaggaacttggaaatggttctcttgggttcttccccttacaggcccacttgttagtctcctacttttgctcctttttggtccatgtctcctaaatctaataacccaatttgtctcctctcgccttcaggccataaagctccagacgaatctcagtgcaggacgccatcctcgcaatattcaagagtcacccttctaaggaggacccctagactgctcgctagtggaacacgacagaggcgaaatcctgccccgtctcccgtggacctggctggatatggtttttgccaatccacagagccatcctgccctgacagctagcaagaggccaagacccacagaacaaccactacagcccctctgtcagcaggaagcagttaaagaagactgaccttcgtccattttcccagataattgggtcttggactcttgaggtggggaaatgttggagcaggtagctagtcagacatgagcagggcaggggagggccccctcaccaggaatgtcaggcaaccatcaggtgatggtcaggcagttgttaagctgtgtctctaacataataatgagtggcagctggcgccagggaactatggcctcccaatagataggaaacacctgaagctggtgatcagccgcttcctgataagatctcaggagttgggtgcgcaggctcaagcatgcaccctaagaggcaaaatagtggcatttaactcatatatgaccttcctttaggaaggcttgactggtaagggaaaaactcctccagtgaacacgtgcacaacttcagtaaaaacactgcacatgcgtcccctcccaagtgctggcaggccactgtgcatgcagacagcccgccccaaagaaaaatcagaggaggagaaatggaaaccccggaaaaatgccaatgtataaaaccccaagtcaagggcctaccaaggcaattggatctctcaagtcacccgcttggctctcttcaagtgcactttgcttccttttgttcttgctctaaaacttttactcctgctctaaaacttgccttggactatcatgctaccttacgcctcccgggccaaattccctcctctcctccggggggcaaggatggagtctgctgcagacccattggatttgctgctggtaacagttccaccatttaggttccagcaccaagcaaactaacacccgactcagtgtaaacagccaaacaagcttaaccaattagaaaccaccatctaacctctaactaggtcctttcaactttaaccaagtattttctttgtcttgcttctgtgggaaccttataaaattttcccccttgtacctctgtagtagagacccagttgcttgcagtttggccctgcctgttcatgaatcacccttgctcaaataaactctctaaaatgctaaaaaaaaaaaaaaaa

SEQ ID NO: 91 - DNA, 1975 bp, Homo sapiens
aactgtcactgtggagaggagagagagaggacagagagcaagtcactcccggctgcctttttcacctctgacagagcccagacaccatgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccagctgcaggaatcaaaaattgaagagagatatatctgaaaactggaataagaagcaaataaatatcatcctgccttcatggaactcagctgtctgtggcttcccatgtctttctccaaagttatccagagggttgtgattttgtctgcttagtatctcatcaacaaagaaatattatttgctaattaaaaagttaatcttcatggccatagcttttattcattagctgtgatttttgttgattaaaacattatagattttcatgttcttgcagtcatcagaagtggtaggaaagcctcactgatatattttccagggcaatcaatgttcacgcaacttgaaattatatctgtggtcttcaaattgtcttttgtcatgtggctaaatgcctaataaacaattcaagtgaaatactaaaaaaaaaaaaaaaaaaaaaa

SEQ ID NO: 92 - DNA, 1825 bp, Homo sapiens
ttttttttttttttttttttttttttaatcttgcactttgaaaccgcgggaccgaggcagggtgcgcgcgtgtggttggtgcctttttttttttttcttcccctccctaaactcctctgtcagtctgtaaacattacctgagaattccccagccgaaacggctgctggggcaagaaacttcttgttagaactttccacctccggcttccccctccacctcttttaccgtcccaaccttaggagacgctttttctcccccagaggagaatttatctttttttttttttttttttttctttttctcacccggtgctttgcatttgggaagaggtgatttcaagagtggccaggtgggacgcctctctcctccttattcggtttactatttattgttcggggtgttttttaattcctgtattgctcggcccggggagtttcgccccctgcccggctccgcggcgcggaggatggtgtggaaacggctgggcgcgctggtgatgttccctctacagatgatctatctggtggtgaaagcagccgtcggactggtgctgcccgccaagctgcgggacctgtcgcgggagaacgtcctcatcaccggcggcgggagaggcatcgggcgtcagctcgcccgcgagttcgcggagcgcggcgccagaaagattgttctctggggccggactgagaaatgcctgaaggagacgacggaggagatccggcagatgggcactgagtgccattacttcatctgtgatgtgggcaaccgggaggaggtgtaccagacggccaaggccgtccgggagaaggtgggtgacatcaccatcctggtgaacaatgccgccgtggtccatgggaagagcctaatggacagtgatgatgatgccctcctcaagtcccaacacatcaacaccctgggccagttctggaccaccaaggccttcctgccacgtatgctggagctgcagaatggccacatcgtgtgcctcaactccgtgctggcactgtctgccatccccggtgccatcgactactgcacatccaaagcgtcagccttcgccttcatggagagcctgaccctggggctgctggactgtccgggagtcagcgccaccacagtgctgcccttccacaccagcaccgagatgttccagggcatgagagtcaggtttcccaacctctttcccccactgaagccggagacggtggcccggaggacagtggaagctgtgcagctcaaccaggccctcctcctcctcccatggacaatgcatgccctcgttatcttgaaaagcatacttccacaggctgcactcgaggagatccacaaattctcaggaacctacacctgcatgaacactttcaaagggcggacatagagacaggatgaagacatgcttgaggagccacggagtttgggggccacagcacctgggcacacacccgagcacctgtccattggcatgcttctgctgggtgagcaggacagctcctgtccccagcgaagaatccggctgcccctgggccagtcccaggacctttgcacaggactgatgggtataactgacccccacagggaggcaggaaaacagccagaagccaccttgacacttttgaacatttccagttctgtagagtttattgtcaattgcttctcaagtctaaccagcctcagcagtgtgcatagaccatttccaggagggtctgtccccagatgctctgcctcccgttccaaaacccactcatcctcagcttgcacaaactggttgaacggcaggaatgaaaaataaagagagatggcttttgtgaaaaaaaaaaaaaaaaaaaa

SEQ ID NO: 93 - DNA, 3856 bp, Homo sapiens
gcaaggcgacagctgtgccagccgggctctggcaggctcctggcagcatggcagtgaagcttgggaccctcctgctggcccttgccctgggcctggcccagccagcctctgcccgccggaagctgctggtgtttctgctggatggttttcgctcagactacatcagtgatgaggcgctggagtcattgcctggtttcaaagagattgtgagcaggggagtaaaagtggattacttgactccagacttccctagtctctcgtatcccaattattataccctaatgactggccgccattgtgaagtccatcagatgatcgggaactacatgtgggaccccaccaccaacaagtcctttgacattggcgtcaacaaagacagcctaatgcctctctggtggaatggatcagaacctctgtgggtcactctgaccaaggccaaaaggaaggtctacatgtactactggccaggctgtgaggttgagattctgggtgtcagacccacctactgcctagaatataaaaatgtcccaacggatatcaattttgccaatgcagtcagcgatgctcttgactccttcaagagtggccgggccgacctggcagccatataccatgagcgcattgacgtggaaggccaccactacgggcctgcatctccgcagaggaaagatgccctcaaggctgtagacactgtcctgaagtacatgaccaagtggatccaggagcggggcctgcaggaccgcctgaacgtcattattttctcggatcacggaatgaccgacattttctggatggacaaagtgattgagctgaataagtacatcagcctgaatgacctgcagcaagtgaaggaccgcgggcctgttgtgagcctttggccggcccctgggaaacactctgagatatataacaaactgagcacagtggaacacatgactgtctacgagaaagaagccatcccaagcaggttctattacaagaaaggaaagtttgtctctcctttgactttagtggctgatgaaggctggttcataactgagaatcgagagatgcttccgttttggatgaacagcaccggcaggcgggaaggttggcagcgtggatggcacggctacgacaacgagctcatggacatgcggggcatcttcctggccttcggacctgatttcaaatccaacttcagagctgctcctatcaggtcggtggacgtctacaatgtcatgtgcaatgtggtgggcatcaccccgctgcccaacaacggatcctggtccagggtgatgtgcatgctgaagggccgcgccagcactgccccgcctgtctggcccagccactgtgccctggcactgattcttctcttcctgcttgcataactgatcatattgcttgtctcagaaaaaaacaccatcagcaaagtgggcctccaaagccagatgattttcattttatgtgtgaataatagcttcattaacacaatcaagaccatgcacattgtaaatacattattcttggataattctatacataaaagttcctacttgttaaaaaagatacaaaccttgtttttccagaaggtaggaaaatcctagctttccatttgtgcagttatatgtcattttctcctttcttttcacgttactcaggatgaactctctgagcagggacctgctcctgcagcaaccaaacttggagtggttattgcagacagacgtggctctgggcccctctctgtcccaccttgcacaaaggaccccctcagaccaggcccttgtctgtgccctgtccacacccaggagccatcctcagtgtctgtggccacaatcctgtactgttccttccatccctgataaaaggaggtctacatgaaagcaaaagctactgtctatttctgacccagctcatggaattttttcatcttatactgagctccagaaaggacgtaacttagcatggatcaccaatcaatcaaaaaataaataaatcactaaggattggagaactcatagaacaaggtgaaagacatgagtgccctcccaaagtctgagtgcacgaaaatttctctcttgccttgaggagcagaaaagcttctgatggacatgggcttctgtgagacttatcacacatagtgtatcgtggcatgaagcccggcacatagcaggccctgcatattgatggacaaatggatggcctgcctgccttccctgtccgttcacctgtgcaaaggcttcctcagacatgccactctgtggctcccaatatagggtgcagacaagagcaatccctgacatgacattatagcctgggaaagggctggctcactgatgagaatgtggaggcatcagcaaggatctcggtgggttgctcagagaggtgatgcactaagccttaatcctggacaccagtacccctgcagcatggcttgctcaacaacagtctttgagtggcatagaattccaaagaaaatggtgctgggtggagaatggagagagcatgatggagcagagtcccagtcactgaccaactaactggtcgtttgattaggaaacagtttggccaaagtaccacctttgagacctaagttcttttgatacctttgagaagagccactgagcctgagttgaaatatttttagcttagtcatctgtgtttgctataggagaaattgtaacacaagaaataactcctttttacatgatcatttatatctatatacatatatatacttgcatacactatcactgcattaaaaaatgagtttgggctgggcatggtggctcacacctataatcccaacactttcggaggccaaggagggacaaacgaacccttgaggccaggagttccagactaacttgggcaacacagggcgacccccatctctacaaaacataaaagatttttaaaaaattagccaggcatggtggcacatgcctgtggtctcagctacttgggaggctgaggcaggagaatcatttgagcccaggaggtcaaggctgcagtgagctttgatcacaccactgcactccagcctgggcaacagagcaagaccccatcctccacccccccaaaaaatagaaagaaaaaaaaagtttgcactaattgaggtacatctgcaagtgagactttttgtcaggaaaaggcaatatatcaggtctcctcaggacgatggaggccttatatggtgtgttaccttgaaaactgaatatcaacgttcaccttgattcaggaaagctgggtgctgtctccatgccatgaatcatgagagcaaaggatcactgcttaaaaatactgaatttaccttcacaaaagatttctaaagatttatgtaatgtgttttaaaagcgccagtaaaccatcggatcaattggaaagaaggcaactcttcagcctttgttatctagctgaaaacaaatgacaactttcaaaacattggcagtagttgttgaaaaagacgtctattgttcaaagtttctttctccttaaaggacggtgttccaatgaattcagtagagcccactttcctccactgtggaggaagaatccctaagagatactcaaatgattaaattaaaattggatcatcaaactcaagagaggcataaacttagacacagtcttgcatttttgtctttcctgaactcttctgccattttcctccttcactcgtcctgaaaatctgcaagttacataataaaactttagatatttgtctgacaaagtgtaattactcaactgaataaatgactgagaacaagttacaaaaggaatcatgaatcctggtaaacaataaagaagattcagacactgagggaaaaaaaataaagctttttacttaaataatgcaaaaaaaaaaaaaaaa

SEQ ID NO: 94 - DNA, 2046 bp, Homo sapiens
ggagcccggggcgggcgagggcgggggtgtcccggctataaagcgtggccgcctcccgcggcgctcgggacagccgtaccccgggcggtcggacgggcgggcgccggtgggagctcgggccgtgcccgctgagagatccagagcgctccgttcccccggggccggagcgggggcgggtgggggcgtaagcccgggggatgctgggctcagtgaagatggaggcccatgacctggccgagtggagctactacccggaggcgggcgaggtctactcgccggtgaccccagtgcccaccatggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcctagcaggggttgggaacatggtggtgggtatggctggagctcacaccacgaagctcttggggcctgatccttctggtgacacttcacttgtcccattggttaacatctgggtgggtctattacttactgtgatgactgctgtctcagtgggcatggtgttgatccacggggtactgtgataaccaccatggatacattttggtggcccactgggtactgtgaggactgctacattgatggatgttattggctaatccactgcatggtttgatggccaccatctcggttggccctttgggtgtgatggtgatagcatttcagtgacatcttctttggccccccccattaggtgctgtgcccacttcttttttggtgtacttggcacagtaggtgccaagttggccaccattctgtgtaacaccttttttggcccattgggtgctttgatggacatcatactgggtaggtgacaacgtcagtgggccaccatgtgccatgatggctgctgcagccccgtgttggccatgtcgtcaccattctctctggcatgggttgggtaggggatggaggtgagaatactccttggttttctctgaagcccaccctttcccccaactctggtccaggagaaaccagaaaaggctggttagggtgtggggaatttctactgaagtctgattctttcccgggaagcggggtactggctgtgtttaatcattaaaggtaccgtgtccgcctcttaaaaaaaaaaaaaaaaaaaaaaaaaaaaaaaaaaaaaaaaaaaaaaaaaaaaaaaaaa

SEQ ID NO: 95 - DNA, 1506 bp, Homo sapiens
atgcaggcgcgctactccgtgtccgaccccaacgccctgggagtggtgccctacctgagcgagcagaattactaccgggctgcgggcagctacggcggcatggccagccccatgggcgtctattccggccacccggagcagtacagcgcggggatgggccgctcctacgcgccctaccaccaccaccagcccgcggcgcctaaggacctggtgaagccgccctacagctacatcgcgctcatcaccatggccatccagaacgcgcccgagaagaagatcaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaagggcagttactggaccctggacccggactcctacaacatgttcgagaacggcagcttcctgcggcgccggcggcgcttcaaaaagaaggacgtgtccaaggagaaggaggagcgggcccacctcaaggagccgcccccggcggcgtccaagggcgccccggccaccccccacctagcggacgcccccaaggaggccgagaagaaggtggtgatcaagagcgaggcggcgtccccggcgctgccggtcatcaccaaggtggagacgctgagccccgagagcgcgctgcagggcagcccgcgcagcgcggcctccacgcccgccggctcccccgacggttcgctgccggagcaccacgccgcggcgcccaacgggctgcctggcttcagcgtggagaacatcatgaccctgcgaacgtcgccgccgggcggagagctgagcccgggggccggacgcgcgggcctggtggtgccgccgctggcgctgccatacgccgccgcgccgcccgccgcctacggccagccgtgcgctcagggcctggaggccggggccgccgggggctaccagtgcagcatgcgagcgatgagcctgtacaccggggccgagcggccggcgcacatgtgcgtcccgcccgccctggacgaggccctctcggaccacccgagcggccccacgtcgcccctgagcgctctcaacctcgccgccggccaggagggcgcgctcgccgccacgggccaccaccaccagcaccacggccaccaccacccgcaggcgccgccgcccccgccggctccccagccccagccgacgccgcagcccggggccgccgcggcgcaggcggcctcctggtatctcaaccacagcggggacctgaaccacctccccggccacacgttcgcggcccagcagcaaactttccccaacgtgcgggagatgttcaactcccaccggctggggattgagaactcgaccctcggggagtcccaggtgagtggcaatgccagctgccagctgccctacagatccacgccgcctctctatcgccacgcagccccctactcctacgactgcacgaaatactga

SEQ ID NO: 96 - DNA, 3130 bp, Homo sapiens
gagtcagtggcttgaaacttttaaaagctctgtgctccaagttacaaaaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacata

SEQ ID NO: 97 - DNA, 2070 bp, Homo sapiens
ccgacacccacgggcggagatcacctgctgccccgcagacccctgtcccttcctcccggaccagcagctagaggatgtccaaacggagttggtgggctggatccagaaagcccccaagagagatgctgaaactctcaggctctgactccagccaaagcatgaatggccttgaagtggctcccccaggtctgatcaccaacttctccctggccacggcagagcaatgtggccaggagacgccactggagaacatgctgttcgcctccttctaccttctggattttatcctggctttagttggcaataccctggctctgtggcttttcatccgagaccacaagtccgggaccccggccaacgtgttcctgatgcatctggccgtggccgacttgtcgtgcgtgctggtcctgcccacccgcctggtctaccacttctctgggaaccactggccatttggggaaatcgcatgccgtctcaccggcttcctcttctacctcaacatgtacgccagcatctacttcctcacctgcatcagcgccgaccgtttcctggccattgtgcacccggtcaagtccctcaagctccgcaggcccctctacgcacacctggcctgtgccttcctgtgggtggtggtggctgtggccatggccccgctgctggtgagcccacagaccgtgcagaccaaccacacggtggtctgcctgcagctgtaccgggagaaggcctcccaccatgccctggtgtccctggcagtggccttcaccttcccgttcatcaccacggtcacctgctacctgctgatcatccgcagcctgcggcagggcctgcgtgtggagaagcgcctcaagaccaaggcagtgcgcatgatcgccatagtgctggccatcttcctggtctgcttcgtgccctaccacgtcaaccgctccgtctacgtgctgcactaccgcagccatggggcctcctgcgccacccagcgcatcctggccctggcaaaccgcatcacctcctgcctcaccagcctcaacggggcactcgaccccatcatgtatttcttcgtggctgagaagttccgccacgccctgtgcaacttgctctgtggcaaaaggctcaagggcccgccccccagcttcgaagggaaaaccaacgagagctcgctgagtgccaagtcagagctgtgagcggggggcgccgtccaggccgagcgcagactgtttaggactcagcagacccagcaagaggcatctgccctttccccagccacctccccagcaagcaacctgaaatctcagcagatgcccaccatttctctagatcgcctagtctcaacccataaaaaggaagaactgacaaaggggatccatcggccacccctctgcaggggcttgtgatggctacaatggctcctagacactcaacgacttcatctgtggcagggagagaggaggccggaagaacaacccctgaacaatggaggcctttctttcccgctaggctcccagcctccttcccgctacagaatcgctcatcggcgaggctcagcagaaagaccctgaaggcaggctgcaaatgacccagaagagggacctgggagtcctggtggggacggggagggagtctcaatactcctttgcagcgcaaggtactctgagtcccctctgtagtgcctctgccagacacacactgcctgagttgaagagacacaggccacacatttcaggctggttgccagcggacgtcagcactcacggcctgcggggactcagcacagctctggattctggatctctcctgctgtaaccccacgcacaagcctgcaacccccagagctctttgacaggctcccaggcctcccagtcctggacaagcatgtgcagtcacgggagctcagctcaggccagggctgggctgtgcacctgcctcccactgacccagacccacttcctccagagaggcctctctccgcctgagctatttcccttgctagtgtgcagatatttccctaacatgtccttttttgtatttgtttgtacggaccataaatataactgtagctttaagactaaaaaaaaa

SEQ ID NO: 98 - DNA, 1933 bp, Homo sapiens
ggctgggggaaatgacccgggagggtcccatgcggctacataaaattggcagccttagaactagtgggaaggcgggtgcgcgaagtcgaggggcggagagagggggccggaggagctgctttctgaatccaagttcgtgggctctctcagaagtcctcaggacggagcagaggtggccggcgggcccggctgactgcgcctctgctttctttccataaccttttctttcggactcgaatcacggctgctgcgaagggtctagttccggacactagggtgcccgaacgcgctgatgccccgagtgctcgcagggcttcccgctaaccatgctgccgccgccgcggcccgcagctgccttggcgctgcctgtgctcctgctactgctggtggtgctgacgccgcccccgaccggcgcaaggccatccccaggcccagattacctgcggcgcggctggatgcggctgctagcggagggcgagggctgcgctccctgccggccagaagagtgcgccgcgccgcggggctgcctggcgggcagggtgcgcgacgcgtgcggctgctgctgggaatgcgccaacctcgagggccagctctgcgacctggaccccagtgctcacttctacgggcactgcggcgagcagcttgagtgccggctggacacaggcggcgacctgagccgcggagaggtgccggaacctctgtgtgcctgtcgttcgcagagtccgctctgcgggtccgacggtcacacctactcccagatctgccgcctgcaggaggcggcccgcgctcggcccgatgccaacctcactgtggcacacccggggccctgcgaatcggggccccagatcgtgtcacatccatatgacacttggaatgtgacagggcaggatgtgatctttggctgtgaagtgtttgcctaccccatggcctccatcgagtggaggaaggatggcttggacatccagctgccaggggatgacccccacatctctgtgcagtttaggggtggaccccagaggtttgaggtgactggctggctgcagatccaggctgtgcgtcccagtgatgagggcacttaccgctgccttggccgcaatgccctgggtcaagtggaggcccctgctagcttgacagtgctcacacctgaccagctgaactctacaggcatcccccagctgcgatcactaaacctggttcctgaggaggaggctgagagtgaagagaatgacgattactactaggtccagagctctggcccatgggggtgggtgagcggctatagtgttcatccctgctcttgaaaagacctggaaaggggagcagggtcccttcatcgactgctttcatgctgtcagtagggatgatcatgggaggcctatttgactccaaggtagcagtgtggtaggatagagacaaaagctggaggagggtagggagagaagctgagaccaggaccggtggggtacaaaggggcccatgcaggagatgccctggccagtaggacctccaacaggttgtttcccaggctggggtgggggcctgagcagacacagaggtgcaggcaccaggattctccacttcttccagccctgctgggccacagttctaactgcccttcctcccaggccctggttcttgctatttcctggtccccaacgtttatctagcttgtttgccctttccccaaactcatcttccagaacttttccctctctcctaagccccagttgcacctactaactgcagtcccttttgctgtctgccgtcttttgtacaagagagagaacagcggagcatgacttagttcagtgcagagagataggtgaggccagctcgagatcttataccactctgtattggacaaaggctagcacagggctaggcaccaataaagatttctaatgatgcacagaaaaaaaaaaaaaaaaaaaaa

SEQ ID NO: 99 - DNA, 3613 bp, Homo sapiens
ttaacaagtgatcgctgctgtctaggattttgtttcttttcgggggaaccttgacttcctttcccaggcaatccctcctgtgctgaactccagaggaaccaggagtcttggggtcttctctggggcagccccaacccccacccccaggctccagccgcgaggactctgtgcacccctcgggccaggcaacagaacttgttccgtggatatttggagcctccacctgccaaacccgagtgattccttttaccaccccccgccccccacccaggatcattcttcccctcctccagctgttgcagcttgagggggaaaaacaagccagccggtggattttctttatttttatttttcgccccgccggggaacggtgaagtgcttcttctgcatgattttggctgaagaatgctctgcatttccttgatttctatggagacctcagagctggttttgcttctgctgacacctcatctagcaccttctctacctcccagggtctttgcctctatctgtggtttggcattgtacctgggtacaggaagcctttgatgaacttaaaaggagagcctggagaatcatcctgatagactttgagtagaaatggctggacatacttcaaaccacatcttaacatggttcgagccatcactagaaggcaagtgctaacagtaaaggcttatttgcattttatttacatttaatggactgagcattggccaatttccatggcagaaaaatatatttcattttctaggcacaacttctggctgtcagacacttgctgcctttgaatcttgcagcaacatcactaaccacatcccagacatatttccaaatttcaacatctacccccaaaacataggtgtctgagagactccagcattttcggacttcttagtcttgagagtgccaggctatttatctcgaccagccaagctctggagagcaatgttgaatccctgagaagagagagcatggggcgtgctgatttaaaaacagaaaatgcaaagttggactgaaaatatccttagtcttccaagcaatctgcttaagggttccaaacttaccttaatttggtgagaaaagaagctgccctatttttctttcttcttcttctacaactggaaccagccatttccgaaaaccaccaccatggaggttgcaatggtgagtgcggagagctcagggtgcaacagtcacatgccttatggttatgctgcccaggcccgggcccgggagcgggagaggcttgctcactccagggcagctgcagcagctgctgttgcagcggccacagctgctgtcgaaggtagcgggggttctggtgggggctcccaccaccaccaccagtcacgcggggcctgtacctcccatgaccctcagagcagccggggtagtcggaggaggaggcgacagcggtctgagaagaagaaagcccactaccggcagagcagcttccctcattgctctgacctgatgcccagtggctctgaggagaagatcctgagggagctgagtgaggaggaggaagatgaggaggaggaggaagaggaggaagaggagggaaggttttactatagtgaagatgaccatggtgatgagtgttcctacacggatctgctgcctcaggatgagggcggtggcggctacagttcagtccgctacagtgactgttgtgaacgtgtggtgataaatgtgtcaggcctacgctttgagacccaaatgaaaactctggcccagtttccagagactttgttgggagaccctgaaaagaggactcagtactttgaccctttgcgcaatgagtatttttttgacaggaaccgccccagctttgatgccatcttgtattattatcaatcaggaggccgcctgaagaggccagtcaatgtcccctttgatatcttcactgaggaggtgaagttctatcagttgggggaggaggccctgttgaagtttcgggaggacgagggctttgtgagagaagaggaagacagggccctccccgagaatgaatttaaaaagcagatttggctcctctttgaatatccagagagctccagtcctgcaaggggcatagccattgtgtccgtcctggtcatcttaatctccattgtcatcttttgcctggaaaccttgcctgagtttagggacgacagggatctcgtcatggcactgagtgctggcgggcatggtgggttgttgaatgatacttcagcaccccatctggagaactcagggcacacaatattcaatgaccccttcttcatcgtggaaacagtctgtattgtatggttttcctttgagtttgtggttcgctgctttgcttgtcccagccaagcactcttcttcaaaaacatcatgaacatcattgacattgtctccattttgccttacttcatcacactgggcactgacctggcccagcaacaggggggtggcaatggtcagcagcagcaggccatgtcctttgccatcctcagaatcattcgtctggtccgagtattccggatcttcaaactctccaggcactccaaaggcctgcagatcctgggccacaccctcagagccagcatgcgggaactgggccttctgatcttcttcctcttcattggggtcatcctcttttctagtgctgtgtattttgcagaggcggatgaacctactacccatttccaaagcatcccagatgcattttggtgggctgtggtgaccatgacaactgtgggctatggggacatgaagcccatcactgtagggggcaagattgtcgggtccctgtgtgccattgcgggtgtcttaaccattgctttgccagtgccagtgattgtctctaactttaactatttctaccacagagagactgaaaatgaggaacagacacagctaacgcagaatgcagtcagttgtccatacctcccctctaatttgctcaagaaatttcggagctctacttcttcttccctgggggacaagtcagagtatctagagatggaagaaggagttaaggaatctctgtgtgcaaaggaggagaagtgtcagggaaagggggatgacagtgagacagataaaaacaactgttctaatgcaaaggctgtggagactgatgtgtgaatctttttccacctgccactgctcccccctcagcatctccaaatatatttatgcatagagagtgcagttatgaaaatgaaatatgcaaatgatccaatgcatacagtagtacactatttaatggttatacatggcataattgttactaaacttgtattacatatcaaataaatgatacatcttggagaagagggaggaataggagcaaatctatctttatatttttattagaatgcaagaattttgcacattaactggaaaagatgttaacagtaaagatggagagagagagtgtgtgcgtgtgtgtgtatatgtgtgtgtgtgtgaagtaaattgtcaatgttagtaattgtgcagtgaagggaaaagttggcattttgaagtatttactatgtaagaactaatgaatctgagcagtcatttatcagtgctttaacagcatatcgtatgtctttggattctgtagttgttttttaaaaattgtaagaaatactgtgta

SEQ ID NO: 100 - DNA, 2065 bp, Homo sapiens
gcagtcgctgccgaccggctggctgggccttgcggcgtgaggaccccggcggcgccgcagtcccgcgagccatggcccagtccggcggggaggctcggcccgggcccaagacggcggtgcagatccgcgtcgccatccaggaggccgaggacgtggacgagttggaggacgaggaggagggggcggagactcggggcgccggggacccggcccggtacctcagccccggctggggcagcgcgagcgaggaggagccgagccgcgggcacagtggcaccactgcaagtggaggtgagaacgagcgtgaggacctggagcaggagtggaagcccccggatgaggagttgatcaagaaactggtggatcagatcgaattctacttttctgatgaaaacctggagaaggacgcctttttgctaaaacacgtgaggaggaacaagctgggatatgtgagcgttaagctactcacatccttcaaaaaggtgaaacatcttacacgggactggagaaccacagcacatgctttgaagtattcagtggtccttgagttgaatgaggaccaccggaaggtgaggaggaccacccccgtcccactgttccccaacgagaacctccccagcaagatgctcctggtctatgatctctacttgtctcctaagctgtgggctctggccaccccccagaagaatggaagggtgcaagagaaggtgatggaacacctgctcaagctttttgggacttttggagtcatctcatcagtgcggatcctcaaacctgggagagagctgccccctgacatccggaggatcagcagccgctacagccaagtggggacccaggagtgcgccatcgtggagttcgaggaggtggaagcagccatcaaagcccatgagttcatgatcacagaatctcagggcaaagagaacatgaaagctgtcctgattggtatgaagccacccaaaaagaaacctgccaaagacaaaaatcatgacgaggagcccactgcgagcatccacctgaacaagtccctgaacaagagagtcgaggagcttcagtacatgggtgatgagtcttctgccaacagctcctctgaccccgagagcaaccccacatcccctatggcgggccgacggcacgcggccaccaacaagctcagcccgtctggccaccagaatctctttctgagtccaaatgcctccccgtgcacaagtccttggagcagccccttggcccaacgcaaaggcgtttccagaaagtccccactggcggaggaaggtagactgaactgcagcaccagccctgagatcttccgcaagtgtatggattattcctctgacagcagcgtcactccctctggcagcccctgggtccggaggcgtcgccaagccgagatggggacccaggagaaaagccccggtacgagtcccctgctctcccggaagatgcagactgcagatgggctacccgtaggggtgctgaggttgcccaggggtcctgacaacaccagaggatttcatggccatgagaggagcagggcctgtgtataaataccttctatttttaatacaagctccactgaaaaccaccttcgttttcaaggttctgacaaacacctggcatgacagaatggaattcgttcccctttgagagattttttattcatgtagacctcttaatttatctatctgtaatatacataaatcggtacgccatggtttgaagaccaccttctagttcaggactcctgttcttcccagcatggccactattttgatgatggctgatgtgtgtgagtgtgatggccctgaagggctgtaggacggaggttccctgggggaagtctgttctttggtatggaatttttctctcttctttggtatggaatttttcccttcagtgactgagctgtcctcgataggccatgcaagggcttcctgagagttcaggaaagttctcttgtgcaacagcaagtagctaagcctatagcatggtgtcttgtaggaccaaatcgatgttacctgtcaagtaaataaataataaaacacccaaaaaaaaaaaaaaaaaa

SEQ ID NO: 101 - DNA, 543 bp, Homo sapiens
gaccttgagggagttaatgtgtaatattctaggatataagcttgaccacgagttgagaccctgagcacaggcctccaggagccgctgggagctgccgccaggagctgtcaccatgacgggggaacttgaggttaagaacatggacatgaagccggggtcaaccctgaagatcacaggcagcatcgccgatggcactgatggctttgtaattaatctgggccaggggacagacaagctgaacctgcatttcaaccctcgcttcagcgaatccaccattgtctgcaactcattggacggcagcaactgggggcaagaacaacgggaagatcacctgtgcttcagcccagggtcagaggtcaagttcacagtgacctttgagagtgacaaattcaaggtgaagctgccagatgggcacgagctgacttttcccaacaggctgggtcacagccacctgagctacctgagcgtaaggggcgggttcaacatgtcctctttcaagttaaaagaataaaagacttccagccgagaaaaaaaaaaaaaaaa

SEQ ID NO: 102 - DNA, 661 bp, Homo sapiens
tggttcttataaaaacctcacagccttccactaacatcccgtaggagcctctctccctactgctgctacacaagaccctgagactgacctgcaggacgaaaccatgaagagcctgatccttcttgccatcctggccgccttagcggtagtaactttgtgttatgaatcacatgaaagcatggaatcttatgaacttaatcccttcattaacaggagaaatgcaaataccttcatatcccctcagcagagatggagagctaaagtccaagagaggatccgagaacgctctaagcctgtccacgagctcaatagggaagcctgtgatgactacagactttgcgaacgctacgccatggtttatggatacaatgctgcctataatcgctacttcaggaagcgccgagggaccaaatgagactgagggaagaaaaaaaatctctttttttctggaggctggcacctgattttgtatccccctgtagcagcattactgaaatacataggcttatatacaatgcttctttcctgtatattctcttgtctggctgcacccctttttcccgcccccagattgataagtaatgaaagtgcactgcagtgagggtcaaaggagagtcaacatatgtgattgttccataataaacttctggtgtgatacttt

SEQ ID NO: 103 - DNA, 629 bp, Homo sapiens
cttctaggtggtgtgggcgaagtttgggactggtttagggcggggacaagaccaagaacacaagtttccttgtactacgggagagagggaggggaggaaattggagaccccagcacccccttgctcactctcttgctcacagtccacgatggcccggtccctggtgtgccttggtgtcatcatcttgctgtctgccttctccggacctggtgtcaggggtggtcctatgcccaagctggctgaccggaagctgtgtgcggaccaggagtgcagccaccctatctccatggctgtggcccttcaggactacatggcccccgactgccgattcctgaccattcaccggggccaagtggtgtatgtcttctccaagctgaagggccgtgggcggctcttctggggaggcagcgttcagggagattactatggagatctggctgctcgcctgggctatttccccagtagcattgtccgagaggaccagaccctgaaacctggcaaagtcgatgtgaagacagacaaatgggatttctactgccagtgagctcagcctaccgctggccctgccgtttcccctccttggctttatgcaaatacaatcagcccagtgcaaaaaaaaaaaaaaaaaa

SEQ ID NO: 104 - DNA, 1073 bp, Homo sapiens
atgtgcacacacatacactcacacgtgtgtgcaggtgcacacctcccccagaggctgcagccaagacgggcatcccacatcagagggatgaatggcaggtctgtcccgccagctgtgtgctctctcccacccgaagaaagcagcagagactcagacggcggagcctggaggagcccacgcagtctgttcccggcacccggtgcgtgtgaagggacttgagggcagcgagatggaatcagcaagagaaaacatcgaccttcaacctggaagctccgaccccaggagccagcccatcaacctgaaccattacgccaccaagaagagcgtggcggagagcatgctggacgtggccctgttcatgtccaacgccatgcggctgaaggcggtgctggagcagggaccatcctctcactactacaccaccctggtcaccctcatcagcctctctctgctcctgcaggtggtcatcggtgtcctgctcgtggtcattgcacggctgaacctgaatgaggtagaaaagcagtggcgactcaaccagctcaacaacgcagccaccatcttggtcttcttcactgtggtcatcaatgttttcattacagccttcggggcacataaaacagggttcctggctgccagggcctcaaggaatcctctctgaatgcagcctgggacccaggttctgggcctggaacttctgcctccttcctccgtgatctgccaggctcgtgggcactttccacagcccaggagagcttctgaaaggacagtatagctgcccttgctccctacccacagcacctgagttaaaaagtgatttttatgttattggtctaagggacttccatcttggtctgaagtcctgagctcagacgcaggtactgccagccataccttcctggtagcatctgctggacctaagtaaggcatgtctgtctaaggccaagtctgcccggcttaaggatgctggttctgactctaccccactgcttccttctgctccaggcctcaattttcccttcttgtaaaatggaatctatatctataaaggtttcttcaaatccaaaaaaaaaaaa

SEQ ID NO: 105 - DNA, 6413 bp, Homo sapiens
agaaatcagagacgctgcctgcctgctcccatctctcgcgcgctctctctctcttctgctctctccctccctttgcaaacattggatttaaacctgctcagaattcagcacagaggaaggcagcagcggtagcagcagcagaagcagtagcaagcccggcagctgagagcaccgcagcgtcgagatgtaccatcctgcctactgggtcgtcttctcggcgacaactgccctgctcttcatcccaggagtgcccgtgcgcagcggagatgccaccttccccaaagctatggacaacgtgacggtccggcagggggagagcgccaccctcaggtgtaccatagatgaccgggtaacccgggtggcctggctaaaccgcagcaccatcctctacgctgggaatgacaagtggtccatagaccctcgtgtgatcatcctggtcaatacaccaacccagtacagcatcatgatccaaaatgtggatgtgtatgacgaaggtccgtacacctgctctgtgcagacagacaatcatcccaaaacgtcccgggttcacctaatagtgcaagttcctcctcagatcatgaatatctcctcagacatcactgtgaatgagggaagcagtgtgaccctgctgtgtcttgctattggcagaccagagccaactgtgacatggagacacctgtcagtcaaggaaggccagggctttgtaagtgaggatgagtacctggagatctctgacatcaagcgagaccagtccggggagtacgaatgcagcgcgttgaacgatgtcgctgcgcccgatgtgcggaaagtaaaaatcactgtaaactatcctccctatatctcaaaagccaagaacactggtgtttcagtcggtcagaagggcatcctgagctgtgaagcctctgcagtccccatggctgaattccagtggttcaaggaagaaaccaggttagccactggtctggatggaatgaggattgaaaacaaaggccgcatgtccactctgactttcttcaatgtttcagaaaaggattatgggaactatacttgtgtggccacgaacaagcttgggaacaccaatgccagcatcacattgtatgggcctggagcagtcattgatggtgtaaactcggcctccagagcactggcttgtctctggctatcagggaccctcttagcccacttcttcatcaagttttgataagaaatcctaggtcctctgagcaacgcctgcttctccatatcacagactttaatctacactgcggagagcaaaccagcttgggcttctttttgtttttttctgttattctagatttgttttctttttgtttttgtttatttgtttgtttgcttttatttccagcttgaatgagtggggttgggggcggggtgggcagggttctaccacgtgtaggataatcattcattggtgtgtccaaaaatggggtctgctcctgctaccttgacccttccctttcctctgcttctctcctcatcatcattcccaacaacatcctctgccacacacaacaaaacgtaagtttcatttgggcaaaaattgagcctcacaataaacaccctgaagacacaacttgacttataacatagtgcacagcaagagctacatccaagtgtcctattatctgtgattattttcttaatgacaatgtacatatgcccccatccatgttaattattatctaattccattagggttcacgtcttttctttctgggacactatcctactatatccatatctatagatttcaatatagatgattgtgccatcttctgtagcccctccgctctactcattccttccaccatctgcagagatttgaagtttggggctatgcatgaaacccaacactaaattttgcaagtcaagtaaccaaaaaagggggaggcattttgaagatagaacctctattttaaaaagagaagttcaactcataaacgtgattgataggtggctgatttatttaggttttgtcaagctatctatcaaagtaatggtacagttacccatctactcaaatatctgatttatctcaccatccaattatctacccacctgtcttcctctctagcaatctatttactgtttatcaatctatcaatgtaattgtctaacactcctttctattctctccctactactcactatcaattcatccccatatgaatctctaaccatattgtatctctcccactgtattcatttatacaccatcagcagacattggcatcttcaaaattatctttcaacttctgtgaaagccaacgatctcacaggttaacaaaatacaaaagcaataccctgtgttgtggactctttaaaatctggtatcctatccacccaagggagacactaacagataggccaaagtagcaagctaatgatcagtcactcactattcccagaagagcctgtgttttctaaaacactttcttgggaagcagatcagcctagaaaagttttgattagcactgtggttttccttttgcacttgaaggacaaaggtgccagcctttatgcttctctcaacccttcaagaaagtacatgtcaggaacctatggctggctttccttagcagcaagaacttgagagaaaaacacatctgtctctgcaatgcaaagtgaagagtccacccgcctgagtgggatgacttcagctagagtctcctttctgctccagttctggtttaatctgtttgaaaactatccagtaaaaagctgatggaggccaattacatggcgggtgtattgacaactctggtatttgtttcaggaagctcttctaagctgagggcacttgagcaactgacttaattttcaagcacttgattaacacaacactgcaaacagaagggagaaagtgtcagtgacacagtttcctctgatgcagctgcttctccaatggctttggggaagaacttcaccagctcttcaggttcaaagcagacccagcatacaaacaagagctgagccacctttgctgtcttgtctcctgggacgagaaggactcatccagcaaagttgcctgggattcaaaataaaggcattgcagaccgcacaggtgtgctgcagggactgatccacagagaggatgagaatgcagcatcaatcgcagacctgccctgcctcagttggaaaaccttttcaggccctcagtctaaaaaataaaaaatatgagcaccattgaattctgtgcccttaatgcttaactggtcttcttcctctggtatcagtgtcctctttgtttttgtccatcaaggcacatgagtgtgacctctgccatggggaaacacacacagagatatctatacatatatacatacatacaaacataggctatcttggcacactaaatgctaagcactgtcttaagaggtagagctggtgtgagtgaaattaatgttacattttccagctgtaaacagacatctgcatttcctagtgagctgccaggagccagattcgggaaccgtaactgatgtgccaggaatggtgcattgattcccagttccagggatgatcatgagcaggcgcaaaatcagaattaaaggtcgcacatagacgtttcagatctgtcaccaccttcagcatctggagttgagttggtgtcagatagtgtatgagaattaaatgtgtcatctgagcatgctactgatgataaatttgttactttggagttgaataaatgtgaaggctgtgaagagtggacagtcttggagaacacagtgcttgaaatggacaagctggacctattcctcactccaagacttgttctacaggaaagggtccatgctcctttggccaagatcatcagaacctctcaacccaacaaggctggcttcagggccactatggaaccctgctgttcccccttccaaaggatactaagatgcccctctggtgggtacctatcccagccacgtttcagagggagagaaatgctacagttgatcctcatctgtctggggtaaagacaacaaagtaaatacaacccaaggcaactggggtactcactgggagtgaaaatgacttcttcacaacagacatatttctgcttctgtgtttttgtgtttctttggtggggatggcttcatgggagagtggctgtcacccatcattttgaagcatatagaacaacaaatgcttacacaagacaatatccacacttttccaacttcacacacggagagtacatggagaatgcctacaggctagatttgttcagggtgccagtagtgggcatggggtgggggcaaggcaggacaaaacatacaagtctgagcaagtacatctcttgcaggttttccacatgaaaaggaagccaaataagtcctgttaggagattaggtgagaggaattagcaatgtagggactctgaaacccttccccttcccaaaacagagttcatatgcacttccaccaaagtaatgccaatgaaagtgctcgtgttaaggctgcagccaagcttgtttttcagtagtttaatgtcaagtgcctgatacagtcgactgcaagtctaaacaagcatgtttagtttttctcattcttgctttaattcagggagggggagatgtagagaagtggttgtgaaaacatgtacaggctttatgcagagcactgcgcatggctgttctgctgcaactgtgctccacgaaacagaagaaaaggtaaggtgttgtgtcacaaagaggccccagtctctttcttcttacatccatgcctcttactagatgatacatttacagattgggcagtttgttctcaaaacctgggtgagaagactattcctggactctagcaacttcaaaactgaggctgggtttcagaatctttttctgcatcaattcagtcaatttgccttcaacaaagagaagtcagcaagttctatttatgctgaaagaactattccatgagaaaagcagagaaccccaaagtgggcaggcaaccccgacgagagcttatccctgtggcggcatcaggagtggctgtacattgaattttcaagtgctggttggctgtcgccagcccatggtaggaggggaggaatggcttaagatgaggtaagatctggtggtggggcatctttcctcaattccatactgactttgatcttgagaaagaaaaactggctatgcattacctaaaaccagtccaaaatgaaacagaccaacacacacacaaaagcaaattgtcaatccctttggaattaagggaagcagcataaggtttttctttttggaaaaaatgcatttattttctttttctccaacagcaagaatcttttgttttcattttgcacgtgaccttatcttggaaactcttatacccaattgcctcccctcctattattcagagcttccctgtctttttacttgaagacaaataagtttgagcacttgagtaaaacttcacaggtgtgtaagtaggaaggcaacattttcaaaaagagaccatatgatgagaacgcctaatgatcaccacatgcaaacaaacaaaactgccagtctcatttcccacatttcttacttaagagaagagaagtaaatgaaaggaagaagaaatagatttgtaattaaagatgtggcaaaaaagatagggctgagccagttcaatttagccttcaggtgcagaatacttagagtccaaagaaatgtggagtggacttaattagatgcagttgtctttatcctgaaagtagtgagctaagcctaatttccagcattttgaaagagattcctttttgtttctttccatggtgccctctttaaggcacagagttgctccacaccactgggtggagaaagaaagattgcgaaccctcgaccatccttttgaggctacattctatgttatttggcagatttataaagctatcagtaataacaatgctatgtactgcaagctgcccttgtgttagttaaagggagcatttttaatcgttcggaaattttcgtgacatgtcaagtgcagttgtgaggactgtgtgggtgaacgaaaatgtgtctgtcaagttcagagtcctttagatttaaaaaaaaattatgacttatcaatggtgccgttatagctgtgtcagacaatgggtgtgcccattctcacaattatccttcaaaaaaaatctatgttcaaatgctttaaaaatttatcacacgatacaagagtatgactttgtcagccttctagagttctttttttcttttattttctttcgtattttttccttcaaaaaatcaatgaagacttgatttctgtcaataattgtatcaagggtgaatatactacctgaattttgtgcatgttacattgtagttgtaaccttttctaattcaggatgaatacgagatggttgtgattgtgcagtgtaccaataaagttcgagaaatttgtaa

SEQ ID NO: 106 - DNA, 3874 bp, Homo sapiens
ctaggcggcggcggccgggtccccaaggctgggcgctgcttgcggaaccgacggggcggagaggagcgtggcgggaggaggagtaggagaagggggctggtcaagggaagtgcgacgtgtctgcggagcctttttatacctccttcccgggagtccggcagccgctgctgctgctgctgctgctgctgccgccgccgccgccgccgtccctgcgtccttcggtctctgctcccgggacccgggctccgccgcagccagccagcatgtcggggatcaagaagcaaaagacggagaaccagcagaaatccaccaatgtagtctatcaggcccaccatgtgagcaggaataagagagggcaagtggttggaacaaggggtgggttccgaggatgtaccgtgtggctaacaggtctctctggtgctggaaaaacaacgataagttttgccctggaggagtaccttgtctcccatgccatcccttgttactccctggatggggacaatgtccgtcatggccttaacagaaatctcggattctctcctggggacagagaggaaaatatccgccggattgctgaggtggctaagctgtttgctgatgctggtctggtctgcattaccagctttatttctccattcgcaaaggatcgtgagaatgcccgcaaaatacatgaatcagcagggctgccattctttgaaatatttgtagatgcacctctaaatatttgtgaaagcagagacgtaaaaggcctctataaaagggccagagctggggagattaaaggatttacaggtattgattctgattatgagaaacctgaaactcctgagcgtgtgcttaaaaccaatttgtccacagtgagtgactgtgtccaccaggtagtggaacttctgcaagagcagaacattgtaccctatactataatcaaagatatccacgaactctttgtgccggaaaacaaacttgaccacgtccgagctgaggctgaaactctcccttcattatcaattactaagctggatctccagtgggtccaggttttgagcgaaggctgggccactcccctcaaaggtttcatgcgggagaaggagtacttacaggttatgcactttgacaccctgctagatggcatggcccttcctgatggcgtgatcaacatgagcatccccattgtactgcccgtctctgcagaggataagacacggctggaagggtgcagcaagtttgtcctggcacatggtggacggagggtagctatcttacgagacgctgaattctatgaacacagaaaagaggaacgctgttcccgtgtttgggggacaacatgtacaaaacacccccatatcaaaatggtgatggaaagtggggactggctggttggtggagaccttcaggtgctggagaaaataagatggaatgatgggctggaccaataccgtctgacacctctggagctcaaacagaaatgtaaagaaatgaatgctgatgcggtgtttgcattccagttgcgcaatcctgtccacaatggccatgccctgttgatgcaggacactcgccgcaggctcctagagaggggctacaagcacccggtcctcctactacaccctctgggcggctggaccaaggatgacgatgtgcctctagactggcggatgaagcagcacgcggctgtgctcgaggaaggggtcctggatcccaagtcaaccattgttgccatctttccgtctcccatgttatatgctggccccacagaggtccagtggcactgcaggtcccggatgattgcgggtgccaatttctacattgtggggagggaccctgcaggaatgccccatcctgaaaccaagaaggatctgtatgaacccactcatgggggcaaggtcttgagcatggcccctggcctcacctctgtggaaatcattccattccgagtggctgcctacaacaaagccaaaaaagccatggacttctatgatccagcaaggcacaatgagtttgacttcatctcaggaactcgaatgaggaagctcgcccgggaaggagagaatcccccagatggcttcatggcccccaaagcatggaaggtcctgacagattattacaggtccctggagaagaactaagcctttggctccagagtttctttctgaagtgctctttgattaccttttctatttttatgattagatgctttgtattaaattgcttctcaatgatgcattttaatcttttataatgaagtaaaagttgtgtctataattaaaaaaaaatatatatatatacacacacacatatacatacaaagtcaaactgaagaccaaatcttagcaggtaaaagcaatattcttatacatttcataataaaattagctctatgtattttctactgcacctgagcaggcaggtcccagatttcttaaggctttgtttgaccatgtgtctagttacttgctgaaaagtgaatatattttccagcatgtcttgacaacctgtactcttccaatgtcatttatcagttgtaaaatatatcagattgtgtcctcttctgtacaattgacaaaaaaaaaaatttttttttctcactctaaaagaggtgtggctcacatcaagattcttcctgatattttacctcatgctgtacaaagccttaatgttgtaatcatatcttacgtgttgaagacctgactggagaaacaaaatgtgcaataacgtgaattttatcttagagatctgtgcagcctatttctgtcacaaaagttatattgtctaataagagaagtcttaatggcctctgtgaataatgtaactccagttacacggtgacttttaatagcatacagtgatttgatgaaaggacgtcaaacaatgtggcgatgtcgtggaaagttatctttcccgctctttgctgtggtcattgtgtcttgcagaaaggatggccctgatgcagcagcagcgccagctgtaataaaaaataattcacactatcagactagcaaggcactagaactggaaaagaccacagaaaacaaagaatccaaccctttcatcttacaggtgaacaaactgtgatgatgcacatgtatgtgttttgtaagctgtgagcaccgtaacaaaatgtaaatttgccattattaggaagtgctggtggcagtgaagaagcacccaggccacttgactcccagtctggtgccctgtctacaccagacaacacaggagctgggtcagattcccctcagctgcttaacaaagttcctcgaacagaaagtgcttacaaagctgccttctcggatactgaaaggtcgagttttctgaactgcactgattttattgcagttgaaaaaaaaaaaaagctattccaaagatttcaagctgttctgagacatcttctgatggctttacttcctgagaggcaatgtttttactttatgcataattcattgttgccaaggaataaagtgaagaaacagcaccttttaatatataggtctctctggaagagacctaaattagaaagagaaaactgtgacaattttcatattctcattcttaaaaaacactaatcttaactaacaaaagttcttttgagaataagttacacacaatggccacagcagtttgtctttaatagtatagtgcctatactcatgtaatcggttactcactactgcctttaaaaaaaaaaaccagcatatttattgaaaacatgagacaggattatagtgccttaaccgatatattttgtgacttaaaaaatacatttaaaactgctcttctgctctagtaccatgcttagtgcaaatgattatttctatgtacaactgatgcttgttcttattttaataaatttatcagagtgaaaaaaaaaaaaaaaaaa

SEQ ID NO: 107 - DNA, 512 bp, Homo sapiens
attcttcccctctctacaaccctctctcctcagcgcttcttctttcttggtttgatcctgactgctgtcatggcgtgccctctggagaaggccctggatgtgatggtgtccaccttccacaagtactcgggcaaagagggtgacaagttcaagctcaacaagtcagaactaaaggagctgctgacccgggagctgcccagcttcttggggaaaaggacagatgaagctgctttccagaagctgatgagcaacttggacagcaacagggacaacgaggtggacttccaagagtactgtgtcttcctgtcctgcatcgccatgatgtgtaacgaattctttgaaggcttcccagataagcagcccaggaagaaatgaaaactcctctgatgtggttggggggtctgccagctggggccctccctgtcgccagtgggcacttttttttttccaccctggctccttcagacacgtgcttgatgctgagcaagttcaataaagattcttggaagttt

SEQ ID NO: 108 - DNA, 683 bp, Homo sapiens
ctgcgcagatgaggggagactcgtcaccaggcgtgcagtgggcactgctgggctcccccatcccgtcctaacccggaacagccccgggcaggaggcgtggaaagtcgagggggtaaaccgcgaatgtgcgttgtgtaagccacggcgcagggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccgctccaagcccagccctcagccatggcatgccccctggatcaggccattggcctcctcgtggccatcttccacaagtactccggcagggagggtgacaagcacaccctgagcaagaaggagctgaaggagctgatccagaaggagctcaccattggctcgaagctgcaggatgctgaaattgcaaggctgatggaagacttggaccggaacaaggaccaggaggtgaacttccaggagtatgtcaccttcctgggggccttggctttgatctacaatgaagccctcaagggctgaaaataaatagggaagatggagacaccctctgggggtcctctctgagtcaaatccagtggtgggtaattgtacaataaattttttttggtcaaattt

SEQ ID NO: 109 - DNA, 3371 bp, Homo sapiens
agcgggttccattcccgggggattggagtagcgttggagtcaccgacgccatcccctcccgcctctggcgtagcaggagcatgcgcttccttcctcacttcctctccaggagggagcgagagtaaagctacgccctggcgcgcagtctccgcgtcacaggaacttcagcacccacagggcggacagcgctcccctctacctggagacttgactcccgcgcgccccaaccctgcttatcccttgaccgtcgagtgtcagagatcctgcagccgcccagtcccggcccctctcccgccccacacccaccctcctggctcttcctgtttttactcctccttttcattcataacaaaagctacagctccaggagcccagcgccgggctgtgacccaagccgagcgtggaagaatggggttcctcgggaccggcacttggattctggtgttagtgctcccgattcaagctttccccaaacctggaggaagccaagacaaatctctacataatagagaattaagtgcagaaagacctttgaatgaacagattgctgaagcagaagaagacaagattaaaaaaacatatcctccagaaaacaagccaggtcagagcaactattcttttgttgataacttgaacctgctaaaggcaataacagaaaaggaaaaaattgagaaagaaagacaatctataagaagctccccacttgataataagttgaatgtggaagatgttgattcaaccaagaatcgaaaactgatcgatgattatgactctactaagagtggattggatcataaatttcaagatgatccagatggtcttcatcaactagacgggactcctttaaccgctgaagacattgtccataaaatcgctgccaggatttatgaagaaaatgacagagccgtgtttgacaagattgtttctaaactacttaatctcggccttatcacagaaagccaagcacatacactggaagatgaagtagcagaggttttacaaaaattaatctcaaaggaagccaacaattatgaggaggatcccaataagcccacaagctggactgagaatcaggctggaaaaataccagagaaagtgactccaatggcagcaattcaagatggtcttgctaagggagaaaacgatgaaacagtatctaacacattaaccttgacaaatggcttggaaaggagaactaaaacctacagtgaagacaactttgaggaactccaatatttcccaaatttctatgcgctactgaaaagtattgattcagaaaaagaagcaaaagagaaagaaacactgattactatcatgaaaacactgattgactttgtgaagatgatggtgaaatatggaacaatatctccagaagaaggtgtttcctaccttgaaaacttggatgaaatgattgctcttcagaccaaaaacaagctagaaaaaaatgctactgacaatataagcaagcttttcccagcaccatcagagaagagtcatgaagaaacagacagtaccaaggaagaagcagctaagatggaaaaggaatatggaagcttgaaggattccacaaaagatgataactccaacccaggaggaaagacagatgaacccaaaggaaaaacagaagcctatttggaagccatcagaaaaaatattgaatggttgaagaaacatgacaaaaagggaaataaagaagattatgacctttcaaagatgagagacttcatcaataaacaagctgatgcttatgtggagaaaggcatccttgacaaggaagaagccgaggccatcaagcgcatttatagcagcctgtaaaaatggcaaaagatccaggagtctttcaactgtttcagaaaacataatatagcttaaaacacttctaattctgtgattaaaattttttgacccaagggttattagaaagtgctgaatttacagtagttaaccttttacaagtggttaaaacatagctttcttcccgtaaaaactatctgaaagtaaagttgtatgtaagctgagattttgtatacagaatccttatttcctcatagacttatattttataatcagaatatgttgctttgaaaaagcctctaatggactgaccttaaaactcatccttcttccactgtctcatccacataagcactccccgaagaattaagggggttctgttttcaaggcatgccaagtactaaagcaccttgcagagcgtgtctattacaagatgtcatttccaccagcagttcccttaggggagctgaaataaattcacattttctcaaagtctcatagctttggaggagccatctgcttttttggctgctctttttagctggctttttattaggctcagtgacataaaaaggatccaggtaaatgggtataggatttgctggatttactaacaatttccccctgttcttaacacttcctattagtgacttttcagacattgagtttacttataaagagagatatttatgtactctctaagaagacaaatgaggtcataaacactgcataaagcaaggcaaaaatgtatgccacatctcagttatctaaactagattagatccaagccaagttttctcaacagagagcaaagggccaggcagtaaggtagaaatagagataaaaatcattccttccttgtgatccaaagctggtcgagcagctttcctggaggaaaaggttaatgaacttcaggtccctgcaactcagcccccaccacaaacacagccctggaaacatacagtggcgcaaggtcctcttgaaatgttaatggttaatgttcccaaaccagagaatgctttgaaaatgtatcattcagtgtaaattaattacatacatatttttctatatatttgtttcaaactgtaaaaataacataatatgtaatttgtgtattagtgagaggtgaagccagctggacttcctgggtcgagtggggccttggagaacttttctgtcttacaagaggattgtaaaatgcacccatcagtgctctgtaaaacacaccaatcagcgctctgtagctagcaataggtttgtaaaatgcacccatcagcactctgtaaaacgcaccaatcagcactctgtaaaatgcaccaatcagcaggattctaaaagtagacaatcacagggaggattgaaaaaaagggcactctgatagggcaaaaacggaacatgggaggggacaaataagggaataaaatctggccaccccagccagcagcagcaacctgttcaggtcgcctgccgctgtggaagctttgtccttttgctcttcataataaaccttgctactgttcaaaaaaaaaaaaaaaaaaaaaaaaaaaaaaaaaaaaaaaaaaaaaaaaaaaaaaaaa

SEQ ID NO: 110 - DNA, 1151 bp, Homo sapiens
gctcctcgggctgcccctcggttgacaatggtctccaggatggtctctaccatgctatctggcctactgttttggctggcatctggatggactccagcatttgcttacagcccccggacccctgaccgggtctcagaagcagatatccagaggctgcttcatggtgttatggagcaattgggcattgccaggccccgagtggaatatccagctcaccaggccatgaatcttgtgggcccccagagcattgaaggtggagctcatgaaggacttcagcatttgggtccttttggcaacatccccaacatcgtggcagagttgactggagacaacattcctaaggactttagtgaggatcaggggtacccagaccctccaaatccctgtcctgttggaaaaacagatgatggatgtctagaaaacacccctgacactgcagagttcagtcgagagttccagttgcaccagcatctctttgatccggaacatgactatccaggcttgggcaagtggaacaagaaactcctttacgagaagatgaagggaggagagagacgaaagcggaggagtgtcaatccatatctacaaggacagagactggataatgttgttgcaaagaagtctgtcccccatttttcagatgaggataaggatccagagtaaagagaagatgctagacgaaaacccacattacctgttaggcctcagcatggcttatgtgcacgtgtaaatggagtccctgtgaatgacagcatgtttcttacatagataattatggatacaaagcagctgtatgtagatagtgtattgtcttcacaccgatgattctgctttttgctaaattagaataagagcttttttgtttcttgggtttttaaaatgtgaatctgcaatgatcataaaaattaaaatgtgaatgtcaacaataaaaagcaagactatgaaaggctcagatttcttgcagtttaaaatggtgtctgaggttgtactattttggccaagtctgtagaaagctgtcatttgattttgattatgtagttcatccagcccttgggcattgttatacaccagtaaagaaggctgtactcaagaggaggagctgacacatttcacttggctgcgtcttaataaacatgaatgcaagcattggc

SEQ ID NO: 111 - DNA, 2206 bp, Homo sapiens
actatattcacaggcttggagccagtgccattcacacttccccctcttctgcagcagacggactgagttcctctaatccctgtgttccttctcccccatctttctaaaacccttctctgagagaggaataactatagcttcagggataatatagctttaaggaaacttttggcagatgtggacgtcgtaacatctgggcagtgttaacagaatcccggaggccgggacagaccaggagccactcgttctaggaatgttaaagtagaaggttttttccaattgatgagaggagcagagaggaaggagaaagaggaggagagagaaaaagggcacaaaataccataaaacagatcccatatttctgcttcccctcacttttagaagttaattgatggctgacttctgaaagtcactttcctttgccctggtacttcaggccatatacatcttttcttgtctccataatcctccctttcaaggatggccagtcagctaactcaaagaggagctctctttctgctgttcttcctaactccggcagtgacaccaacatggtatgcaggttctggctactatccggatgaaagctacaatgaagtatatgcagaggaggtcccacaggctcctgccctggactaccgagtcccccgatggtgttatacattaaatatccaggatggagaagccacatgctactcaccgaagggaggaaattatcacagcagcctgggcacgcgttgtgagctctcctgtgaccggggctttcgattgattggaaggaggtcggtgcaatgcctgccaagccgtcgttggtctggaactgcctactgcaggcagatgagatgccacgcactaccattcatcactagtggcacttacacctgcacaaatggagtgcttcttgactctcgctgtgactacagctgttccagtggctaccacctggaaggtgatcgcagccgaatctgcatggaagatgggagatggagtggaggcgagcctgtatgtgtagacatagatccccccaagatccgctgtccccactcacgtgagaagatggcagagccagagaaattgactgctcgagtatactgggacccaccgttggtgaaagattctgctgatggtaccatcaccagggtgacacttcggggccctgagcctggctctcactttcccgaaggagagcatgtgattcgttacactgcctatgaccgagcctacaaccgggccagctgcaagttcattgtgaaagtacaagtgagacgctgcccaactctgaaacctccgcagcacggctacctcacctgcacctcagcgggggacaactatggtgccacctgtgaataccactgtgatggcggttatgatcgccaggggacaccctcccgggtctgtcagtccagccgccagtggtcaggttcaccaccaatctgtgctcctatgaagattaacgtcaacgtcaactcagctgctggtctcttggatcaattctatgagaaacagcgactcctcatcatctcagctcctgatccttccaaccgatattataaaatgcagatctctatgctacagcaatccacctgtggactggatttgcggcatgtgaccatcattgaactggtgggacagccacctcaggaggtggggcgcatccgggagcaacagctgtcagccaacatcatcgaggagctcaggcaatttcagcgcctcactcgctcctacttcaacatggtgttgattgacaagcagggtattgaccgagaccgctacatggaacctgtcacccccgaggaaatcttcacattcattgatgactacctactgagcaatcaggagttgacccagcgtcgggagcaaagggacatatgcgagtgaacttgagccagggcatggttaaagtcaagggaaaagctcctctagttagctgaaactgggacctaataaaaggaggaaatgttttcccacagttctagggacaggactctgaggtgggtgagtttgacaaatcctgcagtgtttccaggcatccttttaggactgtgtaatagtttccctagaagctaggtagggactgaggacaggccttgggcagtgggttgggggtagaagttcttcctttcctaacccgggcccctgcccagctctccaaagtctttcagaaaagtaaatcctaaattcagtgatgaaaaaaaaaaaaaaaaaa

SEQ ID NO: 112 - DNA, 9448 bp, Homo sapiens
ttccgaacattcttagcatcgctcgcgccgcgccgcgccgcctgagccgagccgagcctctgctgccgccgccgcggccccgccgcccgccgcgggcgcccaccaagcactttgcagactcgcttccaccctgcgggccattccgcgcggcggggcccgggcccggggcggccgcgtccaggcacaggccatgcagtgacgcccccccacccctccacctttgcccggagcgcgggcagcagcccagcgcgccagccggccccggggcaggagcggtgctaggcaggggtggggtggccgggcccagggaccgggagccggggagggagccgggcaccgagcagagggcgggggaagcggcgccgaagtttgcctcggactcgccgggcgctgcggtggctccctgggccgaggactgttgctgccgctgccgccgccgcttcattgcacattcaagtggaaaattttcaggagtcagcagaaacattgtgtccaaaaaagactgagtcgcagttaccaccaaacccaggaggagactctccctggaaaacttcccttccctttcggtttattttcttgaaaaggctccaggcttcggcttggaaaatcccaccgccaaaattgagcccagcagctggagcggcagtgagagccctgccgaaaacatggaaaggatgagtgactctgcagataagccaattgacaatgatgcagaaggggtctggagccccgacatcgagcaaagctttcaggaggccctggctatctatccaccatgtgggaggaggaaaatcatcttatcagacgaaggcaaaatgtatggtaggaatgaattgatagccagatacatcaaactcaggacaggcaagacgaggaccagaaaacaggtgtctagtcacattcaggttcttgccagaaggaaatctcgtgattttcattccaagctaaaggatcagactgcaaaggataaggccctgcagcacatggcggccatgtcctcagcccagatcgtctcggccactgccattcataacaagctggggctgcctgggattccacgcccgaccttcccaggggcgccggggttctggccgggaatgattcaaacagggcagccaggatcctcacaagacgtcaagccttttgtgcagcaggcctaccccatccagccagcggtcacagcccccattccagggtttgagcctgcatcggccccagctccctcagtccctgcctggcaaggtcgctccattggcacaaccaagcttcgcctggtggaattttcagcttttctcgagcagcagcgagacccagactcgtacaacaaacacctcttcgtgcacattgggcatgccaaccattcttacagtgacccattgcttgaatcagtggacattcgtcagatttatgacaaatttcctgaaaagaaaggtggcttaaaggaactgtttggaaagggccctcaaaatgccttcttcctcgtaaaattctgggctgatttaaactgcaatattcaagatgatgctggggctttttatggtgtaaccagtcagtacgagagttctgaaaatatgacagtcacctgttccaccaaagtttgctcctttgggaagcaagtagtagaaaaagtagagacggagtatgcaaggtttgagaatggccgatttgtataccgaataaaccgctccccaatgtgtgaatatatgatcaacttcatccacaagctcaaacacttaccagagaaatatatgatgaacagtgttttggaaaacttcacaattttattggtggtaacaaacagggatacacaagaaactctactctgcatggcctgtgtgtttgaagtttcaaatagtgaacacggagcacaacatcatatttacaggcttgtaaaggactgaacatggttatttatatatatagatatctgtatatacacacacacatatgtgcacacacacactctctctccattatcgaacgactgactgtaaacctcaccacacagggtggtgccctggccccgaggtcaccccgacttttctaaatcttgtttgagtgaagtcattttttcatgtgttcatactatcattgtagctgtgaagttctggtacagttgtaaaaagagaaattgagttgtttctctatgttcttcagatgtgcagcccacaattcctcgggaaaggtgaacctgaacaacccaagtctctctctgcagagccctgtttctaattgtggtagaaaatattgagacagagcatttgccatgggacatttacagcctttatacaaatgtatttagttctcttttttccaacataaaattcttgttttaagatacaagtaaaattaatctttaaatataaatgtaaattagtacacaaaactaagaatctttagacttatctttgtaactaattagggtggaagttatgaaagaatgtaattcactaaattattttttaaatgaaacctttttttttctttttgaaaccaaatgttaaactatagccttaagaaatgcttggtagaagtgtcctaatgagacaaatttgtacttttatcctcaaggttaacactaatctcctaatccattaaactcttgaacaggtattacaaaggaagaaaacttcaccccttatccttaacatatatagtatatttaaaaaatataaaattgtattgtactaatgtgatgatggattatttaatgaaaaagaaaaaatggctctttttgcaataagtagatacatactgaaaaaatctaaacttacaatgtttatagtcttgtgtgtgcagttatattttatatggacgaccaaattttttattaagatgagtaaatatttgaaccactgaattttaataacaaaattttaaaattggcatgaatacggaatactgcactgtgagatgcaaagtatacagaatctgtggctgggagaaaatttcatcaaatagacaagtaaaaggctcatcagttttagcatctctgctccccagaaaattgtaagcatcctcaccagcctgtggatacattctttatttctagtgacccaatatgcatattaacctgctataactagggctatatgtgtaggtatgtgtatacatatacacaaatgcacatatagagttaacacatttagtgaacacttgtttagtgtcactcagtttgctaggtgctgatatgtacgtatatctcaatgtgtctgtagacttagatacatcctcttgaagcacatccatttctttagcgtctctcagtaagttacagtacttgtttgacttaggtttaagaggcccagctacctatctctgaccttttcaaataggctcatttgggagattcttttgccaggagagattcaactttccaatctaagtattccagagcattgcccaggcagagttggtttgatgtggccagatgttttgagttatttcccttaagtgtttcactggggagagaacagggagtgctcctccagcttcccaaagaaatatgtttttgtaagtggtaggaacatgtgcacacaatagaacatgaaataagttttttaacttgtaaaacatgtcaagatttttccaccaagctagaaaataaaaaacttagttctaccacatccaattaacttacacacccccttccctgtctcaacacctgctttgaccctgcttttctattattacatcagtcagcatcttgtggtccctaacatgaggatgtggctggctcgtgggaaacagcaaaacactaagcctgacctctcccaaattgggaagaccagaggagaaagtgcaaaactgtccccatttggaatgcccattccttctagaaaccagttggacagtgctcctctgcccttcataaacagactactgttgggtccctgattccaggctggcctgtgaaggattgccccaggtgtcccctttcacggttgtcacatttacagtgacttctgttgaacacccctcttagggatgtttcttttgctcttatttcctgcatctttccttaagggaagccccatcctctcccaggaccaggagtttatgaccaggcgagcacaaatggctaaaagccaagctgtcctagaacttcagtgggagagctgtctggttcatattctacccaggaatggtacttttcagtgcagccaggagggctcttgggatttcctttccaaagcacaaaaatactgggacccaagaagaacagctagaggacaactctgttggcacagagacggggacagcccagtctgctgacctcacagggtcagctgggcccccctggtgcttcaccacctgcatcctcttgctcagaatgcctttgcagttgagttttctgggtttctatgattgaccttgaggtttactccttgctcttacaacatttctaaggatttttaaaagtttacttcttgtcttgttcttctaaagctttctccaggacagatattttccctgtcttaaccactggtccagtcatcccagtgggcttctctttgtctctcccagattagacctttgggtgagattggcatcacaacatctaatctgagtctgtcttttgtccttcattctgtatggcagtctccctttgttataaaagctttctaaagcatactaaagaagccttcccagagccccgtcttgcttctcttccaggtgctctatcccctcgagaccctctggtgccaggcttgcttcacggccatcttgtgttgtcactgcagagtttggaggccagttttccacagcctaaacagggaggagctgcagaatggggctctggtctctgggcattcatttccctcatagaggctgagaataaaacaaggacttattcacacatgttctagaaccccagaatggcccaagttacctgagaccagggtttctcaaccttgacaccattgacattttggactgggtaattctttgttctgcagagctgtcctttgcactgtaggagatttactaatatccctggcctctacccagtagtaccactagcacctattccccacccagcgtgtctccagatattgtcaaatatcccatcgggtgcaaaatgatccctggtcaagatctgttgcccaagatgttacaggtcacaatgaccacatttgaaattgttttccctttcattttaccctgtgaaagcatctctcctagagccttgcaagaggcaggtgacattgtgtccatatttcttcctgtttcagaacttctgtttcacaacaatttctctctcgctacaagtattctttcactcagcactggggaagttgggaacagctggtcaccatcatccctttaatcaactcacacctgtttaaagagtgtttctgatttgaccttcatcccttagtttactggcgttaaaaaaagtctcagcaattttcattatttctcgtgggtctcattatcaaacctttacttatttcggcatatttcctctgggcttcttctagtttctgccttacaagcaatgctgttctgtaaatttattgaaacctctggaacatttcacctttagagatggaggatggaaggattggtaccagaagagggctaagatacgttttctgtcttgagctgaaagcacagtctactctccttcgttttgtcgatgagaaagttgaggccagaggggaggtgacatgtttagagtcacccagctggttagtgacagaaaaagcgtgagagttgtctaggattcctgccactttggtccctggcctctcctgggggaggctgctgttcttaggtgctctaagcttaatccctcagaatgtgtggacaggtcagcttagaagagatggggagattcaggatccccctgtgccagagcacagcctcaccggatgctgcttcccacactgaagtgtcctgtccgaccattgctatctgaggcatccacaagcaggtaggaaagctggcgagccattttacttcctgaggacaattccccagccacaggctctgagtcaaatttctatttggtaagcatcctagcagcaaagtcctgcactcagaccagccaaaaaacagcccccattccaagtacttggtgtcaaaagtccccgaacgacttttaaacccaagtcttcttaaggtttcagtactgtggtggctttagcagttgtttttgtgcaactataaattatttaaatcatctgagatgacagtcaattttacaaaccaggtacatattaatttgtataattttgtatatgctctggtacactacctgaactaacgaagggtagaactaattctgtttgtcagtgttcacacctgtaacattaggaggatatgtctgcattgcttatttctttatgttggtgtttctgtggcaaagccctgcacatggcatttctgaaaagccttaaatctttaagatgttgcatgtagggtatgcagtgcaaaaggctgcctcagaactgtgagcccttttgtaagctggaagcatttctcttactactgttacttttgtaggaagttttcaattcagagctgccaaagtgttcccgtaagcagtgccttagtaataccttagtcatgccgccagccttttcttacaccaattcctaatgttcatttacgaattggcccaatattggaaacaaaacaagcaaaaattgtcttcatttttgttttgtaagcccattttttctccagttctataggaaactgactgcttggtgtaaaatccgaaactggacacaagtcagttctttcaccacactcaaatgtatataccaaaacaaaaggttgcaacttcatagtttactatgaaaagcaaattgtactttttaatgttgccttttaaattcatgaccaaatacttagctatttgtgaatcttctgcactctagcatgaaagtgcctttggtttgagattccagcttagaaaagtgctgccataataacgataatttgtagagagaccaaaaatattttgagatcaccgtaatgcctttggtttaccgggatgagtaaccaaccacaggcctctgttcacaagagcacgacgtggtccccgcctgctgctagtctgtctgccactgggggcctcccaacatccatagcacacttcagcggaaggaccccagaaactgttgtgtttgtgtgtgctgatgacctagtgtgtcatttcacctcgtcacccagccctgcgtccggatgaggggacttctgcacaaatgacagaatctcggctggtggacagatactacagctttctcctcctccttgtgttcgtgttcagtctctgtggagactttcttttccattcaaatgacagtgcgcacttatctggtttacacaatgataccattttgaaagttggaagcctcaaactgagacgacagtgcagaacaaaacaaaagtgagttagggtcgttaaaattgaagtgttcttcttagggcaaacatgttgactccgagtattgtgtatgaatgtgctacgagaaacttccaaagagcaccattcacaatttggcattttcaaagaatgttccagccctcaaaggggcaactctttaaagtccttgttggcttttatccaaaccttgtagaaattgggaaagctgatagaggtaaggaagacgagtgaaaaggacaagaaggccaaacaccagccaaaaagaaactaggaaaaaaagattttctttgctaatatagatgtaaaaataacatcagacatctttgaaaattagcctctaaactcttaatacatacgttctgtgtgtctctacctggcgtctttaagaatatcctctctgggctctgaaattttaggagtgattcttatccactccaagttgtaagtatttgtagaaatttgtgcaaacaaacaaaaactatcaaatgaaaagaaaatgtactcaacctaacttatagttagcagctggaattctcaactcttccctgccagcactataccacagtgtggaagaaattagtcaaatgcttgttttcctgcttctcttttcaactgttactgtgctttgtttgaaagtagttttctctctcaaagccgttgcttatatcgttaagaatgaaggtttgtgtttaaaatttattgcattgcaaagggtagtttcactgaagtcatgcaccattaaataagatgaaatatttgtatttattgtcctacttcctaagccgtaacttcttttcctctgtgaatttgcattgagtcactcatgctacactacatcgctttagtatttgagatggcatttatgtttcctctcgtttatcatgaaatggggtcagattccatcagattccacctctgtcaggtggactcttgtctgccttccatgatgagattttttttctccttcccctttctttaagagaggctgacagatctaggtgtcaatcaattggaaaccagtctctgattttttttcattagttattttctatcattagtttcactgtgtaaattagatatcaactgcacttctttaaaaaaaaatacatctccctattacctccttgaaagatttacttctgtaggcctttttcaataggctcatgactgcagacaaggaaaaaaaaagtaaaaacaaaaacagtatgtgcctgaaaatgacaaaaaaaaaatttgtaacatttaaaaaagaaacctgaatagcctttaattctttaataatacacttaaattttatgtaaatcggttttcgccacgtgtgtttgttcacattctaaatgacttaatgggattctcacggtctgtgtctttgtgtcacgtgtataaaatgggcttgtgatgtaagcgtttcatctggtcagtggttcctttgatattgtactgctgctgggagtgggctgtggaacctgccttcgggtaactgggttcctcttgggtagattggagagatgggggtgggcgtgggcaaattctcacacatgttttcttaacctatttgcagaaactttcaaaaggcatttgattaaacctcttggcagtacagtattcttgtatttgttaacgtctgtgtttaggtactggtacctttttgttttaaaatgttctaagtgttggctttaaagtgaatttatctttagtatgatagttatatgaaaattataggatttgtgtgcagagaatttttttataaagtgctttgtaaaaaaaaaaaaatgtattctagcttttgcggtacatatgtgtgataactttaatacccatgacagttaagtgcaattatttcatcactctaaaaatgctatttttgtgtcagttcctgcaggtgttttcatgtctttgcaaagtgacacattttgatgccttcttgataaagtggtagacattttgtagctttctagaaactttgtattcatacggtatcaatgaaaaataaagaaaatgaaagtgtgggtcaccttttttatctgcaaaaaaaaaaaaaaaaaa

SEQ ID NO: 113 - DNA, 2889 bp, Homo sapiens
aggcccgggggtcgccggggccacgacttctcggagaccgtcctgcgctctctggagacgcgctgtccgcgcccagggtggtgccatgtggggcgctcgccgctcgtccgtctcctcatcctggaacgccgcttcgctcctgcagctgctgctggctgcgctgctggcggcgggggcgagggccagcggcgagtactgccacggctggctggacgcgcagggcgtctggcgcatcggcttccagtgtcccgagcgcttcgacggcggcgacgccaccatctgctgcggcagctgcgcgttgcgctactgctgctccagcgccgaggcgcgcctggaccagggcggctgcgacaatgaccgccagcagggcgctggcgagcctggccgggcggacaaagacggccccgacggctcggcagtgcccatctacgtgccgttcctcattgttggctccgtgtttgtcgcctttatcatcttggggtccctggtggcagcctgttgctgcagatgtctccggcctaagcaggatccccagcagagccgagccccagggggtaaccgcttgatggagaccatccccatgatccccagtgccagcacctcccgggggtcgtcctcacgccagtccagcacagctgccagttccagctccagcgccaactcaggggcccgggcgcccccaacaaggtcacagaccaactgttgcttgccggaagggaccatgaacaacgtgtatgtcaacatgcccacgaatttctctgtgctgaactgtcagcaggccacccagattgtgccacatcaagggcagtatctgcatcccccatacgtggggtacacggtgcagcacgactctgtgcccatgacagctgtgccacctttcatggacggcctgcagcctggctacaggcagattcagtcccccttccctcacaccaacagtgaacagaagatgtacccagcggtgactgtataaccgagagtcactggtgggttcctttactgaagggagacgaaggcaggggtggattctcgaggtggaagtccgcacatgtcggtggtatttatggcacgattcctttggatggcttcatttgcccccagactgtatgaaaacatctccgaattagcatttctggatatgtttcatccagggtatcattgatttatgatggaaaaccggcctcagctggagatgactgtgatgttgctgatgggtgtataacaaatgcttgagtccgaagtgcccttgagatatggttgacgaaagaattttataaactgataaattaaggatttttattatgttgttattattatttcttttttgttgttgactgcacaggatcaaaatgcctgttatctcccttttacctgggactttttttttttttttttttttttttaatcagacagggtcttgctctgttgcccaggctggagtgcagtggtgcgatctcggctcactgcaacttcagcctcctggattcaggcaacactcctgcctcagcctcccacgtggctgggattacaggtgcctgcccccatggctaattttttgtattttttgtagagatggggtttcaccatgttggctgggctggtctcactctcctgacctcaagcaatctgcctgtctcagcctcccaaagtgctgggattacaggcgtgagccaccgcccccagcctgagcctttttttttttctaatgcatccaaggttaaggggaagacgcaaataacaggactattctaaaaggaaacctgtttgaactctgtgagatcagtcatcagtctcagtattccacaggcacaccttaatttcattgtaaaaagatatatatattttgtctatttttgtgcttttgggggcctattttgtgcttttttaccttatgtagagatcttattacaaagtgattttctacattaaaaagagactgaaataaattgtatagttacttaactaatgaagacatttcagaactctgggatgattttaatcttgaagtagtaggtggtatagtcataaaaccattcatccccttcttgattgtatcttaattttctggctttaaggtgacatctgagaggtaatgcattcttttttatattgaaatcataaactatcacccgctgcttctctgagttacttttaattttgccttgtggttatggtttggcgtttccttctgtttggttttcagagccccatgtctatatagtcctgagtgcaagtaattactatacttgtaaatgaagatcagtatttctgcctagatctgataaaaaaattttcttgtcttagttataaaaattcaaagaaatgtgttacaaagatacttagtatagctcctcagccataacctgagacttgggatgaaatttaaaccagatacgatttactttgcagatcataaggctttttatactcttgttatcaaaatggcttatttttcaggcactaaggattgttaagagaaaagcttttcaacgaaggattgcctttcttctcccacactgttcttgatttcctctctctttcaggcctcaacaggcactgtattcattgccaatgttccaaattatcaaattcaagtgaatttatttgtgtgttctttacttatataaaaaaagataactttaaggatgtgcaagtacatttccaactgctagcacaaccagtattttgtaattaaacaaatcgctgtatggtatggtcttctacacatttatgtctatagatatctatcgatcatctttctattctgtttcatgactgaataatgtaaaaccagtgttggcaattggtatcatcaatgatactcattttttaataaccaaaggcaggggaaaatcattttacttattaataaatattttatgatgtgaa

SEQ ID NO: 114 - DNA, 1918 bp, Homo sapiens
ggtgccactcgcgcgccggccgcgctccgggcttctcttttccctccgacgcgccacggctgcccagacattccggctgccgggtctggagagctccccgaacccctccgcggagaggagcgaggcggcgccagggtggcccccggggcgcgcttggtctcggagaagcggggacgaggccggaggatgagcgactgagggcgacgcgggcactgacgcgagttggggccgcgactaccggcagctgacagcgcgatgagcgactccccagagacgccctagcccggtgtgcgcgccaggcggagcgcgcaggtggggctgggctgttagtggtccgccccacgcgggtcgccggccggcccaggatgggcgctggcaacccgggcccgcgcccgccgctgctacccctgcgcccgctgcgagcccggcgtccggcccgcgccctgcgctcatggacggcggctcccggctggcggcggcgcgcccccgggctgtgaatgcgactcgcccctcggccgcgctccccgcccgcccgcccgccgggacgtggtaggggatgcccagctccactgcgatggcagttggcgcgctctccagttccctcctggtcacctgctgcctgatggtggctctgtgcagtccgagcatcccgctggagaagctggcccaggcaccagagcagccgggccaggagaagcgtgagcacgccactcgggacggcccggggcgggtgaacgagctcgggcgcccggcgagggacgagggcggcagcggccgggactggaagagcaagagcggccgtgggctcgccggccgtgagccgtggagcaagctgaagcaggcctgggtctcccagggcgggggcgccaaggccggggatctgcaggtccggccccgcggggacaccccgcaggcggaagccctggccgcagccgcccaggacgcgattggcccggaactcgcgcccacgcccgagccacccgaggagtacgtgtacccggactaccgtggcaagggctgcgtggacgagagcggcttcgtgtacgcgatcggggagaagttcgcgccgggcccctcggcctgcccgtgcctgtgcaccgaggaggggccgctgtgcgcgcagcccgagtgcccgaggctgcacccgcgctgcatccacgtcgacacgagccagtgctgcccgcagtgcaaggagaggaagaactactgcgagttccggggcaagacctatcagactttggaggagttcgtggtgtctccatgcgagaggtgtcgctgtgaagccaacggtgaggtgctatgcacagtgtcagcgtgtccccagacggagtgtgtggaccctgtgtacgagcctgatcagtgctgtcccatctgcaaaaatggtccaaactgctttgcagaaaccgcggtgatccctgctggcagagaagtgaagactgacgagtgcaccatatgccactgtacttatgaggaaggcacatggagaatcgagcggcaggccatgtgcacgagacatgaatgcaggcaaatgtagacgcttcccagaacacaaactctgactttttctagaacattttactgatgtgaacattctagatgactctgggaactatcagtcaaagaagacttttgatgaggaataatggaaaattgttggtacttttccttttcttgataacagttactacaacagaaggaaatggatatatttcaaaacatcaacaagaactttgggcataaaatccttctctaaataaatgtgctattttcacagtaagtacacaaaagtacactattatatatcaaatgtatttctataatccctccattagagagcttatataagtgttttctatagatgcagattaaaaatgctgtgttgtcaaccgtcaaaaaaaaaaaaaaaaaaaaaaaaaaa

SEQ ID NO: 115 - DNA, 5855 bp, Homo sapiens
agttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctgcacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatccaaaaaaaaaaaaaaaaaa

SEQ ID NO: 116 - DNA, 2837 bp, Homo sapiens
gggagcggccgcccccgccgccgccgcgcgctcgccgggcccgggcggagctgcgcagtcctctcgcagctgcgccaggacagccggcgcgcggccgtgcccacaagttgccggcagctgagcgccgcgcctcctcctgctcgcagccccctacgcccacccggcggcggtggccagcgccaggacgcacatcccgcggacaccgaccccagatgtaaagcgggaccccagcccctcgccccccggcgcgatcgacagtctcgccagcgtctcctctgccaaaacccagggctggaagatgtggcagccggccacggagcgcctgcaggagagatttgcagacacagaagcggcacagagaaggccattgtgaagatcaaggcagaaaccggagttatggcatcataagccaaggaatgccaaggattgctggcaaccacctgatgttagaagagtcgaggacatgttcttctccagagcttttggatggtgtgtggccctgccaacctttacattttggacttccagcctccgaaatgcactttcagaccatgctgaagtctaaattgaatgtcttaacactgaaaaaggaacctctcccagcggtcatcttccatgagccggaggccattgagctgtgcacgaccacaccgctgatgaagacaaggactcacagtggctgcaaggttacctacctgggcaaagtctccaccactggcatgcagtttttgtcaggctgcacagaaaagccagtcattgagctctggaagaagcacacgctagcccgagaggatgtctttccggccaatgccctcctggaaatccggccattccaagtttggctccatcatctcgaccacaaaggggaggccacagtgcacatggataccttccaggtggcccgcatcgcctactgcaccgccgaccacaacgtgagccccaacatcttcgcctgggtctacagggagatcaatgatgacctgtcctaccagatggactgccacgccgtggagtgcgagagcaagctcgaggccaagaaactggcccacgccatgatggaggccttcaggaagactttccacagtatgaagagcgacgggcggatccacagcaacagctcctccgaagaggtttcccaggaattggaatccgatgatggctgaatgaacttgagacgcttcagcaaaggcagcattggtcacggagttcaagggaatagatgagtaagcaacgtttcaaatttgggatgaaaagactgccaaactattggctgaccaaggtttttaaattcagaagagcaattctaaatctaaagaaatgtatcattaaagtaattacgttacattgaaacctgctgctgctgtgactgtgaggagggtgggagtgtggatggggaggaaggttctaggctctcttatttttctcatttcccaatgcctctctgtgggagagctccatgccagttttcaccacgctcaggcaaatactctgcagctgttattggatgggccattccgatctgccttatgaaattccacaagaatgttaggggcacctatgggatctctagtggggtgggcagggtgctgatggggacgctggccgcagggaggaaggaacatctcgggagggccctctgttcctctcccacggcagatgccctcctctgtatgcaaatcagcacagcctttattgagctttacaactaacaacctgatagttggcagttaattcacagttacagataatgcttttatttacataaatataccaagtagtaccctcttattgtattcacttcatctattttcttagaatacttgcaattactaatgaccccttccctttccctcctgctgccctgtccaccctctttccccttctaacatccttagagggatgaaatctcagcatatgttgcaggacaccaaaaggaagaaaacaatcaagcaaataaaataaacagtcaaacaaaccaggagtttaaaacaacaaccccaacaacagaagccttggcaaagaggaataagtgatcagcaagtgaacacactctatgtcaactctccttttatccagctgagatttatggtaacttatttaattaatggtcctgtctgatgcatccttgatggcaagcttcaaatctgatttggtatcaccgaggaaaccttgcccccatcactcagcattgcacttagatacagaatgagttagataaacttggcttgtctagagacccatgtcatcttaacctaaagggaaatcttattgcgttatcataaaattgatgatatcttagggtcagaattgccctttttttttattttgaatgggaagttctcactaaaacaatcctgagatttcttaatttcatggttctttaaatattataaacacagagtcaacatagaatgaaattgtatttgttaaaatacacacattggaggacaagagcagatgactacttttcgaagtaatgctgctccttcctaaaagtctgttttcaatcctggtaatattaggggcactgcggcacctaagaagccttaaatgagagctaatccaatctagagagcgatggtgtcagcatttcggtctgcatatctgtgtgtccgtatctgcgtttgtgtgcgtgtacgtgtgcccctgtgtgtgggcccagttttcaggcatgtagaataagcatggagtcatattgaggaggactcacttcttgaagatatgcttgttgctttacaacatatgtaagctattctttagcataaatgcattcattctttaataaaaatatgtttgcattaataaagctgaggagtttcataaaaaaaaaaaaaaaa

SEQ ID NO: 117 - DNA, 2389 bp, Homo sapiens
aggcgcggttgtgagtagtaccgggagtggggtgatcccgggctaggggagcgcggcggccgcgatcgggcttagtcggagctccgaagggagtgactaggacacccgggtgggctacttttcttccggtgcttttgctttttttttcctttgggctcgggctgagtgtcgcccactgagcaaagattccctcgtaaaacccagagcgaccctcccgtcaattgttgggctcgggagtgtcgcggtgccccgagcgcgccgggcgcggaggcaaagggagcggagccggccgcggacggggcccggagcttgcctgcctccctcgctcgccccagcgggttcgctcgcgtagagcgcagggcgcgcgcgatgaaggcggtgagcccggtgcgcccctcgggccgcaaggcgccgtcgggctgcggcggcggggagctggcgctgcgctgcctggccgagcacggccacagcctgggtggctccgcagccgcggcggcggcggcggcggcagcgcgctgtaaggcggccgaggcggcggccgacgagccggcgctgtgcctgcagtgcgatatgaacgactgctatagccgcctgcggaggctggtgcccaccatcccgcccaacaagaaagtcagcaaagtggagatcctgcagcacgttatcgactacatcctggacctgcagctggcgctggagacgcacccggccctgctgaggcagccaccaccgcccgcgccgccacaccacccggccgggacctgtccagccgcgccgccgcggaccccgctcactgcgctcaacaccgacccggccggcgcggtgaacaagcagggcgacagcattctgtgccgctgagccgcgctgtccaggtgtgcggccgcctgagcccgagccaggagcactagagagggagggggaagagcagaagttagagaaaaaaagccaccggaggaaaggaaaaaacatcggccaacctagaaacgttttcattcgtcattccaagagagagagaggaaagaaaaatacaactttcattctttctttgcacgttcataaacattctacatacgtattctcttttgtctcttcatttataactgctgtgaattgtacatttctgtgttttttggaggtgcagttaaacttttaagcttaagtgtgacaggactgataaatagaagatcaagagtagatccgactttagaagcctactttgtgaccaaggagctcaatttttgttttgaagctttactaatctaccagagcattgtagatattttttttttacatctattgtttaaaatagatgattataacggggcagagaactttcttttctctgcaagaatgttacatattgtatagataaatgagtgacatttcataccatgtatatatagagatgttctataagtgtgagaaagtatatgctttaatagatactgtaattataagatatttttaattaaatatttttttgtaaatattatgtgtgtgtttttttttaatctatgggaatatttcttttggaaaatcatttttcagctcaattacagagctcttgatatcttgaatgtcttttctgtttggcctggctcttaatttgcttttgttttgcccagtatagactcggaagtaacagttatagctagtggtcttgcatgattgcatgagatgtttaatcacaaattaaacttgttctgagtccattcaaatgtgtttttttaaatgtagattgaaatctttgtatttgaagcatacatgttgaaaatacaccttatcagtttttaagtacagggttttatagtgtaatatatacagagtaagtgtttgtttttgtttttcaactgaggtcaaaatggattctgaatgattttgcatatgggatgaggaaatgcttggatccttaaggagtttacgaaatctgctgttttatcaaagtgaaaaaaaattgcttattactcttcattttacactaaagcttaatgtcactaagtttcatgtctgtacagattatttaaatcatggaaatgaaaaaaatgttctctgcttgctaccaaaggacaaactcttggaaatgaacactttctgctttccttcctccaaagaattaataggcaacagtgggagaaaaaaaaggcataatggcaaatccttcaagcagggataaaagtcgatcttcaaacattaacttaagcagaccaaaaattctgatgaccgcatctagattatttttttataaaaatgattttcactatagctatgttacgctaagctactgtcccatctcttgtgatgtgtaacttttacatgtgaatattaaagtagatttctctgtcttgtaaaaaaaaaaaaaaaaaaaaaaaaaaaaaaaaaaaaaaaaaaaaaa

SEQ ID NO: 118 - DNA, 912 bp, Homo sapiens
atctttgagaccaaggcctcaagccaccaaggaaaatgaagggcctctaccaggctgctggccggattcttgttactctggggatcctcagtgtatgctctggagttattgctttctttcctgtcttttcttacaagccttggttcacaggatggagtgttcgaattgcttgtcctatctggaatggagctttggccatcacaactggtgtgcttctactgttggcttacagagagtggacccagaggtacctgggggaagctactttcacctttgtgattctgagcattatgggatgtccacttcattttgcaatagccttggaatctgctctcctgggcccatattgcttctattcattttcagggattgcagggactaattaccttggctatgcagttacctttccttatccatatgcaaaattcccattagcctgtgtggacccaccacactacgaagagtaccacctgacacttcaagccctagacctgtgcctaagctttaccctactctgtacatccttgacagtgttcatcaaactttctgcaagacttatccagaatggacacataaacatgcaactccctgctgggaacccaaaccctttttcaccataaaagtttggacctgattaaagaaggacaacaaaggccaatttgccatcaccaaaggagcagcttgacctggagggatgaggcctggaggccgacagcaggactccgtcagtgattctttcagctcttgaaaatgtccaagaaagacactttctctcacctttttggagcctctagcctgccctgggaagcctggtggactggtgctgagaagagaccacggcccagttggagtcccactctgtcgcccaggctggagtgcagtggcacgatctcagctcactgcaacctccac

(19) United States
(12) Patent Application Publication
(10) Pub. No.: US 2009/0123439 A1
(43) Pub. Date: May 14, 2009

(54) DIAGNOSTIC AND PROGNOSIS METHODS FOR CANCER STEM CELLS

(75) Inventors: Kyuson Yun, Bar Harbor, ME (US); Hyuna Yang, Bar Harbor, ME (US)

Correspondence Address: DAVID S. RESNICK, NIXON PEABODY LLP, 100 SUMMER STREET, BOSTON, MA, 02110-2131, US

(73) Assignee: THE JACKSON LABORATORY, Bar Harbor, ME (US)

(21) Appl. No.: 12/102,558

(22) Filed: Apr. 14, 2008

Related U.S. Application Data
(60) Provisional application No. 61/015,961, filed on Dec. 21, 2007.
(60) Provisional application No. 60/986,746, filed on Nov. 9, 2007.

(51) Int. Cl. A61K 35/12; C12Q 1/68; C12Q 1/02; G01N 33/574; A61P 31/00; C40B 40/10; C40B 40/08

(52) U.S. Cl. 424/93.21; 435/6; 435/29; 435/7.23; 506/18; 506/17

(57) ABSTRACT

The present invention provides methods for diagnosis and prognosis of cancer stem cells (CSC) using expression analysis of one or more groups of genes, and a combination of expression analysis from a biological sample from the subject. The methods of the invention provide a method for accuracy detecting cancer stem cells in a population of cancer cells. The invention also provides methods and kits for diagnosis and prognosis of cancer in a subject using cancer stem cell biomarker expression analysis.

### CROSS REFERENCED APPLICATIONS

This application claims benefit under 35 U.S.C. 119(e) of U.S. Provisional Patent Application Ser. No. 60/986,746 filed on Nov. 9, 2007 and U.S. Provisional Patent Application Ser. No. 61/015,961 filed on Dec. 21, 2007, the contents of which are incorporated herein in their entity by reference.

### FIELD OF THE INVENTION

The present invention relates generally to diagnostic and prognostic methods for identifying cancer stem cells (CSC) in a population of cells. More specifically, the present invention is directed to a method to identify cancer stem cells using an array of biomarkers or a gene expression signature of cancer stem cells. The present invention also relates to uses of such cancer stem cell biomarker for prognostic and diagnostic uses.

### BACKGROUND OF THE INVENTION

Cancer is one of the leading causes of death worldwide and currently available therapies are not very effective against many cancers. Recent identification of cancer stem cells (CSCs) from multiple human cancers provides a possible cellular explanation for this challenge. CSCs constitute only a small fraction of a tumor mass but are thought to be solely responsible for cancer initiation, growth and recurrence. CSCs appear to be inherently more resistant to radiation and chemotherapies, suggesting that CSCs that are self-renewing, multipotent, and tumor-initiating by definition may evade commonly used therapies.

Human CSCs are identified by their unique immunophenotypes that allow prospective isolation of a subset of cancer cells that are then directly tested for tumor-initiation in immune-deficient mice. Because prospective isolation of CSCs from mouse models of cancer has been difficult, there is a brewing controversy over whether the CSC hypothesis is based on an epiphenomenon of transplanting human cells into mice.

The fundamental basis for the cancer stem cell hypothesis is that there is a hierarchical organization of cells within a tumor in which only a subset of cancer cells have the characteristics of stem cells (self-renewal and multipotentiality). In addition, this subset contains the only cells that can initiate a tumor when transplanted (1-4). Because of their cellular characteristics, cancer stem cells are thought to be responsible for metastasis, therapy resistance, and recurrence (5-7). Emerging studies now show that cancer stem cells are indeed more resistant to radiation- and chemo-therapy (8, 9).

Therefore there is a definite need for methods to identify cancer stem cells. Currently there is no validated biomarker or biomarkers for cancer stem cell populations. Gene expression profiling could potentially be used to identify cancers comprising cancer stem cells. Subjects identified with cancers comprising cancer stem cells would more accurately predict therapy outcome and thereby guide more effective treatment decisions.

### SUMMARY OF THE INVENTION

The present invention relates generally to diagnostic and prognostic methods for identifying cancer stem cells (CSC) in a population of cells. More specifically, the present invention is directed to methods to identify cancer stem cells using an array of biomarkers or a gene expression signature of cancer stem cells.

The present invention is based upon the discovery of a group of genes, herein referred to “cancer stem cell biomarkers” or “CSCB” which are set forth in Table 5 that can be used alone, or in combination (i.e. subsets) for identification of cells that are cancer stem cells, using gene expression analysis. Analysis of the increase and/or decrease of expression of these genes can be used for the identification of cancer stem cells. Accordingly, the present invention provides gene groups, the expression pattern or profile of which is useful for methods to identify a cancer stem cell (CSC).

The cancer stem cell biomarkers as disclosed herein are useful for prognostic and diagnostic methods to identify a subject with a cancer which comprises cancer stem cells, and often for identifying a subject with an aggressive form of cancer, or likelihood of recurrent cancer. For example, if a subject is identified as having a cancer which comprises at least one cancer stem cell, the subject is likely to have recurrent cancer. In some embodiments, if the subject who has undergone cancer therapy and has eliminated the tumor and/or reduced the tumor size is categorized is being in remission, if the subject is identified as having a cancer stem cell, the subject is likely to have a recurrence of the cancer. The cancer stem cell biomarkers as disclosed herein are also useful for developing anti-cancer therapies which specifically target and reduce the viability of cancer stem cells. In some embodiments, the cancer stem cell biomarkers as disclosed herein are also useful for monitoring the progression of cancer in a subject and also for assessing the efficacy of treatment of the subject with an anti-cancer therapy. In a similar manner, the cancer stem cell biomarkers as disclosed herein are also useful for monitoring and assessing anti-cancer therapies in preclinical, clinical or other trials, to identify the efficacy of the agent to reduce the cancer stem cell population by a particular therapy or therapeutic regimen.

Here, the inventors have discovered that cancer stem cells exist in “spontaneous” mouse brain tumors, demonstrating that CSCs occur in brain tumors. Furthermore, the inventors have discovered gene expression signatures that distinguish brain cancer stem cells from normal neural stem cells and non-stem cancer cells, and show that genes on this list are expressed in rare cancer cells in primary human glioblastoma multiforme (GBM) samples. The inventors demonstrate that mouse models may be used to examine the role of CSCs in tumor initiation, progression, and invasion in their natural environment and test new therapeutics against CSCs in vivo.

In one embodiment, one group of gene transcripts useful in the identification of cancer stem cells are set forth in Table 5. The inventors have found that taking groups of at least 10 of the genes listed in Table 5 provides a much greater diagnostic capability of identifying cancer stem cells than chance alone.

In some embodiments, one could use more than 10 of the gene transcripts listed in Table 5, for example about 10-46 and any combination therein between, for example 11, 12, 13, 14, 15, 16, 17, 18, 19, 20, 21, 22, 23, 24, and so on. In some instances, discussed in further detail below, the inventors have found that one can enhance the accuracy of the diagnosis by adding certain additional genes to any of these specific groups. When one uses these groups, the genes are compared to the levels of genes of a reference sample. In some embodiments, the maximum gene transcripts is about 10, and in another embodiment the maximum gene transcripts is about 46 genes.

One aspect of the present invention relates to methods to identify a cancer stem cell in a population of cells, the method comprising; measuring a level of expression of at least 6 nucleic acid sequences encoding proteins selected from the group consisting of: (i) 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4, KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik in a biological sample; and (ii) comparing the level of expression of each nucleic acid sequences measured in (i) to a reference expression level for each of the nucleic acid sequence measured, wherein if a difference in the level of the expression of at least 1.5-fold increase for upregulated genes, or at least 0.5-fold decrease (or 50% decrease in expression) for downregulated genes of the measured nucleic acid sequence in the biological sample is detected as compared to the reference expression level, then it indicates the presence of a cancer stem cell in a population of cells. In some embodiments the difference is an increase of at least 1.5-fold as compared to a reference level, and in alternative embodiments the difference is a decrease of at least 0.5-fold (or 50% decrease in expression) in the level as compared to a reference level. Where the difference is an increase of at least 1.5-fold, the increase is an increase of at least 1.5-fold as compared to the reference level and the genes are selected from the group comprising; 2310046A06Rik; 3110035E14Rik; A930001N09Rik; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GPR17; ID4; KAZALD1; KCNA4; LARP6; LGALS3; MGP; MIA; NINJ2; OPCML; PAPSS2; S100A4; S100A6; SCG5; SRPX2; TMEM46 and VWC2. This group of genes is referred to herein as “cancer stem cell upregulated biomarkers” or “upregulated genes”. Where the difference is a decrease of at least a 0.5 fold (or stated another way, a 50% decrease in expression) as compared to a reference level, the genes are selected from the group comprising; AI593442; AI851790; AOX1; ARHGAP29; GJA1; SCG3; TEAD1; WNT5A; and 5033414K04Rik. This group of genes is referred to herein as “cancer stem cell downregulated biomarkers” or “downregulated genes”.

In some embodiments, for at least 6 respective nucleic acid sequences measured the difference is an increase in level of expression by at least 1.5-fold as compared to a reference level. Such genes where an increase in the level of expression of at least 1.5-fold are selected from at least 6 respective nucleic acid sequences selected from the group consisting of; 2310046A06Rik; 3110035E14Rik; A930001N09Rik; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GPR17; ID4; KAZALD1; KCNA4; LARP6; LGALS3; MGP; MIA; NINJ2; OPCML; PAPSS2; S100A4; S100A6; SCG5; SRPX2; TMEM46 and VWC2. In some embodiments, for respective sequences in said at least 6 nucleic acid sequences, the difference is a decrease in level of expression. Such genes where a decrease in the level of expression of at least 0.5-fold (or at least a 50% decrease), or at least 0.4-fold as compared to normal levels (i.e. at least a least a 60% decrease as compared to normal levels), 0.3-fold as compared to normal levels (i.e. at least a least a 70% decrease), 0.2-fold as compared to normal levels (i.e. at least a least a 80% decrease), 0.1-fold as compared to normal levels (i.e. at least a least a 90% decrease) are selected from at least 6 respective nucleic acid sequences selected from the group consisting of; AI593442; AI851790; AOX1; ARHGAP29; GJA1; SCG3; TEAD1; WNT5A; and 5033414K04Rik.

In some embodiments, a biological sample is obtained from a subject at a first time point. In some embodiments, identify a cancer stem cell in a population of cells further comprises measuring a level of expression of at least 6 nucleic acid sequences encoding proteins selected from the group consisting of: 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik and combinations thereof, in a biological sample obtained from a subject at a second timepoint, and comparing the level of expression of each nucleic acid sequences measured in at a first time point to the level expression of each respective nucleic acid sequence measured at a second time point; wherein a difference in the level of expression of at least 1.5-fold increase for upregulated genes or at least 0.5-fold decrease (i.e. 50% decrease in expression) for downregulated genes of said measured nucleic acids at said first timepoint as compared to the level of expression at said second timepoint indicates a different proportion of cancer stem cells as compared to non-stem cancer cells in the biological sample from the first time point to the second time point.

For example, a decrease in the number of upregulated genes that are at least 1.5-fold increased measured at the second timepoint as compared to the number of upregulated genes that are at least 1.5-fold measured at the first timepoint would indicate the subject has a decrease in the proportion of cancer stem cells as compared to non-stem cancer cells in the biological sample from the first time point to the second time point. Alternatively, a decrease in the level of expression of upregulated genes that are at least 1.5-fold increased which are measured at the second timepoint as compared to the level of expression of the same upregulated genes that are at least 1.5-fold measured which are measured at the first timepoint would indicate the subject has a decrease in the proportion of cancer stem cells as compared to non-stem cancer cells in the biological sample from the first time point to the second time point.

Alternatively, an increase in the level of expression of downregulated genes that are at least 0.5-fold decreased (i.e. have at least 50% decrease expression) which are measured at the second timepoint as compared to the level of expression of the same downregulated genes that are at least 0.5-fold (i.e. 50% decrease in expression) which are measured at the first timepoint would indicate the subject has a decrease in the proportion of cancer stem cells as compared to non-stem cancer cells in the biological sample from the first time point to the second time point. Alternatively, an decrease in the number of downregulated genes that are at least 0.5-fold decreased (i.e. 50% decrease in expression) when measured at the second timepoint as compared to the number of downregulated genes that are at least 0.5-fold (i.e. 50% decrease in expression) measured at the first timepoint would indicate the subject has a decrease in the proportion of cancer stem cells as compared to non-stem cancer cells in the biological sample from the first time point to the second time point.

In some embodiments, the level of expression measured is the level of gene transcript expression. In alternative embodiments, the level of expression measured is protein expression.

In some embodiments, the difference in expression is at least about 1.5-fold increase in upregulated genes as compared to a reference expression level. In some embodiments, the difference in expression is at least about 0.5-fold decrease (i.e. at least about a 50% decrease) in the downregulated genes as compared to a reference expression level. In some embodiments, the difference in expression level has a q-value of less than 0.05.

In some embodiments, the levels of expression of at least 10 said nucleic acid sequences are measured, and in some embodiments, at least 20, or a least 30 or at least 40 nucleic acid sequences are measured.

In some embodiments, the nucleic acid sequences encoding the proteins measured are selected from a group of nucleic acid sequences consisting of GenBank Identification Nos; 2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); 5033414K04Rik (SEQ ID NO:45); U16153 (SEQ ID NO:46) and combinations thereof.

In some embodiments, the expression level of subgroups of nucleic acid sequences are measured, for example one such first group can include, CAV1, S100A4, S100A6, COL6A1, COL6A2, WNT5A. In some embodiments, the expression level of subgroups of nucleic acid sequences are measured, for example one such first group can include, but is not limited to MGP, BGN, KAZALD1, COL6A1, SCG5, COL6A2, VWC2, MIA, SCG3. In another embodiment, the level of expression of a second group of genes can be measured can include, but is not limited to, TMEM46, OPCML, NINJ2, ENPP6, CAV1, S100A6, S100A4, GPR17, D930020E02RIK, GJA1, 5033414K04RIK, KCNA4. In another embodiment, the level of expression of a third group of genes can be measured can include, but is not limited to CYTL1, AI851790, WNT5A, PAPSS2, ARHGAP6, D3BWG0562E, ARHGAP29. In another embodiment, the level of expression of a fourth group of genes can be measured can include, but is not limited to FOXC2, FOXA3, A930001N09RIK, LARP6, TEAD1, CASP4. In another embodiment, the level of expression of a fifth group of genes can be measured can include, but is not limited to: DDC, LGALS2, CAPG, SRPX2, DHRS3, BFSP2, AOX1, 3110035E14RIK, 2310046A06RIK, E030011K20RIK, AI593442.

In some embodiments, a biological sample obtained from the subject is selected from the group consisting of: blood, plasma, serum, urine, stool, spinal fluid, nipple aspirates, lymph fluid, external secretions of the skin, respiratory tract, intestinal and genitourinary tracts, bile, saliva, milk, tumors, organs, cancer tissue, a tissue sample, a biopsy sample, surgical resection, primary ascites cells and in vitro cell culture constituents.

In some embodiments, a cancer stem cell identified by the methods as disclosed herein is a brain cancer stem cell. In other embodiments, a cancer stem cell identified by the methods as disclosed herein is, for example but not limited to, a breast cancer stem cell, colon cancer stem cell, ovarian cancer stem cell, a prostate cancer stem cell, a skin cancer stem cell or a melanoma stem cell.

In some embodiments, where the level of expression measured is the level of protein expression measured, protein expression can be measured using an antibody, human antibody, humanized antibody, recombinant antibodies, monoclonal antibodies, chimeric antibodies, protein binding proteins, aptamer, peptide or analogues, or conjugates or fragments thereof. In some embodiments, protein expression can be measured by ELISA, Western blot, FACS, immunohistochemixtry, radioimmunoassay, magnetic bead assays, electrical detection assays (e.g. electrical impedance spectroscopy (EIS)) or by Multiplex Immuno-Assay methods (e.g. Luminex) and kits.

In some embodiments, where the level of expression measured is the level of gene transcript expression measured, protein expression gene transcript expression can be measured at the level of messenger RNA (mRNA). In some embodiments, detection uses nucleic acid or nucleic acid analogues, for example, but not limited to, nucleic acid analogous comprise DNA, RNA, PNA, pseudo-complementary DNA (pcDNA), locked nucleic acid and variants and homologues thereof. In some embodiments, gene transcript expression can be assessed by reverse-transcription polymerase-chain reaction (RT-PCR) or by hybridization or sequencing.

Another aspect of the present invention relates to an array comprising a solid platform, including a nanochip or beads (such as disclosed in U.S. patent Application 2007/0065844A1, which is incorporated herein by reference) and protein-binding molecules attached thereto, wherein the array comprises at least 6 and at most 100 different protein-binding molecules in known positions, wherein at least 6 of the 100 different protein-protein binding molecules having binding affinity for proteins selected from the group of; 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A and 5033414K04Rik.

In another embodiment, the present invention relates to an array comprising a solid platform and protein-binding molecules attached thereto, wherein the array comprises at least 6 and at most 50 different protein-binding molecules in known positions, wherein at least 6 of the 50 different protein-protein binding molecules having binding affinity for proteins selected from the group of; 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik.

In another embodiment, the present invention relates to an array comprising a solid platform and nucleic acid-binding molecules attached thereto, wherein the array comprises at least 6 and at most 100 different nucleic acid-molecules in known positions, wherein at least 6 of the 100 different protein-protein binding molecules having binding affinity for nucleic acids selected from the group consisting of 2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); and 5033414K04Rik (SEQ ID NO:45); U16153 (SEQ ID NO:46).

In another embodiment, the present invention relates to an array comprising a solid platform and nucleic acid-binding molecules attached thereto, wherein the array comprises at most 50 different nucleic acid-molecules in known positions, wherein at least 6 of the 50 different protein-protein binding molecules having binding affinity for nucleic acids selected from the group of 2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); and 5033414K04Rik (SEQ ID NO:45); U16153 (SEQ ID NO:46).

Another aspect of the present invention relates to a kit comprising antisense nucleic acids sequences to fragments of at least 6 genes selected from the group of SEQ ID NO:1 to SEQ ID NO:46. In some embodiments, a kit can comprise protein binding molecules that have a binding affinity for at least six proteins selected from the group of 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik, or fragments or variants thereof. In some embodiments, a kit is an ELISA kit, and in some embodiments, a kit is a Multiplex Immuno-Assay kit.

Another aspect of the present invention relates to a method for identifying a subject at risk of having or developing cancer, the method comprising the steps of: (i) measuring the level of expression of at least 6 nucleic acid sequences encoding proteins selected from the group consisting of: genes 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik in a biological sample; (ii) comparing the level of expression of each of the nucleic acid sequences measured in (i) to a reference expression level for each of the nucleic acid sequence measured; wherein if a difference in the level of the expression of at least 1.5-fold increased for upregulated genes, or at least 0.5-fold decreased (i.e. a 50% decrease in expression) for downregulated genes of the measured nucleic acid sequence in the biological sample is detected as compared to a reference expression level, it indicates the subject likely to be at risk of or having cancer.

Another aspect of the present invention relates to a method for treating a cancer in a subject, the method comprising identifying a cancer stem cell in a population of cells according to the methods as disclosed herein, wherein a clinician reviews the results and if the results indicate a difference in the level of the expression of at least 1.5-fold increase for upregulated genes or at least 0.5-fold decrease (i.e. 50% decrease in expression) for downregulated genes of the nucleic acid sequences measured in the biological sample as compared to a reference expression level, the clinician directs the subject to be treated with an appropriate anti-cancer therapy. In some embodiments, such an anti-cancer agent is an anti-cancer therapy targeting cancer stem cells.

Other aspects of the present invention are use of the cancer stem cell biomarkers, such as the genes selected from the group of: 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik as prognostic and diagnostic markers to identify a subject with an cancer which comprises cancer stem cells, and often for prognosis or identifying a subject with a recurrent form cancer. For example, if a subject is identified as having a cancer which comprises at least one cancer stem cell, the subject is likely to have recurrent cancer. In some embodiments, if the subject who has undergone cancer therapy and has eliminated the tumor and/or reduced the tumor size is categorized is being in remission, if the subject is identified as having a cancer stem cell, the subject is likely to have a recurrence of the cancer. The cancer stem cell biomarkers as disclosed herein are also useful for developing anti-cancer therapies which specifically target and reduce the viability of cancer stem cells. In some embodiments, the cancer stem cell biomarkers as disclosed herein are also useful for monitoring the progression of cancer in a subject and also for assessing the efficacy of treatment of the subject with an anti-cancer therapy. In a similar manner, the cancer stem cell biomarkers as disclosed herein are also useful for monitoring and assessing anti-cancer therapies in clinical or other trials, to identify the efficacy of the agent to reduce the cancer stem cell population by a particular therapy or therapeutic regimen.

Another aspect of the present invention relates to the use as research tool to identify CSCs in animal disease models and monitor disease progression in animal models, also during treatment.

Another aspect of the present invention relates to the identification of novel gene signatures for cancer stem cells (CSCs), which may be tissue-specific.

### BRIEF DESCRIPTION OF FIGURES

FIGS. 1A-1D shows isolation of cancer stem cells from a mouse model of brain tumor FIG. 1A shows a brain section of the verb/p53 mouse model and 1B shows sphere forming cells were isolated from this brain. All tumors examined show similar cellular characteristics. These tumor spheres maintain their cellular characteristics after multiple (greater than 25) passages in vitro and multiple (>4) serial transplantations in immune deficient or syngenic mice.

FIG. 1C shows approximately 1% of these cultured TSC are CD133+D). FIG. 1D shows that the cancer stem cells (TSC) grow robustly in the absence of serum or added growth factors, in contrast to normal stem cells (NSC).

FIGS. 2A-2D shows stem cell marker analysis of normal and cancer stem cells. FIG. 2A-2D show FACS analysis of Normal (2A, 2D) and cancer (2B, 2C) cells stained for ABCG2/BCRP1 (2A, 2B) and CD133/PROM1 (2C, 2D). Gates for positive population were set using unstained control cells from same cultures. Each experiment was repeated at least 5 times.

FIGS. 3A-3B show tumor initiating cells are enriched in the Side Population (SP). FIG. 3A shows C57BL/6 (B6) normal bone marrow cells and cultured TSC from S100βverbB; p53−/− oligodendroglioma were stained with Hoechst 33342 dye to isolate SP and non-SP populations. FIG. 3B shows a table summary of injected SP and non-SP tumor stem cells to form spontaneous oligodendroglioma.

FIG. 4 shows a table of gene ontology (GO) classification of the genes identified by microarray gene expression analysis of SP cells. GO classification of “cancer SP” genes: GO and in terms of molecular function for the 538 cancer SP genes initially identified.

FIGS. 5A-5B shows aCGH analysis of TSC and NSC lines. FIG. 5A shows a schema of how genetic lesions were identified that are associated with the cancer stem cell phenotype, genomic DNA from the same samples (early passage) were extracted and hybridized on Agilent aCGH (105K) chips. C57BL/6 DNA (from brain) was used as reference. Each sample was compared to C57BL/6 (dye-swap) and copy number changes were identified. Similar to gene expression analysis, aberrations associated with p53−/−NSC were subtracted from aberrations associated with T1 (since p53−/− were not transformed at the time of the experiment). Similar analysis was performed with T2. The, aberrations that were common in T1 and T2 were selected and compared to the “cancer SP” gene list from expression analysis. FIG. 5B shows that 41 genes which were identified as having altered gene expression levels and chromosomal copy number changes that were common in the two TSC compared to NSC.

FIGS. 6A-6B shows RT-PCR validation of candidate tumor suppressor and oncogenes. Differential gene expression levels were confirmed by RT-PCR using cDNA from primary and secondary tumor derived TSC. FIG. 6A shows the change for Gadd45g and FIG. 6B shows the fold change for Frat1. 10 out of 10 genes tested so far have been confirmed in this assay. Samples were normalized to 18S and GUS (data not shown). Fold change compared to p53−/− NSC.

FIGS. 7A-7B show the results from the microarray gene expression comparison of SP cells. FIG. 7A shows a schema of SP gene expression comparison shown in FIG. 4A was applied. Biological triplicates of NSC (two p53−/− and one verb;p53−/−) and two independent CSC (CSC1=3447 and CSC2=4346) were analyzed. First, CSC1 vs. NSC and CSC2 vs. NSC were analyzed, then, genes that were common between the two lists were identified as “cancer SP” genes (538 genes when q≦0.05 and log2>1.5). FIG. 7B shows unsupervised clustering of the 538 cancer gene list clearly sorted NSC from two independent CSCs. There appear to be 4 groups of genes that show differential expression patterns.

FIGS. 8A-8C show identification of a brain cancer stem cell gene signature. FIG. 8A shows a schema is shown for identifying the 45-gene cancer stem cell gene signature. Cancer SP vs. non-SP cells were compared to identify genes that are differentially expressed in stem vs. non-stem cells (244 genes). These were then compared to the 538 cancer-SP gene list. 45 common genes on both lists are designated as a brain cancer stem cell gene signature. Unsupervised clustering of the 45 gene list clearly sorted NSC from two CSCs. FIG. 8B shows microarray data from an Affymetrix Genechip expression analysis. FIG. 8C shows a venn-diagram of the distribution of the differentially regulated genes into three categories; SP genes, cancer genes and non-SP genes.

FIGS. 9A-9B show the validation of brain cancer stem cell gene signature. Differential gene expression levels were confirmed by real-time PCR using cDNA from 3 independent primary tumorspheres. FIG. 9A shows RT-PCR results from S100α4 and FIG. 9B shows RT-PCR results for Col6a1. Samples were normalized to internal 18S levels. Relative fold changes compared to p53−/− NSC.

FIGS. 10A-10B shows Id4−/− neurosphere self-renewal is reduced to compared to control. FIG. 10A shows the number of neurospheres in Id4−/− mice is reduced as compared to wild type (B6) mice. FIG. 10B shows that Id4 is expressed higher in brain cancer stem cells (SP=stem) than non-stem cancer cells (G0=non-stem) from the same tissue sample.

FIG. 11A-11G show mammary glands of mice heterozygous for (Id 4+/−) versus mice lacking the Id4 gene (Id 4−/−). FIGS. 11A and 11C show mice heterozygous for (Id 4+/−) and FIGS. 11B and 11D show mice lacking the Id4 gene (Id 4−/−) which were isolated and stained with carmin alum. FIG. 11E shows morphometric measurements of ductal length, and FIG. 11F shows diameter, and FIG. 11G shows the number of branches per gland (n=3).

FIGS. 12A-12B show tumor onset in MMTV-PyMT and MMTV-neu transgenic mice (primary) and in transplanted animals (secondary). FIG. 12A shows primary and secondary tumor onset for MMTV-PyMT mice, where the median onset occurs about 90 days and 30 days respectively for primary and secondary tumors. FIG. 12B shows primary and secondary tumor onset for MMTV-neu mice, where the median onset occurs about 200 days and 75 days respectively for primary and secondary tumors.

FIGS. 13A-13B show Id2 and I4 expression in metastatic mammary tumorspheres. FIG. 13A shows relative Id2 levels, and FIG. 13B shows relative Id4 levels in tumorspheres isolated from Met-MMTV-neu (left bar, non-metastatic) and Met+ MMTV-PyMT (right bar; metastatic) mammary tumors.

FIGS. 14A-14B shows FACS analysis of mammary tumorspheres with CD24 and CD49f. FIGS. 14A and 14B are sister cultures derived from the same tumor, split into two different culture conditions 2 days before analysis. FIG. 14A shows cells in do not form tumors while FIG. 14B shows cells (CD24+CD49f+) to develop into tumors showing CD24+ population containing CSCs (arrow).

FIGS. 15A-15B shows the expression analysis in Mammary and Lung tumors. FIG. 15A shows the relative expression levels of Col6a1 in MMTV-neu (no metastasis) and MMTV-PyMT (lung metastasis) mammary tumorspheres (Mam) and lung metastasis tumorsphere (Lung). FIG. 15B shows the relative expression levels of CSCF1 (=A930001N09Rik) in MMTV-neu (no metastasis) and MMTV-PyMT (lung metastasis) mammary tumorspheres (Mam) and lung metastasis tumorsphere (Lung).

FIGS. 16A-16F show S100A4 and S100A6 expression in human gliomas of different grade. Tissue arrays containing 63 unique samples of human brain gliomas and normal cerebrum were stained with S100A4 antibody. FIG. 16A show s a summary chart showing percentages of S100A4+ cells in gliomas between grade I an IV. FIG. 16B shows a representative image of normal cerebrum, FIG. 16C shows a representative image of well differentiated glioma tissue, FIG. 16D shows a representative image of poorly differentiated glioma tissue, and FIG. 16E shows a representative image of undifferentiated glioma tissue. S100A4 is in red, DAPI in blue. Scale bar=20 μm. FIG. 16F shows that the percentage of S100A6+ cells us under 10% for gliomas of grade I to III, but significantly over 10% for gliomas of grade IV.

FIG. 17 shows results from S100A6 protein detection by ELISA showing that glioma stem cells secrete S100A6 into media. FIG. 17A shows a table of the detected S100A6 protein secreted by glioma CSCs in culture. Non-cancerous neuronal stem cells show no detectable S100A6 protein.

### DETAILED DESCRIPTION

The present invention relates to methods and compositions for the identification of cancers stem cells in a population of cells. The present invention further provides methods to diagnose and prognose cancer in a subject by identifying the presence of cancer stem cells in a population of cells obtained from the subject.

The inventors have discovered a group of genes, herein referred to as “cancer stem cell biomarkers” or “CSCB” which are set forth in Table 5 that can be used in subsets for the identification of cancer stem cells in a population of cells using gene expression analysis. The inventors provide guidance on the increase and/or decrease of expression of those genes for the identification of cancer stem cells. Accordingly, the present invention provides gene groups of the expression pattern or profile of which permit the identification of cancer stem cells (CSC) in a population of cancer cells.

Other aspects of the present invention are use of the cancer stem cell biomarkers as disclosed herein as prognostic and diagnostic markers to identify a subject with an cancer which comprises cancer stem cells, and often for prognosis or identifying a subject with a recurrent form cancer. For example, if a subject is identified as having a cancer which comprises at least one cancer stem cell, the subject is likely to have recurrent cancer. In some embodiments, if the subject who has undergone cancer therapy and has eliminated the tumor and/or reduced the tumor size is categorized is being in remission, if the subject is identified as having a cancer stem cell, the subject is likely to have a recurrence of the cancer. The cancer stem cell biomarkers as disclosed herein are also useful for developing anti-cancer therapies which specifically target and reduce the viability of cancer stem cells. In some embodiments, the cancer stem cell biomarkers as disclosed herein are also useful for monitoring the progression of cancer in a subject and also for assessing the efficacy of treatment of the subject with an anti-cancer therapy. In a similar manner, the cancer stem cell biomarkers as disclosed herein are also useful for monitoring and assessing anti-cancer therapies in clinical or other trials, to identify the efficacy of the agent to reduce the cancer stem cell population by a particular therapy or therapeutic regimen.

In some embodiments, subsets of the 46 genes listed as cancer stem cell biomarkers can be used to identify a cancer stem cell in a population of cells, for example, subsets of at least 6 genes, or at least 10, or at least 20, or at least 30, or at least 40 or more, selected from the group of cancer stem cell biomarkers set forth in Table 5 can be used. In some embodiments, any combination of 6 or more of cancer stem cell biomarkers listed in Table 5 can used in any combination to identify a cancer stem cell in a population of cells.

In some embodiments, the cancer stem cell biomarkers as disclosed herein can be used with other genes to identify a cancer stem cell in a population of cells.

In some embodiments, the present invention provides methods for identifying a subject at risk of having or developing cancer, the method comprising measuring the level of protein expression or gene transcript expression level of at least 6 of the cancer stem cell markers as set forth in Table 5 in a biological sample from a subject, and if the level of protein expression or gene transcript expression level of each is altered in comparison to a reference level, the subject is identified as having increased risk of having or developing cancer. In some embodiments, such a method can be used to identify subjects with cancers comprising cancer stem cells, and thus, are useful in the prognosis and diagnosis of cancer.

Accordingly, in some embodiments the inventors have discovered a group of cancer stem cell biomarkers, or subgroups thereof, for the diagnosis and/or prognosis of cancer in a subject. In some embodiments, the CSC biomarkers are detected using gene expression analysis, and in alternative embodiments, the CSC biomarkers are detected by protein expression analysis. In some embodiments, the group of CSC biomarkers or subgroups thereof, can be detected at the level of gene expression, for example gene transcript level such as mRNA expression. In alternative embodiments, a group of CSC biomarkers or subgroups thereof can be detected at the level of protein expression.

In one aspect of the present invention, the group of CSC useful in the methods and compositions as disclosed herein are set forth in Table 5. For example, the group of CSC biomarkers useful in the methods and compositions as disclosed herein comprise at least 6 genes selected from any of the following: 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik or homologues or variants thereof.

In another aspect, the group of CSC biomarkers useful in the methods and compositions as disclosed herein is set forth in Table 5. The CSC biomarkers were identified using differential gene expression analysis, by comparing expressed genes between normal and cancer SP cells, CSC1 cancer (e.g. 3447; see table 1) SP cell vs. normal SP cell and CSC2 cancer (e.g. 4346; see table 1) SP cell and normal SP cell. P-values were derived by 1000 permutation and the false discovery rate (q-value) was calculated to correct for the multiple hypothesis testing problem. Differentially expressed genes between cancer cells and cancer stem cells (i.e. cancer stem cells with normal SP cells) were selected by two criteria; genes having less than 0.05 q-value and more than 2.6 (1.5 log2) fold change in both comparisons (CSC1 vs. Normal and CSC2 vs. Normal).

In some embodiments, the cancer stem cell biomarkers are a group of genes comprising between 6-46 genes, and all other combinations in between, for example, 7,8, 9, 10, 11, 12, 13, 14, 15, 16, 17, 18, 19, 20, 21, 22, 23, 24 and so forth selected from the group of genes listed in Table 5, and identified by the following GenBank Sequence Identification Numbers (the identification numbers for each gene are separated by a “;” while alternative GenBank Sequence Identification numbers are separated by a “///.”):2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); 5033414K04Rik (SEQ ID NO:45) /// U16153 (SEQ ID NO:46), the expression of which can be used to identify the presence of cancer stem cells in a population of cells, for example in a population of non-stem cancer cells.

TABLE 5

| | Approved | | | | Sequence | |
|---|---|---|---|---|---|---|
| SEQ | Gene | | | Sequence | Accession | |
| ID | Symbol | Approved Gene Name | Location | Accession No ID | No ID | Aliases |
| 1 | 2310046A06Rik | RIKEN cDNA | | 2310046A06Rik | | |
| | | 2310046A06 gene | | | | |
| 2 | 3110035E14Rik | RIKEN cDNA | | 3110035E14Rik | | |
| | | 3110035E14 gene | | | | |
| 3 | A930001N09Rik | RIKEN cDNA | | A930001N09Rik | | |
| | | A930001N09 gene | | | | |
| 4 | AI593442 | expressed sequence | | AI593442 | | |
| | | AI593442 | | | | |
| 5 | AI851790 | expressed sequence | | AI851790 | | |
| | | AI851790 | | | | |
| 6 | AOX1 | aldehyde oxidase 1 | 2q33 | AF017060 | NM_001159 | AO, |
| | | | | | | AOH1 |
| 7 | ARHGAP29 | Rho GTPase activating | 1p22.1 | NM_004815 | | PARG1 |
| | | protein 29 | | | | |
| 8 | ARHGAP6 | Rho GTPase activating | Xp22.3 | AF012272 | NM_013427 | rhoGAPX-1 |
| | | protein 6 | | | | |
| 9 | BFSP2 | beaded filament | 3q21-25 | U48224 | NM_003571 | CP47, |
| | | structural protein 2, | | | | CP49, |
| | | phakinin | | | | LIFL-L, |
| | | | | | | phakinin |
| 10 | BGN | biglycan | Xq28 | AK092954 | NM_001711 | DSPG1, |
| | | | | | | SLRR1A |
| 11 | CAPG | capping protein (actin | 2 | M94345 | NM_001747 | MCP, |
| | | filament), gelsolin-like | | | | AFCP |
| 12 | CASP4 | caspase 4, apoptosis- | 11q22.2-q22.3 | U25804 | NM_001225 | ICE(rel)II, |
| | | related cysteine | | | | ICH-2, |
| | | peptidase | | | | TX |
| 13 | CAV1 | caveolin 1, caveolae | 7q31 | AF125348 | NM_001753 | CAV |
| | | protein, 22 kDa | | | | |
| 14 | COL6A1 | collagen, type VI, alpha 1 | 21q22.3 | M20776 | NM_001848 | |
| 15 | COL6A2 | collagen, type VI, alpha 2 | 21q22.3 | M20777 | NM_058175 | |
| 16 | CYTL1 | cytokine-like 1 | 4p16-p15 | AF193766 | NM_018659 | C17, |
| | | | | | | C4orf4 |
| 17 | D3Bwg0562e | DNA segment, Chr 3, | | D3Bwg0562e | | |
| | | Brigham &Women's | | | | |
| | | Genetics 0562 | | | | |
| | | expressed | | | | |
| 18 | D930020E02Rik | RIKEN cDNA | | D930020E02Rik | | |
| | | D930020E02 gene | | | | |
| 19 | DDC | dopa decarboxylase | 7p11 | NM_000790 | | AADC |
| | | (aromatic L-amino acid | | | | |
| | | decarboxylase) | | | | |
| 20 | DHRS3 | dehydrogenase/reductase | 1p36.1 | AF061741 | NM_004753 | retSDR1, |
| | | (SDR family) member 3 | | | | Rsdr1, |
| | | | | | | SDR1, |
| | | | | | | RDH17 |
| 21 | E030011K20Rik | RIKEN cDNA | | E030011K20Rik | | |
| | | E030011K20 gene | | | | |
| 22 | ENPP6 | ectonucleotide | 4q35.1 | AK057370 | NM_153343 | MGC33971 |
| | | pyrophosphatase/phosphodiesterase 6 | | | | |
| 23 | FOXA3 | forkhead box A3 | 19q13.2-q13.4 | L12141 | NM_004497 | HNF3G |
| 24 | FOXC2 | forkhead box C2 (MFH- | 16q22-16q24 | Y08223 | NM_005251 | MFH-1, |
| | | 1, mesenchyme | | | | FKHL14 |
| | | forkhead 1) | | | | |
| 25 | GJA1 | gap junction protein, | 6q22-q23 | BC026329 | NM_000165 | CX43, |
| | | alpha 1, 43 kDa | | | | ODD, |
| | | | | | | ODOD, |
| | | | | | | SDTY3, |
| | | | | | | ODDD, |
| | | | | | | GJAL |
| 26 | gpr17 | G-protein coupled | 2q21 | NM_005291 | | |
| | | receptor 17 | | | | |
| 27 | KAZALD1 | Kazal-type serine | 10q24.32 | AF333487 | NM_030929 | FKSG40, |
| | | peptidase inhibitor | | | | FKSG28 |
| | | domain 1 | | | | |
| 28 | KCNA4 | potassium voltage-gated | 11p14 | M55514 | NM_002233 | Kv1.4, |
| | | channel, shaker-related | | | | HK1, |
| | | subfamily, member 4 | | | | HPCN2, |
| | | | | | | KCNA4L |
| 29 | LARP6 | La ribonucleoprotein | 15q23 | BC009446 | NM_018357 | acheron, |
| | | domain family, member 6 | | | | FLJ11196 |
| 30 | LGALS3 | lectin, galactoside- | 14q22.3 | M64303 | NM_002306 | MAC-2, |
| | | binding, soluble, 3 | | | | GALIG, |
| | | | | | | LGALS2 |
| 31 | MGP | matrix Gla protein | 12p12.3 | M58549 | NM_000900 | |
| 32 | MIA | melanoma inhibitory | 19q13.32-q13.33 | X75450 | NM_006533 | MIA1 |
| | | activity | | | | |
| 33 | NINJ2 | ninjurin 2 | 12p13 | AF205633 | NM_016533 | |
| 34 | OPCML | opioid binding | 11q25 | BX537377 | NM_001012393 | OPCM, |
| | | protein/cell adhesion | | | | OBCAM |
| | | molecule-like | | | | |
| 35 | PAPSS2 | 3′-phosphoadenosine 5′- | 10q24 | AF091242 | NM_004670 | ATPSK2 |
| | | phosphosulfate synthase 2 | | | | |
| 36 | S100A4 | S100 calcium binding | 1q12-q22 | BC016300 | NM_002961 | P9KA, |
| | | protein A4 | | | | 18A2, |
| | | | | | | PEL98, |
| | | | | | | 42A, |
| | | | | | | FSP1, |
| | | | | | | MTS1, |
| | | | | | | CAPL |
| 37 | S100A6 | S100 calcium binding | 1q21 | BC001431 | NM_014624 | 2A9, |
| | | protein A6 | | | | PRA, |
| | | | | | | CABP, |
| | | | | | | CACY |
| 38 | SCG3 | secretogranin III | 15 | AF078851 | NM_013243 | SGIII |
| 39 | SCG5 | secretogranin V (7B2 | 15q13-q14 | Y00757 | NM_003020 | 7B2, |
| | | protein) | | | | SgV, |
| | | | | | | SGNE1 |
| 40 | SRPX2 | sushi-repeat-containing | Xq21.33-q23 | AF393649 | NM_014467 | SRPUL |
| | | protein, X-linked 2 | | | | |
| 41 | TEAD1 | TEA domain family | 11p15.4 | X84839 | NM_021961 | TEF-1, |
| | | member 1 (SV40 | | | | TCF13, |
| | | transcriptional enhancer | | | | AA |
| | | factor) | | | | |
| 42 | TMEM46 | transmembrane protein | 13q12.13 | NM_001007538 | | bA398O19.2, |
| | | 46 | | | | PRO28631, |
| | | | | | | WGAR9166, |
| | | | | | | C13orf13 |
| 43 | VWC2 | von Willebrand factor C | 7p12.3-p12.2 | AY358393 | NM_198570 | PSST739, |
| | | domain containing 2 | | | | UNQ739 |
| 44 | WNT5A | wingless-type MMTV | 3p21-p14 | L20861 | NM_003392 | |
| | | integration site family, | | | | |
| | | member 5A | | | | |
| 45 | 5033414K04Rik | RIKEN cDNA | | 5033414K04Rik | | |
| | | 5033414K04 gene | | | | |
| | | inhibitor of DNA | | | | |
| 46 | ID4 | binding 4, dominant | 6p22-p21 | U16153 | U28368 | |
| | | negative helix-loop- | | | Y07958 | |
| | | helix protein | | | | |

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The terms “patient”, “subject” and “individual” are used interchangeably herein, and refer to an animal, particularly a human, from whom the biological sample is obtained, and/or a treatment including prophylaxic treatment is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dogs, rodents (e.g. mouse or rat), guinea pigs, goats, pigs, cats, rabbits, cows, and non-mammals such as chickens, amphibians, reptiles, etc. In one embodiment, the subject is human. In another embodiment, the subject is an experimental animal or animal substitute as a disease model.

The term “mammal” is intended to encompass a singular “mammal” and plural “mammals,” and includes, but is not limited to: humans, primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears. Preferably, the mammal is a human subject. As used herein, a “subject” refers to a mammal, preferably a human.

The term “gene” used herein refers to a nucleic acid sequence encoding an amino acid sequence or a functional RNA, such as mRNA, tRNA, rRNA, catalytic RNA, siRNA, miRNA and antisense RNA. A gene can also be an mRNA or cDNA corresponding to the coding regions (e.g. exons and miRNA). A gene can also be an amplified nucleic acid molecule produced in vitro comprising all or a part of the coding region.

The term “gene product” as used herein refers to both an RNA transcript of a gene and a translated polypeptide encoded by that transcript.

The term “expression” as used herein refers to transcription of a nucleic acid sequence, as well as to the production, by translation, of a polypeptide product from a transcribed nucleic acid sequence.

The term “nucleic acid” or “oligonucleotide” or “polynucleotide” used herein can mean at least two nucleotides covalently linked together. As will be appreciated by those skilled in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O— and N-alkylated nucleotides, e.g. N6-methyl adenosine are suitable. The 2′ OH— group can be replaced by a group selected from H. OR, R. halo, SH, SR, NH₂, NHR, NR₂ or CN, wherein R is C—C6 alkyl, alkenyl or alkynyl and halo is F. Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

An “array” broadly refers to an arrangement of agents (e.g., proteins, antibodies, replicable genetic packages) in positionally distinct locations on a substrate. In some instances the agents on the array are spatially encoded such that the identity of an agent can be determined from its location on the array. A “microarray” generally refers to an array in which detection requires the use of microscopic detection to detect complexes formed with agents on the substrate. A “location” on an array refers to a localized area on the array surface that includes agents, each defined so that it can be distinguished from adjacent locations (e.g., being positioned on the overall array, or having some detectable characteristic, that allows the location to be distinguished from other locations). Typically, each location includes a single type of agent but this is not required. The location can have any convenient shape (e.g., circular, rectangular, elliptical or wedge-shaped). The size or area of a location can vary significantly. In some instances, the area of a location is greater than 1 cm², such as 2 cm², including any area within this range. More typically, the area of the location is less than 1 cm2, in other instances less than 1 mm², in still other instances less than 0.5 mm², in yet still other instances less than 10,000 μm², or less than 100 μm².

As used herein, the term “treating” includes reducing or alleviating at least one adverse effect or symptom of a condition, disease or disorder associated with cancer. As used herein, the term treating is used to refer to the reduction of a symptom and/or a biochemical marker of cancer by at least 10%. As a non-limiting example, a treatment can be measured by a change in a cancer stem cell biomarker as disclosed herein, for example a change in the expression level of a cancer stem cell biomarker by at least 10% in the direction closer to the reference expression level for that cancer stem cell biomarker. By way of an example only, if a downregulated cancer stem cell biomarker in a biological sample from the subject is about 30% of the level of the reference level, an increase in the same cancer stem cell biomarker to about 40% of the reference level would be considered a reduction in a biological marker of the cancer by at least 10% and would be considered an effective treatment.

The term “effective amount” as used herein refers to the amount of therapeutic agent or pharmaceutical composition to reduce or stop at least one symptom or marker of the disease or disorder, for example a symptom or marker of cancer. For example, an effective amount using the methods as disclosed herein would be considered as the amount sufficient to reduce a symptom or marker of the disease or disorder or cancer by at least 10%. An effective amount as used herein would also include an amount sufficient to prevent or delay the development of a symptom of the disease, alter the course of a symptom disease (for example but not limited to, slowing the progression of a symptom of the disease), or reverse a symptom of the disease.

As used herein, the terms “administering,” and “introducing” are used interchangeably and refer to the placement of the agents as disclosed herein into a subject by a method or route which results in at least partial localization of the agents at a desired site. Compounds can be administered by any appropriate route which results in an effective treatment in the subject.

The term “therapeutically effective amount” refers to an amount that is sufficient to effect a therapeutically or prophylactically significant reduction in a symptom associated with the cancer. A therapeutically or prophylatically significant reduction in a symptom is, e.g. at least about 10%, about 20%, about 30%, about 40%, about 50%, about 60%, about 70%, about 80%, about 90%, about 100%, about 125%, about 150% or more as compared to a control, the subject prior to treatment, or a non-treated subject. In some embodiments where the condition is cancer, the term “therapeutically effective amount” refers to the amount that is safe and sufficient to prevent or delay the development and further spread of metastases in cancer patients. The amount can also cure or cause the cancer to go into remission, slow the course of cancer progression, slow or inhibit tumor growth, slow or inhibit tumor metastasis, slow or inhibit the establishment of secondary tumors at metastatic sites, or inhibit the formation of new tumor metastasis.

The terms “treat” and “treatment” refer to both therapeutic treatment and prophylactic or preventative measures, wherein the object is to prevent or slow down the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total). “Treatment” can also mean prolonging survival as compared to expected survival if not receiving treatment. Those in need of treatment include those already diagnosed with cancer as well as those likely to develop secondary tumors due to metastasis.

As used herein, the term “biological sample” refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the subject, but the term can also refer to non-cellular biological material, such as non-cellular fractions of blood, saliva, or urine, that can be used to measure gene expression levels. Biological samples include, but are not limited to, tissue biopsies, needle biopsies, scrapes (e.g. buccal scrapes), whole blood, plasma, serum, lymph, bone marrow, urine, saliva, sputum, cell culture, pleural fluid, pericardial fluid, ascitic fluid or cerebrospinal fluid. Biological samples also include tissue biopsies and cell cultures. A biological sample or tissue sample can refer to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples can be used. Samples may be paraffin-embedded or frozen tissue. The sample can be obtained by removing a sample of cells from a subject, but can also be accomplished by using previously isolated cells (e.g. isolated by another person), or by performing the methods of the invention in vivo.

The term “vectors” is used interchangeably with “plasmid” to refer to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but not limited to, plasmids, episomes, bacteriophages or viral vectors, and such vectors can integrate into the host's genome or replicate autonomously in the particular cell. Other forms of expression vectors known by those skilled in the art which serve the equivalent functions can also be used. Expression vectors comprise expression vectors for stable or transient expression of encoded sequences.

The terms “polypeptide” and “protein” are used interchangeably to refer to a polymer of amino acid residues, and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, for purposes of the present invention, a “polypeptide” refers to a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, has a well-defined conformation. Proteins, as opposed to peptides, generally consist of chains of 50 or more amino acids. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 50 amino acids in length.

The terms “homology”, “identity” and “similarity” refer to the degree of sequence similarity between two peptides or between two optimally aligned nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with the sequences as disclosed herein.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or I) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85% sequence identity, preferably at least 90% to 95% sequence identity, more usually at least 99% sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which can include deletions or additions which total 20 percent or less of the reference sequence over the comparison window. The reference sequence can be a subset of a larger sequence. The term “similarity”, when used to describe a polypeptide, is determined by comparing the amino acid sequence and the conserved amino acid substitutes of one polypeptide to the sequence of a second polypeptide.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan.

The term “substantially homologous” refers to sequences that are at least 90%, at least 95% identical, at least 96%, identical at least 97% identical, at least 98% identical or at least 99% identical. Homologous sequences can be the same functional gene in different species. Determination of homologs of the genes or peptides of the present invention can be easily ascertained by the skilled artisan.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

By “specifically binds” or “specific binding” is meant a compound or antibody that recognizes and binds a desired polypeptide but that does not substantially recognize and bind other molecules in a sample, for example, a biological sample, which naturally includes a polypeptide of the invention.

By “substantially pure” or is meant a cell, nucleic acid, polypeptide, or other molecule that has been separated from the components that naturally accompany it. Typically, a cell population is substantially pure when it is at least about 60%, or at least about 70%, at least about 80%, at least about 90%, at least about 95%, or even at least about 99%, by weight, free from the other cells with which it is naturally associated. For example, a substantially pure polypeptide may be obtained by extraction from a natural source, by expression of a recombinant nucleic acid in a cell that does not normally express that protein, or by chemical synthesis.

By a “decrease”, “reduction” or “inhibition” used in the context of the level of expression or activity of a gene refers to a reduction in protein or nucleic acid level. For example, such a decrease may be due to reduced RNA stability, transcription, or translation, increased protein degradation, or RNA interference. Preferably, this decrease is at least about 5%, at least about 10%, at least about 25%, or when “decrease” is used in the context of a decrease the expression of a cancer stem cell biomarker as compared to a reference expression level, a decrease is preferably at least about 50% (i.e. 0.5 fold of the reference level), at least about 60% (i.e. 0.4 fold of the reference level), at least about 70% (i.e. 0.3 fold of the reference level), at least about 80% (i.e. 0.2 fold of the reference level), at least about 90% (i.e. 0.1 fold of the reference level) or at least 100% (i.e. complete inhibition), or any integer in between of the level of expression or activity under control conditions (i.e. normal expression levels).

By an “increase” in the expression or activity of a gene or protein is meant a positive change in protein or nucleic acid level. For example, such an increase may be due to increased RNA stability, transcription, or translation, or decreased protein degradation. Preferably, this increase is at least 5%, at least about 10%, at least about 25%, at least about 50%, at least about 75%, at least about 80%, at least about 100%, or when “increase” is used in the context of an increase in the expression of a cancer stem cell biomarker as compared to a reference expression level, an increase is preferably at least about 150% (i.e. 1.5-fold), at least about 200% (i.e. 2-fold), or at least about 300% (i.e. 3-fold) or at least about 500% (i.e. 5-fold), or at least about 10,000% (i.e. 10-fold) or more over the level of expression or activity under control conditions.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean ±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Other features and advantages of the invention will be apparent from the following Detailed Description, the drawings, and the claims.

### General: Cancer Stem Cell Biomarkers.

Accordingly, the methods and compositions as disclosed herein provide gene groups that can be used to identify a cancer stem cell in a population of cells, for example from a population of non-stem cell cancer cells.

In some embodiments the present invention provides groups of genes, the expression profile of which provides a diagnostic and/or prognostic test to determine if a subject has a cancer that comprises cancer stem cells. For example, in one embodiment, the present invention provides groups of genes, the expression profiles of which can distinguish a subject with a cancer comprising cancer stem cells from a subject with cancer not comprising cancer stem cells.

In one embodiment, the present invention provides an early asymptomatic screening system for cancer stem cells in a subject by analysis of at least 6 of the gene expression profiles as disclosed in Table 5 herein. Such screening can be performed, for example in subjects suspected to have, or that have been diagnosed with cancer. In some embodiments, the subjects have had treatment for cancer, and the methods and compositions as disclosed herein are useful to monitor a cancer in a subject that is in remission, and/or identify if a subject is likely to a have reoccurrence of a cancer.

As early detection of cancer and early treatment increases the chance that the treatment is successful, the gene and protein expression analysis system of the present invention provides vastly improved methods to detect cancers comprising cancer stem cells, and in particular cancers comprising cancer stem cells which may be refractory or non-responsive to some cancer therapies. Detection of cancers comprising cancer stem cells cannot yet be discovered by any other means currently available.

In some embodiments, the levels of gene transcript or protein expression of at least 6 cancer stem cell biomarkers as disclosed herein are measured in a biological sample, for example a biological sample from a subject, and the expression of the group and/or a subgroup of CSC biomarkers in a biological sample from the subject is compared to a reference level of the expression of the group and/or subgroup of CSC biomarkers, for example, expressed in a reference biological sample. In some embodiments, the reference expression level can be from a reference biological sample or a group of reference samples, for example a biological sample comprising non-cancer cells or non-stem cell cancer cells, such as normal tissue from the subject, or a biological sample from a subject that does not have cancer, for example not comprising cancer stem cells.

As used herein the term “reference level” refers to the level of a CSC biomarker in at least one reference biological sample, or a group of reference biological samples from at least one normal subject or a group of normal subjects or subjects not with cancer, or from biological samples not comprising non-stem cancer cells. A reference expression level can be normalized to 100%. When the reference expression level is normalized to 100%, a 2-fold difference refers to 200% expression level, and a 3-fold difference refers to a 300% expression level etc. Similarly, when a reference expression is normalized to 100%, a 0.3-fold difference refers to a 30% expression level of the reference expression level (i.e. a 70% decrease), or a 0.1-fold difference refers to a 10% expression level of the reference expression level (i.e. a 90% decrease), etc. A difference in the level of expression a CSC biomarker, (such as an increase or decrease in the level of expression of a CSC biomarker) in the biological sample as compared with a reference expression level of the same CSC biomarker indicates a positive CSC biomarker signal in the biological sample.

In some embodiments, an increase in the level of expression of a CSC biomarker which is upregulated in the biological sample and the reference expression level can be at least about a 1.5 fold difference, at least a 2.0 fold difference, at least about 2.5 fold difference, at least about 3 fold difference, at least about 5 fold difference, or between 5-10 fold different, or 10-20 fold or greater than 20 fold, or any integer in between. Such upregulated genes include, for example, 2310046A06Rik; 3110035E14Rik; A930001N09Rik; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GPR17; ID4; KAZALD1; KCNA4; LARP6; LGALS3; MGP; MIA; NINJ2; OPCML; PAPSS2; S100A4; S100A6; SCG5; SRPX2; TMEM46 and VWC2.

In some embodiments, an decrease in the level of expression of a CSC biomarker which is downregulated in the biological sample and the reference expression level can be at least about a 0.5 fold of the reference expression level (i.e. at least a 50% decrease), or at least about a 0.4 fold of the reference expression level (i.e. at least a 60% decrease), or at least about 0.3-fold of the reference expression level (i.e. at least a 70% decrease), or at least about 0.2 fold of the reference expression level (i.e. at least a 80% decrease), at least about 0.1 fold of the reference expression level (i.e. at least a 90% decrease), or between 0.5-0.1 fold different (i.e. at least a 50% to 90% decrease), or 0 fold of the reference expression level (i.e. 100% decrease). Such downregulated genes include, for example; AI593442; AI851790; AOX1; ARHGAP29; GJA1; SCG3; TEAD1; WNT5A; and 5033414K04Rik.

Stated another way, a decrease in the level of expression of a CSC biomarker which is downregulated in the biological sample as compared to the reference expression level, which is normalized to 100% for the purposes of this example, is a decrease in the expression of a CSC biomarker (such as AI593442; AI851790; AOX1; ARHGAP29; GJA1; SCG3; TEAD1; WNT5A; and 5033414K04Rik) of at least about 50% decrease in expression, at least about 60% decrease in expression, at least about 70% decrease in expression, at least about 80% decrease in expression, at least about 90% decrease in expression as compared to level of the reference expression.

Stated a further way, a decrease in the level of expression of a CSC biomarker which is downregulated in the biological sample as compared to the reference expression level, relates to the level of expression of a CSC biomarker, such as AI593442; AI851790; AOX1; ARHGAP29; GJA1; SCG3; TEAD1; WNT5A; and 5033414K04Rik of at least about 0.5-fold (i.e. 50%) of the reference level expression, at least about 0.4-fold (i.e. 40%) of the reference level expression, at least about 0.3-fold (i.e. 30%) of the reference level expression, at least about 0.2-fold (i.e. 20%) of the reference level expression, at least about 0.1-fold (i.e. 10%) of the reference level expression, when the reference level expression is normalized to 100%.

For example, a reference expression level for a CSC biomarker such as 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; or 5033414K04Rik can be normalized to 100%.

In some embodiments, a different level of expression of at least 6 CSC biomarkers selected from a group that have increased expression, the group consisting of 2310046A06Rik; 3110035E14Rik; A930001N09Rik; BFSP2; BGN; CAPG; CASP4; CAV1; COL6A1; COL6A2; CYTL1; D3Bwg0562e; DDC; DHRS3; E030011K20Rik; ENPP6; FOXA3; FOXC2; GPR17; ID4, KAZALD1; KCNA4; LARP6; LGALS3; MGP; MIA; NINJ2; OPCML; PAPSS2; S100A4; S100A6; SCG5; SRPX2; TMEM46; VWC2. In some embodiments, a different level of expression of at least 6 CSC biomarkers selected from a group that have decreased expression, the group consisting of; AI593442; AI851790; AOX1; ARHGAP29; ARHGAP6 D930020E02Rik; GJA1; SCG3; TEAD1; WNT5A; and 5033414K04Rik.

In some embodiments, a different level of expression of at least 6 CSC biomarkers selected from the group of: 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; 5033414K04Rik, where there is at least a 1.5 fold difference, or at least 2.0 fold or at least 3.0 fold, or at least 5.0 fold, or between 5-10 fold different, or 10-20 fold or greater than 20 fold difference in the level expression of upregulated genes in the biological sample, or at least 0.5 fold (i.e. at least a 50% decrease), or at least about a 0.4 fold (i.e. at least a 60% decrease), or at least about 0.3-fold (i.e. at least a 70% decrease), or at least about 0.2 fold (i.e. at least a 80% decrease), at least about 0.1 fold (i.e. at least a 90% decrease) the expression of the reference expression level, or between 0.5-0.1 fold (i.e. at least a 50% to 90% decrease) the expression of the reference expression level, of the downregulated genes; 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; 5033414K04Rik identifies the presence of a cancer stem cell in a population of cells.

It should be noted, that the fold change of expression level of one CSC biomarker compared to its corresponding reference expression level, and the fold change of a different CSC biomarker compared to its corresponding reference expression level can be different. For example, the present invention encompasses identification of a cancer stem cell in a population of cells if the level of each CSC biomarker tested in the biological sample is different by least 1.5-fold for upregulated genes, or at least 0.5-fold (i.e. a 50% decrease) for downregulated genes as compared to the reference expression level for the same CSC biomarker in a tissue of same origin.

As an example only, in assessing the expression level of 6 CSC biomarkers measured in a biological sample from a subject, the level of expression of one CSC biomarker can be increased by about 2.0 fold, a second CSC biomarker can be increased by about 14.0 fold and a third CSC biomarker can be increased by about 2.6 fold, a fourth CSC biomarker can be increased by about 4.2 fold, a fifth CSC biomarker can be increased by about 9.1 fold, a sixth CSC biomarker can be increased by about 2.1 fold as compared to their corresponding reference expression levels for each of the six CSC biomarker assessed.

Alternatively, and by way of example only, if one assessing the expression level of 6 CSC biomarkers in a biological sample from a subject where some of the CSC biomarkers measured are upregulated genes and some CSC biomarkers measured are downregulated genes, the level of expression of one CSC downregulated biomarker can be a decreased by at least about 0.5 fold (i.e. 50% decrease), a second CSC upregulated biomarker can be increased by about 14.0 fold and a third CSC downregulated biomarker can be decreased by about 0.5 fold, a fourth CSC downregulated biomarker can be decreased by about 0.2 fold, a fifth CSC upregulated biomarker can be increased by about 9.1 fold, a sixth CSC upregulated biomarker can be increased by about 2.1 fold as compared to their corresponding reference expression levels for each of the six CSC biomarker assessed. As discussed above and throughout the specification, such upregulated genes can be selected from the group of, for example, 2310046A06Rik; 3110035E14Rik; A930001N09Rik; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GPR17; ID4; KAZALD1; KCNA4; LARP6; LGALS3; MGP; MIA; NINJ2; OPCML; PAPSS2; S100A4; S100A6; SCG5; SRPX2; TMEM46 and VWC2, and downregulated genes can be selected from the group of, for example AI593442; AI851790; AOX1; ARHGAP29; GJA1; SCG3; TEAD1; WNT5A; and 5033414K04Rik.

In some embodiments, reference expression levels useful in the methods as disclosed herein can be biological samples obtained from a subject or a group of subjects who do not have cancer, in particular from a subject who does not have cancer comprising cancer stem cells. In some embodiments, the reference expression levels useful in the methods as disclosed herein are from the same tissue origin, but from a tissue without cancer and/or cancer stem cells.

In some embodiments, reference expression levels can be obtained from biological samples from the same subject, for example the reference expression level can be the expression level in a biological sample obtained from the subject at one time point, such as at an earlier time point (i.e. a first timepoint), which us useful as a reference expression level for comparison with a biological sample from the same subject obtained at a later (i.e. second) time point. Such embodiments are useful for prognosis, as well as monitoring the presence of CSC in a subject over a defined time period, for example from the time when the reference expression level (i.e. first biological sample) was obtained to the time when the second biological sample was obtained from the same subject. Such embodiments are useful to monitor disease progression of cancer in a subject, and in particular to assess a cancer treatment, such as a cancer treatment aimed or targeted to reduce cancer stem cells in a subject.

In some embodiments, reference expression levels useful in the methods as disclosed herein are obtained from a population group, which refers to a group of individuals or subjects sharing a common ethno-geographic origin. Reference expression levels can be reference expression levels from populations such as groups of subjects or individuals who are predicted to have representative levels of expression of the gene transcripts and/or proteins encoded by the CSC biomarkers listed in Table 5 found in the general population. Preferably, the reference expression level is from a population with representative levels of expression of the gene transcripts and/or proteins encoded by the CSC biomarkers listed in Table 5 in the population at a certainty level of at least 85%, preferably at least 90%, more preferably at least 95% and even more preferably at least 99%.

In another embodiment, the present invention provides a group of genes that can be used as predictors of the presence of CSC in a subject. A group of genes comprising between 6 and 46, and all combinations in between, for example 7, 8, 9, 10, 11, 12, 13, 14, 15, 16, 17, 18, 19 and 20 gene transcripts selected from the group consisting of genes selected from Table 5, and identified by the following GenBank Sequence Identification numbers (the identification numbers for each gene are separated by a “;” while alternative GenBank Sequence ID numbers are separated by “///”):2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); 5033414K04Rik (SEQ ID NO:45); U16153 (SEQ ID NO:46) the expression profile of which can be used to diagnose cancer comprising CSC in a biological sample from a subject, when the expression pattern is compared to the reference level or expression pattern of the same group of genes in a reference biological sample who does not have, or is not at risk of developing, cancer comprising cancer stem cells.

In another embodiment, the level of expression of a subgroup (subgroup) can be compared with the corresponding reference level. Subgroups of CSC biomarkers can be at least 6 up to any number of genes selected from the CSC biomarkers set forth in Table 5, of about 6 to 8, 6 to 15, 10 to 15 or 15 to 20, 21-30, 31-40 or any number of genes between 6 and 46.

The level of expression of groups of CSC biomarkers are compared with their corresponding reference levels. In some embodiments, the groups can be based on cellular localization or function of the gene. Examples of such categories are set forth in Table 3. In some embodiments, one such group of CSC biomarkers can comprise the genes MGP, BGN, KAZALD1, COL6A1, SCG5, COL6A2, VWC2, MIA, and SCG3. In another embodiment, a group of CSC can be selected from TMEM46, OPCML, NINJ2, ENPP6, CAV1, S100A6, S100A4, GPR17, ID4, D930020E02RIK, GJA1, 5033414K04RIK, and KCNA4. In another embodiment, a group of CSC can be selected from CYTL1, AI851790, WNT5A, PAPSS2, ARHGAP6, D3BWG0562E, and ARHGAP29. In another embodiment, a group of CSC can be selected from FOXC2, FOXA3, A930001N09RIK (4.5×), LARP6 (5.4×), TEAD1 (0.3×), and CASP4. In another embodiment, a group of CSC can be selected from DDC, LGALS2, CAPG, SRPX2, DHRS3, BFSP2, AOX1, 3110035E14RIK, 2310046A06RIK, E030011K20RIK, and AI593442.

In some embodiments, a subgroup of CSC biomarkers useful in the diagnostic and prognostic methods and compositions to identify CSC in a population of cells can be combined with other biomarker genes, for example but not limited to other biomarker genes for cancer. In some embodiments, the group of CSC biomarkers or subgroup thereof can be combined with any number of other genes, for example other biomarker genes such as cancer biomarkers comprising a group of about 1, about 5, about 1-5, about 5-10, about 10-15, about 15-20, about 20-25, about 25-30 about 35-40 about 40-45 about 45-50 can be used in combination with the CSC biomarkers as disclosed herein to increase accuracy of identification of a population of cells comprising cancer stem cells from a population of cells comprising non-stem cancer cells.

In one embodiment, the present invention provides a method to identify the presence of cancer stem cells in a subject by identifying a group of at least six CSC biomarkers which are expressed at a different level by least 1.5-fold for upregulated genes, or at least 0.5-fold (i.e. a 50% decrease) for downregulated genes as compared to a corresponding reference expression level. In one embodiment, the group consists of at least 6 or as many as 46 CSC biomarker genes selected from the group of nucleic acid sequences consisting of: 2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); 5033414K04Rik are (SEQ ID NO:45); U16153 (SEQ ID NO:46).

In another embodiment, the present invention provides a method for diagnosing whether a subject has a cancer comprising CSC or if a subject has increased likelihood of having a reoccurrence of cancer, the method comprising obtaining a biological sample from the subject and measuring expression of the gene transcript or the protein expression level of at least 6 CSC biomarkers selected from the group of CSC biomarkers listed in Table 5, and comparing the level of gene transcript or protein expression level of the same group of CSC biomarkers with reference expression levels for that group. A difference in level of expression in the group of CSC biomarkers analyzed is indicative of the subject having a different risk of having a cancer comprising cancer stem cells as compared to the subject from which the reference biological sample was obtained. More specifically, a different expression level of at least 1.5-fold for upregulated genes, or at least 0.5-fold (i.e. a 50% decrease) for downregulated genes of a group of at least 6 CSC biomarkers or more as listed in Table 5, in the biological sample from the subject as compared to the reference biological sample identifies the subject having the presence of cancer stem cells.

In some embodiments, when the subject is identified to be at risk of having cancer stem cells using the methods as disclosed herein, the subject can be selected for frequent follow up measurements of the levels of expression of least 6 CSC biomarkers as listed in Table 5 to allow early treatment of cancer and prevention of cancer reoccurrence.

Accordingly, in some embodiments, the present invention provides methods to identify subjects who are at a lesser risk of cancer reoccurrence, as by analyzing the expression levels of at least 6 CSC biomarkers according to the methods as disclosed herein, one can identify subjects not having cancer stem cells and thus less likely to have cancer reoccurrence. Such subjects can be selected to not undergo as frequent follow up measurements for levels of expression of the CSC biomarkers as compared to subjects identified to have cancer stem cells.

Determining Expression Level by Measuring mRNA

In one embodiment, the level of expression of CSC biomarker can be determined by measuring the gene transcript expression, such as level of mRNA of the CSC biomarkers as disclosed herein. In some embodiments, gene transcript expression can be measured by contacting a biological sample with nucleic acid agents, such as for example oligonucleotides, which hybridize under stringent conditions to the nucleic acids of SEQ ID NO:1 to SEQ ID NO:46, and quantifying the level of hybridization as a measure of the level of gene transcript expression. One can use any method to measure gene transcript expression available in the art. Some examples of such methods are briefly discussed herein

Real time PCR is an amplification technique that can be used to determine levels of mRNA expression. (See, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes can be obtained commercially from, for example, Perkin Elmer/Applied Biosystems (Foster City, Calif.). To quantitate the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

The TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, at world wide web 2 site: “perkin-elmer dot com”).

In another embodiment, real-time quantitative PCR can be performed using intercalating fluorescent dyes like SYBR Green I and measuring the signal intensity after amplification, which can be assayed for example in the LightCycler Real Time PCR System (Roche) or ABI 7900HT Fast Real Time PCR System (Applied Biosystems).

In another embodiment, detection of RNA transcripts can be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting enzyme mRNA transcripts is described in reference Pabic et. al. Hepatology, 37(5): 1056-1066, 2003, which is herein incorporated by reference in its entirety.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be counterstained with haematoxylin or Nuclear Fast Red to demonstrate the histological composition of the sample, and dark field imaging with a suitable light filter shows the developed emulsion. Non-radioactive labels such as digoxigenin, digoxin, biotin, rhodamine or fluorescein can also be used.

Alternatively, mRNA expression can be detected on a DNA array, chip, beads, microspheres or a microarray. Oligonucleotides corresponding to enzyme are immobilized on a chip which is then hybridized with labeled nucleic acids of a test sample obtained from a patient. Positive hybridization signal is obtained with the sample containing enzyme transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the tissue sample to be tested, reverse transcribed, and fluorescent-labeled cDNA probes are generated. The microarrays capable of hybridizing to enzyme cDNA are then probed with the labeled cDNA probes, the slides scanned and fluorescence intensity measured. This intensity correlates with the hybridization intensity and expression levels.

To monitor mRNA levels, for example, a cell lysate is applied to beads which capture the target RNAs by cooperative hybridization followed by signal amplification and detection.

Methods of “quantitative” amplification are well known to those of skill in the art. For example, quantitative PCR can involve simultaneously co-amplifying a known quantity of a control sequence using the same primers. This provides an internal standard that can be used to calibrate the PCR reaction. Detailed protocols for quantitative PCR are provided, for example, in Innis et al. (1990) PCR Protocols, A Guide to Methods and Applications, Academic Press, Inc. N.Y. One of ordinary skill in the art can design primers for use in quantitative RT-PCR which can be used to amplify a fragment of the nucleic acid of the CSC biomakers as disclosed herein. By way of an example only, appropriate primers to amplify CSC biomarker expression in a biological sample from mouse include, for example, primers of SEQ ID NOs: 47 to SEQ ID NO: 72 which are disclosed in the Examples. One of ordinary skill in the art can design primers to amplify a fragment of the nucleic acid of the CSC biomakers as disclosed herein from human samples, by using primers specific to the human nucleic acid sequence of the CSC biomarker at corresponding regions of the human gene to where the primers 47-72 hybridize to the mouse homologue of the CSC biomarker.

Alternatively, mRNA expression can be detected by high throughput sequencing methods (e.g. SOLiD RNA expression by NimbleGen).

### Determining Expression Level by Measuring Protein

In some embodiments, the levels of CSC biomarker can be determined by measuring the protein expression of the CSC biomarkers as disclosed herein. In some embodiments, protein expression can be measured by contacting a biological sample with an aptamer, antibody-based binding moiety or protein-binding molecule that specifically binds to a CSC biomarker selected from the group of 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4, KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik or fragments or variants thereof. Formation of the protein-protein or antibody-protein complex is then detected by a variety of methods known in the art.

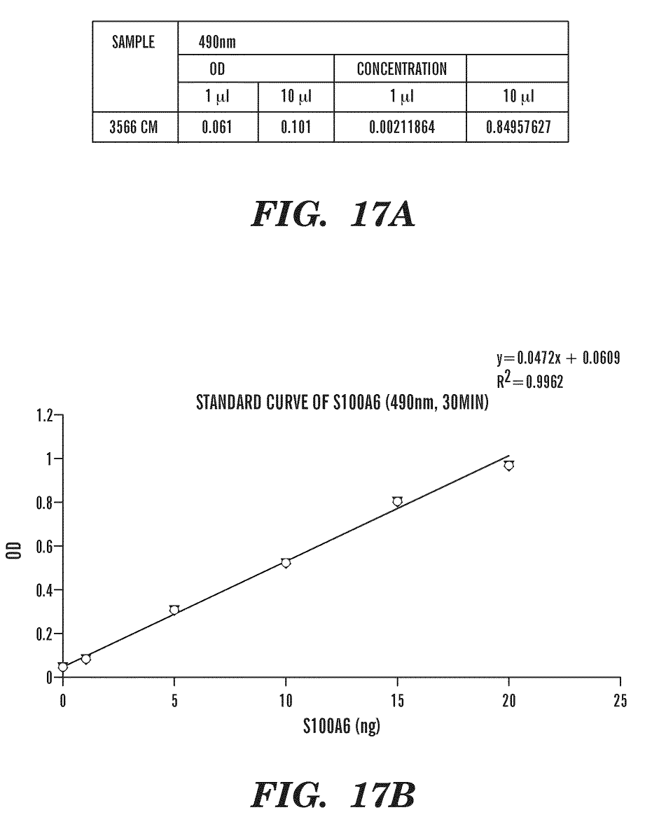

One of ordinary skill in the art can correlate the level of gene expression of a mRNA transcript of a stem cell biomarkers as disclosed herein with the level of protein expression of the cancer stem cell biomarker. For example, one can determine the gene expression by measuring the mRNA transcripts in a biological sample by any method known in the art, or by the methods as disclosed herein, and also measure the protein expression of the cancer stem cell marker using protein expression methods commonly known by persons of ordinary skill in the art, such as ELISA methods used to determine the protein expression of the cancer stem cell biomarker S100A6 as disclosed in the examples and FIG. 17.

The term “protein-binding molecule” refers to an agent, or protein which specifically binds to an protein, such as an a protein-binding molecule which specifically binds a cancer cell biomarker protein, as disclosed herein. Protein-binding molecules are well known in the art, and includes polypeptides, peptides (such as aptamers), antibodies, antibody-based binding moieties, protein-binding peptides, chemicals, non-immunoglobulin and immunoglobulin molecules, and immunologically active determinants of immunoglobulin molecules, such as for example molecules that contain an antigen binding site which specifically binds a cancer cell biomarker protein, and such like molecules. The region on the protein which binds to the protein-binding molecule is referred to as the epitope, and the protein which is bound to the protein-binding molecule is often referred to in the art as an antigen.

The term “antibody-based binding moiety” or “antibody” includes immunoglobulin molecules and immunologically active determinants of immunoglobulin molecules, e.g., molecules that contain an antigen binding site which specifically binds to the biomarker proteins. The term “antibody-based binding moiety” is intended to include whole antibodies, e.g., of any isotype (IgG, IgA, IgM, IgE, etc), and includes fragments thereof which are also specifically reactive with the biomarker proteins. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's can be covalently or non-covalently linked to form antibodies having two or more binding sites. Thus, “antibody-based binding moiety” includes polyclonal, monoclonal, or other purified preparations of antibodies and recombinant antibodies. The term “antibody-based binding moiety” is further intended to include humanized antibodies, bispecific antibodies, and chimeric molecules having at least one antigen binding determinant derived from an antibody molecule. In a preferred embodiment, the antibody-based binding moiety is detectably labeled. In some embodiments, a “protein-binding molecule” is a co-factor or binding protein that interacts with the protein to be measured, for example a co-factor or binding protein to a CSC biomarker protein. In some embodiments, a protein-binding molecule can be, for example, but not limited to, an antibody substructure, minibody, adnectin, anticalin, affibody, affilin, avibodies, avimer, knottin, fynomer, phylomer, SMIP, versabodies, glubody, C-type lectin-like domain protein, designed ankyrin-repeate proteins (DARPin), tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1,1-set immunoglobulin domain of myosin-binding protein C, 1-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin). The protein-binding molecules can be used in a similar way as antibodies (for example see Zahnd et al. J. Biol. Chem. 2006, Vol. 281, Issue 46, 35167-35175).

The term “labeled antibody” or “labeled protein-binding molecule”, as used herein, includes antibodies or protein-binding molecules that are labeled by a detectable means and include, but are not limited to, antibodies that are enzymatically, radioactively, fluorescently, and chemiluminescently labeled. Antibodies or protein-binding molecules can also be labeled with a detectable tag, such as biotin, c-Myc, HA, VSV-G, HSV, FLAG, V5, or HIS. The detection and quantification of biomarker proteins present in the tissue samples correlate to the intensity of the signal emitted from the detectably labeled antibody.

In one embodiment, the antibody-based or protein-based binding moiety is detectably labeled by linking the antibody to an enzyme. The enzyme, in turn, when exposed to its substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection can also be accomplished using any of a variety of other immunoassays. For example, by radioactively labeling an antibody or protein-binding molecule, it is possible to detect the antibody or protein-binding molecule through the use of radioimmune assays. The radioactive isotope can be detected by such means as the use of a gamma counter or a scintillation counter or by audioradiography. Isotopes which are particularly useful for the purpose of the present invention are ³H, ¹³¹I, ³⁵S, ¹⁴C, and preferably ¹²⁵I.

It is also possible to label an antibody or protein-binding molecule with a fluorescent compound. When the fluorescently labeled antibody or protein-binding molecule is exposed to light of the proper wavelength, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody or protein-binding molecule can also be detectably labeled using fluorescence emitting metals such as 152Eu, or others of the lanthanide series. These metals can be attached to the antibody or protein-binding molecule using such metal chelating groups as diethylenetriaminepentaacetic acid (DTPA) or ethylenediaminetetraacetic acid (EDTA).

An antibody or protein-binding molecule also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are gold, luminol, luciferin, isoluminol, theromatic acridinium ester, imidazole, acridinium salt and oxalate ester.

As mentioned above, levels of enzyme protein can be detected by immunoassays, such as enzyme linked immunoabsorbant assay (ELISA), radioimmunoassay (RIA), Immunoradiometric assay (IRMA), Western blotting, FACS, immunocytochemistry or immunohistochemistry, each of which are described in more detail below. Immunoassays such as ELISA, FACS or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

Immunoassays

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-enzyme) is linked to a solid phase (i.e. a microtiter plate) and exposed to a biological sample containing antigen (e.g. enzyme). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme-linked antibody reacts with a substrate to generate a colored reaction product that can be measured.

In a “competitive ELISA”, antibody or protein-binding molecule is incubated with a sample containing antigen (i.e. enzyme). The antigen-antibody mixture is then contacted with a solid phase (e.g. a microtiter plate) that is coated with antigen (i.e., enzyme). The more antigen present in the sample, the less free antibody that will be available to bind to the solid phase. A labeled (e.g., enzyme linked) secondary antibody is then added to the solid phase to determine the amount of primary antibody bound to the solid phase.

In an “immunohistochemistry assay” a section of tissue is tested for specific proteins by exposing the tissue to antibodies or protein-binding molecules that are specific for the protein that is being assayed. The antibodies or protein-binding molecules are then visualized by any of a number of methods to determine the presence and amount of the protein present. Examples of methods used to visualize antibodies or protein-binding molecules are, for example, through enzymes linked to the antibodies or protein-binding molecules (e.g., luciferase, alkaline phosphatase, horseradish peroxidase, or beta-galactosidase), or chemical methods (e.g., DAB/Substrate chromagen). The sample is then analyzed microscopically, most preferably by light microscopy of a sample stained with a stain that is detected in the visible spectrum, using any of a variety of such staining methods and reagents known to those skilled in the art.

Alternatively, “Radioimmunoassays” can be employed. A radioimmunoassay is a technique for detecting and measuring the concentration of an antigen using a labeled (e.g. radioactively or fluorescently labeled) form of the antigen. Examples of radioactive labels for antigens include 3H, 14C, and 125I. The concentration of antigen enzyme in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody or protein-binding molecule. The higher the concentration of antigen in the sample, the lower the concentration of labeled antigen that will bind to the antibody or protein-binding molecule.

In a radioimmunoassay, to determine the concentration of labeled antigen bound to antibody or protein-binding molecule, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

In some embodiments, such immunoassays can also be performed as multiplex immuno-assays allowing the simultaneous analysis of many antigens. One such techniques uses beads and is known as Luminex technology, another example is the indirect layered peptide array (iLPA) described by Gannot et al. (Journal of Molecular Diagnostics 2007, Vol. 9, No. 3, 297-304)

Other techniques to detect CSC biomarker protein levels in a biological sample can be performed according to a practitioner's preference, and based upon the present disclosure and the type of biological sample (i.e. plasma, urine, tissue sample etc). One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled anti-enzyme antibodies can then be used to assess enzyme levels, where the intensity of the signal from the detectable label corresponds to the amount of enzyme present. Levels can be quantified, for example by densitometry.

In one embodiment, CSC biomarker proteins as disclosed herein, and/or their mRNA levels in the tissue sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of CSC biomarker mRNA or protein level will typically depend on the detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Antibodies, antisera and protein-binding molecules which have binding affinity for CSC biomarker proteins.

In one embodiment, the diagnostic method of the invention uses antibodies or anti-sera, or protein-binding molecules for determining the expression levels of CSC biomarker proteins, for example antibodies with affinities for 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik. The antibodies for use in the present invention can be obtained from a commercial source such as R&D Systems, Abcam or prepared using standard technologies known in the art, e.g. monoclonal hybridoma by immunizing a mouse, polyclonal by immunization a mouse, rabbit, sheep, or other mammal or a chick with a protein, peptide or DNA, Alternatively, antibodies useful in the methods of the present invention can be produced by standard methods commonly known by persons of ordinary skill in the art. In alternative embodiments, commercially available antibodies can be used in the methods as disclosed herein, for example, but not limited to, such commercial antibodies can include; MIA from R&D Systems cat no. MAB2050 (monoclonal) or AF2050 (polyclonal); WNT5a from Cell Signaling cat no 2392; COL6A1 from e.g. Abcam cat no. ab6588; COL6A2 from Novus Biologicals cat no H00001292-M01; FOXC2 from e.g. Abcam cat no. ab5060; FOXA3 from e.g. Abcam cat no. ab11975; S100A4 from e.g. Abcam cat no. ab27957; S100A6 from Abnova Corporation cat.no. H00006277-M16; OPCML e.g. from R&D Systems cat no. AF2777; MGP from e.g. Abcam cat no ab11975; GPR17e.g. from Abcam cat no. ab12544. In some embodiments, the antibodies can be polyclonal or monoclonal antibodies. Methods for the production of enzyme antibodies are disclosed in PCT publication WO 97/40072 or U.S. Application. No. 2002/0182702, which are herein incorporated by reference.

The terms “protein-binding molecule” refers to a agent or protein which specifically binds to an protein, such as an a protein-binding molecule which specifically binds a cancer stem cell biomarker protein. Protein-binding molecules are well known in the art, and include antibodies, protein-binding peptides and the like. The region on the protein which binds to the protein-binding molecule is referred to as the epitope, and the protein which is bound to the protein-binding molecule is often referred to in the art as an antigen.

The terms “specifically binds,” “specific binding affinity” (or simply “specific affinity”), “specifically recognize,” and “immunoreacts with” and other related terms when used to refer to binding between a protein and an antibody, refers to a binding reaction that is determinative of the presence of the protein in the presence of a heterogeneous population of proteins and other biologics. Stated another way, if a molecule “specifically binds” to a protein, it means the molecule recognizes and binds a desired polypeptide but that does not substantially recognize and bind other molecules in a sample. Thus, under designated conditions, a specified antibody binds preferentially to a particular protein and does not bind in a significant amount to other proteins present in the sample. An antibody that specifically binds to a protein has an association constant of at least 10³ M⁻¹ or 10⁴ M⁻¹, sometimes 10⁵ M⁻¹ or 10⁶ M⁻¹, in other instances 10⁶ M⁻¹ or 10⁷ M⁻¹, preferably 10⁸ M⁻¹ to 10⁹ M⁻¹, and more preferably, about 10¹⁰ M⁻¹ to 10¹¹ M⁻¹ or higher. Protein-binding molecules with affinities greater than 10⁸ M⁻¹ are useful in the methods of the present invention. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display or ribosome display library by methods well known in the art. For example, U.S. Pat. No. 5,702,892 (U.S.A. Health & Human Services) and WO 01/18058 (Novopharm Biotech Inc.) disclose bacteriophage display libraries or ribosome display and selection methods for producing antibody binding domain fragments. Protein binding molecules can also be readily obtained by using antigenic portions of the protein to screen a protein binding library, such as phage display or ribosome display library by methods well known in the art.

Detection of antibodies for affinity for a CSC biomarker protein can be achieved by direct labeling of the antibodies themselves, with labels including a radioactive label such as ³H, ¹⁴C, ³⁵S, ¹²⁵I, or ¹³¹I, a fluorescent label, a hapten label such as biotin, or an enzyme such as horse radish peroxidase or alkaline phosphatase. Alternatively, unlabeled primary antibody is used in conjunction with labeled secondary antibody, comprising antisera, polyclonal antisera or a monoclonal antibody specific for the primary antibody. In a preferred embodiment, the primary antibody or antisera is unlabeled, the secondary antisera or antibody is conjugated with biotin and enzyme-linked strepavidin is used to produce visible staining for histochemical analysis.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarily determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention. The antibodies can be polyclonal or monoclonal and can be isolated from any suitable biological source, e.g., murine, rat, sheep and canine. Additional sources are identified infra. The term “antibody” is further intended to encompass digestion fragments, specified portions, derivatives and variants thereof, including antibody mimetics or comprising portions of antibodies that mimic the; structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′)2 fragment, a bivalent fragment comprising two Fab fragments linked by a disulfide bridge at the hinge region; a Ed fragment consisting of the VH and CH, domains; a Fv fragment consisting of the VL and VH domains of a single arm of an antibody, a dAb fragment (Ward et al. (1989) Nature 341:544-546), which consists of a VH domain; and an isolated complementarily determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl. Acad Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “antibody variant” is intended to include antibodies produced in a species other than a mouse. It also includes antibodies containing post translational modifications to the linear polypeptide sequence of the antibody or fragment. It further encompasses fully human antibodies. The term “antibody derivative” is intended to encompass molecules that bind an epitope as defined above and which are modifications or derivatives of a native monoclonal antibody of this invention. Derivatives include, but are not limited to, for example, bispecific, multi specific, heterospecific, trispecific, tetraspecific, multi specific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the present invention can include amino acid residues not encoded by human germline immunoglobulin sequences (e.g., mutations introduced by random or site-specific mutagenesis in vitro or by somatic mutation in viva). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, CL, CH domains (e.g., CH1, CH2, CH3), hinge, (Via, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain); genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody can be at least about 95%, or even at least about 96%, or least about 97%, or least about 98%, or least about 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody can display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human monoclonal antibody” refers to antibodies displaying a single binding specificity which have variable and constant regions derived from human germline immunoglobulin sequences. The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in viva somatic mutagenesis) and thus the amino acid sequences of the VH and VL regions of the recombinant antibodies are sequences that, while derived from and related to human germline VH and VL sequences, can not naturally exist within the human antibody germline repertoire in vivo. As used herein, “isotype” refers to the antibody class (e.g., IgM or IgG1) that is encoded by heavy chain constant region genes.

### Cancers and Cancer Stem Cells

In some embodiments, the biological sample obtained from the subject is from a biopsy tissue sample, body fluid or blood, and in some embodiments, the sample is from a tumor or cancer tissue sample. The level of expression can be determined by methods known by the skilled artisan, for example by northern blot analysis or RT-PCR, or using the methods as disclosed in the methods section of the Examples.

Cancer treatments promote tumor regression by inhibiting tumor cell proliferation, inhibiting angiogenesis (growth of new blood vessels that is necessary to support tumor growth) and/or prohibiting metastasis by reducing tumor cell motility or invasiveness.

In some embodiments, the identification of cancer stem cells in a population of cells is useful to identify subjects likely to have cancer reoccurrence, or having refractory cancers (such as cancers which to not respond to existing therapies or come back after a period of cancer remission).

In some embodiments, a biological sample is obtained from a subject with cancer. In some embodiments, the subject has adult or pediatric cancer, including solid phase tumors/malignancies, locally advanced tumors, human soft tissue sarcomas, metastatic cancer, including lymphatic metastases, blood cell malignancies including multiple myeloma, acute and chronic leukemia's, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastic tumors, neuroblastoma, medulloblastoma, astrocytic brain tumors, gliomas, metastatic tumor cell invasion in the central nervous system, neuroendocrine tumors, bone cancers including osteomas, skin cancers including melanoma, tumor progression of human skin keratinocytes, squamous cell carcinoma (including head and neck squamous cell carcinoma), basal cell carcinoma, hemangiopericytoma and Kaposi's sarcoma.

In some embodiments, the cancer stem cell markers are useful to identify a cancer comprising cancer stem cells. In some embodiments, the cancer stem cell is a brain cancer stem cell. In some embodiments, the cancer stem cell is a breast cancer stem cell, or a colon cancer stem cell, or an ovarian cancer stem cell, or a melanoma cancer stem cell. In other embodiments, the cancer stem cell as identified using the CSC biomarkers as disclosed herein can give rise to any type of cancer, for example but not limited to, the cancers such as, breast cancer, lung cancer, head and neck cancer, bladder cancer, stomach cancer, cancer of the nervous system, bone cancer, bone marrow cancer, brain cancer, colon cancer, colorectal cancer, esophageal cancer, endometrial cancer, gastrointestinal cancer, genital-urinary cancer, stomach cancer, lymphomas, melanoma, glioma, glioblastoma, bladder cancer, pancreatic cancer, gum cancer, kidney cancer, retinal cancer, liver cancer, nasopharynx cancer, ovarian cancer, oral cancers, bladder cancer, hematological neoplasms, follicular lymphoma, cervical cancer, multiple myeloma, B-cell chronic lymphcylic leukemia, B-cell lymphoma, osteosarcomas, thyroid cancer, prostate cancer, colon cancer, prostate cancer, skin cancer including melanoma, stomach cancer, testis cancer, tongue cancer, or uterine cancer.

In other embodiments, the cancer stem cell as identified using the CSC biomarkers as disclosed herein can give rise to other cancers including, but not limited to, bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor.

### Uses of the Cancer Stem Cell Biomarkers

In one embodiment, in view of the currently limited options for treatment of reoccurring cancers, the CSC biomarkers or subgroups thereof as disclosed herein are useful for identifying the presence of cancer stem cells in a population of cells. In some embodiments, a subject identified to have a cancer comprising cancer stem cells can be administered a therapeutic regimen to eliminate the cancer stem cells. In some embodiments, the CSC biomarkers or subgroups thereof as disclosed herein are useful for identifying subjects with poor-prognosis, in particular subjects with localized CSCs that are likely to relapse (i.e. cancer reoccurrence) and metastasize. Accordingly, subjects identified with an increased likelihood of CSC can be administered therapy, for example systematic therapy. In some embodiments, a subject identified to have a cancer comprising cancer stem cells can be administered an more aggressive cancer treatment regimen, for example, multiple anti-cancer therapies simultaneously, such as, but not limited to administration of anti-cancer agents and radiotherapy or surgical resection.

In some embodiments, the compositions and methods as disclosed herein can also be used to identify subjects in need of frequent follow-up by a physician or clinician to monitor the cancer and risk of relapse, as well as cancer progression. For example, if a subject is identified to have a cancer comprising cancer stem cells using the methods and compositions as disclosed herein, the subject can initiate treatment earlier, when the disease may potentially be more sensitive to treatment, or the subject can initiate a treatment specifically aimed at eliminating the cancer stem cells.

In further embodiments, the methods and compositions as disclosed herein are useful for identifying subjects with cancer stem cells expressing at least 6 CSC biomarkers or subgroups thereof, which is useful to identify subjects most suitable or amenable to be enrolled in clinical trial for assessing a therapy specifically aimed at eliminating the cancer stem cells. Such an embodiment will permit more effective subgroup analyses and follow-up studies. Furthermore, the expression of the group of CSC biomarkers as disclosed herein can be used to monitor such subjects enrolled in a clinical trial to provide a quantitative measure for the therapeutic efficacy of a therapy aimed at eliminating the cancer stem cells in which is subject to the clinical trial.

One aspect of the present invention relates to an assay to identify agents that reduce the self-renewal capacity of cancer stem cell populations as disclosed herein as compared to cancer cell populations. In some embodiments, the assay involves contacting a cancer stem cell with an agent, and measuring the proliferation of the cancer stem cell, whereby an agent that decreases the proliferation of the cancer stem cell as compared to a reference agent or absence of an agent identifies an agent that inhibits the self-renewal capacity of the cancer stem cell. Such an agent can be used for development of therapies for the treatment of cancers comprising cancer stem cells. In some embodiments, an assay as disclosed herein can encompass comparing the results of the rate of proliferation of a cancer cell population in the presence of the same agent, where an agent useful for selection as a therapy for the treatment of cancer in a subject is an agent that inhibits the self-renewal capacity of a population of cancer stem cells to a greater extent, for example greater than 10%, or greater than about 20%, or greater than 30% as compared to the ability of the agent to inhibit the self-renewal capacity of a population of cancer cells, for example cancer brain cell.

In one embodiment, one can use the cancer stem cell biomarkers as disclosed herein whether these genes regulate self-renewal, proliferation, migration, survival, quiescence, and differentiation of cancer stem cells. In some embodiments, one can manipulate the expression of the cancer stem cells as disclosed herein to using to use antagonists and/or agonist to determine if the expression of the cancer stem cell biomarker contributes wholly or in part to the self-renewal, proliferation, migration, survival, quiescence, and differentiation of cancer stem cells, and if inhibition or activation of such cancer stem cell biomarker protein or mRNA is useful as a therapeutic strategy for treating cancer comprising cancer stem cells. For example, one can use an inhibitor (i.e. antagonists) to inhibit or decrease the expression or protein of a cancer stem cell upregulated biomarker or in alternatively, use agonists or activator to increase the expression of cancer stem cell downregulated biomarker as disclosed herein to assess if the cancer stem cell biomarker protein contributes wholly, or in part, to the self-renewal, proliferation, migration, survival, quiescence, and differentiation of cancer stem cells.

Such gain-of-function studies are well known in by the skilled artisan, and include for example, using lentiviral expression vectors to express the cancer stem cell downregulated biomarkers and see the effect on the self-renewal, proliferation, migration, survival, quiescence, and differentiation of cancer stem cells as compared to cancer stem cells without the expression of the cancer stem cell downregulated biomarkers. If the self-renewal, proliferation, migration, survival, quiescence, and differentiation of cancer stem cells is reduced in such gain-of function studies, it indicates the reduced expression of the cancer stem cell downregulated biomarker being tested contributes wholly or in part to the proliferation, migration, survival, quiescence, and differentiation of cancer stem cells.

Alternatively, loss-of-function studies are well known in by the skilled artisan, and include for example, using lentiviral expression vectors expressing a RNAi, such as a siRNA, shRNA or microRNA or using aptamers to a cancer stem cell upregulated biomarkers and see the effect on the self-renewal, proliferation, migration, survival, quiescence, and differentiation of cancer stem cells as compared to cancer stem cells without the expression of the cancer stem cell upregulated biomarkers. If the self-renewal, proliferation, migration, survival, quiescence, and differentiation of cancer stem cells is reduced in such loss-of function studies, it indicates the increased expression of the cancer stem cell upregulated biomarker being tested contributes wholly or in part to the proliferation, migration, survival, quiescence, and differentiation of cancer stem cells.

Such loss-of-function studies and gain of function studies can be performed by persons of ordinary skill in the art. By way of an example only, cancer stem cells from mouse and human gliomas can be cultured as described herein. A viral vector, such as a lentivirus encoding either cDNA for gain-of-function or RNAi, such as siRNA for loss-of-function studies can be used to infect cancer stem cells. The lentivirus can be tested on cancer stem cells both in vitro or in vivo and the effects of increased (gain of function) or decreased (loss of function) gene expression of the cancer stem cell biomarker on the cancer stem cell can be determined by comparing cancer stem cells transfected with a control lentivirus or non-transfected cancer stem cells.

Examples of assays in which such gain-of function and/or loss-of function studies can be performed are:

1) self-renewal assay as disclosed herein in the Examples, where a secondary sphere assay and serial tumor transplantation is used to identify cancer stem cell biomarkers which contribute to wholly or in part, to the self-proliferative capacity of cancer stem cells.

2) overall proliferation assay such as the MTT, WST, XTT or MTS proliferation assay or [3H]-thymidine incorporation assay as disclosed herein and in the Examples, as well as determining the % BrdU+, phospho-H3, Ki67+ cells present in a population of cancer stem cells, or alternatively one of ordinary skill in the art can measure the overall growth rate of cultures and transplanted tumors in the presence of lentivirus expressing siRNA to upregulated cancer stem cell biomarkers or alternatively lentivirus expressing the downregulated cancer stem cell biomarkers, or functional fragments thereof. A decrease in the proliferation of cancer stem cells to non-stem cancer cell identifies the cancer stem cell biomarker protein being tested contributes to wholly or in part to the proliferation of cancer stem cells.

3) analysis of cancer stem cells propensity to differentiate: One of ordinary skill in the art can use a viral vector, such as a lentivirus encoding either cDNA of a downregulated CSC biomarker for gain-of-function, or alternatively a RNAi, such as siRNA, shRNA and microRNA or an aptamer targeting the inhibition of an upregulated CSC biomarker for loss-of-function studies to transfect cancer stem cells and determine the % of differentiation of cancer stem cells to non-stem cancer cells in cultures and in tumors, both in vitro and in vivo. An increase in the differentiation of cancer stem cells to non-stem cancer cell identifies the cancer stem cell biomarker protein being tested contributes to wholly or in part to the differentiation of cancer stem cells.

4) sensitivity to chemotherapy and radiation therapies: One of ordinary skill in the art can use a viral vector, such as a lentivirus encoding either cDNA of a downregulated CSC biomarker for gain-of-function, or alternatively a RNAi, such as siRNA, shRNA and microRNA or an aptamer targeting the inhibition of an upregulated CSC biomarker for loss-of-function studies to transfect cancer stem cells and determine the % surviving cancer stem cells in the presence of, or post treatment with a chemotoxic agents and/or radiation treatment in vivo and in vitro. A decrease in the % surviving cancer stem cells after treatment identifies the cancer stem cell biomarker protein being tested contributes to wholly or in part to the resistance of cancer stem cells to specific chemotherapeutic and radiotherapeutic cancer therapies.

5) migration: One of ordinary skill in the art can use a viral vector, such as a lentivirus encoding either cDNA of a downregulated CSC biomarker for gain-of-function, or alternatively a RNAi, such as siRNA, shRNA and microRNA or an aptamer targeting the inhibition of an upregulated CSC biomarker for loss-of-function studies to transfect cancer stem cells and determine, using in vitro migration assays and measurement of migrating cancer cells from the tumor core. A decrease in the migration of cancer stem cells from the tumor core identifies the cancer stem cell biomarker protein being tested contributes to wholly or in part to the migration of cancer stem cells.

6) tumor initiation: One of ordinary skill in the art can use a viral vector, such as a lentivirus encoding either cDNA of a downregulated CSC biomarker for gain-of-function, or alternatively a RNAi, such as siRNA, shRNA and microRNA or an aptamer targeting the inhibition of an upregulated CSC biomarker for loss-of-function studies to transfect cancer stem cells and determine, using a limiting dilution assays the ability of cancer stem cells to form tumors. One would measure tumor initiation efficiency, and if there is a decrease in the tumor-forming efficacy, it identifies the cancer stem cell biomarker protein being tested contributes to wholly or in part to the ability of the cancer stem cell to form a tumor.

One of ordinary skill in the art can design RNAi agents or aptamers for used to decrease the expression of upregulated cancer stem cell biomarkers as disclosed herein. In some embodiments, shRNAs can be purchased from OpenBiosystems and for each gene, 4-5 different shRNAs are generated and tested (by RT-PCR) to determine how much knock-down (i.e. inhibition) can be achieved. Depending on the efficiency of each sequence, one will use 1-3 different shRNA to inhibit the gene expression of the selected upregulated cancer stem cell biomarker by at least 90%.

If from the loss of function studies an upregulated cancer stem cell biomarker is identified to contribute to wholly or in part to the proliferation, migration, survival, quiescence, and differentiation of cancer stem cells, the siRNA can be used as a therapeutic strategy for the treatment and/or prevention of cancer in a subject with cancer comprising cancer stem cells.

Also encompassed in the present invention is use of the cancer stem cells as disclosed herein in assays to identify agents which kill and/or decrease the rate of proliferation of cancer stem cells. In some embodiments, such an assay can comprising both a population of cancer stem cells and a population of non-stem cancer cells, and adding to the media of the population of cancer stem cells and to the population of non-stem cancer cells one or more of the same agents. Once can measure and compare the rate of proliferation of the population of cancer stem cells with the population of non-stem cancer cells using methods such as, for example the MTT, WST, XTT or MTS assay or CFU assay, and an agent identified to decrease the rate of proliferation and/or attenuate proliferation by about 10%, or about 20% or about 30% or greater than 30% and/or kill about 10% or about 20% or about 30% or greater than 30% of the population of cancer stem cells as compared to a population non-stem cancer cells identifies an agent that is useful for a therapy for the treatment of cancer comprising cancer stem cells. Effectively, the assay as disclosed herein can be used to identify agents that selectively inhibit the cancer stem cells as compared to non-stem cancer cell populations. Agents useful in such an embodiment can be any agent such as, for example nucleic acid agents, such as RNAi agents (RNA interference agents), nucleic acid analogues, small molecules, proteins, peptidomimetics, antibodies, peptides, aptamers, ribozymes, and variants, analogues and fragments thereof.

Mouse models of human cancer are becoming increasingly important, often irreplaceable, tools for in vivo cancer studies. For example, S100β-promoter-driven expression of verb-B in engineered mice produces spontaneous, highly infiltrative oligodendrogliomas that cannot be replicated by simply xenografting human brain tumor cell lines into a host mouse brain (1). Accordingly, in one embodiment, the cancer stem cell biomarkers as disclosed herein are useful to identify cancers in animal models cancer which comprise cancer stem cells, as well as useful in the assays to for identifying agents which target and kill and/or decrease the rate of proliferation of cancer stem cells in any animal model of cancer.

Such animal models of cancer commonly known by persons of ordinary skill in the art. Some examples of animal models of cancer are discussed below.

Mouse Models of Human Cancer

Tumor stem cells were first identified and studied in humans, but little is known about the corresponding cells in other mammals. Kondo et al. reported that the side-population (SP) in the rat C6 glioblastoma cell line is enriched in tumor-initiating cells (18), suggesting that tumor stem cells also exist in rodents. Side-population is a cellular phenotype associated with many stem cells by virtue of their expressing multi-drug resistance proteins that extrude the Hoechst dye 33342. All live cells, except SP cells, take up this dye, which emits in both red and blue UV wavelengths. Zhou et al reported that a MDR protein, ABCG2/BCRP1, is necessary and sufficient to confer the SP phenotype (19, 20). However, others including the present inventors, found that SP but not BCRP1+ cells are stem cells (21), suggesting that BCRP1+ cells and SP are not necessarily overlapping populations.

Oligodendroglioma Model

Mice in which the S100β-promoter drives expression of the verbB gene develop oligodendrogliomas (1). VerbB is an activated form of EGFR, which is commonly upregulated in human brain cancer. The S100β promoter is active in glial cells. On the p53−/− background, both tumor incidence and tumor grade increases and this model generates a highly infiltrative brain tumor, similar to the human brain cancer. Importantly, this model not only replicates the tumor histology but also the chromosomal abnormalities associated with human oligodendroglioma (loss of 1p and 19q) (1).

Mouse Models of Breast Cancer

The MMTV-neu transgene used in this study was generated by the Muller laboratory to express unactivated rat neu (ERBB2) from the mouse mammary tumor virus (MMTV) promoter/enhancer (Guy, C. T. et al. Expression of the neu protooncogene in the mammary epithelium of transgenic mice induces metastatic disease; Proc Natl Acad Sci USA 89, 10578-82 (1992)). These transgenic mice develop focal tumors between 4 to 10 months of age in a pregnancy-independent manner with varying metastatic potential. While most mice that develop mammary tumors at an early age do not develop metastasis, 72% of the animals that survive beyond 8 months develop lung metastasis. These longer-surviving animals develop estrogen receptor (ER)-negative, luminal cell-restricted mammary tumors (Cardiff, R. D. et al. The mammary pathology of genetically engineered mice: the consensus report and recommendations from the Annapolis meeting; Oncogene 19, 968-88; 2000).

Another model are the transgenic MMTV-PyMT mice, also generated by the Muller group, express polyomavirus middle T antigen driven by the MMTV promoter/enhancer (Guy, C. T., Cardiff, R. D. & Muller, W. J. Induction of mammary tumors by expression of polyomavirus middle T oncogene: a transgenic mouse model for metastatic disease. Mol Cell Biol 12, 954-61;1992). By 3 months of age, 100% of these mice develop multifocal mammary adenocarcinomas. 94% of the mice develop lung metastasis by 3 months of age, making this a robust and reliable metastatic breast cancer model. Also, four histologically distinct stages of breast cancer progression that mirror a frequent course of the human disease were characterized previously (Lin, E. Y. et al. Progression to malignancy in the polyoma middle T oncoprotein mouse breast cancer model provides a reliable model for human diseases. Am J Pathol 163, 2113-26; 2003), making the MMTV-PyMT mouse an excellent model for examining molecular and cellular changes associated with each stage of tumor progression. Interestingly early stage tumor in MMTV-PyMT mice are ER-positive but most cells become ER-negative after the transition to invasive carcinoma stage. Considering that normal mouse mammary stem cells are ER-,PR-, Erb2/Her2-cells (Asselin-Labat, M. L. et al. Steroid hormone receptor status of mouse mammary stem cells; J Natl Cancer Inst 98, 1011-4; 2006).

Use of these, and other animal models of cancer can be assessed for the cancer stem cell biomarkers as disclosed herein and identify additional cancers which comprise cancer stem cells which can be identified by the methods and cancer stem cell biomarkers as disclosed herein. Cancers identified to comprise cancer stem cells would more accurately predict therapy outcome and thereby guide more effective treatment decisions.

In further embodiments, the cancer stem cells identified using the methods as disclosed herein can be used in assay to for the study and understanding of signalling pathways of cancer stem cells. The use of cancer stem cell of the present invention is useful to aid the development of therapeutic applications for cancers, such as cancers comprising cancer stem cells such as brain cancers. In some embodiments, the use of such cancer stem cells identified using the methods as disclosed herein enable the study of brain cancers. For example, the ovarian cancer stem cells can be used for generating animal models of cancers comprising cancer stem cells as described in the Examples herein, which can be used for an assay to test for therapeutic agents that inhibit the proliferation of cancer stem cells as compared to non-stem cancer cells. Such a model us also useful in aiding the understanding of cancer stem cells in the development of, and reoccurrence of cancer.

In some embodiments, the cancer stem cells can also be used to identify additional markers that characterize them as cancer stem cells as compared to non-stem cancer cell populations. Such markers can be cell-surface markers or surface markers or other markers, for example mRNA or protein markers intracellular within the cell. Such markers can be used as additional agents in the diagnosis of cancers comprising cancer stem cells in subjects with cancers.

In further embodiments, the cancer stem cells and CSC biomarkers as identified by the methods as disclosed herein can be used to prepare antibodies or a protein-binding molecules that are specific markers of cancer stem cells disclosed herein. Polyclonal antibodies can be prepared by injecting a vertebrate animal with cells of this invention in an immunogenic form. Production of monoclonal antibodies is described in such standard references as U.S. Pat. Nos. 4,491,632, 4,472,500 and 4,444,887, and Methods in Enzymology 73B:3 (1981). Specific antibody molecules or protein-binding molecules can also be produced by contacting a library of immunocompetent cells or viral particles with the target antigen, and growing out positively selected clones. See Marks et al., New Eng. J. Med. 335:730, 1996, and McGuiness et al., Nature Biotechnol. 14:1449, 1996. A further alternative is reassembly of random DNA fragments into antibody encoding regions, as described in EP patent application 1,094,108 A.

The antibodies or protein-binding molecules in turn can be used as diagnostic applications to identify a subject with cancers comprising cancer stem cells, or alternatively, antibodies or protein-binding molecules can be used as therapeutic agents to prevent the proliferation and/or kill the cancer stem cells.

The antibodies or protein-binding molecules can be used for the evaluation of protein expression for example in Western blot, ELISA or multiplex systems like Luminex.

In another embodiment, the cancer stem cells as identified by the methods as disclosed herein can be used to prepare a cDNA library of relatively enriched with cDNAs that are preferentially expressed in cancer stem cells as compared to non-stem cancer cells. For example, cancer stem cells can be collected and then mRNA is prepared from the cell pellet or cell lysate by standard techniques (Sambrook et al., supra). After reverse transcribing the cDNA, the preparation can be subtracted with cDNA from, for example non-stem cancer cells in a subtraction cDNA library procedure. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the molecular size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population. SuperSAGE may also be used.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680. Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505. Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined. Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes. Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992. General methods in molecular and cellular biochemistry can also be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

Sequencing technologies may also be used to determine gene expression, e.g. CAGE (cap analysis gene expression) or NimbleGen Sequence capture.

### Methods of Treatment

The invention further provides methods of treating subjects identified as having a cancer comprising a cancer stem cell using the methods of the present invention, wherein the biological sample obtained from the subject is identified to have at least 2.0 fold difference level of expression of at least 6 CSC biomarkers as listed in Table 5 as compared to their corresponding reference expression level.

This invention also provides a method for selecting a therapeutic regimen or determining if a certain therapeutic regimen is more appropriate for a subject identified to have a cancer comprising cancer stem cells by the methods as disclosed herein. For example, an aggressive anti-cancer therapeutic regime can be pursued in a subject identified to have CSCs, where the subject is administered a therapeutically effective amount of an anti-cancer agent to treat or eliminate the CSC. In alternative embodiments, a prophylactic anti-cancer therapeutic regimen can be pursued in a subject that has a cancer in remission but is identified to have the presence of cancer stem cells, and thus a likelihood that the cancer will relapse. In such an embodiment, a subject can be administered a prophylactic dose or maintenance dose of an anti-cancer agent to eliminate the cancer stem cells or prevent the cancer stem cells giving rise to cancer. In alternative embodiments, a subject can be monitored for the presence of CSC using the methods and compositions as disclosed herein, and if on a first (i.e. initial) testing the subject is identified as having CSC, the subject can be administered an anti-cancer therapy, and on a second (i.e. follow-up testing), the subject is identified as not having CSC or the subject has less than 2.0 fold difference in the level of expression of at least 6 CSC biomarkers as compared to the reference level (i.e. the first or initial) testing, the subject can be administered reduced anti-cancer therapy, for example at a maintenance dose.

In general, a therapy is considered to “treat” a subject identified to have cancer stem cells if it provides one or more of the following treatment outcomes: reduction of the number of cancer stem cells or delay recurrence of the cancer from the cancer stem cells after the initial therapy; increased median survival time or decreased metastases. The method is particularly suited to determining which subjects will be responsive or experience a positive treatment outcome to a particular chemotherapeutic regimen. In some embodiments, an anti-cancer therapy is, for example, administration of a chemotherapeutic agent such as a fluropyrimidine drug such as 5-FU or a platinum drug such as oxaliplatin or cisplatin. Alternatively, the chemotherapy can include administration of a topoisomerase inhibitor such as irinotecan. In a yet further embodiment, the therapy comprises administration of an antibody (as broadly defined herein), ligand or small molecule that binds the Epidermal Growth Factor Receptor (EGFR) or other receptor associate with cancer growth or development. As used herein, the term “treatment” refers to treating a condition that has already manifested in the subject. Treatment is performed generally on a subject who is suffering from a condition or physical dysfunction. Such subjects are said to be in need of treatment. Manifestation of a condition would be by the appearance of one or more symptoms of the condition. Treatment is also used to refer to a slowing of onset and/or severity of additional symptoms wherein the subject already has one or more symptoms. The skilled artisan will realize that complete cure is not necessary to qualify as treatment. As such, subjects suitable for treatment include those who exhibit one or more symptoms of a condition and are at risk for developing additional symptoms of a condition. Such subjects also include those with one or more symptoms of a condition, but who have not been diagnosed with the condition by a qualified medical professional. Successful treatment is evidenced by amelioration of one or more symptoms of the condition or dysfunction as discussed herein

The term “prevention” is used to refer to a situation wherein a subject does not yet have the specific condition being prevented, meaning that it has not manifested in any appreciable form. Prevention encompasses prevention or slowing of onset and/or severity of a symptom, (including where the subject already has one or more symptoms of another condition). Prevention is performed generally in a subject who is at risk for development of a condition or physical dysfunction. Such subjects are said to be in need of prevention.

In one embodiment, the methods of prevention described herein, further comprise selection of such a subject at risk for a condition (e.g., cancer) by identifying the subject as having cancer stem cells using the methods as disclosed herein. Such a subjects can be then administered an appropriate anti-cancer therapy as disclosed herein, to thereby prevent the cancer from developing.

In one embodiment of the invention, the subject is also undergoing another therapy. Such therapies include, without limitation, other therapies or administration of anti-cancer agents to treat or prevent cancer. Such therapies are commonly known by persons of ordinary skill in the art and are discussed herein.

In some embodiments, the anti-cancer therapy is a chemotherapeutic agent, radiotherapy etc. Such anti-cancer therapies are disclosed herein, as well as others that are well known by persons of ordinary skill in the art and are encompassed for use in the present invention. In some embodiments the anti-cancer therapy, or cancer prevention strategy is targets the EGF/EGFR pathway, and in other embodiments, the anti-cancer therapy or cancer prevention strategy does not target the EGF/EGFR pathway.

The term “anti-cancer agent” or “anti-cancer drug” is any agent, compound or entity that would be capable of negatively affecting the cancer in the subject, for example killing cancer cells, inducing apoptosis in cancer cells, reducing the growth rate of cancer cells, reducing the number of metastatic cells, reducing tumor size, inhibiting tumor growth, reducing blood supply to a tumor or cancer cells, promoting an immune response against cancer cells or a tumor, preventing or inhibiting the progression of cancer, or increasing the lifespan of the subject with cancer. In some embodiments, appropriate anti-cancer therapies for administration to a subject identified to have cancer stem cells is any agent, compound or entity that would be capable of negatively affecting the cancer stem cell, for example kill the cancer stem cell, inducing apoptosis in the cancer stem cells, reducing the differentiation and propagation of the cancer stem cell, and preventing the cancer stem cell from producing progeny cancer cells. Anti-cancer therapy includes biological agents (biotherapy), chemotherapy agents, and radiotherapy agents. The combination of chemotherapy with biological therapy is known as biochemotherapy.

Treatment can include prophylaxis, including agents which slow or reduce the CSC from giving rise to cancerous cells in a subject. In other embodiments, the treatments are any means to prevent the proliferation of the cancer stem cells themselves, or their differentiation into cancerous cells. In some embodiments, an anti-cancer treatment includes an agent which suppresses the EGF-EGFR pathway, for example but not limited to inhibitors and agents of EGFR. Inhibitors of EGFR include, but are not limited to, tyrosine kinase inhibitors such as quinazolines, such as PID 153035, 4-(3-chloroanilino)quinazoline, or CP-358,774, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines (Traxler et al., (1996) J. Med Chem 39:2285-2292), curcumin (diferuloyl methane) (Laxmin arayana, et al., (1995), Carcinogen 16:1741-1745), 4,5-bis(4-fluoroanilino)phthalimide (Buchdunger et al. (1995) Clin. Cancer Res. 1:813-821; Dinney et al. (1997) Clin. Cancer Res. 3:161-168); tyrphostins containing nitrothiophene moieties (Brunton et al. (1996) Anti Cancer Drug Design 11:265-295); the protein kinase inhibitor ZD-1 839 (AstraZeneca); CP-358774 (Pfizer, Inc.); PD-01 83805 (Warner-Lambert), EKB-569 (Torrance et al., Nature Medicine, Vol. 6, No. 9, September. 2000, p. 1024), HKI-272 and HKI-357 (Wyeth); or as described in International patent application WO05/018677 (Wyeth); W099/09016 (American Cyanamid); W098/43960 (American Cyanamid); WO 98/14451; WO 98/02434; W097/38983 (Warener Labert); W099/06378 (Warner Lambert); W099/06396 (Warner Lambert); W096/30347 (Pfizer, Inc.); W096/33978 (Zeneca); W096/33977 (Zeneca); and W096/33980 (Zeneca), WO 95/19970; U.S. Pat. App. Nos. 2005/0101618 assigned to Pfizer, 2005/0101617, 20050090500 assigned to OSI Pharmaceuticals, Inc.; all herein incorporated by reference. Further useful EGFR inhibitors are described in U.S. Pat. App. No. 20040127470, particularly in tables 10, 11, and 12, and are herein incorporated by reference.

In another embodiment, the anti-cancer therapy includes a chemotherapeutic regimen further comprising radiation therapy. In an alternate embodiment, the therapy comprises administration of an anti-EGFR antibody or biological equivalent thereof.

In some embodiments, the anti cancer treatment comprises the administration of a chemotherapeutic drug selected from the group consisting of fluoropyrimidine (e.g., 5-FU), oxaliplatin, CPT-11, (e.g., irinotecan) a platinum drug or an anti EGFR antibody, such as the cetuximab antibody or a combination of such therapies, alone or in combination with surgical resection of the tumor. In yet a further aspect, the treatment compresses radiation therapy and/or surgical resection of the tumor masses. In one embodiment, the present invention encompasses administering to a subject identified as having, or increased risk of developing CSC an anti-cancer combination therapy where combinations of anti-cancer agents are used, such as for example Taxol, cyclophosphamide, cisplatin, gancyclovir and the like. Anti-cancer therapies are well known in the art and are encompassed for use in the methods of the present invention. Chemotherapy includes, but is not limited to an alkylating agent, mitotic inhibitor, antibiotic, or antimetabolite, anti-angliogenic agents etc. The chemotherapy can comprise administration of CPT-11, temozolomide, or a platin compound. Radiotherapy can include, for example, x-ray irradiation, w-irradiation, δ-irradiation, or microwaves.

The term “chemotherapeutic agent” or “chemotherapy agent” are used interchangeably herein and refers to an agent that can be used in the treatment of cancers and neoplasms, for example brain cancers and gliomas and that is capable of treating such a disorder. In some embodiments, a chemotherapeutic agent can be in the form of a prodrug which can be activated to a cytotoxic form. Chemotherapeutic agents are commonly known by persons of ordinary skill in the art and are encompassed for use in the present invention. For example, chemotherapeutic drugs for the treatment of tumors and gliomas include, but are not limited to: temozolomide (Temodar), procarbazine (Matulane), and lomustine (CCNU). Chemotherapy given intravenously (by IV, via needle inserted into a vein) includes vincristine (Oncovin or Vincasar PFS), cisplatin (Platinol), carmustine (BCNU, BiCNU), and carboplatin (Paraplatin), Mexotrexate (Rheumatrex or Trexall), irinotecan (CPT-11); erlotinib; oxalipatin; anthracyclins-idarubicin and daunorubicin; doxorubicin; alkylating agents such as melphalan and chlorambucil; cis-platinum, methotrexate, and alkaloids such as vindesine and vinblastine.

In another embodiment, the present invention encompasses combination therapy in which subjects identified as having, or at increased risk of developing CSC using the methods as disclosed herein are administered an anti-cancer combination therapy where combinations of anti-cancer agents are used are used in combination with cytostatic agents, anti-angiogenic agents such as anti-VEGF agents and/or p53 reactivation agent. A cytostatic agent is any agent capable of inhibiting or suppressing cellular growth and multiplication. Examples of cytostatic agents used in the treatment of cancer are paclitaxel, 5-fluorouracil, 5-fluorouridine, mitomycin-C, doxorubicin, and zotarolimus. Other cancer therapeutics include inhibitors of matrix metalloproteinases such as marimastat, growth factor antagonists, signal transduction inhibitors and protein kinase C inhibitors.

As used herein the term “anti-VEGF agent” refers to any compound or agent that produces a direct effect on the signaling pathways that promote growth, proliferation and survival of a cell by inhibiting the function of the VEGF protein, including inhibiting the function of VEGF receptor proteins. The term “agent” or “compound” as used herein means any organic or inorganic molecule, including modified and unmodified nucleic acids such as antisense nucleic acids, RNAi agents such as siRNA or shRNA, microRNA, peptides, peptidomimetics, receptors, ligands, and antibodies. Preferred VEGF inhibitors, include for example, AVASTIN® (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4-Bromo-2,6-difluoro-benzyloxy)-5-[3-(4-pyrrolidin 1-yl-butyl)-ureido]-isothiazole-4-carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which inhibits VEGF-R2 and -R1 (AstraZeneca), CEP-7055 (Cephalon Inc., Frazer, Pa.), PKC 412 (Novartis), AEE788 (Novartis), AZD-2171), NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), IM862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat (AEterna Zentaris Inc; Quebec City, Calif.) and combinations thereof.

The compounds used in connection with the treatment methods of the present invention are administered and dosed in accordance with good medical practice, taking into account the clinical condition of the individual subject, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

As used herein, the terms “treat” or “treatment” or “treating” refers to both therapeutic treatment and prophylactic or preventative measures, wherein the object is to prevent or slow the development of the disease, decrease the number of cancer stem cells in a subject, reduce the reoccurrence of cancer, or spread of cancer, or reducing at least one effect or symptom of a condition, disease or disorder associated with inappropriate proliferation or a cell mass, for example cancer. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those identified to have cancer stem cells identified by the methods ad disclosed herein, or subjects already diagnosed with cancer, as well as those likely to develop secondary tumors due to metastasis or presence of cancer stem cells.

The term “effective amount” as used herein refers to the amount of therapeutic agent such as a anti-cancer agent, to alleviate at least one or more symptom of the disease or disorder, and relates to a sufficient amount of pharmacological composition to provide the desired effect. The phrase “therapeutically effective amount” as used herein means a sufficient amount of an anti-cancer therapy to treat a disorder and preferably to eliminate or reduce the number of cancer stem cells, at a reasonable benefit/risk ratio applicable to any medical treatment. The term “therapeutically effective amount” therefore refers to an amount of an anti-cancer agent as disclosed herein that is sufficient to effect a therapeutically or prophylatically significant reduction in the number of cancer stem cells as identified using the cancer stem cell biomarkers as disclosed herein, and/or reduce a symptom of cancer. Alternatively a reverse the level of expression of the cancer cell biomarker at least about 10% towards the direction of the reference level would be considered a therapeutically or prophylatically significant amount (i.e. if the cancer stem cell biomarker is an upregulated gene, a decrease in the expression of such a cancer stem cell biomarker would be considered a therapeutically or prophylatically significant amount, whereas if the cancer stem cell biomarker is a downregulated gene, an increase in the expression of such a cancer stem cell biomarker would be considered a therapeutically or prophylatically significant amount).

A therapeutically or prophylatically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, however, that the total daily usage of the compositions and formulations as disclosed herein will be decided by the attending physician within the scope of sound medical judgment. The exact amount required will vary depending on factors such as the type of disease being treated.

With reference to the treatment of a subject with a cancer with a pharmaceutical composition comprising at least one pyrazoloanthrones as disclosed herein, the term “therapeutically effective amount” refers to the amount that is safe and sufficient to prevent or delay the development and further growth of a tumor or the spread of metastases in cancer patients. The amount can thus cure or cause the cancer to go into remission, slow the course of cancer progression, slow or inhibit tumor growth, slow or inhibit tumor metastasis, slow or inhibit the establishment of secondary tumors at metastatic sites, or inhibit the formation of new tumor metastases. The effective amount for the treatment of cancer depends on the tumor to be treated, the severity of the tumor, the drug resistance level of the tumor, the species being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of cancer and tumor, for example treatment of a rodent with a cancer, and any treatment or administration of the compositions or formulations that leads to a decrease of at least one symptom of the cancer, for example a reduction in the size of the tumor or a slowing or cessation of the rate of growth of the tumor indicates effective treatment. In embodiments where the compositions are used for the treatment of cancer, the efficacy of the composition can be judged using an experimental animal model of cancer, e.g., mice or rats including genetically modified mice or rats, or preferably, transplantation of tumor cells into an animal model. When using an experimental animal model, efficacy of treatment is evidenced when a reduction in a symptom of the cancer, for example a reduction in the size of the tumor or a slowing or cessation of the rate of growth of the tumor occurs earlier in treated, versus untreated animals or longer survival time of the animal. By “earlier” is meant that a decrease, for example in the size of the tumor occurs at least 5% earlier, but preferably more, e.g., one day earlier, two days earlier, 3 days earlier, or more.

As used herein, the term “treating” when used in reference to a cancer treatment is used to refer to the reduction of a symptom and/or a biochemical marker of cancer, for example a reduction in at least one upregulated cancer stem cell biomarker by at least about 10%, or an increase in at least one downregulated cancer stem cell biomarker by at least about 10% would be considered an effective treatment. A reduction in the rate of proliferation of the cancer stem cells by at least about 10% would also be considered effective treatment by the methods as disclosed herein. As alternative examples, a reduction in a symptom of cancer, for example, a slowing of the rate of growth of cancer stem cells by at least about 10% or a cessation of the cancer stem cells differentiating into non-stem cancer cells, or a reduction of the differentiation of cancer stem cells to non-stem cancer stem cells by at least about 10% would also be considered as affective treatments by the methods as disclosed herein. In some embodiments, it is preferred, but not required that the therapeutic agent actually kill the tumor.

The methods of the present invention are useful for the early detection of subjects susceptible to developing cancer, for example the cancer stem cell biomarkers can be used to identify subject having cancer stem cells and likely to develop cancer. Thus, in such subjects anti-cancer treatment may be initiated early, e.g. before or at the beginning of the onset of symptoms, for example before the onset of cancer symptoms. Accordingly, the cancer stem cell biomarkers as disclosed herein are useful for the identification of a subject who is at risk of developing cancer and such a subject can be selected to be administered anti-cancer therapies to prevent the development of cancer.

In alternative embodiments, the cancer stem cell biomarkers are useful to identify a subject with cancer which comprises cancer stem cells. In such an embodiment, and anti-cancer treatment may be administered to a subject that has, or is at risk of developing cancer. In alternative embodiments, the treatment may be administered prior to, during, concurrent or post development of cancer, for example, treatment can be administered to a subject that has had cancer and the cancer is in remission but the subject is identified to possess CSC. Dosages are known to those of skill in the art and can be determined by a physician.

In some embodiments, where a subject is identified as having CSC using the CSC biomarkers and methods as disclosed herein, a clinician can recommended a treatment regimen to reduce or lower the expression levels of the CSC biomarkers in the subject. Accordingly, the methods of the present invention provide preventative methods to reduce the risk of a subject developing cancer by differentiation of the cancer stem cells. In such an embodiment, an agent could reduce the protein and/or gene transcript expression level of at least 2 of the CSC biomarkers as listed in Table 5, but preferably by reducing the protein and/or gene transcript levels of about 3, about 4, about 5, about 6, about 7, about 8, about 9, about 10, about 11 or more CSC biomarkers as listed in Table 5 in the subject.

In another embodiment, a subject identified as having CSC using the methods as disclosed herein can be monitored for levels of CSC biomarker expression in a biological sample before, during and after an anti-cancer therapy or treatment regimen. Where a subject is identified to still have a level of a CSC biomarker in the biological sample that is least 1.5-fold for upregulated genes, or at least 0.5-fold (i.e. a 50% decrease) for downregulated genes as compared to the first measurement, (and thus still has CSC and is at risk of having or developing cancer) after a period of time of being administered such a treatment regimen, then the treatment regimen could be modified, for example the subject could be administered (i) a different anti-cancer therapy or anti-cancer drug (ii) a different amount such as an increased amount or dose of a anti-cancer therapy or anti-cancer drug or (iii) a combination of anti-cancer therapies etc.

### Kits

In some embodiments, the present invention provides diagnostic methods for determining the likelihood of a subject having cancer stem cells by gene expression analysis of at least 6 gene transcripts of the CSC biomarkers as listed in Table 5. In some embodiments, the methods use probes or primers comprising nucleotide sequences which bind under stringent conditions to the different nucleic acid sequences selected from the group of 2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); and 5033414K04Rik (SEQ ID NO:45); U16153 (SEQ ID NO:46) or a subgroup thereof. Accordingly, the invention provides kits for performing these methods.

The kit can comprise at least 6 probes or 6 primer-pairs which are capable of specifically hybridizing to at least 6 genes selected from the group of CSC biomarkers as disclosed in Table 5 and instructions for use. Preferred kits amplify all or a portion of at least 6 gene transcripts selected from the group of CSC biomarkers as disclosed in Table 5. Such kits are suitable for detection of level of transcript expression by, for example, fluorescence detection, by electrochemical detection, by radioactive detection or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a biological sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention.

In alternative embodiments, the present invention provides diagnostic methods for determining the likelihood of a subject having or developing cancer or CSC by protein expression analysis of at least 6 proteins encoded by the CSC biomarkers as listed in Table 5.

In some embodiments, the biological samples used in the diagnostic kits include cells, protein or membrane extracts of cells, or biological fluids such as sputum, blood, serum, plasma, or urine. The biological sample used in the above described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the reference biological samples as well as positive and negative controls, reagents, primers, sequencing markers, probes and antibodies described herein for determining the protein and/or gene transcript expression level of at least 6 CSC biomarkers as disclosed herein, in order to determine a subject's likelihood of having or being at risk of having or developing cancer.

As amenable, these kit components may be packaged in a manner customary for use by those of skill in the art. For example, these suggested kit components may be provided in solution or as a liquid dispersion or the like.

The invention also provides diagnostic and experimental kits which include antibodies for determining the protein expression level encoded by at least 6 CSC biomarkers as disclosed herein, in order to determine a subject's likelihood of having or being at risk of developing CSC. In such kits, the antibodies may be provided with means for binding to detectable marker moieties or substrate surfaces. Alternatively, the kits may include the antibodies or protein binding proteins already bound to marker moieties or substrates. The kits may further include reference biological samples as well as positive and/or negative control reagents as well as other reagents for adapting the use of the antibodies to particular experimental and/or diagnostic techniques as desired. The kits may be prepared for in vivo or in vitro use, and may be particularly adapted for performance of any of the methods of the invention, such as ELISA. For example, kits containing antibody bound to multi-well microtiter plates can be manufactured.

In some embodiments, the kits as disclosed herein can optionally comprise quality control genes and/or protein-binding molecules to house keeping genes. For example, such quality control genes can determine the sensitivity of the reaction, by for example having a serial dilution of a nucleic acid in the kit, and/or protein-binding molecule which hybridizes and/or specifically binds to a house keeping gene which is typically expressed at high levels in virtually all cells. One can use any house keeping genes or a combination of house keeping genes expressed at different levels in cells. Such house keeping genes are well known by persons of ordinary skill in the art, and include for example but are not limited to GAPDH, beta-actin, 18S and the like. Use of such quality control genes and/or protein binding molecules in the kits as disclosed herein are useful to determine the quality and/or integrity of the biological sample being analyzed, for example to monitor contaminants in the biological sample, monitor mRNA transcript degradation and/or protein degradation, as well as determine DNA contamination and/or protein contamination in a RNA biological sample.

### Methods to Identify Cancer Stem Cell Biomarkers

Another aspect of the present invention related to methods to identify cancer stem cell biomarkers. In one embodiment, the methods comprise the step of obtaining a plurality of tumor cells from a subject, where the subject can be a human subject, or alternatively a mouse model of cancer. The methods also involves obtaining a plurality of organ matched, non-tumor cells, for example if the tumor is a lung tumor, the organ matched non-tumor cells can be obtained from lung tissue, which could be obtained from the same subject as the tumor was derived from (i.e. allogenic) or from a different subject. The tumor cells and non-tumor cells are cultured in single cell suspension at a clonal density of about 1 cell/ul in vitro for a sufficient period of time for them to form spherical cell aggregates, commonly known in the art as spheres. Cells which maintain secondary spheres for multiple passages, for example at least about 20, about 21, about 22, about 23, about 24, about 25, about 26, about . . . 30, about . . . 35 passages are selected for further analysis, as the ability of the cells to form spheres is indicative of their self-renewal capacity, with the spheres from the tumor tissue referred to as TSC (tumor stem cell) and the spheres from the normal organ matched tissue is referred to as SC (stem cells). The selected TSC and SC which maintain self-renewal capacity over at least about 20 passages in vitro are transplanted into a suitable animal model, for example a mouse model or rodent model of cancer. The TSC which give rise to rapid tumor formation in a shorter period of time as compared to the animals transplanted with the SC are removed from the animal model and serial transplanted into a second appropriate animal model. On formation of a tumor by the TSC or SC, the cells are removed and serially transplanted into another animal until multiple passages have occurred, for example at least 3, at least 4, at least 5, at least 6, at least 7, at least 8 or more serial passage procedures. The TSC and SC are harvested and selected based on their side-population classification using flow cytometry methods commonly known by persons of ordinary skill in the art and as disclosed herein. The SP population of TSC are selected and separated from the non-SP TSC cell population and subjected to differential gene expression analysis by methods commonly known by persons of ordinary skill in the art. Genes which are differentially expressed in the SP population of TSC as compared to the non-SP TSC population of cells are identified as potential stem cancer cell biomarkers for that cancer stem cells from the cancer tissue from which they were initially derived.

In some embodiments, the method to identify cancer stem cell biomarkers as described herein are useful to identify cancer stem cell biomarkers of any type of cancer. For example, a plurality of tumor cells can be obtained from cancers selected from the group; adult or pediatric cancer, including solid phase tumors/malignancies, locally advanced tumors, human soft tissue sarcomas, metastatic cancer, including lymphatic metastases, blood cell malignancies including multiple myeloma, acute and chronic leukemia's, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastic tumors, neuroblastoma, medulloblastoma, astrocytic brain tumors, gliomas, metastatic tumor cell invasion in the central nervous system, neuroendocrine tumors, bone cancers including osteomas, skin cancers including melanoma, tumor progression of human skin keratinocytes, squamous cell carcinoma (including head and neck squamous cell carcinoma), basal cell carcinoma, hemangiopericytoma and Kaposi's sarcoma.

In some embodiments, the methods to identify cancer stem cell biomarkers are useful to identify cancer stem cells biomarkers from the following group of cancer stem cells; a breast cancer stem cell, or a colon cancer stem cell, or an ovarian cancer stem cell, or a melanoma cancer stem cell. In other embodiments, the cancer stem cell as identified using the CSC biomarkers as disclosed herein can give rise to any type of cancer, for example but not limited to, the cancers such as, breast cancer, lung cancer, head and neck cancer, bladder cancer, stomach cancer, cancer of the nervous system, bone cancer, bone marrow cancer, brain cancer, colon cancer, colorectal cancer, esophageal cancer, endometrial cancer, gastrointestinal cancer, genital-urinary cancer, stomach cancer, lymphomas, melanoma, glioma, glioblastoma, bladder cancer, pancreatic cancer, gum cancer, kidney cancer, retinal cancer, liver cancer, nasopharynx cancer, ovarian cancer, oral cancers, bladder cancer, hematological neoplasms, follicular lymphoma, cervical cancer, multiple myeloma, B-cell chronic lymphcylic leukemia, B-cell lymphoma, osteosarcomas, thyroid cancer, prostate cancer, colon cancer, prostate cancer, skin cancer including melanoma, stomach cancer, testis cancer, tongue cancer, or uterine cancer.

In other embodiments, the cancer stem cell as identified using the CSC biomarkers as disclosed herein can give rise to other cancers including, but not limited to, bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor.

Other objects, features and advantages will become apparent from the following detailed description. It should be understood, however, that the detailed description and specific examples, while indicating specific embodiments of the invention, are given by way of illustration only, since various changes and modifications within the spirit and scope if the invention will become apparent to those skilled in the art from this detailed description.

The invention now being generally described, it will be more readily understood by reference to the following examples which are included merely for purposes of illustration of certain aspects and embodiments of the present invention, and are not intended to limit the invention

The following examples are provided to illustrate certain embodiments of the invention. They are not intended to limit in any way the remainder of the disclosure.

### EXAMPLES

The examples presented herein relate to methods and compositions for the identification of cancer stem cells in a population of cells by measuring expression levels of at least 6 cancer stem cell biomarkers as disclosed herein. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Methods

Isolation and Culture of Primary Tumorspheres: Primary cells from S100β-verbB;p53−/− animal brain tumors were isolated and grown in modified DME/F-12 with Neurocult Proliferation Supplement (Stemcell Technologies) or B27 (Invitrogen) and penicillin/streptomycin. Normal neural stem cells were isolated from the SVZ region of p53−/− or S100β-verbB;p53−/− animals and cultured in same medium supplemented with 20 ng/ml EGF and 10 ng/ml bFGF. Self-renewal assays were performed by plating single cells at 1 cell/μl density and counting the number of spheres that formed after 6 days. All animal procedures were approved by the Animal Care and Use Committee at The Jackson Laboratory.

FACS and Immunohistochemical Analysis: Normal and tumor tissues were dissociated with Accutase (Invitrogen) digestion and mechanical trituration. Dissociated cells were stained using a standard FACS protocol. Antibodies used: CD133 (Chemicon and Miltenyi) and BCPR1(Chemicon). For SP sorting, cells were incubated with Hoechst 33342 at a concentration of 5 μg/ml at 37° C. for 45 min. C57BL/6 (B6) bone marrow control cells were incubated for 90 min. Cells were resuspended in ice-cold culture medium containing 2 μg/ml Hoechst 33342 for sorting. Standard immunofluorescence protocols were used on tissues that were fixed in 4% parafomaldehyde (PFA) overnight. Antibodies used were: BCRP1 (Chemicon), SOX2 (Chemicon), TUBB3 (Promega), GFAP (Chemicon), NG2 (Chemicon), OLIG2 (Chemicon), and S100A6 (LabVision). Fluorescent sections were imaged using a Zeiss (Axiovert 200M) microscope with Apotome optical sectioning.

In the case of mammary tissue non-epithelial cells will be removed with magnetic beads bound to antibodies against CD31 Ter119, and CD45, and the remaining “Lin-” mammary epithelial cells will be labeled with antibodies against CD24 and CD49f (EasySep, StemCell Technologies).

Intracranial and Flank injections: Tumor cells were injected into the flank or brain of NOD-SCID immune-deficient mice. For intracranial injections, cells were injected using a stereotaxic device (bregma: −2.5, −1, −4).

Real-Time PCR analysis: RNA was treated with DNAse prior to cDNA conversion (using iScript from BioRad). Real-time PCR was performed using SYBR Green Supermix from BioRad on a LightCycler PCR machine (Roche). Relative fold changes were obtained by first normalizing all samples internally to 18S levels and then comparing them relative to NSC. The primers used were are shown in Table 11:

| | | |
|---|---|---|
| PRIMER | Tm | PRIMER SEQUENCE (SEQ ID NO) |

| | | |
|---|---|---|
| S100A4 (forward) | 60.4 | TTTGAGGGCTGCCCAGATAAGGAA |
| | | (SEQ ID NO: 47) |
| S100A4 (reverse) | 59.1 | CACATGTGCGAAGAAGCCAGAGTA |
| | | (SEQ ID NO: 48) |
| Snail2 (forward) | | ACTACAGCGAACTGGACACACACA |
| | | (SEQ ID NO: 49) |
| Snail2 (reverse) | | AGTAATAGGGCTGTATGCTCCCGA |
| | | (SEQ ID NO: 50) |
| Col6a1 (forward) | 60.1 | ATCTAGATCCCGCCCTTGGTTTGT |
| | | (SEQ ID NO: 51) |
| Col6a1 (reverse) | 59.7 | CGGAAACTGCAGTGATGGTGTGAA |
| | | (SEQ ID NO: 52) |
| Slit3 (forward) | | GCTGACCAATCACACCTTCAGCAA |
| | | (SEQ ID NO: 53) |
| Slit 3 (reverse) | | TCATTTCCATGGAGGGTCAGCACT |
| | | (SEQ ID NO: 54) |
| Bgn RT Forward | 60 | AAC AAC ATC ACC AAG GTG GGC |
| | | ATC |
| | | (SEQ ID NO: 55) |
| Bgn RT Reverse | 60.2 | AGT AGG GCA CAG GGT TGT TGA |
| | | AGA |
| | | (SEQ ID NO: 56) |
| Foxc2 RT Forward | 59.6 | AAC GAG TGC GGA TTT GTA ACC |
| | | AGG |
| | | (SEQ ID NO: 57) |
| Foxc2 RT Reverse | 59.8 | TTG GCA GTA ACA GTT GGG CAA |
| | | GAC |
| | | (SEQ ID NO: 58) |
| Gja1 RT forward | 60.1 | TGG TCC TCA CCC TCA CCA AAT |
| | | GAT |
| | | (SEQ ID NO: 59) |
| Gja1 RT reverse | 59.8 | AAT ATT GAG CAT GGC TTG CCT |
| | | CCC |
| | | (SEQ ID NO: 60) |
| Cav1-2 RT forward | 60.3 | TGT ACC GTG CAT CAA GAG CTT |
| | | CCT |
| | | (SEQ ID NO: 61) |
| Cav1-2 RT reverse | 60.3 | GTG CTG ATG CGG ATG TTG CTG |
| | | AAT |
| | | (SEQ ID NO: 62) |
| Gpr17 RT forward | 60.1 | AGA GAG CCT GAT GCG AGA ACT |
| | | TGT |
| | | (SEQ ID NO: 63) |
| Gpr17 RT reverse | 60.3 | TCA CCA CAT GCT GGC ACA TTC |
| | | AAC |
| | | (SEQ ID NO: 64) |
| Susd5 RT forward | 60.3 | TGT GGT GAT CTT GGA ACC CAG |
| | | GAA |
| | | (SEQ ID NO: 65) |
| Susd5 RT reverse | 59.8 | TTT ACA TGA TGC TGT GGG ATG |
| | | CCG |
| | | (SEQ ID NO: 66) |
| Mgp RT forward | 58.1 | CCC TTC ATC AAC AGG AGA AAT |
| | | GCC |
| | | (SEQ ID NO: 67) |
| Mgp RT reverse | 59.1 | CTT GTT GCG TTC CTG GAC TCT |
| | | CTT |
| | | (SEQ ID NO: 68) |
| A930001N09Rik | 61.5 | GTTTAAACAAACAAACCGAGGCAGCAT |
| Pmel 5′ | | GGA |
| | | (SEQ ID NO: 69) |
| A930001N09Rik | 62.5 | GTT TAA ACG CAG TCT GCC ATA |
| Pmel 3′ | | CCA GTT GCA TT |
| | | (SEQ ID NO: 70) |
| S100a6 RT forward | 59.9 | TGA GCA AGA AGG AGC TGA AGG |
| | | AGT |
| | | (SEQ ID NO: 71) |
| S100a6 RT reverse | 59.3 | TTC TGA TCC TTG TTA CGG TCC |
| | | AGA |
| | | (SEQ ID NO: 72) |

Microarray data analysis: Probe intensity data from 15 MOUSE430_{—}2 Affymetrix GeneChip arrays were analyzed by R software (www.r-project.org). Affy probe was re-mapped by using custom CDF file (Dai et al., 2005) from Brain Array (which is found on the world-wide web at site: “brainarray-dot-mbni-dot-med-dot-umich-dot-edu/Brainarray” accommodate updated genome and transcription annotation. Perfect match intensities were normalized and summarized by robust multi-array average (RMA) method (Irizarray et al., 2003). To identify differentially expressed genes between normal and cancer SP cells, CSC1 cancer (3447) SP cell vs. normal SP cell and CSC2 cancer (4346) SP cell and normal SP cell were compared. In both comparisons, Fs statistics (Cui et al., 2005), a modified F statistics with a shrinkage estimate of variance estimation were calculated by MAANOVA (Wu, 2002). P-values were derived by 1000 permutation and the false discovery rate (q-value) was calculated to correct for the multiple hypothesis testing problem (Storey, 2002). Differentially expressed genes between cancer and normal SP cells were selected by two criteria; genes having less than 0.05 q-value and more than 2.6 (1.5 log2) fold change in both comparisons (CSC1 vs. Normal and CSC2 vs. Normal). Biological relationships amongst differentially expressed genes were studied by Ingenuity Systems software (which can be used and found by one of ordinary skill in the art at world-wide web site: “ingenuity-dot-com”).

### Example 1

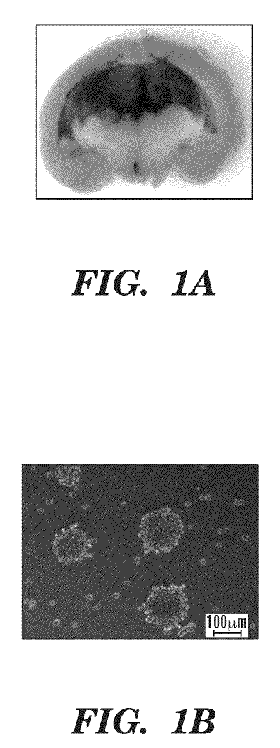

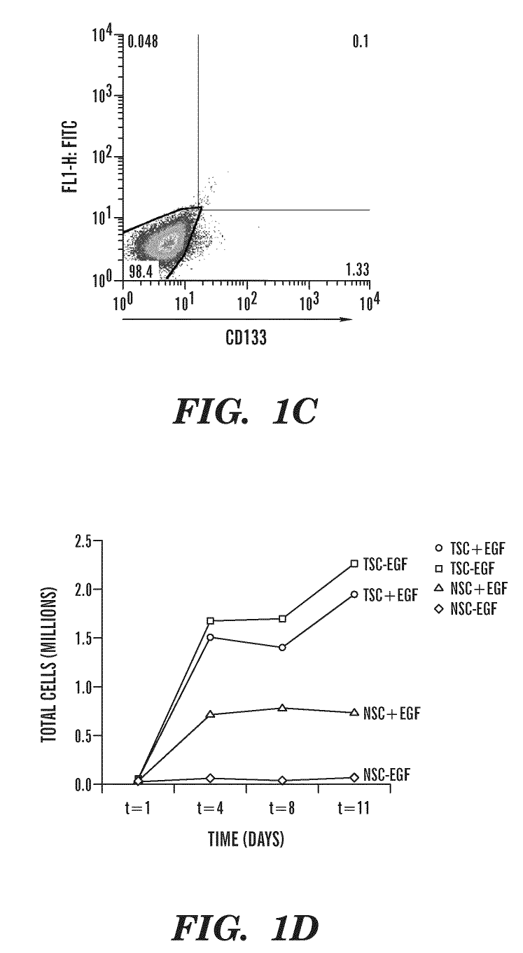

To identify CSC in mouse cancer models, the inventors used a transgenic mouse model of oligodendroglioma in which the S100β-promoter drives the expression of the verbB gene (10). In the Trp53−/− (p53−/−) mutant background, S100β-verbB;p53−/− animals develop “spontaneous”, oligodendrogliomas (FIG. 1A) that faithfully recapitulate the human disease at high frequency. Unlike transplanted neoplasms from xenografted human brain cancer cell lines, brain tumors in S100β-verbB;p53−/− animals are highly infiltrative, aggressive oligodendrogliomas with extensive vascularization and necrosis (data not shown). Hence, this animal model (maintained on an inbred genetic background) provides an excellent opportunity to test whether mouse primary brain tumors contain cancer stem cells, like human brain tumors and importantly, to determine the molecular differences between normal and cancer stem cells of the nervous system.

To identify distinguishing cellular phenotypes of normal and cancer stem cells, the inventors isolated and characterized normal neural stem cells (neurospheres) and brain cancer stem cells (tumorspheres) from S100β-verbB;p53−/− mice and their littermate controls (FIG. 1B). These tumorspheres were discovered to grossly resemble normal neurospheres (data not shown) isolated from the subventricular zone as well as previously described cancer stem cells isolated from human patients (11-15). However, tumorspheres differed from normal neurospheres in 3 important aspects. 1) Normal neural stem cells (NSC) absolutely require the growth factor, EGF, for growth while cancer stem cells (CSC) from S100β-verbB;p53−/− mice grew in the absence of added growth factors or serum, demonstrating growth factor independence (see FIG. 1D). 2) NSC formed round even edged spheres while CSC were more loosely attached, exhibiting an uneven periphery (data not shown). 3) NSC never initiated tumors when injected into mice while CSC consistently formed tumors (Table 1).

Defining features of stem cells are their multipotentiality and self-renewal capacity. To test whether tumorspheres are capable of self-renewal, the inventors plated dissociated single cells at a clonal density (1 cell/μl). Approximately 15% of the cancer cells gave rise to secondary spheres (data not shown), indicating that these are self-renewing cells. This capacity for self-renewal is maintained even after 25 passages in vitro. Multipotentiality of CSC is demonstrated by the inventors observation that they gave rise to cells expressing markers of all neural lineages, i.e; NG2+ (oligodendrocytes), GFAP+ (astrocytes), and Tubb3+ (neurons) expressing cells when cultured in differentiation promoting conditions (FIG. 1F,G,H). However, the numbers of tumorsphere derivatives expressing neuronal and astrocytic markers were greatly reduced when compared to NSC (not shown), and the morphology of these cells was abnormal, consistent with their cancer origin. The inventors discovered, of oligodendroglioma-derived cells, greater than 90% of the tumorsphere cells expressed premature oligodendrocyte markers such as NG2 and OLIG2 even at the time of plating (data not shown). In addition, unlike NSC, a fraction of CSC continued to proliferate even in differentiation promoting conditions, consistent with their transformed state. To examine clonal stem cells, the inventors isolated and characterized individual clones of CSC and observed similar results.

TABLE 1
Cancer stem cell and normal neural stem cell injections in NOD-SCID
mice. Number of tumors observed in injected animals by harvest date
is shown.

| | | # of cells | # of animal | | |
|---|---|---|---|---|---|
| Cells injected | Genotype | injected | with tumors | Harvest date | |
| 3447 tumorsphere cells | VerbBp53+/− | 2 × 10\hat{ }5 | 3/3 | 20 | days |
| | | 1000 | 3/3 | 25-42 | days |
| | | 500 | 3/3 | 35-42 | days |
| | | Single sphere | 4/4 | 28 | days |
| 4346 tumorpshere cells | VerbBp53−/− | 3.5 × 10\hat{ }5 | 3/3 | 20 | days |
| 3143 tumorpshere cells | VerbBp53−/− | 1 × 10\hat{ }5 | 2/2 | 37-52 | days |
| 2670 tumorpshere cells | VerbBp53−/− | 1 × 10\hat{ }5 | 3/3 | 30 | days |
| 1394 tumorpshere cells | VerbBp53+/− | 1 × 10\hat{ }5 | 5/5 | 37 | days |
| 2649 tumorpshere cells | VerbBp53+/− | 1 × 10\hat{ }5 | 5/5 | 37 | days |
| VerbB; p53 neurosphere | VerbBp53−/− | 1 × 10\hat{ }5 | 0/2 | 90 | days |
| cells | | | | | |
| | | Single sphere | 0/4 | 90 | days |

### Example 2

Another defining characteristic of cancer stem cells is that they initiate a tumor when transplanted in a suitable host. Tumorsphere cells isolated from multiple independent tumors generate neoplasms that resemble the original tumor 100% of the time when injected into NOD.CB17-Prkdc^{scid}/J (NOD-SCID) immune-deficient mice or C57BL/6J wildtype mice (Table 1). Even injections of individual tumorspheres (consisting of approximately 100-200 cells) consistently gave rise to rapid tumor formation (less than 4 weeks), suggesting that each tumorsphere contains at least one cancer initiating cell (shown for 3447 in Table 1). Histological analysis and molecular marker expression (data not shown) show identical expression patterns between primary and secondary (injected) tumors. These tumors can be serially transferred through animals over multiple passages (>6 passages), demonstrating in vivo self-renewal ability. At each passage, tumorspheres were isolated and characterized. These tumorspheres gave rise to new tumors when injected, and their cellular characteristics, in terms of growth rate and marker gene expression, were identical to the original tumorsphere (not shown).

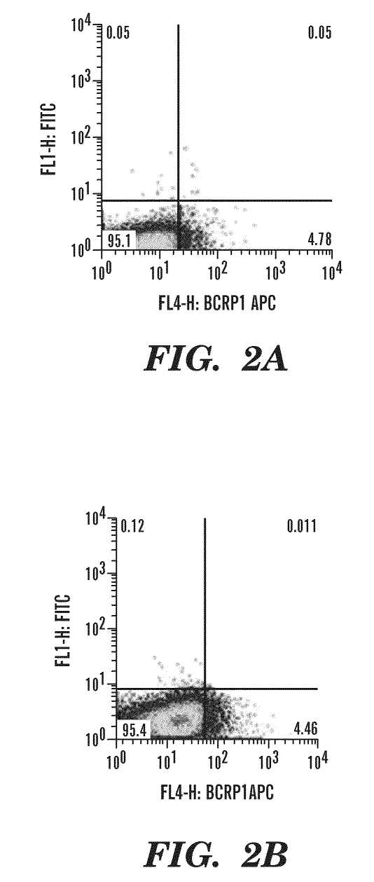

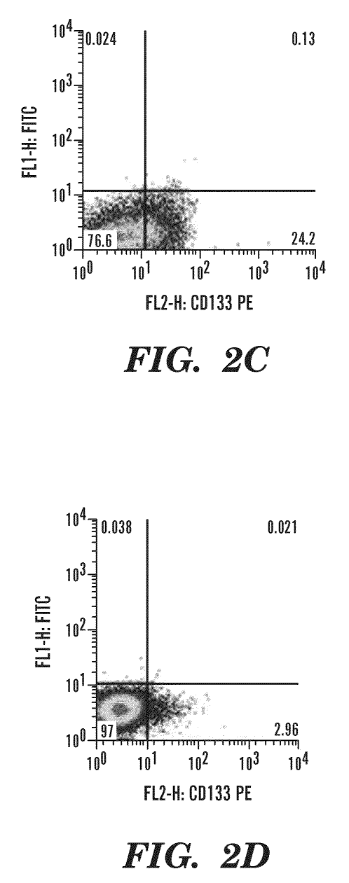

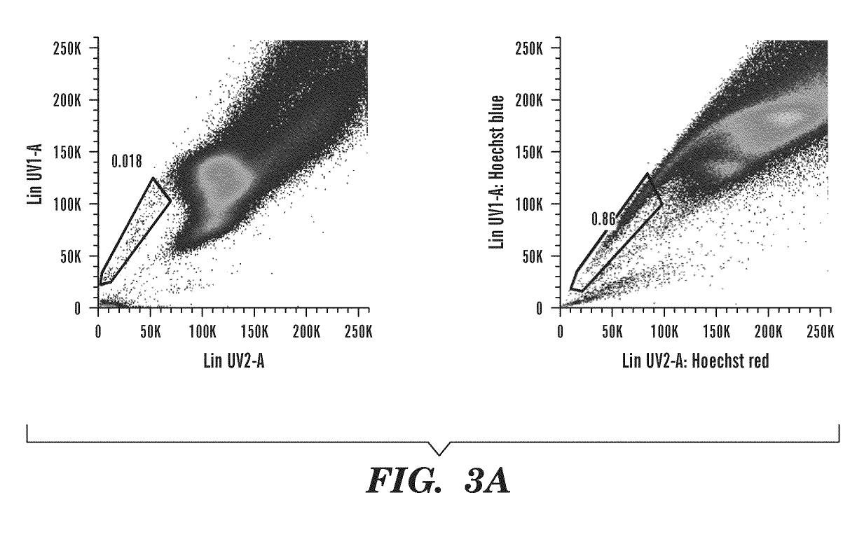

To determine whether the tumors contain cells expressing stem cell markers, the inventors examined expression patterns of CD133, BCRP1/ABCG2, SSEA1 and SOX2. High levels of SOX2, a neural stem cell marker, were found in tumors (FIG. 1C: CD133). Interestingly, cells in the leading edge of invasive streams express high levels of Sox2 (data not shown). Sox2 may not be a unique marker for cancer stem cells since the majority of the cancer cells express Sox2, in contrast to normal brain (data not shown). ABCG2/BCRP1 was expressed in 2-5% of the normal and tumor sphere cells (FIG. 2). The inventors observed weak but consistent expression of CD133 in approximately 1-3% of tumorsphere cells, in contrast to approximately 20-25% CD133+ cells in neurosphere cultures. Interestingly, CD44 and c-Kit, stem cell markers in other tissues, were expressed in 60-80% cells in both tumorsphere and neurosphere cultures (not shown), consistent with the idea that CD44 is a marker of glial progenitors rather than stem cells (16).

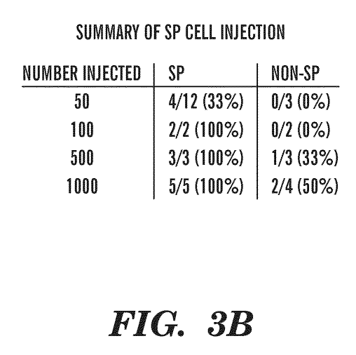

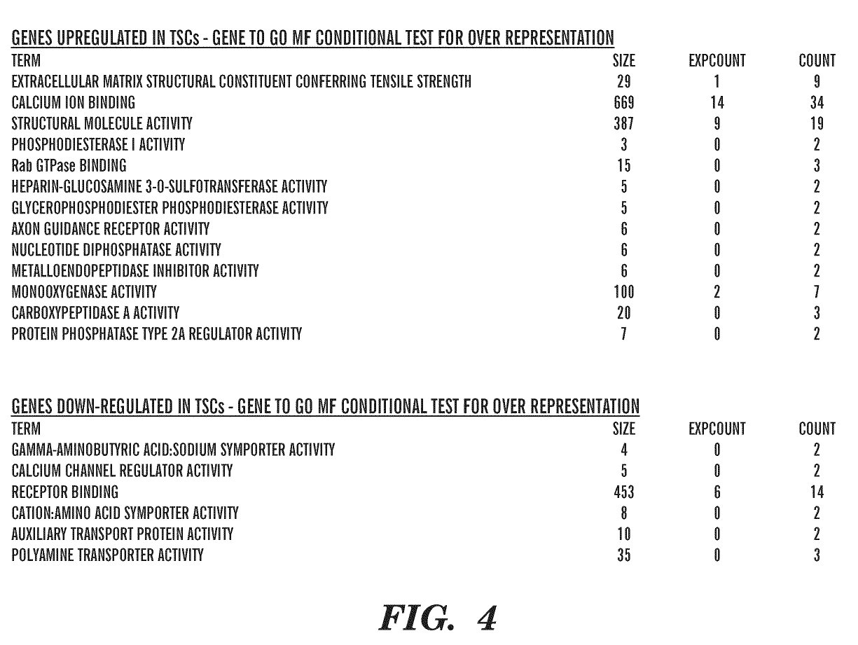

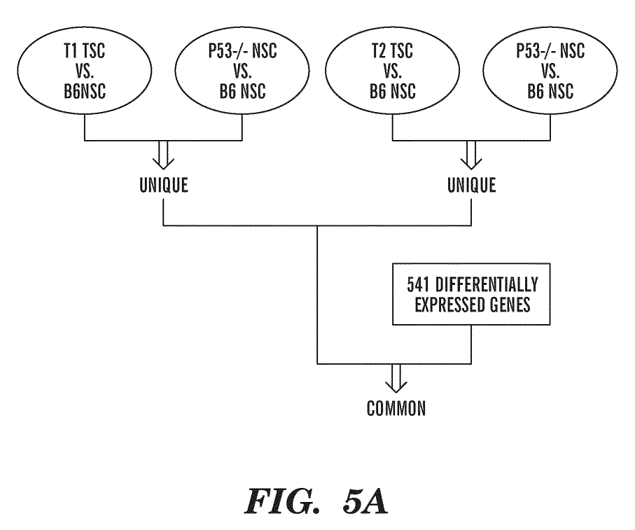

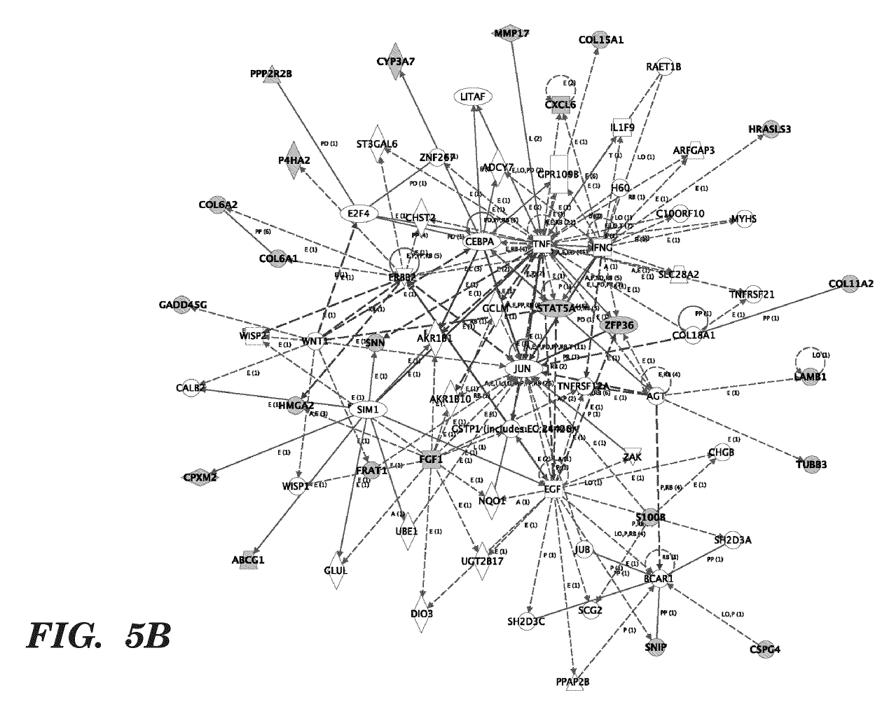

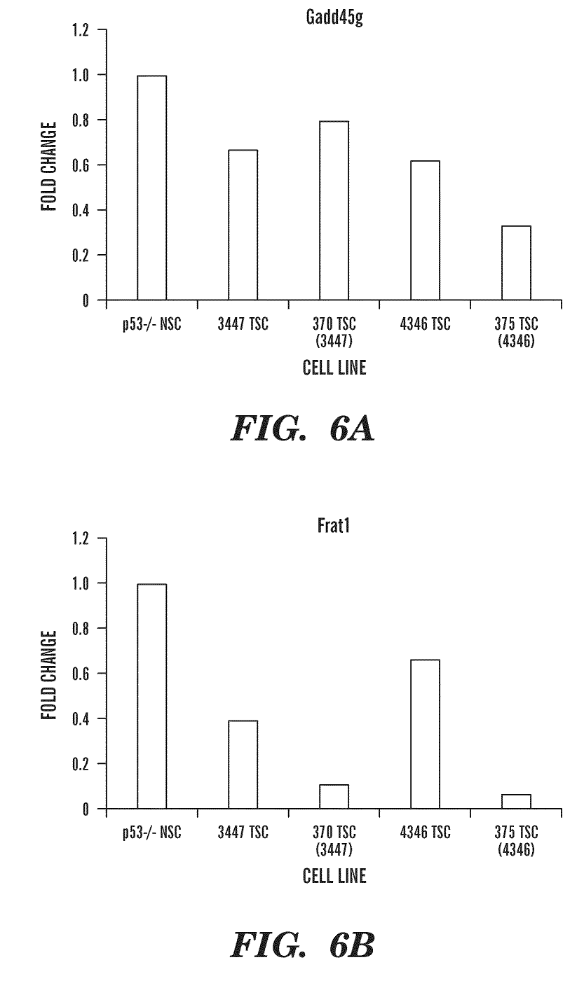

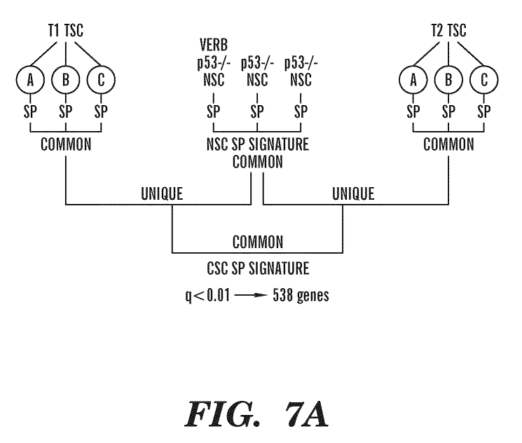

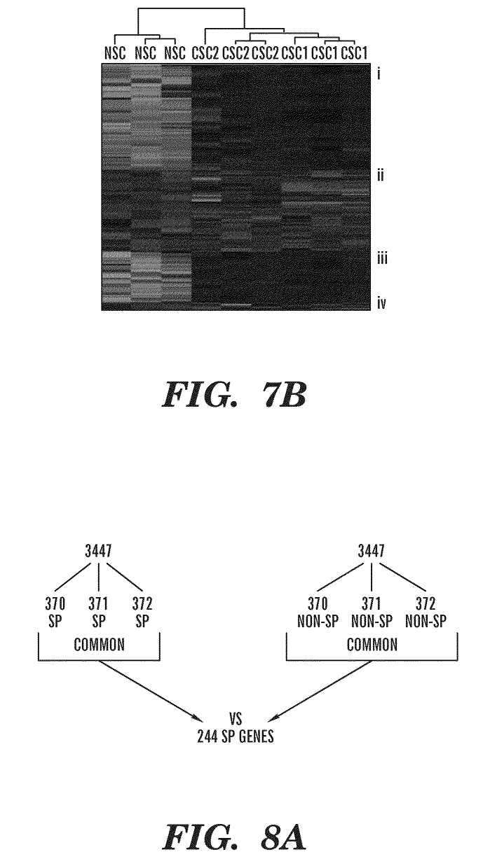

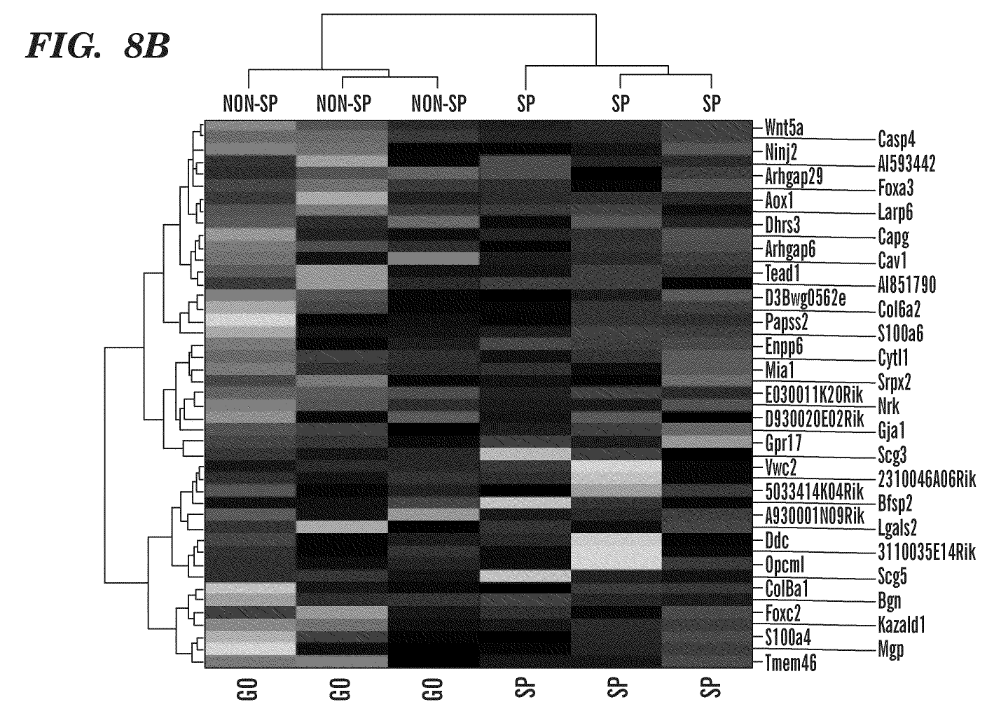

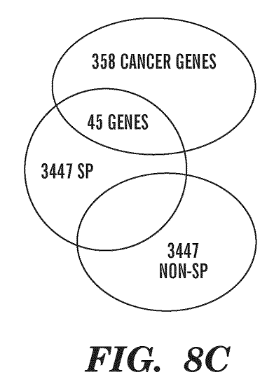

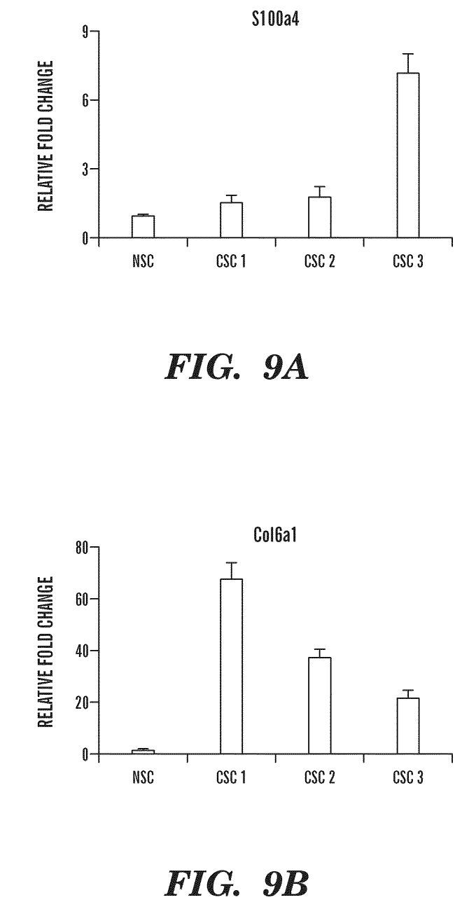

To determine whether cancer-initiating cells are enriched in a specific subpopulation of cells, the inventors sorted for the side-population (SP) cells using normal bone marrow as the control (data not shown). SP cells appear negative for the nuclear dye Hoechst 33342 and this staining method has been previously used by others to isolate normal and cancer stem cells from multiple tissue types (17-22). The inventors isolated and injected SP and non-SP cells from the same tumorsphere cultures and compared their tumor-initiating abilities. As few as 50 SP cells initiated a rapid tumor growth in ˜30% of host animals, while 500-1000 non-SP cells were required to give rise to tumors with similar frequency (FIG. 3 and Table 2), suggesting that tumor-initiating cells are enriched in the SP population. SP cells also retain self-renewal ability better than non-SP cells, suggesting that CSCs are enriched in the SP population in this cancer model. These observations indicate that there are cancer stem cells in spontaneous mouse tumors, suggesting that the etiology of brain cancer at the cellular level is similar between mouse and human.

TABLE 2
SP vs non-SP cell injection comparison. Numbers of animals giving rise
to tumors by 60 days post injection. In parenthesis are percentages of
injected animals developing tumors. A summary from 4 independent
FACS sort and injections.

| | Animals injected with | Animals injected with |
|---|---|---|
| # of cells injected | SP cells | non-SP cells |
| 50 | 4/12 (33%) | 0/3 (0%) |
| 100 | 2/2 (100%) | 0/2 (0%) |
| 500 | 3/3 (100%) | 1/3 (33%) |
| 1000 | 5/5 (100%) | 2/4 (50%) |

### Example 3

For future development of targeted therapeutics against cancer stem cells, understanding the molecular difference between cancer stem cells and normal stem cells and non-stem cancer cells is absolutely essential. To identify genes that distinguish cancer stem cells from normal stem cells, SP and non-SP cells were isolated from neurospheres (derived from S100β-verbB;p53−/− and p53−/− control animals) and tumorspheres (derived from two independent brain tumors in S100β-verbB;p53−/− animals) (data not shown). SP and non-SP cells were directly sorted into a lysis buffer at the time of sorting to fix the both cellular state as well as genetic background in this transcriptome comparison. Labeled probes were prepared from these cDNA and hybridized onto MOUSE430_{—}2 Affymetrix GeneChip arrays. 538 significantly differentially expressed genes showed consistent gene expression differences between the two independent cancer SP and normal SP populations (q-value<0.05 and log2 fold change>1.5) (data not shown). 345 genes were over-expressed and 193 genes were under-expressed in both cancer derived SP cells compared to normal SP cells (Table 6). Unsupervised clustering of the data set comparing cancer and normal SP cells clearly segregated the cancer SP cells and normal SP cells, indicating profound gene expression differences (data not shown). For example, there were significant expression level changes in components of the Wnt and Notch signaling pathways (DKK3, Wifl, Fzdb, Wnt7a, Wnt5, Hey2, and HESL), suggesting deregulation of these pathways in cancer stem cells (Table 8).

### Example 4

To filter the gene list for stem cell relevant genes, the inventors examined genes that are differentially expressed between cancer initiating (SP) and non-initiating (non-SP) cells from the same tumorsphere cultures (data not shown). The inventors first identified 244 genes whose fold change between cancer SP vs. cancer non-SP is greater than 2 fold. This list included Nanog and Myc, which showed higher levels of expression in SP cells compared to non-SP cells (not shown), consistent with higher self-renewal abilities of SP cells in vitro. When the inventors compared the two gene lists (cancer SP vs. normal SP and cancer SP vs cancer non-SP), 46 genes were common to both gene lists (data not shown). The list of 46 differentially expressed genes are referred to herein as the “CSC biomarker” or “cancer stem cell biomarker” list and is a list of genes for cancer stem cells, such as brain cancer stem cell gene signature. An unsupervised clustering analysis segregated non-SP and SP samples (data not shown). Notably, 23 of the 46 genes encode either secreted or membrane proteins and extracellular matrix components (Table 3), demonstrating that a major distinguishing feature of cancer initiating cells from normal stem cells and non-stem cancer cells is their ability to interact with their microenvironment.

This list also includes many genes with known function in cancer, such as Cav1, S100A4, and S100A6. In particular, S100A4/Metastasin and S100A6/Calcyclin Ca+ binding proteins, which have demonstrated roles in metastasis in other solid tumors (23, 24) were highly expressed in cancer SP cells (data not shown). To test the hypothesis that S100A6 and S100A4 expression is associated with brain cancer stem cells, the inventors examined tumors arising from intracranial xenografts of primary human GBM and human brain cancer cell lines (DAOY, SF767, and HOG). S100A6 expressing cells were found in a small subset of tumor cells, often positioned in the periphery of the tumor (data not shown). While this observation is consistent with S100A6 being a potential cancer stem cell marker, whether S100A6+ cells are brain cancer stem cells in human remains to be directly tested.

TABLE 3
46 CSC biomarkers: cancer stem cell gene signature Average fold
change between normal SP and cancer SP from the microarray analysis are indicated in
parenthesis. Genes that were validated by the inventors using RT-PCR are shown in bold. The
value is the difference in expression as compared to the reference expression level (which is
normalized to 100%). For clarity purposes only, a 2-fold (2.0X) difference refers to 200% of the
reference expression level, and a 3-fold (3.0X) difference refers to 300% of the reference
expression level etc. Similarly, a 0.3-fold (0.3X) difference refers to a 30% expression level of
the reference expression level (i.e. a 70% decrease), or a 0.1-fold (0.1X) difference refers to a
10% expression level of the reference expression level (i.e. a 90% decrease), etc.

| Category | N = 46 | Genes |
|---|---|---|
| Extracellular | 9 | Mgp(99.5X), Bgn(102X), Kazald1(19X), Col6a1(15.7X), |
| | | Scg5 (8.5X), Col6a2(14.6X), Vwc2(4.2X), Mia1(5.9X), Scg3 |
| | | (0.2X) |
| Membrane/cell signaling | 12 | Tmem46(6.5X), Opcml (6.2X), Ninj2(8.5X), Enpp6 (6.3X), |
| | | Cav1(15.7X), S100a6(31.5X), S100a4(14.7X), Gpr17(8.7X), |
| | | D930020E02Rik (0.1X), Gja1(0.1X), 5033414K04Rik |
| | | (0.2X), Kcna4 (12.9X) |
| Secreted | 3 | Cytl1(16.1X), AI851790 (0.2X), Wnt5a (0.2X), |
| DNA/RNA binding | 5 | Foxc2(32.6X), Foxa3(10.6X), A930001N09Rik(4.5X), Larp6 |
| | | (5.4X), Tead1 (0.3X) |
| Kinase/phosphatase/GTPase | 4 | Papss2 (39.7X), Arhgap6 (13.2), D3Bwg0562e (6.2X), |
| | | Arhgap29 (0.3X), |
| Apoptosis | 1 | Casp4(12.4X) |
| Novel genes | 4 | 3110035E14Rik (12.1X), 2310046A06Rik (8.2X), |
| | | E030011K20Rik (5X), Ai593442 (0.1X) |
| Others | 7 | Ddc(20.4X), Lgals2 (11.7X), Capg(15X), Srpx2 (7.4X), |
| | | Dhrs3 (4.1X), Bfsp2 (15.1X), Aox1 (0.3X), ID4 |

The inventors examined other genes on the 538 cancer-SP gene list that are associated with metastasis in other cancer types or migration of maturing neurons. Specifically, the inventors examined Snail2/Slug and Slit3 by RT-PCR (data not shown). Analysis of multiple independent S100β-verbB;p53−/− tumors confirmed significantly higher levels of Snail2 and Slit3 expression in tumorspheres compared to neurospheres (data not shown). Interestingly, SNAIL2/SLUG is not normally expressed in the brain. These observations demonstrate that infiltrative brain cancer cells may activate ectopic pathways to mediate local invasion, for example by employing the same pathways used by metastatic breast cancer cells.

As disclosed herein, the inventors demonstrate that cancer stem cells exist in mouse models, which supports the generality of cancer stem cells. The inventors have demonstrated, in a model of oligodenodroglioma, cancer-initiating cells are enriched in the side-population (SP). Kondo et al. have shown that cancer-initiating cells of the C6 rat glioma cell line are enriched in the SP (18), and Kim and Morshead have shown that normal neural stem cells are enriched in the SP population in NSC cultures (19). Prospective identification of SP cells as cancer stem cells from a mouse tumor allowed us to isolate and compare normal and cancer SP cells for a comparative transcriptome analysis. The inventors have demonstrated herein, two major variables that complicate other similar studies, namely genetic background and cellular heterogeneity, have been eliminated to reduce the background noise level. This was critical in limiting the number of genes that are differentially expressed in cancer stem cells.

From the cancer stem cell gene signature analysis, the inventors demonstrate a major difference between cancer stem and normal stem cells is the ability of cancer stem cells to interact with the surrounding microenvironment. In addition to S100A4 and S100A6, Col6A1 and Col6A2 are also more highly expressed in cancer SP cells compared to normal SP and non-stem cancer cells (data not shown). S100A4 and Col6A1 have been identified in two independent screens that were aimed to identify genes that are differentially expressed in hair follicle stem cells (25, 26). S100A6 is expressed in the ependymal layer in the normal brain (not shown), where CD133, Sox2, and Nestin (markers of normal stem cells) are also expressed.

TABLE 4
Table 4. List of CSC Biomarkers and fold change as compared to reference level
of expression:

| SEQ | Mouse | | | | |
|---|---|---|---|---|---|
| ID NO | Symbol | FoldChgD-N | Fold ChgI-N | Fold ChgI-D | Mouse Name |
| 1 | 2310046A06Rik | | | | RIKEN cDNA 2310046A06 |
| | | | | | gene |
| 2 | 3110035E14Rik | | | | RIKEN cDNA 3110035E14 |
| | | | | | gene |
| 3 | A930001N09Rik | | | | RIKEN cDNA A930001N09 |
| | | | | | gene |
| 4 | AI593442 | | | | expressed sequence AI593442 |
| 5 | AI851790 | | | | expressed sequence AI851790 |
| 6 | Aox1 | −4.16986304 | −4.46914855 | −1.06437018 | aldehyde oxidase 1 |
| 7 | Arhgap29 | 1.591072968 | 1.72907446 | 1.07922824 | Rho GTPase activating protein |
| | | | | | 29 |
| 8 | Arhgap6 | 3.249009585 | 3.36358566 | 1.03526492 | Rho GTPase activating protein 6 |
| 9 | Bfsp2 | −1.68179283 | −1.65863909 | 1.01395948 | beaded filament structural |
| | | | | | protein 2, phakinin |
| | Bfsp2 | −1.67017584 | −1.71713087 | −1.02101213 | beaded filament structural |
| | | | | | protein 2, phakinin |
| | Bfsp2 | 2.265767771 | 2.29739671 | 1.00695555 | beaded filament structural |
| | | | | | protein 2, phakinin |
| 10 | Bgn | 11.15794933 | 17.8765942 | 1.59107297 | Biglycan |
| 11 | Capg | | | | capping protein (actin |
| | | | | | filament), gelsolin-like |
| 12 | Casp4 | −1.36604026 | −1.38510947 | −1.01395948 | caspase 4, apoptosis-related |
| | | | | | cysteine peptidase |
| | Casp4 | 4.823231311 | 4.65893435 | −1.02811383 | caspase 4, apoptosis-related |
| | | | | | cysteine peptidase |
| 13 | Cav1 | −8.5741877 | −19.5622444 | −2.26576777 | caveolin, caveolae protein 1 |
| 14 | Col6a1 | 5.205367422 | 5.38893431 | 1.03526492 | procollagen, type VI, alpha 1 |
| | Col6a1 | 8.876555777 | 9.06307108 | 1.02101213 | procollagen, type VI, alpha 1 |
| | Col6a1 | 38.8542363 | 57.6800296 | 1.47426922 | procollagen, type VI, alpha 1 |
| 15 | Col6a2 | −10.9283221 | −10.6294865 | 1.02101213 | procollagen, type VI, alpha 2 |
| | Col6a2 | −2.15845647 | −1.18920712 | 1.80250093 | procollagen, type VI, alpha 2 |
| 16 | Cytl1 | | | | cytokine like 1 |
| 17 | D3Bwg0562e | | | | DNA segment, Chr 3, Brigham |
| | | | | | &Women's Genetics 0562 |
| | | | | | expressed |
| 18 | D930020E02Rik | | | | RIKEN cDNA D930020E02 |
| | | | | | gene |
| 19 | Ddc | | | | dopa decarboxylase |
| 20 | Dhrs3 | 1.474269217 | 1.04971668 | −1.40444488 | Dehydrogenase/reductase |
| | | | | | (SDR family) member 3 |
| 21 | E030011K20Rik | | | | RIKEN cDNA E030011K20 |
| | | | | | gene |
| 22 | Enpp6 | | | | Ectonucleotide |
| | | | | | pyrophosphatase/phosphodiesterase 6 |
| 23 | Foxa3 | | | | forkhead box A3 |
| 24 | Foxc2 | 4.9588308 | 5.0280535 | 1.01395948 | forkhead box C2 |
| 25 | Gja1 | −10.1260528 | −11.3924016 | −1.11728714 | gap junction membrane |
| | | | | | channel protein alpha 1 |
| | Gja1 | −2.84810039 | −6.23331664 | −2.17346973 | gap junction membrane |
| | | | | | channel protein alpha 1 |
| 26 | Gpr17 | 8.397733469 | 8.51496146 | 1.00695555 | G protein-coupled receptor 17 |
| 27 | Kazald1 | 1.635804117 | 1.5691682 | −1.03526492 | Kazal-type serine peptidase |
| | | | | | inhibitor domain 1 |
| 28 | Kcna4 | −4.16986304 | −3.97236998 | 1.04246576 | potassium voltage-gated |
| | | | | | channel, shaker-related |
| | | | | | subfamily, member 4 |
| 29 | Larp6 | | | | La ribonucleoprotein domain |
| | | | | | family, member 6 |
| 30 | Lgals2 | | | | lectin, galactose-binding, |
| | | | | | soluble 2 |
| 31 | Mgp | −1.35660433 | −3.03143313 | −2.21913894 | matrix Gla protein |
| 32 | Mia1 | −4.19886673 | −5.81589007 | −1.37554182 | melanoma inhibitory activity 1 |
| 33 | Ninj2 | | | | ninjurin 2 |
| 34 | Opcml | 1.464085696 | 1.4240502 | −1.02101213 | opioid binding protein/cell |
| | | | | | adhesion molecule-like |
| | Opcml | 2.566851795 | 2.62078681 | 1.02101213 | opioid binding protein/cell |
| | | | | | adhesion molecule-like |
| 35 | Papss2 | 2.67585511 | 2.41161566 | −1.10190512 | 3′-phosphoadenosine 5′- |
| | | | | | phosphosulfate synthase 2 |
| 36 | S100a4 | −4.89056111 | −3.70635225 | 1.3103934 | S100 calcium binding protein |
| | | | | | A4 |
| 37 | S100a6 | | | | S100 calcium binding protein |
| | | | | | A6 (calcyclin) |
| 38 | Scg3 | | | | secretogranin III |
| 39 | Scg5 | | | | secretogranin V |
| 40 | Srpx2 | −1.67017584 | −1.34723358 | 1.2397077 | sushi-repeat-containing |
| | | | | | protein, X-linked 2 |
| 41 | Tead1 | −29.040613 | −28.6408023 | 1.00695555 | TEA domain family member 1 |
| 42 | Tmem46 | | | | Transmembrane protein 46 |
| 43 | Vwc2 | | | | von Willebrand factor C |
| | | | | | domain containing 2 |
| 44 | Wnt5a | 1.658639092 | 1.8276629 | 1.0942937 | von Willebrand factor C |
| | | | | | domain containing 3 |
| | Wnt5a | 1.931872658 | 1.93187266 | 0.99971368 | von Willebrand factor C |
| | | | | | domain containing 4 |

The inventors demonstrate the isolation of cancer stem cells from a mouse model of brain cancer, demonstrating they express oligodendroglioma markers from a S100β-verbB;p53−/− animal, and grow as tumorspheres in serum-free medium (FIG. 1D). The inventors also demonstrate that neural stem cells grow as neurospheres in serum-free medium containing bFGF and EGF (FIGS. 1B and D). The inventors demonstrate different growth rates, as shown in FIG. 1D growth-curve comparing neurospheres and tumorspheres grown in the presence or absence of EGF, plated 1E5 cells on day 0. The inventors assessed self-renewal using an assay based on the percent of single cells giving rise to secondary spheres when plated at a clonal density of a parental (3447) and two clonally derived tumorspheres show self-renewal ability (data not shown). The inventors demonstrated that the tumorspheres induced to differentiate on coated cover slips for 1 day and 3 days (data not shown). The expression of NG 2 (early oligodendrocyte marker) was assessed, as well as GFAP (an astrocyte marker), PH3 (an M-phase proliferating cell marker), TUBB3 (neuronal marker) (data not shown).

The inventors demonstrate that transplanted tumors resemble the original tumor. The inventors demonstrated that primary and secondary (derivative of primary tumors injected into NOD-SCID mice) tumors stained with H&E expressed markers of oligodendroglioma (Olig2 and NG2) and stem cells (Sox2 in red and BCRP1. The inventors discovered that a primary tumor showing densely packed SOX2+ cells within tumor, compared to surrounding normal tissue, and that SOX2 expression in a normal brain in the ependymal layer and SVZ region, and invading cancer cells that express SOX2 demarcate the tumor boundary (data not shown). The inventors also demonstrated using transcriptome analysis of normal SP and cancer SP cells, and Hoechst 33342 staining of bone marrow control cells and tumorsphere cells, showing SP tail in gate (data not shown). The SP cells were purified from 6 tumorsphere cultures (biological triplicates derived from transplanting two independent primary tumors) and 3 independent normal neural stem cell cultures from two p53−/− and one S100β-verbB;p53−/− animal. Gene expression was analyzed on MOUSE430_{—}2 Affymetrix GeneChip. The inventors discovered 538 differentially expressed genes by comparing two independent cancer SP and normal SP cells with q-value<0.05 and log2 change>1.5 (“cancer genes”). Using unsupervised clustering of the 538 gene expression profile segregates into 4 groups i-iv, as disclosed in Table 7 for GO analysis of each group. The inventors also identified 244 “SP genes” using gene expression comparison between cancer SP and cancer non-SP cells from 3447 tumor derived lines. The inventors compared the “SP gene” list with the “cancer gene” list to identify common genes to identify a resulting common gene list, herein termed “cancer stem cell biomarkers” (also see Table 3), which consists of 46 genes which segregate when unsupervised clustering analysis was used.

The inventors then validated some of the differential gene expression using RT-PCR and differential protein expression using immunofluoresence microscopy. Using real-time RT-PCR analysis using RNA from normal (NSC) and 3 independent cancer stem cell cultures (CSC1, CSC2, and CSC3) of genes S100A4, Col6a1, Snail2 and Slit3 the inventors demonstrated a relative fold change to NSC, normalized to internal 18S levels (data not shown). Other genes validated by RT-PCR are listed in Table 8. The inventors further validated the genes using immunofluorescence analysis of DAOY, SF767 and HOG xenographed human brain cancer stem cells using an antibody against S100A6 show specific staining in cancer cells, and discovered that were on the periphery (data not shown) or invading cluster of cancer cells (data not shown). The markers used in the analysis include, S100A6, GFAP+ reactive host astrocytes in green and DAPI (data not shown).

The inventors also demonstrate that normal and cancer stem cells in the mouse mammary gland are different. They demonstrate Id4± and Id4−/− in mammary glands stained with carmin alum, as well as morphometric measurements of ductal length, diameter and number of branches, per gland (n=3) are different (data not shown). The inventors also discovered using FACS scan analysis of mammary tumorspheres with CD24 and CD49f, that in sister cultures derived from the same tumor, and split into two different culture conditions 2 days before analysis, some cells do not form tumors while other cells that are CD24+CD49f+ do form tumors (data not shown). The inventor also demonstrate that mammary tumorspheres for Id2 and Id4 expression, and determined Id2 and Id4 levels in tumorspheres isolated from Met− MMTV-neu and Met+ MMTV-PyMT mammary tumors, as well as Id4 expression levels in brain cancer stem vs. non-stem cells from same (data not shown).

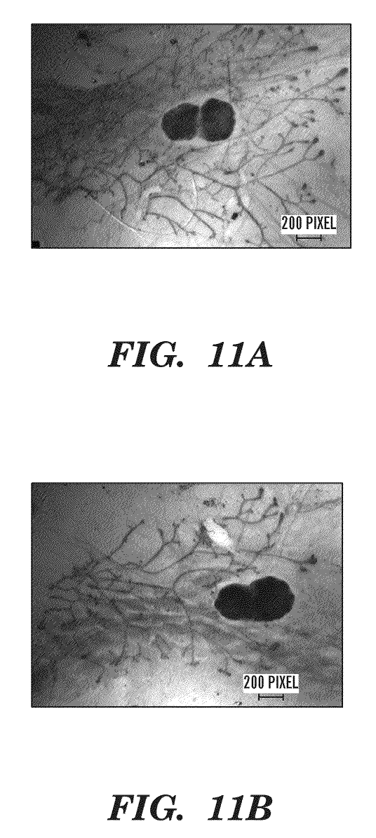

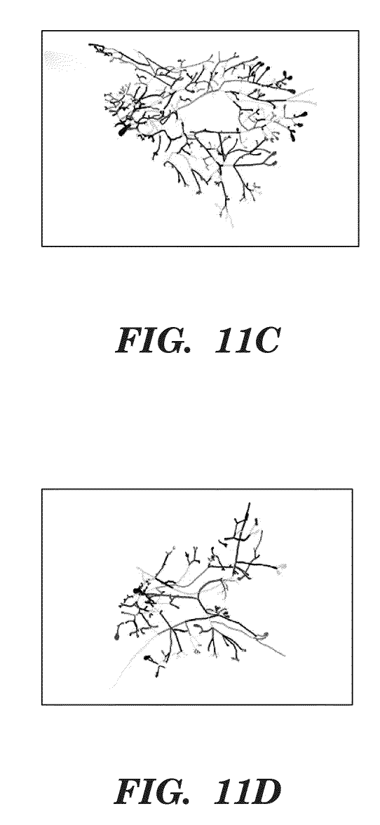

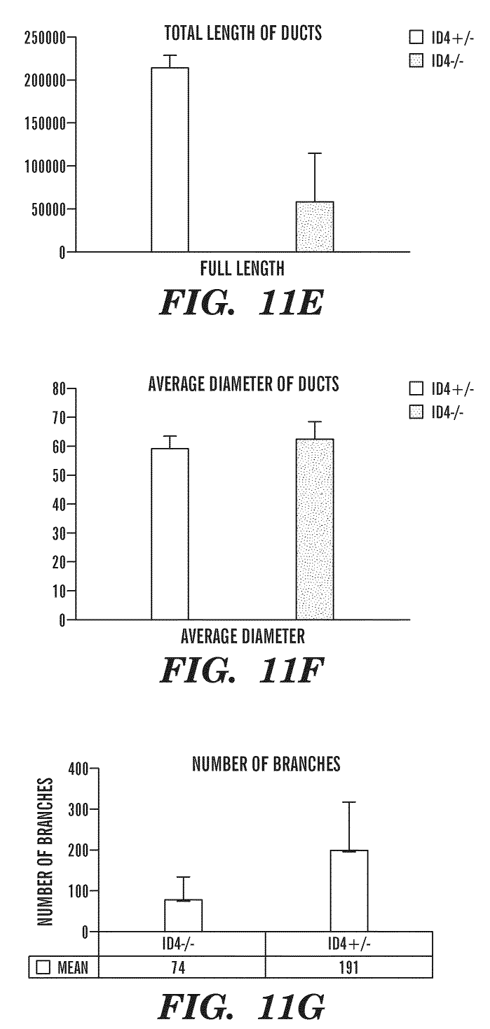

Id (Inhibitor of DNA binding or Inhibitor of Differentiation) genes are members of the basic helix-loop-helix family (bHLH) of transcription factors. Id4 is highly expressed in the developing nervous system and is required for expansion of the neuroepithelium and to inhibit precocious differentiation of neural stem cells (Yun, K., Mantani, A., Garel, S., Rubenstein, J. & Israel, M. A. Id4 regulates neural progenitor proliferation and differentiation in vivo. Development 131, 5441-8 (2004)). This in vivo analysis revealed that Id4 functions to either promote or inhibit cell cycle progression in a cell-context dependent manner, underscoring the importance of understanding the cellular context in which Id genes function. When analyzing Id4 null mice, the inventors have observed that Id4 is required for normal mammary gland development, as Id4−/− females have significantly delayed or compromised mammary gland development at puberty, as seen by the reduced ductal length and branching of the mammary gland (see FIG. 11).

### Example 5

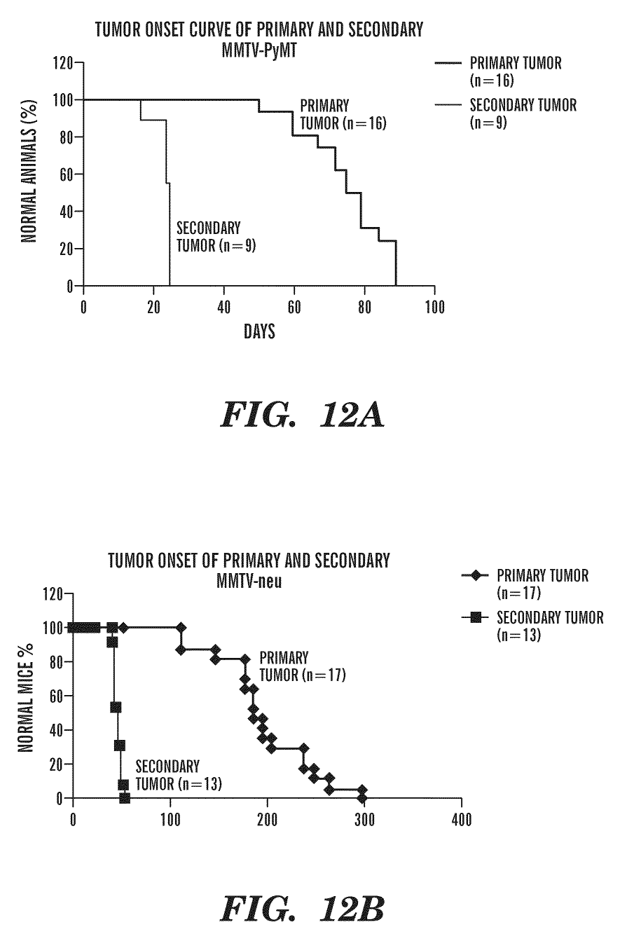

Analysis of the metastatic potential of the CSCs of the primary tumor. Tumorspheres were isolated and characterized (maintained in serum-free mammosphere culture conditions) from primary tumors of metastasis-bearing (Met+) MMTV-PyMT and non-metastasis bearing (Met−) MMTV-neu mice. Lungs of MMTV-neu mice were examined and no metastasis was observed at the time of harvest. When transplanted into the mammary fat pad of immunodeficient NOD-scid immune-deficient recipient mice, Met+ tumorspheres formed mammary tumors as well as lung metastasis within 1 month after injection (FIG. 12). Met− tumorspheres formed primary tumors in the mammary fat pad over an equivalent time course (FIG. 12), but these mice had not formed visible metastasis in the lung when harvested (at equivalent sizes of the mammary tumor and time course as Met+ tumors). This model can be used to isolate CSCs with different potential to metastasize.

### Example 6

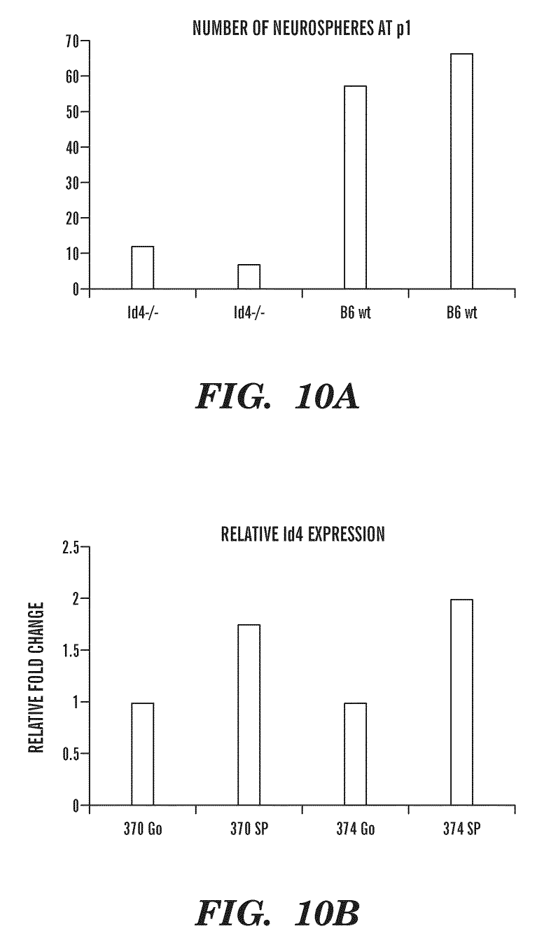

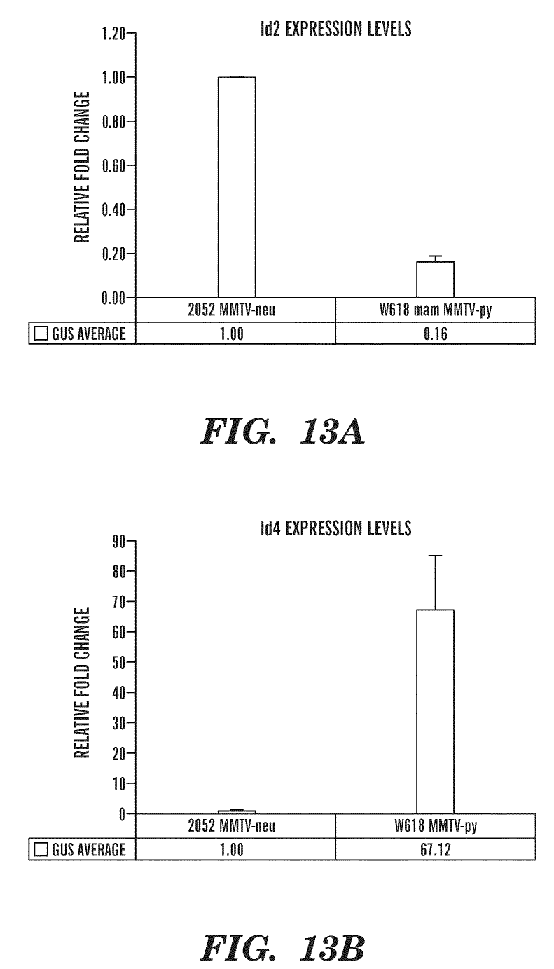

Id2 and I4 Expression in metastatic mammary tumorspheres. Id2 and Id4 levels were examined in mammary tumorspheres isolated from a Met− MMTV-neu and a Met+ MMTV-PyMT mice (as described above and in FIG. 12). A higher level of Id4 expression and lower level of Id2 expression in Met+ mammary tumorspheres, consistent with the proposed functions of Id2 (pro-differentiation) and Id4 (pro-proliferation) in mammary gland development was detected (see FIG. 10B and FIG. 13).

### Example 7

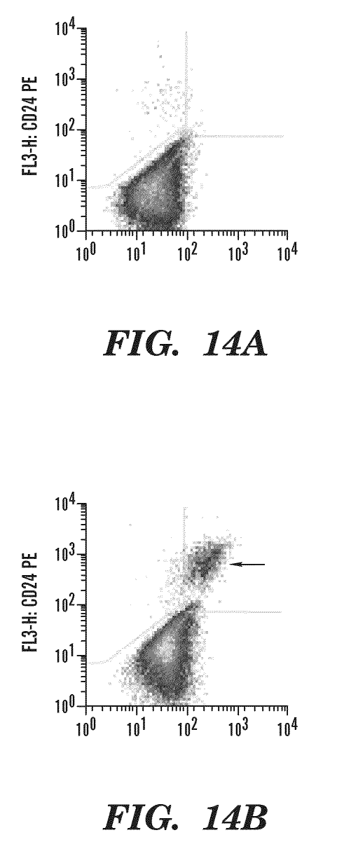

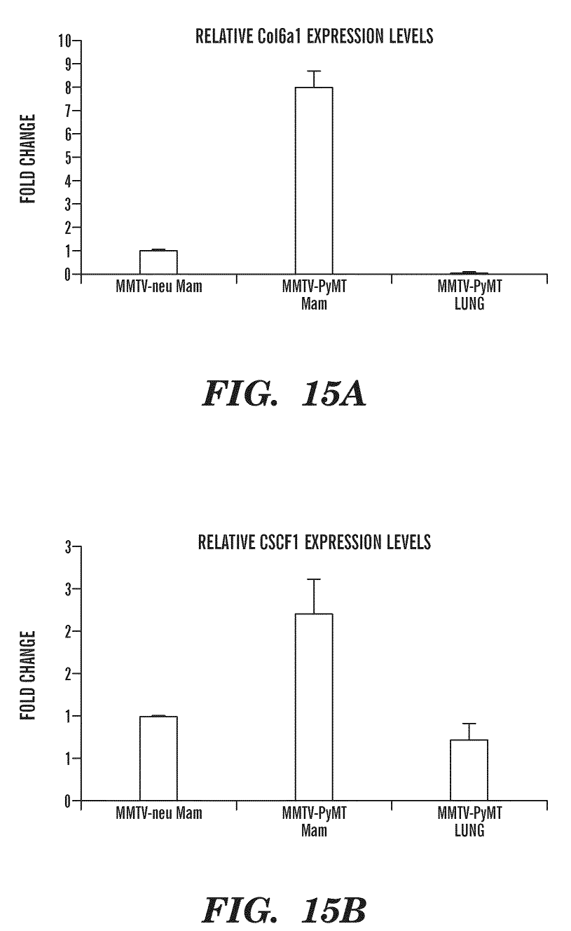

Analysis of the cell population in mammary tumorspheres. Tumorspheres were isolated from primary tumors of metastasis-bearing (Met+) MMTV-PyMT and non-metastasis bearing (Met−) MMTV-neu mice. Cells from the tumorspheres were cultured in serum-free mammosphere culture conditions and characterized by FACS for the cell surface markers CD24+ and CD49f+ (FIG. 14). CD24+CD49f+ cells were injected can be injected into NOD-scid immune-deficient recipient mice and there potential for tumor initiation and metastasis can be analyzed.

### Example 8

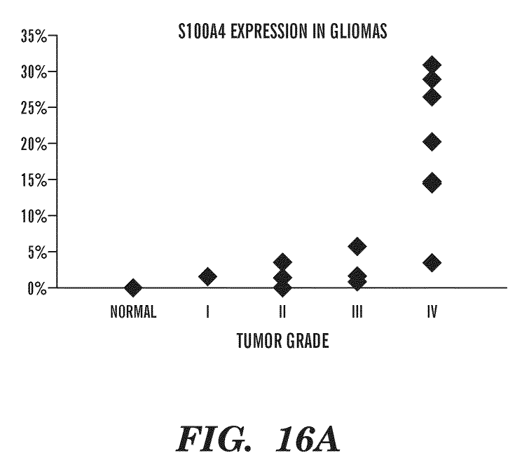

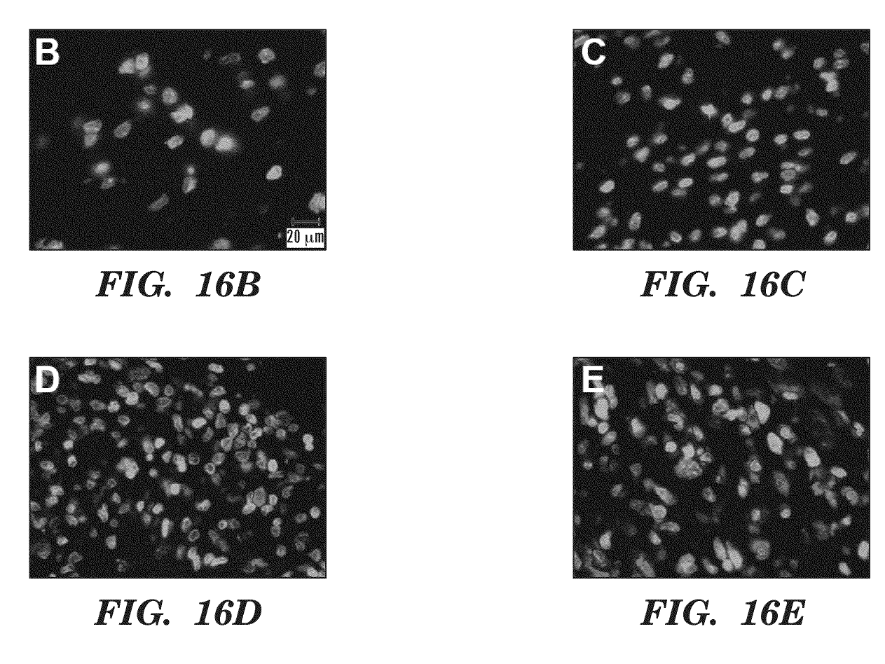

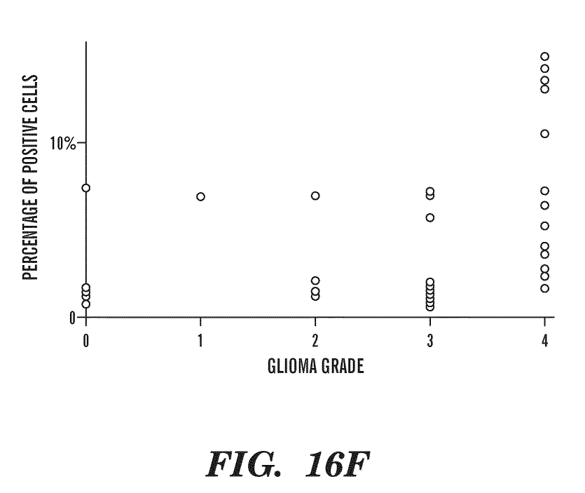

Analysis of human glioma tissue arrays. Tissue arrays containing 63 unique samples of human brain gliomas and normal cerebrum were stained with the S100A4 and S100A6 antibody using standard immunohistochemical techniques and a red fluorescent detection. The tissue was counterstained with DAPI to visualize the nuclei of the cells. In FIG. 16A shows a summary chart for S100A4+ cells in different grade gliomas and FIG. 16F for S100A6+. Representative images of normal cerebrum (FIG. 16B), well differentiated (FIG. 16C), poorly differentiated (FIG. 16D), and undifferentiated glioma tissue (FIG. 16E) are shown, which demonstrates that the most S100A4 and S100A6 positive cells can be identified in undifferentiated glioma tissue (FIG. 16E and FIG. 16F).

TABLE 6
Ingenuity networks generated by 345 genes over-expressed in cancer SP
(using q-value 0.05 and 1.5 log2 fold change) (A) and by 193 genes under-expressed in cancer
SP (using q-value 0.05 and 1.5 log2 fold change) (B). Genes in bold are on our gene list.

| | | | |
|---|---|---|---|
| Network | | | |
| id | Genes | # genes | Top functions |

A. Table 6A.

| | | | |
|---|---|---|---|
| 1 | ACSL1, ADAMTS5, AGC1, ASPN, CAV1, | 32 | Cellular Assembly |
| | CCND3, CDKN1A, COL11A1, COL11A2, | | and Organization, |
| | COL2A1, CTF1, FBXO7, FXYD1, GJB2, GNAO1, | | Cellular Function |
| | HOXA10, IAPP, MMP17, NKX2-2, P53CP, | | and Maintenance, |
| | PDGFRA, PPFIBP1, RECK, S100A1, S100A4, | | Connective Tissue |
| | S100A6, S100B, SNAI2, SREBF1, STAT5A, TFPI, | | Development and |
| | TIMP2, TIMP3, TUBB3, UCP2 | | Function |
| 2 | ABLIM3, ACLY, ARFGAP3, CAV1, CCND3, | 20 | Cancer, Cellular |
| | CD2, CDKN1A, CDKN2A, CXCL14, DECR1, | | Growth and |
| | EHD3, FGF2, FGFR3, GPNMB, GRIA1, GRIA3, | | Proliferation, |
| | HLA-A, HMGB2, IFNG, ITGB3, KCNK1, | | Cardiovascular |
| | KIAA1276, MDM2 (includes EG: 246362), MLANA, | | System |
| | NFYB, PCSK2, PDGFRA, RAB3C, SILV, SLIT3, | | Development and |
| | STAT5A, TCFL5, TENC1, TIMP2, TIMP3 | | Function |
| 3 | AP1S2, AP2B1, CAPG, CCND3, CCT5, CD82, | 20 | Cellular Assembly |
| | CD1D, CGI-38, CHI3L1, CHST6, CSPG4, EMP3, | | and Organization, |
| | ENPP1, FABP5, GP5, HSPA1B, IL3, IL4, IL1B, | | Cell-To-Cell |
| | LGALS2, MBP, MIA, MMP16, MYO1C, P2RX7, | | Signaling and |
| | PCSK2, PLB1, PLCD1, PRKCA, SCG5, SLC1A1, | | Interaction, |
| | SNCA, SPI1, TGM2, TIMP2 | | Cellular Growth |
| | | | and Proliferation |
| 4 | ADAM28, ANXA6, ARHGEF6, BGN, CAV1, | 20 | Cell Morphology, |
| | CCND3, CNTN1, CPXM2, DAG1, DDC, ELA1, | | Nervous System |
| | ELN, ENO3, FDPS, FGF19 (includes EG: 9965), | | Development and |
| | FLOT2, FOXP3, FYN, ID4, ITM2A, KRAS, MBP, | | Function, |
| | MCAM, MMP10, NRK, PAK3, PLP1, SCN1B, | | Developmental |
| | SGCA, SGCB, SGCD, SIM1, SREBF1, SYT9, | | Disorder |
| | THRB, UGT8 | | |
| 5 | AURKB, BAG1, BIRC5, CASP3, CASP4, CAV1, | 19 | Cancer, Cell |
| | CCND3, CD82, CDC42, CDKN1A, CYFIP2, | | Death, |
| | DOCK9, ELL, FBLN1, FMOD, FOXM1, HS3ST1, | | Neurological |
| | LAMA4, MET, P2RX4, PHLDA3, PKN1, PLXNB3, | | Disease |
| | POU4F1, RACGAP1, ROBO1, SLIT2, SNCA, | | |
| | SNCB, SREBF1, TP53, UBE2C, UNC5C, WASL, | | |
| | WASPIP | | |
| 6 | ABCG1, ACVRL1, AGC1, AXL, BYSL, CAV1, | 18 | Cell Morphology, |
| | CDKN1A, COL2A1, COL4A2, CRK, CTSK, | | Connective Tissue |
| | CXCL12, EFNA1, EPHA4, HOXA2, HSPG2 | | Development and |
| | (includes EG: 3339), IRF6, KRT8, KRT18, KRT19, | | Function, Cellular |
| | MMP11, NR1H2, NR4A1, PGCP, PKD1, PKN1, | | Assembly and |
| | PRELP, RHOA, ROCK1, STARD13, TGFB1, | | Organization |
| | TGM2, TRO, TROAP, UGDH | | |
| 7 | ADRA2A, ADRB3, AKT1, ARRB1, ATP1A2, | 17 | Lipid Metabolism, |
| | CAV1, CAV2, CCND3, CEBPA, CFD, CYP3A4, | | Small Molecule |
| | CYP3A5, CYP3A7, FOXA3, FOXC2, FXYD5, | | Biochemistry, |
| | INS1, MBTPS1, MICAL1, MYO5A, MYRIP, | | Cellular |
| | PDGFRA, PLIN, PSCD3, PTGER4, RAB27A, | | Development |
| | RAB27B, SEPT5 (includes EG: 5413), SNCA, SRC, | | |
| | SREBF1, STAT5A, STX4, SYT4, SYTL2 | | |
| 8 | ADIPOQ, AFP, ATBF1 (includes EG: 463), CAPN3, | 16 | Cancer, Cellular |
| | CAPN6, CAV1, EGF, EMB, FOS, FOXD1, GDF2, | | Growth and |
| | GIT1, GLI1, HAS2, HHIP, MYH10, NANOG, | | Proliferation, |
| | NDRG2, PALM2-AKAP2, POU5F1, PRKACA, | | Tissue |
| | PRKAR1A, PRKG2, RARG, RIMS1, SLC8A1, | | Development |
| | SNAP25, SNIP, SOX9, STAT5A, TIMP3, VIL2, | | |
| | WASF1, WIF1, WWP2 | | |
| 9 | ANKH, CAV1, CCND3, CDH11, CRABP2, | 15 | Organism |
| | CRYL1, CSNK1E, CTNNB1, DKK3, FGF1, | | Development, |
| | FREM2, GRIA4, GRIP1, GRIP2, HAPLN1, | | Cancer, Cell Death |
| | HOXA3 (includes EG: 3200), JARID1A, MGP, | | |
| | NCOA5, PPP2CA, PPP2CB, PPP2CBP, PPP2R4, | | |
| | PPP2R1A, PPP2R1B (includes EG: 5519), PPP2R2A, | | |
| | PPP2R2B, PPP2R2C, PPP2R3A (includes EG: 5523), | | |
| | PPP2R5B, RARA, S100A13, SPP1, VDR, WISP1 | | |
| 10 | ADAM9, ADAM10, ADAM12, ADAM17, ALDOA, | 15 | Cell Death, |
| | ANKS1B, APP, CDKN1A, CLDN1, CLDN2, | | Cellular |
| | CYP2J2, ENPP2, EPHB1, EYA2, G6PD, | | Movement, |
| | HERPUD1, HSPG2 (includes EG: 3339), IFI35, IL15, | | Skeletal and |
| | IL7R, JUN, M6PR, MST1, MSX1, MYOD1, PAX3, | | Muscular System |
| | PTPN3 (includes EG: 5774), S100B, SH3D19, | | Development and |
| | SH3GL3, SIX1, SLC12A2, SNCA, TIMP3, WNK4 | | Function |
| 11 | ARNT, C1QL1, CAV1, CCNA1, CCND3, | 15 | Tissue |
| | CDKN1A, COL9A1, COL9A2, COL9A3, DGKA, | | Development, |
| | E2F1, ETV4, F2R, FLT1, GDF2, GJB1, HES1, | | Cardiovascular |
| | HEY2, LPPR4, MMP12, NOTCH1, NR4A1, NRG2, | | System |
| | NRP1, NRP2, PLAG1, PLG, RBPSUH, RLBP1, | | Development and |
| | SEMA3A, SEMA3D, SEMA3E, STARD8, STK23, | | Function, Cellular |
| | VEGF | | Movement |
| 12 | ACHE, ALDH1A7, APOE, BCHE, CARD6, | 14 | Hematological |
| | CDKN1A, CLDN11, COL15A1, COLQ, CPM, | | Disease, Cellular |
| | CTSG, DHRS3, FRZB, GP1BA, GP1BB, IRF5, | | Movement, |
| | KDR, MAP4K4, MAPK11, MAPK12, MAPK13, | | Immunological |
| | MMP1, MMP3, NFE2L2, PDRG1, PF4, POU2F1, | | Disease |
| | PROC, RIPK1, RIPK2, SERPINA3, ST3GAL5, | | |
| | TDRD7, TNF, TRADD | | |
| 13 | ACTA1, CD200, CD200R1, CDKN1A, DAP, | 13 | Cancer, Cellular |
| | DOK1, DOK2, ERBB2, EREG, F2R, FLJ36748, | | Development, |
| | GALNT3, GDPD3, GRB7, GSN, ID4, HNRPC, | | Cellular Growth |
| | KLK3, MMP1, MMP14, NUP214, NXF3, NXT1, | | and Proliferation |
| | P4HA2, PDE8A, RET, SDK1, SOX10, STUB1, | | |
| | TERT, TPD52, TPD52L1, USF2, VIL2, WNT5A, | | |
| | XPO1 | | |
| 14 | ANXA1, ANXA2, BGN, BIRC5, CALD1, CDH11, | 13 | Genetic Disorder, |
| | CDKN1A, CHI3L1, COL6A1, COL6A2, COL6A3, | | Skeletal and |
| | CTSB, DRD1, DRD2, DYSF, FMR1, GPRASP1, HD | | Muscular |
| | (includes EG: 3064), HRAS, IL2RB, LECT1, | | Disorders, Cancer |
| | M6PRBP1, MAP2K6, MUC2, ODZ3, PCYT1A, | | |
| | RAD9A, S100A4, SERPIND1, SMAD7, SP3, STK10, | | |
| | TAGLN, TIMP1, TNC | | |

B. Table 6B.

| | | | |
|---|---|---|---|
| 1 | A2M, ADM, AGT, BTG1, CCL13, CD53, CDH22, | 22 | Cellular Growth and |
| | CEBPD, CENTD1, CREM, CYP2J2, FZD9, | | Proliferation, Cell |
| | GABARAPL2, GJA1, GLDC, HRASLS3, ID4, | | Death, Cancer |
| | IFNG, IL15, ITGA5, JUN, KIR2DL3, KLRB1C, | | |
| | LAMB1, LMO1, LYN, MAPK10, MCC (includes | | |
| | EG: 4163), NFKBIB, PEA15, PPP1R1A, PRKCA, | | |
| | PRKCB1, TNFRSF12A, WNT7A, ZFP36 | | |
| 2 | AOX1, ARL4C, C9ORF26, CCL13, CEBPD, | 20 | Cell-To-Cell |
| | CMA1, CXCL6, DCAMKL1, EMX2, FAM19A2, | | Signaling and |
| | FLJ20701, GADD45G, GJA1, HLA-DRA, HRAS, | | Interaction, Cellular |
| | IL6, IL13, KITLG (includes EG: 4254), KRAS, | | Growth and |
| | MBP, NFKBIZ, NFYB, OXTR, PDPN, RFX2, | | Proliferation, |
| | RFX3, RFX4, RPL30, SORT1, TFF3, THRSP, | | Hematological |
| | TNFSF4, TPM1, TSLP, WNT5A | | System Development |
| | | | and Function |
| 3 | ADAM17, AGTR1B, ANGPT2, C5ORF13, | 17 | Cellular Movement, |
| | CREM, CSPG2, DLL1, EFNB2, EGFR, EMP2, | | Drug Metabolism, |
| | FGF1, FUT8, GJB1, GPC1, GPD2, GRB10, | | Small Molecule |
| | GRM5, HMGA2, HOXB7, HTATIP2, IGFBP2, | | Biochemistry |
| | ITGA5, LRIG1, MGAT3, NOTCH3, NTS | | |
| | (includes EG: 4922), NTSR2, PPAP2B, PTGS1, | | |
| | SNAI2, STC1, SULF2, TNC, VAV3, VEGF | | |
| 4 | A2M, ALOX5AP, APOE, BIK, C6, C7, C9, CA2, | 17 | Hematological |
| | CCL13, CEBPD, CTSE, CXCL6, EIF2S3Y, | | System Development |
| | FGF19 (includes EG: 9965), GABRA1, GABRB3, | | and Function, Tissue |
| | GABRG1, GAS1, HOXC8, ID4, KITLG (includes | | Development, |
| | EG: 4254), LCAT, LPL, LYN, MEIS2, MME, | | Neurological Disease |
| | MS4A2, OGG1, PBX1, PRKCB1, PROM1, | | |
| | SLC4A1, TEAD1 (includes EG: 7003), THY1, | | |
| | VLDLR, ZNF202 | | |
| 5 | AKAP5, AXIN1, BMP2, CAMK2B, CNKSR3, | 17 | Cell-To-Cell |
| | CRMP1, CTNNB1, DLG4, DMP1, FGF1, FRAT1, | | Signaling and |
| | FZD4, FZD9, GRASP, GRIN1, GRM3, GSK3B, | | Interaction, Nervous |
| | HAP1, HD (includes EG: 3064), HTRA1, KCNJ16, | | System Development |
| | LPHN2, MAP3K10, MAPK10, NDP, NPTX1, | | and Function, |
| | NRCAM (includes EG: 4897), OPN3, PEG12, | | Neurological Disease |
| | PRKCB1, PURB, SHANK2, SLC6A1, SLC6A2, | | |
| | SRF (includes EG: 6722) | | |
| 6 | ADM, AKR1B1, AKT1, BCL2, CALCRL, CCL13, | 17 | Cell Morphology, |
| | CCND2, CDKN2B, CDX1 (includes EG: 1044), | | Cellular |
| | CHGA, CX3CL1, EGFR, ELAVL2, F2, FOXG1B, | | Development, Cell- |
| | GCG, HTRA2, IAPP, IER2, ITGA5, KITLG | | To-Cell Signaling |
| | (includes EG: 4254), LYN, MBOAT2, MLLT7, | | and Interaction |
| | NNAT, POU3F4, RAB3B, RAMP1, RDH5, | | |
| | RHOB, SCG3, SLC2A1, SNAP23, STX11, TCOF1 | | |
| | (includes EG: 6949) | | |
| 7 | ALOX5, ARHGAP29, CASP4, CCND2, CEBPD, | 16 | Cell Death, Cancer, |
| | CHUK, CREM, CX3CL1, DKK1, FGD6, GBP2, | | Cellular |
| | GBP4, HBEGF, HDC, IL3, ING1, ITGA7, LTBP1, | | Development |
| | MAP3K2, MEN1, MSX1, MYO6, MYST1, NDN, | | |
| | PDE1B, RBBP5, SFN, SLC7A11, TNFSF13B, | | |
| | TP53, TP73L, UPP1, YAP1, YWHAG, ZFP36 | | |
| 8 | AFP, BTBD11, CTSC, D13BWG1146E, DNER, | 15 | Immune and |
| | DUSP6, EGFR, EREG, GNAI3, GNAZ, GNB5, | | Lymphatic System |
| | GSTA4, JAG2, KITLG (includes EG: 4254), MNT, | | Development and |
| | MT1A, NBL1, NCAM1, PTGS1, RGS7, RGS20, | | Function, Cellular |
| | ROBO1, SLIT1, SLIT2, SNN, TERT, TG, THRSP, | | Movement, Cellular |
| | TM4SF1, TNF, TP73, TP53I11, UGCG, WNT5A, | | Development |
| | YWHAQ | | |

TABLE 7
GO analysis of 538 cancer genes for molecular function (A) and biological processes (B).

| | | | | | | |
|---|---|---|---|---|---|---|
| ID | Pvalue | OddsRatio | ExpCount | Count | Size | Term |

A. Table 7A.
Group i: Gene to GO MF Conditional Test for over Representation

| | | | | | | | |
|---|---|---|---|---|---|---|---|
| 1 | GO:0030020 | 0.00 | 13.65 | 0 | 5 | 29 | extracellular matrix structural |
| | | | | | | | constituent conferring tensile |
| | | | | | | | strength |
| 2 | GO:0004528 | 0.00 | 129.00 | 0 | 2 | 3 | phosphodiesterase I activity |
| 3 | GO:0008467 | 0.00 | 42.99 | 0 | 2 | 5 | heparin-glucosamine 3-O- |
| | | | | | | | sulfotransferase activity |
| 4 | GO:0008889 | 0.00 | 42.99 | 0 | 2 | 5 | glycerophosphodiester |
| | | | | | | | phosphodiesterase activity |
| 5 | GO:0004180 | 0.00 | 7.65 | 1 | 4 | 38 | carboxypeptidase activity |
| 6 | GO:0004182 | 0.00 | 11.43 | 0 | 3 | 20 | carboxypeptidase A activity |
| 7 | GO:0008046 | 0.00 | 32.24 | 0 | 2 | 6 | axon guidance receptor activity |
| 8 | GO:0004551 | 0.00 | 32.24 | 0 | 2 | 6 | nucleotide diphosphatase |
| | | | | | | | activity |
| 9 | GO:0005509 | 0.01 | 1.97 | 10 | 19 | 669 | calcium ion binding |
| 10 | GO:0019899 | 0.01 | 4.18 | 1 | 5 | 83 | enzyme binding |

Group ii: Gene to GO MF Conditional Test for over Representation

| | | | | | | | |
|---|---|---|---|---|---|---|---|
| 1 | GO:0005332 | 0.00 | 87.34 | 0 | 2 | 4 | gamma-aminobutyric |
| | | | | | | | acid:sodium symporter |
| | | | | | | | activity |
| 2 | GO:0005416 | 0.00 | 29.10 | 0 | 2 | 8 | cation:amino acid |
| | | | | | | | symporter activity |
| 3 | GO:0005102 | 0.01 | 2.48 | 5 | 12 | 453 | receptor binding |
| 4 | GO:0015203 | 0.01 | 8.23 | 0 | 3 | 35 | polyamine transporter |
| | | | | | | | activity |

Group iii: Gene to GO MF Conditional Test for over Representation

| | | | | | | | |
|---|---|---|---|---|---|---|---|
| 1 | GO:0030020 | 0.00 | 23.78 | 0 | 4 | 29 | extracellular matrix structural |
| | | | | | | | constituent conferring tensile |
| | | | | | | | strength |
| 2 | GO:0005509 | 0.00 | 3.76 | 4 | 15 | 669 | calcium ion binding |
| 3 | GO:0008191 | 0.00 | 72.54 | 0 | 2 | 6 | metalloendopeptidase inhibitor |
| | | | | | | | activity |
| 4 | GO:0043167 | 0.00 | 1.99 | 16 | 31 | 2762 | ion binding |
| 5 | GO:0004497 | 0.01 | 6.15 | 1 | 4 | 100 | monooxygenase activity |
| 6 | GO:0008387 | 0.01 | Inf | 0 | 1 | 1 | steroid 7-alpha-hydroxylase |
| | | | | | | | activity |
| 7 | GO:0005502 | 0.01 | Inf | 0 | 1 | 1 | 11-cis retinal binding |
| 8 | GO:0003979 | 0.01 | Inf | 0 | 1 | 1 | UDP-glucose 6-dehydrogenase |
| | | | | | | | activity |
| 9 | GO:0000156 | 0.01 | Inf | 0 | 1 | 1 | two-component response regulator |
| | | | | | | | activity |
| 10 | GO:0004114 | 0.01 | 15.25 | 0 | 2 | 21 | 3′,5′-cyclic-nucleotide |
| | | | | | | | phosphodiesterase activity |

Group iv: Gene to GO MF Conditional Test for over Representation

| | | | | | | | |
|---|---|---|---|---|---|---|---|
| 1 | GO:0001968 | 0.00 | Inf | 0 | 1 | 1 | fibronectin binding |
| 2 | GO:0005112 | 0.00 | 948.83 | 0 | 1 | 2 | Notch binding |
| 3 | GO:0050780 | 0.00 | 474.38 | 0 | 1 | 3 | dopamine receptor binding |
| 4 | GO:0005246 | 0.01 | 237.15 | 0 | 1 | 5 | calcium channel regulator |
| | | | | | | | activity |
| 5 | GO:0004697 | 0.01 | 189.70 | 0 | 1 | 6 | protein kinase C activity |

B. Table 7B.
Group i: Gene to GO BP Conditional Test for over Representation

| | | | | | | | |
|---|---|---|---|---|---|---|---|
| 1 | GO:0007155 | 0.00 | 3.34 | 7 | 21 | 445 | cell adhesion |
| 2 | GO:0006817 | 0.00 | 9.69 | 1 | 7 | 53 | phosphate transport |
| 3 | GO:0006820 | 0.00 | 3.85 | 2 | 8 | 140 | anion transport |
| 4 | GO:0042552 | 0.00 | 14.38 | 0 | 3 | 16 | myelination |
| 5 | GO:0042553 | 0.00 | 14.38 | 0 | 3 | 16 | cellular nerve ensheathment |
| 6 | GO:0048169 | 0.00 | 41.32 | 0 | 2 | 5 | regulation of long-term neuronal |
| | | | | | | | synaptic plasticity |
| 7 | GO:0001508 | 0.00 | 12.46 | 0 | 3 | 18 | regulation of action potential |
| 8 | GO:0042423 | 0.01 | 24.79 | 0 | 2 | 7 | catecholamine biosynthesis |
| 9 | GO:0006836 | 0.01 | 6.25 | 1 | 4 | 44 | neurotransmitter transport |
| 10 | GO:0007399 | 0.01 | 2.23 | 7 | 14 | 418 | nervous system development |
| 11 | GO:0042551 | 0.01 | 8.12 | 0 | 3 | 26 | neuron maturation |
| 12 | GO:0048167 | 0.01 | 17.70 | 0 | 2 | 9 | regulation of synaptic plasticity |

Group ii: Gene to GO BP Conditional Test for over Representation

| | | | | | | | |
|---|---|---|---|---|---|---|---|
| 1 | GO:0007154 | 0.00 | 2.46 | 20 | 45 | 1960 | cell communication |
| 2 | GO:0007166 | 0.00 | 2.46 | 11 | 26 | 1012 | cell surface receptor linked |
| | | | | | | | signal transduction |
| 3 | GO:0045665 | 0.00 | 35.85 | 0 | 3 | 10 | negative regulation of neuron |
| | | | | | | | differentiation |
| 4 | GO:0008347 | 0.00 | 165.98 | 0 | 2 | 3 | glial cell migration |
| 5 | GO:0007413 | 0.00 | 165.98 | 0 | 2 | 3 | axonal fasciculation |
| 6 | GO:0007417 | 0.00 | 5.40 | 1 | 7 | 118 | central nervous system |
| | | | | | | | development |
| | GO:0030182 | 0.00 | 3.99 | 2 | 9 | 204 | neuron differentiation |
| | GO:0000902 | 0.00 | 3.01 | 4 | 10 | 297 | cellular morphogenesis |
| | GO:0030900 | 0.00 | 7.00 | 1 | 4 | 52 | forebrain development |
| 10 | GO:0051093 | 0.00 | 6.46 | 1 | 4 | 56 | negative regulation of |
| | | | | | | | development |
| 11 | GO:0006760 | 0.00 | 23.70 | 0 | 2 | 9 | folic acid and derivative |
| | | | | | | | metabolism |
| 12 | GO:0006944 | 0.01 | 20.73 | 0 | 2 | 10 | membrane fusion |
| 13 | GO:0001676 | 0.01 | 18.43 | 0 | 2 | 11 | long-chain fatty acid |
| | | | | | | | metabolism |
| 14 | GO:0006874 | 0.01 | 7.82 | 0 | 3 | 35 | calcium ion homeostasis |
| 15 | GO:0048731 | 0.01 | 2.34 | 5 | 12 | 455 | system development |
| 16 | GO:0048812 | 0.01 | 4.09 | 1 | 5 | 108 | neurite morphogenesis |
| 17 | GO:0007611 | 0.01 | 7.36 | 0 | 3 | 37 | learning and/or memory |

Group iii: Gene to GO BP Conditional Test for over Representation

| | | | | | | | |
|---|---|---|---|---|---|---|---|
| 1 | GO:0030199 | 0.00 | 112.03 | 0 | 3 | 7 | collagen fibril organization |
| 2 | GO:0001502 | 0.00 | 64.00 | 0 | 3 | 10 | cartilage condensation |
| 3 | GO:0001501 | 0.00 | 7.17 | 1 | 8 | 185 | skeletal development |
| 4 | GO:0006029 | 0.00 | 37.31 | 0 | 3 | 15 | proteoglycan metabolism |
| 5 | GO:0006817 | 0.00 | 12.32 | 0 | 4 | 53 | phosphate transport |
| 6 | GO:0030048 | 0.00 | 73.59 | 0 | 2 | 6 | actin filament-based |
| | | | | | | | movement |
| 7 | GO:0007155 | 0.00 | 3.67 | 3 | 10 | 445 | cell adhesion |
| 8 | GO:0009888 | 0.00 | 4.81 | 2 | 7 | 233 | tissue development |
| 9 | GO:0001656 | 0.00 | 14.42 | 0 | 3 | 34 | metanephros development |
| 10 | GO:0030500 | 0.00 | 36.78 | 0 | 2 | 10 | regulation of bone |
| | | | | | | | mineralization |
| 11 | GO:0001655 | 0.00 | 10.63 | 0 | 3 | 45 | urogenital system |
| | | | | | | | development |
| 12 | GO:0043062 | 0.00 | 10.14 | 0 | 3 | 47 | extracellular structure |
| | | | | | | | organization and biogenesis |
| 13 | GO:0045664 | 0.01 | 19.60 | 0 | 2 | 17 | regulation of neuron |
| | | | | | | | differentiation |
| 14 | GO:0008366 | 0.01 | 18.38 | 0 | 2 | 18 | nerve ensheathment |
| 15 | GO:0046850 | 0.01 | 18.38 | 0 | 2 | 18 | regulation of bone |
| | | | | | | | remodeling |
| 16 | GO:0043071 | 0.01 | Inf | 0 | 1 | 1 | positive regulation of non- |
| | | | | | | | apoptotic programmed cell |
| | | | | | | | death |
| 17 | GO:0045908 | 0.01 | Inf | 0 | 1 | 1 | negative regulation of |
| | | | | | | | vasodilation |
| 18 | GO:0016244 | 0.01 | Inf | 0 | 1 | 1 | non-apoptotic programmed |
| | | | | | | | cell death |
| 19 | GO:0007399 | 0.01 | 3.02 | 3 | 8 | 418 | nervous system development |
| 20 | GO:0030182 | 0.01 | 4.15 | 1 | 5 | 204 | neuron differentiation |

Group iv: Gene to GO BP Conditional Test for over Representation

| | | | | | | | |
|---|---|---|---|---|---|---|---|
| 1 | GO:0048747 | 0.00 | 67.18 | 0 | 2 | 25 | muscle fiber development |
| 2 | GO:0048637 | 0.00 | 61.80 | 0 | 2 | 27 | skeletal muscle development |
| 3 | GO:0046698 | 0.00 | Inf | 0 | 1 | 1 | metamorphosis (sensu |
| | | | | | | | Insecta) |
| 4 | GO:0001946 | 0.00 | Inf | 0 | 1 | 1 | lymphangiogenesis |
| 5 | GO:0048748 | 0.00 | Inf | 0 | 1 | 1 | eye morphogenesis (sensu |
| | | | | | | | Endopterygota) |
| 6 | GO:0048749 | 0.00 | Inf | 0 | 1 | 1 | compound eye development |
| | | | | | | | (sensu Endopterygota) |
| 7 | GO:0008583 | 0.00 | Inf | 0 | 1 | 1 | mystery cell fate |
| | | | | | | | differentiation (sensu |
| | | | | | | | Endopterygota) |
| 8 | GO:0007455 | 0.00 | Inf | 0 | 1 | 1 | eye-antennal disc |
| | | | | | | | morphogenesis |
| 9 | GO:0007444 | 0.00 | Inf | 0 | 1 | 1 | imaginal disc development |
| 10 | GO:0045063 | 0.00 | Inf | 0 | 1 | 1 | T-helper 1 cell differentiation |
| 11 | GO:0007220 | 0.00 | 719.00 | 0 | 1 | 2 | Notch receptor processing |
| 12 | GO:0001654 | 0.00 | 25.24 | 0 | 2 | 63 | eye development |
| 13 | GO:0006816 | 0.00 | 22.62 | 0 | 2 | 70 | calcium ion transport |
| 14 | GO:0042095 | 0.01 | 239.62 | 0 | 1 | 4 | interferon-gamma |
| | | | | | | | biosynthesis |
| 15 | GO:0007275 | 0.01 | 4.44 | 2 | 7 | 1664 | development |
| 16 | GO:0000186 | 0.01 | 143.74 | 0 | 1 | 6 | activation of MAPKK |
| | | | | | | | activity |
| 17 | GO:0007528 | 0.01 | 143.74 | 0 | 1 | 6 | neuromuscular junction |
| | | | | | | | development |
| 18 | GO:0030335 | 0.01 | 143.74 | 0 | 1 | 6 | positive regulation of cell |
| | | | | | | | migration |

TABLE 8
Real Time PCR validation using primary and secondary tumors.
Indicated are the fold change values of CSC compared to NSC, normalized to 18s. Standard
deviations are in parentheses.

| | | | | CSC 1 | CSC 2 |
|---|---|---|---|---|---|
| | CSC 1 | CSC 2 | CSC 3 | secondary | secondary |
| Dkk3 (n = 1) | 897.64 | 7.41 | | 62.8 | 82.7 |
| Susd5 (n = 2) | 530.9 (+/−65.7) | 84.9 (+/−4.2) | 383.1 (+/−23.8) | | |
| Wif1 (n = 1) | 258.97 | 7.50 | | 167.7 | 151.0 |
| Slit3 (n = 2) | 163.8 (+/−37.7) | 41.1 (+/−27.1) | 59 (+/−9.6) | | |
| Foxc2 (n = 2) | 119.43 | 1.99 | | 43.61 (+/−12.83) | 19.12 (+/−1.46) |
| Hey2 (n = 1) | 68.9 | 2.5 | | 2.61 | 7.19 |
| Col6a1 (n = 3) | 67.44 (+/−5.7) | 36.90 (+/−3.1) | 21.34 (+/−2.3) | | |
| Snai2 (n = 3) | 39.7 (+/−7.4) | 4.1 (+/−0.74) | 8.7 (+/−0.75) | | |
| Prickle1 (n = 1) | 10.29 | 13.06 | | 11.9 | 13.1 |
| Cdkn1a (n = 1) | 10.17 | 4.0 | 16.0 | | |
| Ldoc11 (n = 1) | 5.04 | 5.90 | | 3.52 | 2.38 |
| A93001N09Rik | 4.6 | 2.7 | 3.9 | | |
| (n = 1) | | | | | |
| Mmp16 (n = 1) | 3.6 | 1.2 | 12.0 | | |
| Mmp17 (n = 2) | 2.63 (+/−0.14) | 0.85 (+/−0.38) | 2.55 (+/−1.83) | | |
| Tcfl5 (n = 2) | 2.39 | 3.07 | 1.41 | 1.07 (+/−0.32) | 0.35 (+/−0.32) |
| Ccnd3 (n = 1) | 2.3 | 1.2 | 11.7 | | |
| Mettl7a (n = 1) | 2.29 | 1.75 | | 2.08 | 1.58 |
| Slit2 (n = 2) | 1.7 (+/−0.76) | 1.7 (+/−0.43) | 17.3 (+/−14.64) | | |
| S100a4 (n = 3) | 1.58 (0.30) | 1.83 (+/−0.44) | 7.24 (+/−0.80) | | |
| Zfp36 (n = 2) | 1.00 (+/−0.02) | 0.71 (+/−0.092) | 1.19 | 0.21 | 0.34 |
| Stat5a (n = 2) | 0.83 (+/−0.28) | 0.64 (+/−0.62) | 1.67 | 1.54 | 0.95 |
| Igfbp2 (n = 1) | 0.60 | 0.0035 | | 0.0013 | 0.0005 |
| Gadd45g (n = 1) | 0.46 | 0.62 | | 0.83 | 0.32 |
| Abca13 (n = 1) | 0.40 | 0.70 | | | |
| Frat1 (n = 1) | 0.31 | 0.66 | | 0.08 | 0.06 |
| Sall3 (n = 1) | 0.30 | 0.16 | | 0.09 | 0.11 |
| S100a6 (n = 2) | 0.27 (+/−0.04) | 4.98 (+/−0.67) | 6.23 (+/−0.24) | | |
| Hrasls3 (n = 2) | 0.26 (+/−0.04) | 0.65 (+/−0.10) | | | |
| Ephb1 (n = 2) | 0.21 (+/−0.057) | 0.15 (+/−0.007) | 5.2 (+/−5.38) | | |
| Foxg1 (n = 1) | 0.07 | 0.07 | | 0.0001 | 0.0195 |
| Scg3 (n = 1) | 0.02 | 0.09 | 0.27 | | |
| Robo1 (n = 1) | 0.005 | 0.11 | 0.32 | | |
| Bgn (n = 1) | | | | 503 | 147 |
| Mamdc2 (n = 2) | | | | 0.006 (+/−0.001) | 0.034 (+/−0.015) |

TABLE 9
Subgroups of CSC markers upegulated in cancer stem cells as compared to non-stem
cancer cells.
Table 9: Gene symbol-in both sp stringent and spgo_t1

| | | fold | |
|---|---|---|---|
| | function | change | fold change |
| Mgp (matrix gla protein) | calcification, mineralization | 113.0555 | 85.8701 |
| Bgn (biglycan) | extracellular matrix, | 84.0721 | 120.0073 |
| | connective tissue metabolism | | |
| Foxc2 (Forkhead box C2, Fkh14, | lymphangiogenesis, cardiac | 43.6352 | 21.5747 |
| Hfhbf3, MFH-1, Mfh1) | development, adipocytes | | |
| | regulation | | |
| Papss2 | sulfate-activating enzyme | 30.8244 | 48.5215 |
| Ddc (Dopa decarboxylase, Aadc, | catecholamine biochemistry | 18.9885 | 21.8111 |
| aromatic L-amino acid decarboxylase) | (dopamine, serotonin and | | |
| | norepinephrine synthesis) | | |
| Kazald1 (Kazal-type serine peptidase | insulin-like growth factor | 15.9197 | 22.0810 |
| inhibitor domain 1, Bono1, Igfbp-rp10) | binding | | |
| S100a6 (calcyclin) | calcium-binding protein | 13.7827 | 49.1524 |
| S100a4 (pEL-98, mts1, p9Ka, CAPL, | calcium-binding protein | 13.0958 | 16.3816 |
| calvasculin, FspI) | | | |
| Col6a1 | extracellular matrix | 11.8299 | 19.5567 |
| Arhgap6 (Rho GTPase activating | GTPase-activating protein, | 11.3820 | 15.0650 |
| protein 6) | cytoskeletal protein | | |
| 3110035E14Rik | unknown | 10.7163 | 13.5067 |
| Lgals2 (Galectin-2, lectin, galactose- | apoptosis | 9.5199 | 13.9632 |
| binding, soluble 2) | | | |
| Casp4 (caspase 4) | | 9.2320 | 15.5698 |
| tmem46 (transmembrane protein 46, | inhibitor of Wnt and FGF | 8.3970 | 4.6304 |
| 9430059P22Rik, mShisa, shisa) | signaling | | |
| D3Bwg0562e (mKIAA0455) | unknown | 8.3043 | 4.1055 |
| Scg5 (secretogranin V, 7B2, Sgne-1, | molecular chaperone for | 7.7904 | 9.2184 |
| Sgne1) | PCSK2/PC2 | | |
| Col6a2 | extracellular matrix | 7.4843 | 21.7447 |
| Cytl1(cytokine like protein 1, protein | chondrogenesis | 7.4435 | 24.7756 |
| C17, C17) | | | |
| Opcml (Opioid-binding cell adhesion | cell adhesion, tumor | 7.3989 | 5.0782 |
| molecule, OBCAM, OPCM) | suppressor | | |
| Foxa3 (Forkhead box protein A3, | transcription activator for a | 6.7880 | 14.4403 |
| FKHH3, HNF-3G, MGC10179, | number of liver genes | | |
| TCF3G) | | | |
| Ninj2 (ninjurin 2, Nerve injury-induced | homophilic adhesion; neurite | 6.4597 | 10.5655 |
| protein 2) | outgrowth | | |
| Kcne4 (minimum potassium ion | modulates the gating kinetics | 6.3232 | 19.4254 |
| channel-related peptide 3, MGC20353, | and enhances stability of the | | |
| MIRP3) | potassium channel complex. | | |
| Capg (capping protein (actin filament), | macrophage phagocytosis, | 5.7438 | 24.3536 |
| gelsolin-like, gCap39, mbh1) | tumor suppressor | | |
| 2310046A06Rik | unknown | 5.4145 | 11.0592 |
| Srpx2 (Sushi-repeat-containing protein, | involved in the formation of | 4.7904 | 9.9199 |
| X-linked 2, SRPUL, RESDX) | functional neural circuits and | | |
| | in the development of CNS | | |
| | functions involved in | | |
| | locomotor activity | | |
| Enpp6 (E-NPP6, Ectonucleotide | enzyme | 4.7689 | 7.7495 |
| pyrophosphatase/phosphodiesterase | | | |
| family member 6 precursor) | | | |
| A930001N09Rik | transcription factor | 4.7194 | 4.3594 |
| E030011K20Rik | unknown | 4.1361 | 5.9198 |
| Dhrs3 (dehydrogenase/reductase (SDR | oxidoreductase activity for all- | 4.0098 | 4.2742 |
| family) member 3, retSDR1, Rsdr1) | trans-retinal | | |
| Vwc2 (von Willebrand factor C domain | neurogenesis, BMP antagonist | 3.7705 | 4.7001 |
| containing 2, BRORIN, MGC131845, | | | |
| PSST739, UNQ739) | | | |
| Bfsp2 (beaded filament structural | Cytoskeleton, eye lens | 3.4412 | 26.8379 |
| protein 2, phakinin, CP47, CP49, LIFL- | | | |
| L, MGC142078, MGC142080) | | | |
| Larp6 (La ribonucleoprotein domain | RNA binding | 3.3974 | 7.4683 |
| family, member 6, Acheron, Achn, | | | |
| FLJ11196) | | | |
| Cav1 (caveolin 1, CAV, MSTP085, | scaffolding protein | 3.1876 | 28.1129 |
| VIP21) | | | |
| Mia1 (melanoma inhibitory activity 1, | chondrogenesis | 3.1183 | 8.7770 |
| Cdrap, melanoma inhibitory activity, | | | |
| MIA) | | | |
| Gpr17 (R12, G protein-coupled receptor | cell-to-cell communication | 2.8738 | 14.5006 |
| 17) | | | |

TABLE 10
Subgroups of CSC biomarkers downregulated in cancer stem cells as compared to
non-stem cancer cells.
Table 10: Gene symbol-in both sp stringent and spgo_t1

| | Function | fold change | fold change |
|---|---|---|---|
| Tead1 (transcriptional enhancer factor- | Transcription factor, | 0.3326 | 0.2395 |
| 1, TEA domain family member 1, | cardiac development | | |
| Gtrgeo5, mTEF-1, Tcf13, TEAD-1, | | | |
| TEF-1, NTEF-1, AA) | | | |
| Aox1 (aldehyde oxidase 1, Aox-1, Aox- | metabolizes retinaldehyde | 0.2825 | 0.2825 |
| 2, Aox2, MGC: 13774, MoRO, retinal | into retinoic acid | | |
| oxidase) | | | |
| AI851790 (TAFA2) | brain-specific chemokine | 0.2701 | 0.1007 |
| | or neurokine | | |
| Arhgap29 (Rho GTPase activating | tumor suppressor | 0.2606 | 0.3128 |
| protein 29, Parg1) | | | |
| 5033414K04Rik | unknown | 0.1891 | 0.2576 |
| AI593442 | unknown | 0.1863 | 0.0994 |
| Wnt5a (wingless-related MMTV | signaling molecule, tumor | 0.1610 | 0.1541 |
| integration site 5A) | suppressor | | |
| Scg3 (gamma sarcoglycan, 35 kD | component of the | 0.1542 | 0.2357 |
| dystrophin-associated glycoprotein) | sarcoglycan complex, | | |
| D930020E02Rik (HERV-FRD | involved in trophoblast | 0.0832 | 0.1334 |
| GC06M011210, HERV-FRD provirus | cell fusion | | |
| ancestral Env polyprotein, syncytin 2) | | | |
| Gja1 (gap junction protein, alpha-like, | gap junction | 0.0174 | 0.2353 |
| connexin-43, CX43, GJAL, DFNB38, | | | |
| SDTY3) | | | |

### REFERENCES

The references cited herein and throughout the application are incorporated herein by reference.

1. E. I. Fomchenko, E. C. Holland, Exp Cell Res 306, 323 (Jun. 10, 2005).

2. M. S. Wicha, S. Liu, G. Dontu, Cancer Res 66, 1883 (Feb. 15, 2006).

3. S. K. Singh, I. D. Clarke, T. Hide, P. B. Dirks, Oncogene 23, 7267 (Sep. 20, 2004).

4. T. Reya, S. J. Morrison, M. F. Clarke, I. L. Weissman, Nature 414, 105 (Nov. 1, 2001).

5. F. Behbod, J. M. Rosen, Carcinogenesis 26, 703 (April 2005).

6. M. Al-Hajj, M. W. Becker, M. Wicha, I. Weissman, M. F. Clarke, Curr Opin Genet Dev 14, 43 (February 2004).

7. M. Zhang, J. M. Rosen, Curr Opin Genet Dev 16, 60 (February 2006).

8. G. Liu et al., Mol Cancer 5, 67 (2006).

9. S. Bao et al., Nature 444, 756 (Dec. 7, 2006).

10. W. A. Weiss et al., Cancer Res 63, 1589 (Apr. 1, 2003).

11. R. Galli et al., Cancer Res 64, 7011 (Oct. 1, 2004).

12. X. Yuan et al., Oncogene 23, 9392 (Dec. 16, 2004).

13. H. D. Hemmati et al., Proc Natl Acad Sci USA 100, 15178 (Dec. 9, 2003).

14. S. K. Singh et al., Cancer Res 63, 5821 (Sep. 15, 2003).

15. S. K. Singh et al., Nature 432, 396 (Nov. 18, 2004).

16. Y. Liu et al., Dev Biol 276, 31 (Dec. 1, 2004).

17. L. Patrawala et al., Cancer Res 65, 6207 (Jul. 15, 2005).

18. T. Kondo, T. Setoguchi, T. Taga, Proc Natl Acad Sci USA 101, 781 (Jan. 20, 2004).

19. M. Kim, C. M. Morshead, J Neurosci 23, 10703 (Nov. 19, 2003).

20. B. Lassalle et al., Development 131, 479 (January 2004).

21. M. A. Goodell, S. McKinney-Freeman, F. D. Camargo, Methods Mol Biol 290, 343 (2005).

22. M. A. Goodell et al., Nat Med 3, 1337 (December 1997).

23. S. C. Garrett, K. M. Varney, D. J. Weber, A. R. Bresnick, J Biol Chem 281, 677 (Jan. 13, 2006).

24. D. M. Helfman, E. J. Kim, E. Lukanidin, M. Grigorian, Br J Cancer 92, 1955 (Jun. 6, 2005).

25. E. Fuchs, T. Tumbar, G. Guasch, Cell 116, 769 (Mar. 19, 2004).

26. R. J. Morris et al., Nat Biotechnol 22, 411 (April 2004).

1. A method to identify a cancer stem cell in a population of cells, the method comprising;
(i) measuring a level of expression of at least 6 nucleic acid sequences encoding proteins selected from the group consisting of: 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4, KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik in a biological sample;
(ii) identifying which of the genes measured in step (i) are cancer stem cell upregulated biomarkers selected from the group of; 2310046A06Rik; 3110035E14Rik; A930001N09Rik; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GPR17; ID4; KAZALD1; KCNA4; LARP6; LGALS3; MGP; MIA; NINJ2; OPCML; PAPSS2; S100A4; S100A6; SCG5; SRPX2; TMEM46 and VWC2;
(iii) identifying which of the genes measured in step (i) are cancer stem cell downregulated biomarkers selected from the group of; AI593442; AI851790; AOX1; ARHGAP29; GJA1; SCG3; TEAD1; WNT5A; and 5033414K04Rik
(iv) comparing the level of expression of each nucleic acid sequences measured in (i) to a reference expression level for each of the nucleic acid sequence measured;
wherein an increase in the level of the expression of at least 1.5-fold of said measured nucleic acid sequences for a cancer stem cell upregulated biomarker as compared to said reference expression level indicates the presence of a cancer stem cell in a population of cells, or
wherein an decrease in the level of the expression of at least 0.5-fold fold of said measured nucleic acid sequences for a cancer stem cell downregulated biomarker as compared to said reference expression level indicates the presence of a cancer stem cell in a population of cells.

2. The method of claim 1, wherein for respective sequences in said at least 6 nucleic acid sequences, the difference is an increase in level of expression.

3. The method of claim 1, wherein for respective sequences in said at least 6 nucleic acid sequences, the difference is a decrease in level of expression

4. The method of claim 1, wherein the level of expression is the level of gene transcript expression.

5. The method of claim 1, wherein the level of expression is the level of protein expression.

6. The method of claim 1, wherein the increase in expression level of a cancer stem cell upregulated biomarker is at least 2.0-fold as compared to a reference expression level.

7. The method of claim 1, wherein the decrease in expression level of a cancer stem cell downregulated biomarker is at least 0.4-fold as compared to a reference expression level.

8. The method of claim 1, wherein the increase or decrease in expression level of a cancer stem cell upregulated biomarker or a cancer stem cell downregulated biomarker has a q-value of less than 0.05.

9. The method of claim 1, wherein the levels of expression of at least 10 said nucleic acid sequences are measured.

10. The method of claim 1, wherein the levels of expression of at least 20 said nucleic acid sequences are measured.

11. The method of claim 1, wherein the levels of expression of at least 30 said nucleic acid sequences are measured.

12. The method of claim 1, wherein the levels of expression of at least 40 said nucleic acid sequences are measured.

13. The method of claim 1, wherein the nucleic acid sequences encoding said proteins are selected from a group of nucleic acid sequences consisting of GenBank Identification Nos; 2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); 5033414K04Rik (SEQ ID NO:45); U16153 (SEQ ID NO:46).

14. The method of claim 1, wherein the biological sample is obtained from a subject at a first time point.

15. The method of claim 1, further comprising:
(v) measuring a level of expression of at least 6 nucleic acid sequences encoding proteins selected from the group consisting of: 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik in a biological sample obtained from a subject at a second timepoint;
(vi) comparing the level of expression of each nucleic acid sequences measured in (i) to the level expression of each respective nucleic acid sequence measured in (v);
wherein an increase in the level of the expression of at least 1.5-fold of said measured nucleic acid sequences for a cancer stem cell upregulated biomarker at said second timepoint as compared to the level of expression at said first timepoint indicates an increase in the proportion of cancer stem cells as compared to the non-cancer stem cells from first timepoint to the second timepoint; or
wherein a decrease in the level of the expression of at least 0.5-fold of said measured nucleic acid sequences for a cancer stem cell downregulated biomarker at said second timepoint as compared to the level of expression at said first timepoint indicates an increase in the proportion of cancer stem cells as compared to the non-cancer stem cells from first timepoint to the second timepoint.

16. The method of either claim 1 or 2, wherein said 6 nucleic acid sequences encoding the proteins are selected from a group that have increased expression, the group consisting of 2310046A06Rik; 3110035E14Rik; A930001N09Rik; BFSP2; BGN; CAPG; CASP4; CAV1; COL6A1; COL6A2; CYTL1; D3Bwg0562e; DDC; DHRS3; E030011K20Rik; ENPP6; FOXA3; FOXC2; GPR17; ID4, KAZALD1; KCNA4; LARP6; LGALS3; MGP; MIA; NINJ2; OPCML; PAPSS2; S100A4; S100A6; SCG5; SRPX2; TMEM46; VWC2.

17. The method of either claim 1 or 3, wherein said 6 nucleic acid sequences encoding the proteins are selected from a group that have decreased expression, the group consisting of; AI593442; AI851790; AOX1; ARHGAP29; ARHGAP6 D930020E02Rik; GJA1; SCG3; TEAD1; WNT5A; and 5033414K04Rik.

18. The method of claim 1 or 2, wherein at least 2 of said nucleic acid sequences encode proteins S100A4 and S100A6.

19. The method of claim 1, wherein said 6 nucleic acid sequences encoding the proteins are selected from the first group consisting of: Mgp, Bgn, Kazald1, Col6a1, Scg5, Col6a2, Vwc2, Mia, Scg3.

20. The method of claim 1, wherein said 6 nucleic acid sequences encoding the proteins are selected from the second group consisting of: Tmem46, Opcm1, Ninj2, Enpp6, Cav1, S100a6, S100a4, Gpr17, D930020E02Rik, Gja1, 5033414K04Rik, Kcna4.

21. The method of claim 1, wherein said 6 nucleic acid sequences encoding the proteins are selected from the third group consisting of: Cytl1, AI851790, Wnt5a, Papss2, Arhgap6, D3Bwg0562e, Arhgap29.

22. The method of claim 1, wherein said 6 nucleic acid sequences encoding the proteins are selected from the group fourth consisting of: Foxc2, Foxa3, A930001N09Rik(4.5×), Larp6 (5.4×), Tead1 (0.3×), CASP4.

23. The method of claim 1, wherein said 6 nucleic acid sequences encoding the proteins are selected from the fifth group consisting of: Ddc, Lgals2, Capg, Srpx2, Dhrs3, Bfsp2, Aox1, 3110035E14Rik, 2310046A06Rik, E030011K20Rik, Ai593442.

24. The method of claim 1, wherein said 6 nucleic acid sequences encoding the proteins are selected from the sixth group consisting of: A930001N09Rik; BGN; CAV1; COL6A1; CYTL1; FOXC2; GJA1; MGP; S100A4; S100A6 and SCG3.

25. The method of claim 1, wherein said 6 nucleic acid sequences encoding the proteins are selected from at least one nucleic acid sequence listed in each group according to any of the claims 18, 19, 20, 21, 22, 23 or 24.

26. The method of claim 1, wherein the biological sample is selected from the group consisting essentially of: blood, plasma, serum, urine, stool, spinal fluid, nipple aspirates, lymph fluid, external secretions of the skin, respiratory tract, intestinal and genitourinary tracts, bile, saliva, milk, tumors, organs, cancer tissue, a tissue sample, a biopsy sample, primary ascites cells and in vitro cell culture constituents.

27. The method of claim 26, wherein the biological sample is a human biological sample.

28. The method of claim 1, wherein the cancer stem cell is a brain cancer stem cell.

29. The method of claim 1, wherein the cancer stem cell is selected from a group consisting of: a breast cancer stem cell, colon cancer stem cell, ovarian cancer stem cell, a prostate cancer stem cell, and a melanoma stem cell.

30. The method of claim 5, wherein protein expression is measured using an antibody, human antibody, humanized antibody, recombinant antibodies, monoclonal antibodies, chimeric antibodies, protein binding proteins, aptamer, peptide or analogues, or conjugates or fragments thereof.

31. The method of claim 30, wherein measuring is by ELISA.

32. The method of claim 4, wherein the gene transcript expression is measured at the level of messenger RNA (mRNA).

33. The method of claim 32, wherein detection uses nucleic acid or nucleic acid analogues.

34. The method of claim 30, wherein the nucleic acid analogous comprise DNA, RNA, PNA, pseudo-complementary DNA (pcDNA), locked nucleic acid and variants and homologues thereof.

35. The method of claim 4, wherein the gene transcript expression is assessed by reverse-transcription polymerase-chain reaction (RT-PCR).

36. An array comprising a solid platform and protein-binding molecules attached thereto, wherein the array comprises at least 6 and at most 100 different protein-binding molecules in known positions, wherein at least 6 of the 100 different protein-protein binding molecules having binding affinity for proteins selected from the group of; 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A and 5033414K04Rik.

37. An array comprising a solid platform and protein-binding molecules attached thereto, wherein the array comprises at least 6 and at most 50 different protein-binding molecules in known positions, wherein at least 6 of the 50 different protein-protein binding molecules having binding affinity for proteins selected from the group of; 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik.

38. An array comprising a solid platform and nucleic acid-binding molecules attached thereto, wherein the array comprises at least 6 and at most 100 different nucleic acid-molecules in known positions, wherein at least 6 of the 100 different protein-protein binding molecules having binding affinity for nucleic acids selected from the group consisting of 2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); and 5033414K04Rik (SEQ ID NO:45); U16153 (SEQ ID NO:46).

39. An array comprising a solid platform and nucleic acid-binding molecules attached thereto, wherein the array comprises at most 50 different nucleic acid-molecules in known positions, wherein at least 6 of the 50 different protein-protein binding molecules having binding affinity for nucleic acids selected from the group of 2310046A06Rik (SEQ ID NO:1); 3110035E14Rik(SEQ ID NO:2); A930001N09Rik (SEQ ID NO:3); AI593442 (SEQ ID NO:4); AI851790 (SEQ ID NO:5); AF017060 /// NM_{—}001159 (SEQ ID NO:6); NM_{—}004815 (SEQ ID NO:7); AF012272 /// NM_{—}013427 (SEQ ID NO:8); U48224 /// NM_{—}003571 (SEQ ID NO:9); AK092954 /// NM_{—}001711 (SEQ ID NO:10); M94345 /// NM_{—}001747 (SEQ ID NO:11); U25804 /// NM_{—}001225 (SEQ ID NO:12); AF125348 /// NM_{—}001753 (SEQ ID NO:13); M20776 /// NM_{—}001848 (SEQ ID NO:14); M20777 /// NM_{—}058175 (SEQ ID NO:15); AF193766 /// NM_{—}018659 (SEQ ID NO:16); D3Bwg0562e (SEQ ID NO:17); D930020E02Rik (SEQ ID NO:18); NM_{—}000790 (SEQ ID NO:19); AF061741 /// NM_{—}004753 (SEQ ID NO:20); E030011K20Rik (SEQ ID NO:21); AK057370 /// NM_{—}153343 (SEQ ID NO:22 L12141 /// NM_{—}004497 (SEQ ID NO:23 Y08223 /// NM_{—}005251 (SEQ ID NO:24 BC026329 /// NM_{—}000165 (SEQ ID NO:25 NM_{—}005291 (SEQ ID NO:26 AF333487 /// NM_{—}030929 (SEQ ID NO:27 M55514 /// NM_{—}002233 (SEQ ID NO:28); BC009446 /// NM_{—}018357 (SEQ ID NO:29); M64303 /// NM_{—}002306 (SEQ ID NO:30); M58549 /// NM_{—}000900 (SEQ ID NO:31); X75450 /// NM_{—}006533 (SEQ ID NO:32); AF205633 /// NM_{—}016533 (SEQ ID NO:33); BX537377 /// NM_{—}001012393 (SEQ ID NO:34); AF091242 /// NM_{—}004670 (SEQ ID NO:35); BC016300 /// NM_{—}002961 (SEQ ID NO:36); BC001431 /// NM_{—}014624 (SEQ ID NO:37); AF078851 /// NM_{—}013243 (SEQ ID NO:38); Y00757 /// NM_{—}003020 (SEQ ID NO:39); AF393649 /// NM_{—}014467 (SEQ ID NO:40); X84839 /// NM_{—}021961 (SEQ ID NO:41); NM_{—}001007538 (SEQ ID NO:42); AY358393 /// NM_{—}198570 (SEQ ID NO:43); L20861 /// NM_{—}003392 (SEQ ID NO:44); and 5033414K04Rik (SEQ ID NO:45); U16153 (SEQ ID NO:46).

40. A kit comprising antisense nucleic acids sequences to fragments of at least 6 genes selected from the group of SEQ ID NO:1 to SEQ ID NO:46.

41. A kit comprising protein binding molecules that have binding affinity for at least six proteins selected from the group of 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik.

42. The kit of claim 41, wherein the kit is an ELISA kit.

43. The kit of any of claims 41 or 42, wherein the kit is a Multiplex Immuno-Assay kit.

44. A method for identifying a subject at risk of having or developing cancer, the method comprising the steps of:
(i) measuring the level of expression of at least 6 nucleic acid sequences encoding proteins selected from the group consisting of: genes 2310046A06Rik; 3110035E14Rik; A930001N09Rik; AI593442; AI851790; AOX1; ARHGAP29; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GJA1; GPR17; ID4; KAZALD1; KCNA4; LARP6; LGALS3; MGP; MIA; NINJ2; OPCML; PAPSS2; S100A4; S100A6; SCG3; SCG5; SRPX2; TEAD1; TMEM46; VWC2; WNT5A; and 5033414K04Rik in a biological sample;
(ii) identifying which of the genes measured in step (i) are cancer stem cell upregulated biomarkers selected from the group of; 2310046A06Rik; 3110035E14Rik; A930001N09Rik; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GPR17; ID4; KAZALD1; KCNA4; LARP6; LGALS3; MGP; MIA; NINJ2; OPCML; PAPSS2; S100A4; S100A6; SCG5; SRPX2; TMEM46 and VWC2;
(iii) identifying which of the genes measured in step (i) are cancer stem cell downregulated biomarkers selected from the group of; AI593442; AI851790; AOX1; ARHGAP29; GJA1; SCG3; TEAD1; WNT5A; and 5033414K04Rik
(iv) comparing the level of expression of each nucleic acid sequences measured in (i) to a reference expression level for each of the nucleic acid sequence measured;
wherein an increase in the level of the expression of at least 1.5-fold of said measured nucleic acid sequences for a cancer stem cell unregulated biomarker as compared to said reference expression level indicates a subject likely to be at risk of, or having cancer, or wherein an decrease in the level of the expression of at least 0.5-fold fold of said measured nucleic acid sequences for a cancer stem cell downregulated biomarker as compared to said reference expression level indicates a subject likely to be at risk of, or having cancer.

45. A method for treating a cancer in a subject, the method comprising identifying a cancer stem cell in a population of cells obtained from the subject according to claim 44, wherein a clinician reviews the results and if the results indicate an increase in the level of the expression of a cancer stem cell upregulated biomarker at least 1.5-fold, or a decrease in the level of the expression of a cancer stem cell downregulated biomarker of at least 0.5-fold in the biological sample from the subject as compared to said reference expression level, the clinician directs the subject to be treated with an appropriate anti-cancer therapy.

46. The method of claim 45, wherein the anti-cancer agent is an anti-cancer therapy targeting cancer stem cells.

47. The method of claims 44 or 45, wherein the subject is a human subject.

48. A method to identify a cancer stem cell in a population of cells, the method comprising;
(i) measuring a level of gene expression of at least 2 nucleic acid sequences encoding proteins selected from the group consisting of: 2310046A06Rik; 3110035E14Rik; A930001N09Rik; ARHGAP6; BFSP2; BGN; CAPG; CASP4; CAV1; COL6A1; COL6A2; CYTL1; D3Bwg0562e; D930020E02Rik; DDC; DHRS3; E030011K20Rik; ENPP6; FOXA3; FOXC2; GPR17; ID4; KAZALD1; KCNA4; LARP6; LGALS3; MGP; MIA; NINJ2; OPCML; PAPSS2; S100A4; S100A6; SCG5; SRPX2; TMEM46 and VWC2 in a biological sample;
(ii) identifying which of the genes measured in step (i) are cancer stem cell upregulated biomarkers selected from the group of comparing the level of expression of each nucleic acid sequences measured in (i) to a reference expression level for each of the nucleic acid sequence measured;
wherein an increase in the level of the expression of at least 1.5-fold of said measured nucleic acid sequences for a cancer stem cell upregulated biomarker as compared to said reference expression level indicates the presence of a cancer stem cell in a population of cells.

49. The method of claim 48, wherein said 2 nucleic acid sequences encoding the proteins are selected from the group fourth consisting of S100A4 and S100A6.

50. The method of claim 48, wherein the gene expression is measured at the level of RNA.

51. The method of claim 48, wherein the gene expression is measured at the level of protein expression.

52. The method of claim 51, wherein protein expression is measured using an antibody, human antibody, humanized antibody, recombinant antibodies, monoclonal antibodies, chimeric antibodies, protein binding proteins, aptamer, peptide or analogues, or conjugates or fragments thereof.

53. The method of claim 51, wherein measuring is by ELISA or Multiplex Immunoassay.
